(19) United States
(12) Patent Application Publication
(10) Pub. No.: US 2008/0064736 A1
(43) Pub. Date: Mar. 13, 2008

(54) Substituted 2-Pyrrolidone Derivatives as Fungicides and Insecticides

(75) Inventors: Stefan Hillebrand, Neuss (DE); Oliver Guth, Leverkusen (DE); Welf-Burkhard Wiese, Wermelskirchen (DE); Klaus Kunz, Dusseldorf (DE); Astrid Ullmann-Koppold, Leverkusen (DE); Amos Mattes, Langenfeld (DE); Peter Schreier, Koln (DE); Ulrike Wachnedorff-Neumann, Neuwied (DE); Karl-Heinz Kuck, Langenfeld (DE); Peter Losel, Leverkusen (DE); Olga Malsam, Rosrath (DE); Peter Reinemer, Munchen (DE); Marc Stadler, Wuppertal (DE); Stefan Seip, Schwelm (DE); Anke Mayer-Bartschmid, Wulfrath (DE); Hartwig Muller, Velbert (DE); Kevin Bacon, Kobe (JP); Mark Wilhelm Drewes, Langenfeld (DE); Kerstin Ilg, Koln (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: Bayer Cropscience AG, Monheim (DE)

(21) Appl. No.: 11/572,086

(22) PCT Filed: Jul. 9, 2005
(86) PCT No.: PCT/EP05/07442

§ 371 Date: Jul. 20, 2007

(30) Foreign Application Priority Data
Jul. 12, 2004 (EP) 04016320.6

(51) Int. Cl. A01N 43/36; A01P 3/00; A01P 7/04; C07D 207/28; C07D 491/044

(52) U.S. Cl. 514/421; 514/423; 548/453; 548/534

(57) ABSTRACT

The use of a compound of formula (I) or a salt thereof, where the symbols have the meanings given in the description, for the control of phytopathogenic mircroorganisms of harmful animals.

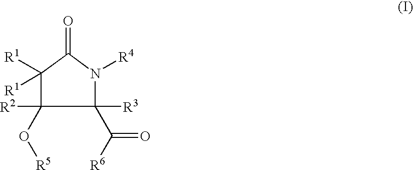

The invention relates to substituted heterocycles, processes for their preparation and their use for controlling phytopathogenic fungi and harmful insects.

Certain compounds with 5- or 6-membered Lactame rings are known for their fungicidal activity, e.g. Procymidone, Cycloheximide and Capsimycin (see “The Pesticide Manual”, 13 ed., C. D. S. Tomlin (Ed.), British Crop Protection Council, Farnham 2003.

However, since modern pesticides must meet a wide range of demands, for example regarding level, duration and spectrum of action, use spectrum, toxicity, combination with other active substances, combination with formulation auxiliaries or synthesis, and since the occurrence of resistances is possible, the development of such substances can never be regarded as concluded, and there is constantly a high demand for novel compounds which are advantageous over the known compounds, at least as far as some aspects are concerned.

It has now surprisingly been found that certain substituted heterocycles are particularly active as fungicides and insecticides.

Such compounds have been partly described in Yakugaku Zasshi, Pharmaceutical Society of Japan 92 (1972) 1507 and in WO-A 04/071382 as proteasom inhibitors. A utility against plant pests and plant pathogens is not disclosed.

Accordingly, in one aspect of the invention there is provided the use of a compound of formula (I) or a salt thereof, 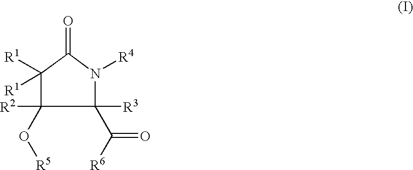
wherein the symbols have the following meanings:
R¹ is the same or different H, halogen, unsubstituted or substituted (C₁-C₂₀)-alkyl, unsubstituted or substituted (C₂-C₁₂)-alkenyl, unsubstituted or substituted (C₂-C₁₂)-alkynyl, unsubstituted or substituted (C₆-C₁₂)-aryl, unsubstituted or substituted heterocyclyl, unsubstituted or substituted (C₃-C₈)-cycloalkyl, unsubstituted or substituted (C₃-C₈)-cycloalkenyl, O-R″, S(O)ₙR″, SO₂NR₂″, COOR″, COSR′, CSOR′, —O—COR″, —O—CSR′, —CO—R″ or CONR₂″, or the two substituents R¹ together form a 3 to 8 membered ring which is a carbocyclic ring or contains one or two heteroatom units;
R′ is the same or different (C₁-C₁₂)-alkyl, (C₂-C₁₂)-alkenyl, (C₂-C₁₂)-alkynyl, (C₃-C₈)-cyclo-alkyl, (C₃-C₈)-cycloalkenyl, (C₆-C₁₂)-aryl, or heterocyclyl, all of which are unsubstituted or substituted;
R″ is the same or different H or R′;
n is 0, 1 or 2;
R² is H, unsubstituted or substituted (C₁-C₈)-alkyl, unsubstituted or substituted (C₂-C₈)-alkenyl, unsubstituted or substituted (C₂-C₈)-alkynyl, unsubstituted or substituted (C₃-C₆)-cycloalkyl, unsubstituted or substituted (C₃-C8)-cycloalkenyl, unsubstituted or substituted (C₆-C₁₂)-aryl, or unsubstituted or substituted heterocyclyl;
R³ is H, unsubstituted or substituted (C₁-C₁₂)-alkyl, unsubstituted or substituted (C₂-C₁₂)-alkenyl, unsubstituted or substituted (C₂-C₁₂)-alkynyl, unsubstituted or substituted (C₃-C₆)-cycloalkyl, unsubstituted or substituted (C₃-C₈)-cycloalkenyl, unsubstituted or substituted (C₆-C₁₂)-aryl, unsubstituted or substituted heterocyclyl, COOR″, CSOR″, COSR″, —CO—R″ or CONR₂″;
R⁴ H, unsubstituted or substituted (C₁-C₁₂)-alkyl, unsubstituted or substituted (C₃-C₁₂)-alkenyl, unsubstituted or substituted (C₃-C₁₂)-alkynyl, unsubstituted (C₆-C₁₂)-aryl, unsubstituted or substituted heterocyclyl, unsubstituted or substituted (C₃-C₈)-cycloalkyl, unsubstituted or substituted (C₃-C₈)-cycloalkenyl, S(O)ₙR′, COOR′, CSOR′, COSR′, —CO—R′, CONR₂″ or G;
R⁵ is H, unsubstituted or substituted (C₁-C₁₂)-alkyl, unsubstituted or substituted (C₂-C₁₂)-alkenyl, unsubstituted or substituted (C₂-C₁₂)-alkynyl, unsubstituted or substituted (C₃-C₈)-cycloalkyl, unsubstituted or substituted (C₃-C₈)-cycloalkenyl, unsubstituted or substituted (C₆-C₁₂)-aryl, or unsubstituted or substituted heterocyclyl, SO₂R′, COR′, COOR′, COSR′, CSOR′, CONR₂″ or G;
G is Si[(C₁-C₆)-alkyl]ₓ[(CH2)ₜ-phenyl-(CH₃)ᵤ]_{y}[(O)ᵥ—((C₁-C₄)-alkylene)-(O—(C₁-C4)-alkylene)_{w}-H]_{z}, where x, y, z are 0, 1, 2 or 3 and x+y+z=3, t, u are 0 or 1, v, w are 0 or 1 and v+w is 1 or 2;
R⁶ is OR″, SR″ or NR₂″
or
R⁵ and R⁶ together form a bond,
for the control of phytopathogenic microorganisms or harmful animals.

In a further aspect of the invention there is provided a process for controlling phytopathogenic microorganisms and harmful animals, which comprises applying a compound of formula (I) as described above to the phytopathogenic microorganism or harmful animal or their habitat.

In yet a further aspect of the invention there is provided a compound of formula (Ia) or a salt thereof, 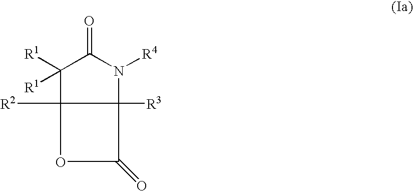
wherein the symbols have the following meanings:
R¹ is the same or different H, halogen, unsubstituted or substituted (C₁-C₂₀)-alkyl, unsubstituted or substituted (C₂-C₁₂)-alkenyl, unsubstituted or substituted (C₂-C₁₂)-alkynyl, unsubstituted or substituted (C₆-C₁₂)-aryl, unsubstituted or substituted heterocyclyl, substituted or unsubstituted (C₃-C₈)-cycloalkyl, unsubstituted or substituted (C₃-C₈)-cycloalkenyl, O—R″, S(O)ₙR″, COOR″, CSOR′, COSR′, —O—COR′, —O—CSR′, —CO—R″, CONR₂″, SO₂NR₂″, or the two substituents R¹ together form a 3 to 8 membered ring which is a carbocyclic ring or contains one or two heteroatom units;
n is 0, 1 or 2;
R′ is the same or different (C₁-C₁₂)-alkyl, (C₂-C₁₂)-alkenyl, (C₂-C₁₂)-alkynyl, (C₃-C₈)-cycloalkyl, (C₃-C₈)-cycloalkenyl, (C₆-C₁₂)-aryl, or heterocyclyl, all of which are unsubstituted or substituted;
R″ is the same or different H or R′;
R² is unsubstituted or substituted (C₁-C₆)-alkyl, unsubstituted or substituted (C₂-C₆)-alkenyl, unsubstituted or substituted (C₂-C₆)-alkynyl, unsubstituted or substituted (C₃-C₆)-cycloalkyl, unsubstituted or substituted (C₃-C₈)-cycloalkenyl, unsubstituted or substituted (C₆-C₁₂)-aryl, or unsubstituted or substituted heterocyclyl;
R³ is H, unsubstituted or substituted (C₁-C₆)-alkyl, unsubstituted or substituted (C₂-C₆)-alkenyl, unsubstituted or substituted (C₂-C₆)-alkynyl, unsubstituted or substituted (C₃-C₆)-cycloalkyl, unsubstituted or substituted (C₃-C₈)-cycloalkenyl, unsubstituted or substituted (C₆-C₁₂)-aryl, or unsubstituted or substituted heterocyclyl, COOR″, CSOR′, COSR′, —CO—R′ or CONR₂″;
and
R⁴ is H, unsubstituted or substituted (C₁-C20)-alkyl, unsubstituted or substituted (C₂-C₁₂)-alkenyl, unsubstituted or substituted (C₂-C₁₂)-alkynyl, unsubstituted or substituted (C₆-C₁₂)-aryl, unsubstituted or substituted heterocyclyl, unsubstituted or substituted (C₃-C₈)-cycloalkyl, unsubstituted or substituted (C₃-C₆)-cycloalkenyl, S(O)ₙR′, COOR′, CSOR′, COSR′, —CO—R′, CONR₂″ or G; and
G is Si[(C₁-C₆)-alkyl]ₓ[(CH₂)ₜ-phenyl-(CH₃)ᵤ]_{y}[(O)ᵥ—((C₁-C₄)-alkylene)-(O—(C₁-C₄)-alkylene)_{w}-H]_{z},
   where x, y, z are 0, 1, 2 or 3 and x+y+z=3, t, u are 0 or 1, v, w are 0 or 1 and v+w is 1 or 2;
   with the proviso, that the following compounds are excluded:

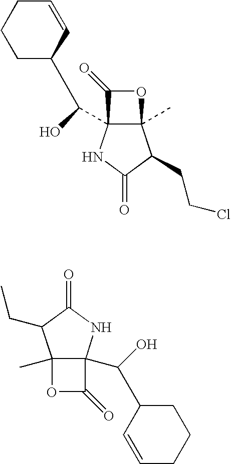

In yet a further aspect of the invention there is provided a compound of formula (Ib) or a salt thereof, 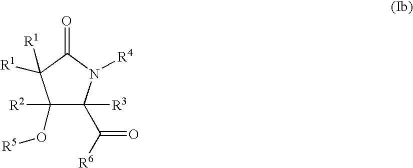
wherein the symbols have the following meanings:
R¹ is the same or different H, halogen, unsubstituted or substituted (C₁-C₂₀)-alkyl, unsubstituted or substituted (C₂-C₁₂)-alkenyl, unsubstituted or substituted (C₂-C₁₂)-alkynyl, unsubstituted or substituted (C₆-C₁₂)-aryl, unsubstituted or substituted heterocyclyl, substituted or unsubstituted (C₃-C₈)-cycloalkyl, unsubstituted or substituted (C₃-C₈)-cycloalkenyl, O—R″, S(O)ₙR″, COOR″, CSOR′, COSR′, —O—COR′, —O—CSR′, —CO—R″ or CONR2″, or the two substituents R¹ together form a 3 to 8 membered ring which is a carbocyclic ring or contains one or two heteroatom units;
n is 0, 1 or 2;
R′ is the same or different (C₁-C₁₂)-alkyl, (C₂-C₁₂)-alkenyl, (C₂-C₁₂)-alkynyl, (C₃-C₈)-cycloalkyl, (C₃-C₈)-cycloalkenyl, (C₆-C₁₂)-aryl, or heterocyclyl, all of which are unsubstituted or substituted;
R″ is the same or different H or R′;
R² is unsubstituted or substituted (C₁-C₅)-alkyl, unsubstituted or substituted (C₂-C₆)-alkenyl, unsubstituted or substituted (C₂-C₆)-alkynyl, unsubstituted or substituted (C₃-C₆)-cycloalkyl, unsubstituted or substituted (C₃-C₆)-cycloalkenyl, unsubstituted or substituted (C₆-C₁₂)-aryl, or unsubstituted or substituted heterocyclyl;
R³ is H, unsubstituted or substituted (C₁-C₆)-alkyl, unsubstituted or substituted (C₂-C₆)-alkenyl, unsubstituted or substituted (C₂-C₆)-alkynyl, unsubstituted or substituted (C₃-C₆)-cycloalkyl, unsubstituted or substituted (C₃-C₆)-cycloalkenyl, unsubstituted or substituted (C₆-C₁₂)-aryl, or unsubstituted or substituted heterocyclyl, CSOR′, COSR′, —CO—R″ or CONR₂″;
R⁴ is H, unsubstituted or substituted (C₁-C₁₂)-alkyl, unsubstituted or substituted (C₂-C₁₂)-alkenyl, unsubstituted or substituted (C₂-C₁₂)-alkynyl, unsubstituted or substituted (C₆-C₁₂)-aryl, unsubstituted or substituted heterocyclyl, unsubstituted or substituted (C₃-C₈)-cycloalkyl, unsubstituted or substituted (C₃-C₆)-cycloalkenyl, S(O)ₙR′, COOR′, CSOR′, —CO—R′, CON′R₂″or G.
R5 is H, unsubstituted or substituted (C₁-C₁₂)-alkyl, unsubstituted or substituted (C₂-C₁₂)-alkenyl, unsubstituted or substituted (C₂-C₁₂)-alkynyl, unsubstituted or substituted (C₃-C₈)-cycloalkyl, unsubstituted or substituted (C₃-C₈)-cycloalkenyl, unsubstituted or substituted (C₆-C₁₂)-aryl, unsubstituted or substituted heterocyclyl, SO₂R′, COR′, COOR′, COSR′, CSOR′, CONR₂″ or G;
G is Si[(C₁-C₆)-alkyl]ₓ[(CH₂)ₜ-phenyl-(CH₃)ᵤ]_{y}[(O)ᵥ—((C₁-C₄)-alkylene)-(O—(C₁-C₄)-alkylene)_{w}-H]_{z},
   where x, y, z are 0, 1, 2 or 3 and x+y+z=3, t, u are 0 or 1, v, w are 0 or 1 and v+w is 1 or 2;
R⁶ is OR″, SR″ or NR₂″;
excluding compounds where
   R¹═H, R²═CH₃, R³═H, R⁴═H, C₆H₅, CH₂—C₆H₅, C₂H₅, R⁵═H and R⁶═NH₂, NHC₆H₅, NHCH₂C₆H₅.

Depending inter alia on the nature of substituents, the compounds of the formula (I) can be present as optical isomers or isomer mixtures of varying composition which, if appropriate, can be separated in a customary manner. The present invention provides both the pure isomers and the isomer mixtures, their preparation and use as well as compositions comprising them. However, herein below for the sake of simplicity only compounds of the formula (I) are referred to although this means both the pure compounds and, if appropriate, also mixtures having varying proportions of isomeric compounds.

Salts for the purposes of the invention are preferably agrochemically acceptable salts of the compounds according to the invention.

Agrochemically acceptable salts of the compounds (I) include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, propionic acid, lactic acid, tartaric acid, malic acid, citric acid, fumaric acid, maleic acid and benzoic acid.

Agrochemically acceptable salts of the compounds (I) also include salts of customary bases, such as for example and preferably alkali metal salts (for example sodium and potassium salts, alkaline earth metal salts (for example calcium and magnesium salts) and ammonium salts derived from ammonia or organic amines having 1 to 16 carbon atoms, such as illustratively and preferably ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, dihydroabietylamine, arginine, lysine, ethylenediamine and methylpiperidine.

In the present specification, including the accompanying claims, the aforementioned substituents have the following meanings:

Halogen means fluorine, chlorine, bromine or iodine. The term “halo” before the name of a radical means that this radical is partially or completely halogenated, that is to say, substituted by F, Cl, Br, or I, in any combination, preferably by F or Cl.

Alkyl groups and portions thereof (unless otherwise defined) may be straight- or branched-chain.

The expression “(C₁-C₆)-alkyl” is to be understood as meaning an unbranched or branched hydrocarbon radical having 1, 2, 3, 4, 5 or 6 carbon atoms, such as, for example a methyl, ethyl, propyl, isopropyl, 1-butyl, 2-butyl, 2-methylpropyl or tert-butyl radical.

Alkyl radicals and also in composite groups, unless otherwise defined, preferably have 1 to 4 carbon atoms.

(C₁-C₆)-Haloalkyl means “(C₁-C₆)alkyl” in which one or more hydrogen atoms are replaced by the same number of identical or different halogen atoms, such as monohaloalkyl, perhaloalkyl, CF₃, CHF₂, CH₂F, CHFCH₃, CH₂CF₃, CF₂CF₃, CF₂CHF₂, CHClCH₂F, CCl₃, CHCl₂ or CH₂CH₂Cl.

The expression “(C₁-C₆)-haloalkylene” is to be understood to mean an alkylene group mentioned under the expression “(C₁-C₆)-alkylene”, in which one or more hydrogen atoms are replaced by the same number of identical or different halogen atoms.

“(C₁-C₆)Alkoxy” means an alkoxy group whose carbon chain has the meaning given under the expression “(C₁-C₆)alkyl”. “Haloalkoxy” is, for example, OCF₃, OCHF₂, OCH₂F, OCF₂CF₃, OCH₂CF₃ or OCH₂CH₂Cl.

“(C₂-C₆)Alkenyl” means an unbranched or branched non-cyclic carbon chain having a number of carbon atoms which corresponds to this stated range and which contains at least one double bond which can be located in any position of the respective unsaturated radical. “(C₂-C₆)Alkenyl” accordingly denotes, for example, the vinyl, allyl, 2-methyl-2-propenyl, 2-butenyl, pentenyl, 2-methylpentenyl or the hexenyl group.

“(C₂-C₆)Alkynyl” means an unbranched or branched non-cyclic carbon chain having a number of carbon atoms which corresponds to this stated range and which contains one triple bond which can be located in any position of the respective unsaturated radical. “(C₂-C₆)Alkynyl” accordingly denotes, for example, the propargyl, 1-methyl-2-propynyl, 2-butynyl or 3-butynyl group.

Cycloalkyl groups preferably have from three to seven carbon atoms in the ring and are optionally substituted by halogen, (C₁-C₄)-haloalkyl or (C₁-C₄)-alkyl.

The group “G” Si[(C₁-C₆)-alkyl]ₓ[(CH₂)ₜ-phenyl-(CH₃)ᵤ]_{y}[(O)ᵥ—((C₁-C₄)-alkylene)-(O—(C₁-C4)-alkylene)_{w}-H]_{z}, where x, y, z are 0, 1, 2 or 3 and x+y+z=3, t, u are 0 or 1, v, w are 0 or 1 and v+w is 1 or 2; means for example the SiMe₃, SiEt₃, SiMe₂t-Bu, SiMe(t-Bu)₂, Si-i-Pr₃, Si-t-BuPh₂, SiMePh₂, SiPh₃, SiMe₂(C(CH₃)₂—CH(CH₃)₂), SiEt₂i-Pr, SiMe₂i-Pr, SiMe₂i-Bu, SiBz₃, Si(CH₂—C₆H₄—CH₃)₃, SiPh₂(O-i-Pr), SiPh₂(O-t-Bu), SitBuPh(OMe), or SiMe₂(O—C₂H₄—OMe) group.

“Heterocyclyl” preferably means a saturated, unsaturated or aromatic 3 to 10 membered ring system comprising one or more heteroatoms, preferably from the group consisting of N, O and S(O)ₘ (m =0, 1, 2), where at least one carbon atom is present in the ring.

More preferred “heterocyclyl” denotes a thiophene, furan, pyrrole, thiazole, oxazole, imidazole, isothiazole, isoxazole, pyrazole, 1,3,4-oxadiazole, 1,3,4-thiadiazole, 1,3,4-triazole, 1,2,4-oxadiazole, 1,2,4-thiadiazole, 1,2,4-triazole, 1,2,3-triazole, 1,2,3,4-tetrazole, benzo[b]thiophene, benzo[b]furan, indole, benz[c]thiophene, 1,3,-benzodioxole, 1,3-benzodioxane, beno[c]furan, isoindole, benzoxazole, benzothiazole, benzimidazole, benzisoxazole, benzisothiazole, benzopyrazole, benzothiadiazole, benzotriazole, dibenzofuran, dibenzothiophene, carbazole, pyridine, pyrazine, pyrimidine, pyridazine, 1,3,5-triazine, 1,2,4-triazine, 1,2,4,5-tetrazine, quinoline, isoquinoline, quinoxaline, quinazoline, cinnoline, 1,8-naphthyridine, 1,5-naphthyridine, 1,6-naphthyridine, 1,7-naphthyridine, phthalazine, pyridopyrimidine, purine, pteridine, 4H-quinolizine, morpholine, piperidine, piperazine, pyrroline, pyrrolidine, oxazoline, tetrahydrofuran, tetrahydropyran, isoxazolidine, oxazolidine, thiazoline, thiazolidine, oxirane or oxetane radical.

Particularly preferred “heterocyclyl” means pyrrolyl, imidazolyl, pyrazolyl, 1,3,4-triazolyl, 1,2,4-oxadiazolyl, oxazolyl, tetrazolyl, pyridyl, pyrazinyl, pyrimidinyl, 1,3,5-triazinyl, morpholinyl, piperidinyl, thiophenyl or thiazolyl.

“Substituted” if not otherwise defined in its respective context means, e.g. substituted by one or more, preferably one to three and in the case of halogen up to the maximum number, substituents from the group consisting of halogen, R′″, OR′″, S(O)ₙR′″, SO₂NR₂′″, NR₂′″, COOR′″, NHCOR′″, NHCOOR′″, G, CN, NO₂, (C₃-C₆)-cycloalkyl, unsubstituted or substituted by halogen and/or (C₁-C₄)-alkyl, (C₆-C₁₂)-aryl and/or heterocyclyl, the later two unsubstituted or substituted by one or more substituents from the group consisting of halogen, (C₁-C₄)-alkyl, (C₁-C₄)-haloalkyl, (C₁-C₄)-alkoxy, (C₁-C₄)-haloalkoxy, NO₂, and CN,
where
n is 0, 1 or 2;
R′″ is H, (C₁-C₄)-alkyl, (C₁-C₄)-haloalkyl, (C₁-C₄)-alkoxy, (C₁-C₄)-haloalkoxy, (C₂-C₄)-alkenyl, (C₂-C₄)-haloalkenyl, (C₂-C₄)-alkynyl, (C₂-C₄)-haloalkynyl, (C₃-C₆)-cycloalkyl, (C₃-C₆)-cycloalkenyl, heterocyclyl and (C₆-C₁₀)-aryl, which are both unsubstituted or substituted by one to five substituents from the group consisting of halogen, (C₁-C₄)-alkyl, (C₁-C₄)-haloalkyl, (C₁-C₄)-alkoxy, (C₁-C₄)-haloalkoxy, NO₂, and CN; and
G has the meaning given above.

“Substituted” preferably means substituted by one or more, preferably one to three and in case of halogen up to maximum number, substituents from the group F, Cl, Br, I, R′″, OR′″, S(O)ₙR′″, SO₂NR₂′″, NR₂′″, COOR′″, NHCOR′″, NHCOOR′″, G, CN, NO₂,
where
R′″ is H, (C₁-C₄)-alkyl, (C₁-C₄)-haloalkyl, (C₁-C₄)-alkoxy, (C₁-C₄)-haloalkoxy, (C₂-C₄)-alkenyl, (C₂-C₄)-haloalkenyl, (C₂-C₄)-alkynyl, (C₂-C₄)-halogalkynyl, (C₃-C₆)-cycloalkyl, (C₃-C₆)-cycloalkenyl, heterocyclyl and (C₆-C₁₀)-aryl, of which the last four groups are unsubstituted or substituted by one to five substituents from the group consisting of halogen, (C₁-C₄)-alkyl, (C₁-C₄)haloalkyl, (C₁-C₄)-alkoxy, (C₁-C₄)-haloalkoxy, NO₂, and CN;
G is Si[(C₁-C₆)-alkyl]ₓ[(CH₂)ₜ-phenyl-(CH₃)ᵤ]_{y}[(O)ᵥ—((C₁-C₄)-alkylene)-(O—(C₁-C₄)-alkylene)₂-H]_{z},
   where x, y, z are 0, 1, 2 or 3 and x+y+z=3, t, u are 0 or 1, v, w are 0 or 1 and v+w is 1 or 2;
n is 0, 1 or 2.

More preferred “substituted” means substituted by one or more, preferrably one to three and in case of halogen up to maximum number, substituents from the group F, Cl, Br, R′″, OR′″, S(O)ₙR′″, SO₂NR₂′″, NR₂′″, COOR′″, NHCOR′″, NHCOOR′″; G, CN and NO₂,
where
R′″ is H, (C₁-C₄)-alkyl, (C₁-C₄)-haloalkyl, (C₁-C₄)-alkoxy, (C₁-C₄)-haloalkoxy, (C₂-C₄)-haloalkenyl, (C₂-C₄)-alkynyl, (C₂-C₄)-halogalkynyl, (C₃-C₆)-cycloalkyl, (C₃-C₆)-cycloalkenyl, heterocyclyl and (C₆-C₁₀)-aryl, of which the last four groups are unsubstituted or substituted by one to four substituents from the group consisting of fluoro, chloro, bromo, (C₁-C₄)-alkyl, (C₁-C₄)haloalkyl, (C₁-C₄)-alkoxy, (C₁-C₄)-haloalkoxy, NO₂, and CN;
   SiMe₃, SiEt₃, SiMe₂t-Bu, SiMe(t-Bu)₂, Si-i-Pr₃, Si-t-BuPh₂, SiMePh₂, SiPh₃, SiMe₂(C(CH₃)₂—CH(CH₃)₂), SiEt₂i-Pr, SiMe₂i-Pr, SiMe₂i-Bu, SiBz₃, Si(CH₂—C₆H₆—CH₃)₃, SiPh₂(O-i-Pr), SiPh₂(O-t-Bu), Sit-BuPh(OMe), or SiMe₂(O—C₂H₄—OMe);
n is 0, 1 or 2.

Particularly preferred “substituted” means substituted by one to three and in the case of halogen up to maximum number substituents from the group fluoro, chloro, R′″, OR′″, NR₂′″, G, NO₂ and CN,
where
R′″ is H, (C₁-C₄)-alkyl, (C₁-C₄)-haloalkyl, (C₁-C₄)-alkoxy, (C₁-C₄)-haloalkoxy, (C₂-C₄)-haloalkenyl, (C₂-C₄)-alkenyl, (C₂-C₄)-haloalkenyl, (C₂-C₄)-alkynyl, (C₂-C₄)-halogalkynyl, (C₃-C₆)-cycloalkyl, heterocyclyl and phenyl, which are both unsubstituted or substituted by one to three substituents from the group consisting of fluoro, chloro, (C₁-C₄)-alkyl, trifluoromethyl, difluoromethyl, (C₁-C₄)-alkoxy, (C₁-C₄)-haloalkoxy, NO₂, and CN; and
G is SiMe₃, SiEt₃, SiMe₂t-Bu, SiMe(t-Bu)₂, Si-i-Pr₃, Si-t-BuPh₂, SiMePh₂, SiPh₃, SiMe₂(C(CH₃)₂—CH(CH₃)₂), SiEt₂i-Pr, SiMe₂i-Pr, SiMe₂i-Bu, SiBz₃, Si(CH₂—C₆H₄—CH₃)₃, SiPh₂(O-i-Pr), SiPh₂(O-t-Bu), Sit-BuPh(OMe), or SiMe₂(O—C₂H₄—OMe);

The symbols in formula (I) preferably have the following meanings:
R¹ is preferably the same or different H, chloro, bromo, unsubstituted or substituted (C₁-C₁₂)-alkyl, unsubstituted or substituted (C₂-C₁₀)-alkenyl, unsubstituted or substituted (C₂-C₁₀)-alkynyl, unsubstituted or substituted (C₆-C₁₂)-aryl, unsubstituted or substituted heterocyclyl, unsubstituted or substituted (C₃-C₆)-cycloalkyl, OR″, COOR″ or —CO—R″ or the two substituents R¹ together form a 3 to 6 membered ring which is a carbocyclic ring or contains one or two heteroatom units.
R² is preferably H, unsubstituted or substituted (C₁-C₆)-alkyl, unsubstituted or substituted (C₂-C₆)-alkenyl, unsubstituted or substituted (C₂-C₆)-alkynyl, unsubstituted or substituted (C₃-C₆)-cycloalkyl, unsubstituted or substituted phenyl, or unsubstituted or substituted heterocyclyl.
R³ is preferably H, unsubstituted or substituted (C₁-C₁₂)-alkyl, unsubstituted or substituted (C₂-C₁₂)-alkenyl, unsubstituted or substituted (C₂-C₁₂)-alkynyl, unsubstituted or substituted (C₃-C₆)-cycloalkyl, unsubstituted or substituted (C₃-C₆)-cycloalkenyl, unsubstituted or substituted (C₆-C₁₂)-aryl, unsubstituted or substituted heterocyclyl, COOR″, —CO—R″ or CONR₂″.
R⁴ is preferably H, unsubstituted or substituted (C₁-C₁₂)-alkyl, unsubstituted or substituted (C₃-C₁₀)-alkenyl, unsubstituted or substituted (C₃-C₁₀)-alkynyl, SO₂R′, COOR′, —COR′, CONR₂″ or G.
R⁵ is preferably H, unsubstituted or substituted (C₁-C₁₂)-alkyl, unsubstituted or substituted (C₂-C₁₂)-alkenyl, unsubstituted or substituted (C₂-C₁₂)-alkynyl, unsubstituted or substituted (C₃-C₈)-cycloalkyl, unsubstituted or substituted phenyl, unsubstituted or substituted heterocyclyl, SO₂R′, COR′, COOR′, COSR′, CSOR′, CONR₂″ or G.
G is preferably Si[(C₁-C₆)-alkyl]ₓ[(CH₂)ₜ-phenyl-(CH₃)ᵤ]_{y}[(O)ᵥ—((C₁-C₄)-alkylene)-(O—(C₁-C₄)-alkylene)_{w}-H]_{z},
   where x, y, z are 0, 1, 2 or 3 and x+y+z=3, t, u are 0 or 1, v, w are 0 or 1 and v+w is 1 or 2.
R⁶ is preferably OR″, SR″ or NR₂″
or
R⁵ and R⁶ preferably together form a bond.
R′ is preferably the same or different (C₁-C₁₀)-alkyl, (C₂-C₁₀)-alkenyl, (C₂-C₁₀)-alkynyl, (C₃-C₆)-cycloalkyl, (C₃-C₆)-cycloalkenyl, phenyl, or heterocyclyl, all of which are unsubstituted or substituted.
R″ is preferably the same or different and is H or R′.
n is preferably 0, 1 or 2.

Preferred are compounds of formula (I) where all symbols have the preferred meanings and “substituted” has the preferred meanings.

More preferred compounds of formula (I) are compounds of formula (II), 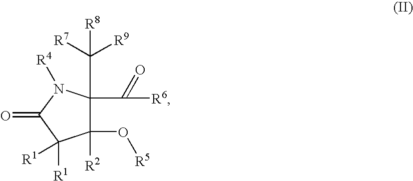
where the symbols have the following meanings:
R¹ is the same or different H, chloro, bromo, unsubstituted or substituted (C₁-C₁₀)-alkyl, unsubstituted or substituted (C₂-C₈)-alkenyl, unsubstituted or substituted (C₂-C₈)-alkynyl, unsubstituted or substituted phenyl, unsubstituted or substituted heterocyclyl, unsubstituted or substituted (C₃-C₆)-cycloalkyl, COOR″, OR″ or —CO—R″ or the two substituents R¹ together form a 3 to 6 membered carbocyclic ring;
R² H, unsubstituted or substituted (C₁-C₆)-alkyl, unsubstituted or substituted (C₂-C₄)-alkenyl, unsubstituted or substituted (C₂-C₄)-alkynyl, unsubstituted or substituted (C₃-C₆)-cycloalkyl, unsubstituted or substituted phenyl, or unsubstituted or substituted heterocyclyl;
R⁴ is H, unsubstituted or substituted (C₁-C₁₂)-alkyl, unsubstituted or substituted (C₃-C₆)-alkenyl, unsubstituted or substituted (C₃-C₆)-alkynyl, SO₂R′, COOR′, —COR′ or G;
R⁵ is H, unsubstituted or substituted (C₁-C₈)-alkyl, unsubstituted or substituted (C₂-C₆)-alkenyl, unsubstituted or substituted (C₂-C₆)-alkynyl, unsubstituted or substituted (C₃-C₆)-cycloalkyl, SO₂R′, COOR′, —COR′, CONR₂″, unsubstituted or substituted phenyl, unsubstituted or substituted heterocyclyl, or G;
G is Si[(C₁-C₆)-alkyl]ₓ[(CH₂)ₜ-phenyl-(CH₃)ᵤ]_{y}[(O)ᵥ—((C₁-C₄)-alkylene)-(O—(C₁-C₄)-alkylene)_{w}-H]_{z},
   where x, y, z are 0, 1, 2 or 3 and x+y+z=3, t, u are 0 or 1, v, w are 0 or 1 and v+w is 1 or 2;
R⁶ is OR″, SR″ or NR₂″;
or
R⁵ and R⁶ together form a bond;
R⁷ is H, fluoro, chloro, O—R″, SR″, NR″₂, —O—COR′, —S—COR′, —O—CSR′, —O—SO₂R′, —O—COOR′, —O—CSOR′, —O—CONR₂″, NO₂, or CN;
R⁸ is H, unsubstituted or substituted (C₁-C₄)-alkyl, unsubstituted or substituted (C₂-C₄)-alkenyl, unsubstituted or substituted (C₂-C₄)-alkynyl, unsubstituted or substituted (C₃-C₆)-cycloalkyl, unsubstituted or substituted phenyl, or unsubstituted or substituted heterocyclyl;
or
R⁷ and R⁸ are together ═O or ═S;
R⁹ is H, halogen, unsubstituted or substituted (C₁-C₁₂)-alkyl, unsubstituted or substituted (C₂-C₁₀)-alkenyl, unsubstituted or substituted (C₂-C₁₀)-alkynyl, unsubstituted or substituted (C₆-C₁₂)-aryl, unsubstituted or substituted heterocyclyl, unsubstituted or substituted (C₃-C₈)-cycloalkyl, unsubstituted or substituted (C₃-C₈)-cycloalkenyl, O—R′, or SR′;
or
R⁸ and R⁹ together form a 3 to 8 membered ring which is a carbocyclic ring or contains one or two heteroatom units,
R′ is the same or different (C₁-C₈)-alkyl, (C₂-C₆)-alkenyl, (C₂-C₆)-alkynyl, (C₃-C₆)-cycloalkyl, phenyl, or heterocyclyl, all of which are unsubstituted or substituted; and
R″ is the same or different H or R″;
G SiMe₃, SiEt₃, SiMe₂t-Bu, SiMe(t-Bu)₂, Si-i-Pr₃, Si-t-BuPh₂, SiMePh₂, SiPh₃, SiMe₂(C(CH₃)₂—CH(CH₃)₂), SiEt₂i-Pr, SiMe₂i-Pr, SiMe₂i-Bu, SiBz₃, Si(CH₂—C₆H₄—CH₃)₃, SiPh₂(O-i-Pr), SiPh₂(O-t-Bu), Sit-BuPh(OMe), or SiMe₂(O—C₂H4—OMe);
n is 0, 1 or 2.

Preferred are compounds of formula (II) in which “substituted” has the preferred meanings. More preferred are compounds of formula (II) in which “substituted” has the more preferred meanings.

Particularly preferred are compounds of formula (II) in which the symbols have the following meanings:
R¹ is particularly preferred the same or different H, chloro, bromo, (C¹-C₈)-alkyl, (C₂-C₆)-alkenyl, (C₂-C₈)-alkynyl, phenyl, wherein the last four groups are unsubstituted or substituted with one to three substituents from the group fluoro, chloro, methyl, ethyl, isopropyl, tert-butyl, trifluoromethyl, trifluoromethoxy, methoxy, ethoxy, NO₂, and CN, heterocyclyl, unsubstituted or substituted with fluoro, chloro, methyl, ethyl, isopropyl, tert-butyl, trifluoromethyl, trifluoromethoxy, methoxy, ethoxy, NO₂, or CN, (C₃-C₆)-cycloalkyl, (C₃-C₈)-cycloalkenyl, both unsubstituted or substituted with one to three substituents from the group fluoro, chloro, methyl, trifluoromethyl, methoxy, NO₂, and CN, or the two substituents R together form a 3 to 6 membered carbocyclic ring.
R² is particularly preferred H, methyl, ethyl, propyl, isopropyl, allyl, propargyl, cyclopropyl, cyclobutyl, cyclopentyl, cyclohexyl, phenyl, unsubstituted or substituted with one to three substituents from the group fluoro, chloro, methyl, ethyl, isopropyl, tert-butyl, trifluoromethyl, trifluoromethoxy, methoxy, ethoxy, NO₂, and CN, or heterocyclyl, unsubstituted or substituted with one to three substituents from the group fluoro, chloro, methyl, ethyl, trifluoromethyl, methoxy, NO₂, and CN.
R⁴ is particularly preferred H, unsubstituted or substituted (C₁-C₁₂)-alkyl, unsubstituted or substituted (C₃-C₆)-alkenyl, unsubstituted or substituted (C₃-C₆)-alkynyl, SO₂R′, COOR′, —COR′, CONR₂″ or G.
R⁵ is particularly preferred H, (C₁-C₆)-alkyl, (C₂-C₆)-alkenyl, (C₂-C₈)-alkynyl, unsubstituted or substituted (C₃-C₆)-cycloalkyl, SO₂R′, COOR′, —COR′, CONR₂″, or G.
R⁶ is particularly preferred OR″, SR″ or NR₂″,
or particularly preferred
R⁵ and R⁶ together form a bond.
R⁷ is particulary preferred hydroxyl, mercapto, SCH₃, fluoro, chloro, bromo, methyl, ethyl, methoxy, trifluoromethoxy, ethoxy, —O—SO₂R′, —O—COOR′, —O—CONR₂″, CN, NR″ or O-G.
R⁸ is particularly preferred H, unsubstituted or substituted (C₁-C₄)-alkyl, unsubstituted or substituted (C₂-C₄)-alkenyl, unsubstituted or substituted (C₂-C₄)-alkynyl, unsubstituted or substituted (C₃-C₆)cycloalkyl, phenyl, unsubstituted or substituted with one to three substituents from the group fluoro, chloro, methyl, ethyl, isopropyl, tert-butyl, trifluoromethyl, trifluoromethoxy, methoxy, ethoxy, NO₂ and CN, heterocyclyl, unsubstituted or substituted with one to three substituents from the group fluoro, chloro, methyl, ethyl, isopropyl, tert-butyl, trifluoromethyl, trifluoromethoxy, methoxy, ethoxy, NO₂ and CN;
or particularly preferred
R⁷ and R⁸ are together ═O or ═S.
R⁹ is particularly preferred H, halogen, unsubstituted or substituted (C₁-C₈)-alkyl, unsubstituted or substituted (C₂-C₆)-alkenyl, propargyl, unsubstituted or substituted (C₆-C₁₀)-aryl, unsubstituted or substituted heterocyclyl, unsubstituted or substituted (C₃-C₆)-cycloalkyl, unsubstituted or substituted (C₃-C₆)-cycloalkenyl;
or particularly preferred
R⁸ and R⁹ together form a 3 to 6 membered ring which is a carbocyclic ring or contains one or two heteroatom units.
G is particularly preferred SiMe₃, SiEt₃, SiMe₂t-Bu, SiMe(t-Bu)₂, Si-i-Pr₃, Si-t-BuPh₂, SiMePh₂, SiPh₃, SiMe₂(C(CH₃)₂—CH(CH₃)₂), SiEt₂i-Pr, SiMe₂i-Pr, SiMe₂i-Bu, SiBz₃, Si(CH₂—C₆H₄—CH₃)₃, SiPh₂(O-i-Pr), SiPh₂(O-t-Bu), Sit-BuPh(OMe), or SiMe₂(O—C₂H₄—OMe).
R′ is particulary preferred the same or different (C₁-C₆)-alkyl, (C₂-C₄)-alkenyl, (C₂-C₄)-alkynyl, (C₃-C₆)-cycloalkyl, phenyl, or heterocyclyl, all of which are unsubstituted or substituted.
R″ is particularly preferred the same or different H or R′.

Particulary preferred are compounds of formula (II) in which all the symbols and the term “substituted” have the particulary preferred meanings.

Further particulary preferred are compounds of formula (III), 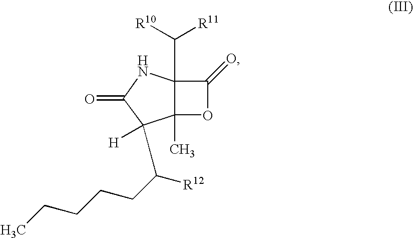
wherein
R¹⁰ represents hydrogen or hydroxy,
R¹¹ represents cyclohexyl or cyclohex-2-enyl,
R¹² represents hydrogen or hydroxy,

Further particularly preferred are compounds according to formula (IIIa) 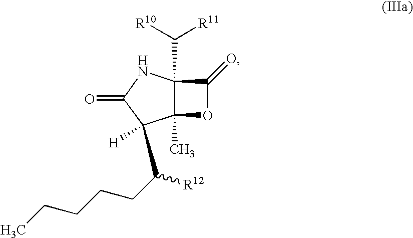
wherein
R¹⁰, R¹¹ and R¹² have the meaning described above.

Further particulary preferred are the following to compounds according to formula (I),

### (1R,4R,5S)-1-[(S)-(1S)-2-cyclohexen-1-yl(hydroxy)methyl]-4-hexyl-5-methyl-6-oxa-2-azabicyclo[3.2.0]heptane-3,7-dione

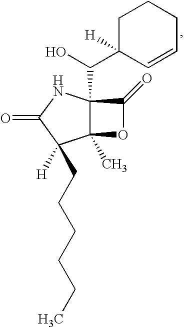

### (1R,4R,5S)-1-[(S)-(1S)-2-cyclohexen-1-yl(hydroxy)methyl]-4-[1-hydroxy-hexyl]-5-methyl-6-oxa-2-azabicyclo[3.2.0]heptane-3,7-dione

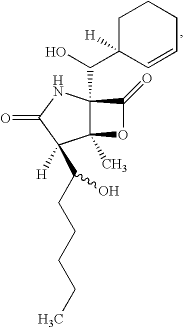

and

### (1R,4R,5S)-1-[(1R)-2-cyclohexen-1-ylmethyl]-4-hexyl-5-methyl-6-oxa-2-aza-bicyclo[3.2.0]-heptane-3,7-dione

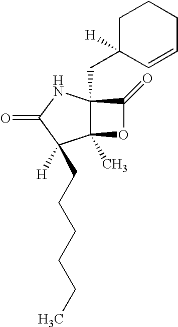

In another particulary preferred embodiment, the present invention relates to compounds according to formula

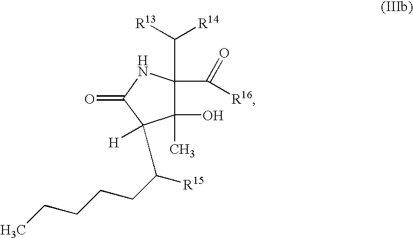

wherein
R¹³ represents hydrogen or hydroxy,
R¹⁴ represents cyclohexyl or cyclohex-2-enyl,
   wherein cyclohexyl can be substituted with 0 to 2 hydroxy groups,
R¹⁵ represents hydrogen or hydroxy,
and
R¹⁶ represents hydroxy or a substituent of the formula 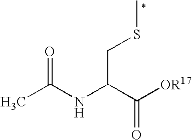
wherein
   R¹⁷ represents hydrogen or methyl,
   and
   * represents the connection position to the molecule.

Further particulary preferred compounds according to formula 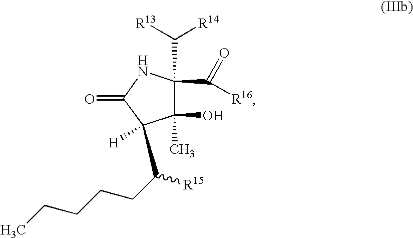
wherein
R¹³, R¹⁴, R¹⁵ and R¹⁶ have the meaning described above.

Further particularly preferred are compounds according to formula (III), such as

### (3S,4R)-2-[(S)-(1S)-2-cyclohexen-1-yl(hydroxy)methyl]-3-hydroxy-4-[1-hydroxy-hexyl]-3-methyl-5-oxo-D-proline

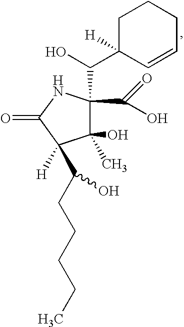

### N-acetyl-S-({(2R,3S,4R)-2-[(S)-(1S)-2-cyclohexen-1-yl(hydroxy)methyl]-4-hexyl-3-hydroxy-3-methyl-5-oxo-2-pyrrolidinyl}carbonyl)cysteine

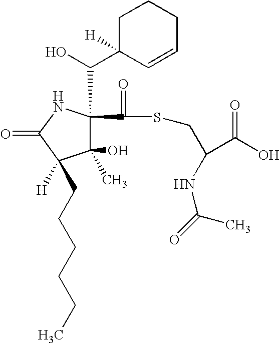

and

### methyl N-acetyl-S-({(2R,3S,4R)-2-[(S)-(1S)-2-cyclohexen-1-yl(hydroxy)methyl]-4-hexyl-3-hydroxy-3-methyl-5-oxo-2-pyrrolidinyl}carbonyl)cysteinate

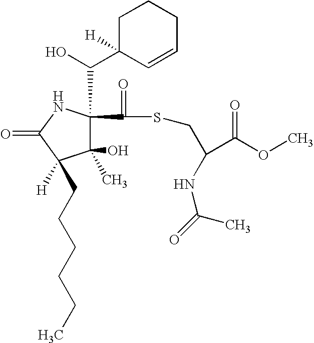

In the compounds of formula (Ia) the symbols and indices preferably have the following meanings:
R¹ is preferably the same or different H, chloro, bromo, unsubstituted or substituted (C₁-C₁₂)-alkyl, unsubstituted or substituted (C₂-C₁₀)-alkenyl, unsubstituted or substituted (C₂-C₁₀)-alkynyl, unsubstituted or substituted (C₆-C₁₂)-aryl, unsubstituted or substituted heterocyclyl, unsubstituted or substituted (C₃-C₆)-cycloalkyl, unsubstituted or substituted (C₃-C₈)-cycloalkenyl, OR″, COOR″ or —CO—R″ or the two substituents R¹ together form a 3 to 6 membered ring which is a carbocyclic ring or contains one or two heteroatom units.
R² is preferably unsubstituted or substituted (C₁-C₆)-alkyl, unsubstituted or substituted (C₂-C₆)-alkenyl, unsubstituted or substituted (C₂-C₆)-alkynyl, unsubstituted or substituted (C₃-C₆)-cycloalkyl, unsubstituted or substituted (C₃-C₈)-cycloalkenyl, unsubstituted or substituted phenyl, or unsubstituted or substituted heterocyclyl.
R³ is preferably H, unsubstituted or substituted (C₁-C₁₂)-alkyl, unsubstituted or substituted (C₂-C₁₂)-alkenyl, unsubstituted or substituted (C₂-C₁₂)-alkynyl, unsubstituted or substituted (C₃-C₆)-cycloalkyl, unsubstituted or substituted (C₆-C₁₂)-aryl, unsubstituted or substituted heterocyclyl, COOR″, —CO—R″ or CONR₂″.
R⁴ is preferably H, unsubstituted or substituted (C₁-C₁₂)-alkyl, unsubstituted or substituted (C₃-C₁₀)-alkenyl, unsubstituted or substituted (C₃-C₁₀)-alkynyl, SO₂R′, COOR′, —COR′, CONR₂″ or G.
G is Si[(C₁-C₆)-alkyl]ₓ[(CH2)ₜ-phenyl-(CH₃)ᵤ]_{y}[(O)ᵥ—((C₁-C₄)-alkylene)-(O—(C₁-C₄)-alkylene)_{w}-H]_{z},
   where x, y, z are 0, 1, 2 or 3 and x+y+z=3, t, u are 0 or 1, v, w are 0 or 1 and v+w is 1 or 2.
R′ is preferably the same or different (C₁-C₁₀)-alkyl, (C₂-C₁₀)-alkenyl, (C₂-C₁₀)-alkynyl, (C₃-C₆)-cycloalkyl, phenyl, or heterocyclyl, all of which are unsubstituted or is substituted.
R″ is preferably the same or different and is H or R′.

Preferred are compounds of formula (Ia) where all symbols have the preferred meanings and “substituted” has the preferred meanings.

More preferred compounds of formula (Ia) are compounds of formula (IIa), 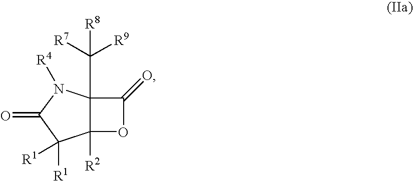
where the symbols have the following meanings:
R¹ is the same or different H, chloro, bromo, unsubstituted or substituted (C₁-C₁₀)-alkyl, unsubstituted or substituted (C₂-C₈)-alkenyl, unsubstituted or substituted (C₂-C₈)-alkynyl, unsubstituted or substituted phenyl, unsubstituted or substituted heterocyclyl, unsubstituted or substituted (C₃-C₆)-cycloalkyl, unsubstituted or substituted (C₃-C₆)-cycloalkenyl, COOR″, OR″ or —CO—R″ or the two substituents R¹ together form a 3 to 6 membered carbocyclic ring;
R² unsubstituted or substituted (C₁-C₆)-alkyl, unsubstituted or substituted (C₂-C₄)-alkenyl, unsubstituted or substituted (C₂-C₄)-alkynyl, unsubstituted or substituted (C₃-C₆)-cyclo-alkyl, unsubstituted or substituted phenyl, or unsubstituted or substituted heterocyclyl;
R⁴ is H, unsubstituted or substituted (C₁-C₁₂)-alkyl, unsubstituted or substituted (C₃-C₆)-alkenyl, unsubstituted or substituted (C₃-C₆)-alkynyl, SO₂R′, COOR′, —COR′, or CONR₂″, or G;
G is Si[(C₁-C6)-alkyl]ₓ[(CH2)ₜ-phenyl-(CH₃)ᵤ]_{y}[(O)ᵥ—((C₁-C₄)-alkylene)-(O—(C₁-C₄)-alkylene)_{w}-H]_{z},
   where x, y, z are 0, 1, 2 or 3 and x+y+z=3, t, u are 0 or 1, v, w are 0 or 1 and v+w is 1 or 2;
R⁷ is H, fluoro, chloro, O—R″, SR″, NR″₂, —O—COR′, —S—COR′, —O—CSR′, —O—SO₂R′, —O—COOR′, —O—CSOR′, —O—CONR₂″, NO₂, or CN;
R⁸ is H, unsubstituted or substituted (C₁-C₄)-alkyl, unsubstituted or substituted (C₂-C₄)-alkenyl, unsubstituted or substituted (C₂-C₄)-alkynyl, unsubstituted or substituted (C₃-C₆)-cycloalkyl, unsubstituted or substituted phenyl, or unsubstituted or substituted heterocyclyl;
or
R⁷ and R⁸ are together ═O or ═S;
R⁹ is H, halogen, unsubstituted or substituted (C₁-C₁₂)-alkyl, unsubstituted or substituted (C₂-C₁₀)-alkenyl, unsubstituted or substituted (C₂-C₁₀)-alkynyl, unsubstituted or substituted (C₆-C₁₂)-aryl, unsubstituted or substituted heterocyclyl, unsubstituted or substituted (C₃-C₈)-cycloalkyl, unsubstituted or substituted (C₃-C₈)-cycloalkenyl, O—R′, or SR′;
or
R⁸ and R⁹ together form a 3 to 8 membered ring which is a carbocyclic ring or contains one or two heteroatom units,
R′ is the same or different (C₁-C₈)-alkyl, (C₂-C₆)-alkenyl, (C₂-C₆)-alkynyl, (C₃-C₆)-cycloalkyl, phenyl, or heterocyclyl, all of which are unsubstituted or substituted; and
R″ is the same or different H or R″; and
G is SiMe₃, SiEt₃, SiMe₂t-Bu, SiMe(t-Bu)₂, Si-i-Pr₃, Si-t-BuPh₂, SiMePh₂, SiPh₃, SiMe₂(C(CH₃)₂—CH(CH₃)₂), SiEt₂i-Pr, SiMe₂i-Pr, SiMe₂i-Bu, SiBz₃, Si(CH₂—C₆H₄—CH₃)₃, SiPh₂(O-i-Pr), SiPh₂(O-t-Bu), Sit-BuPh(OMe), or SiMe₂(O—C₂H₄—OMe).

Preferred are compounds of formula (IIa) in which “substituted” has the preferred meanings. More preferred are compounds of formula (IIa) in which “substituted” has the more preferred meanings.

Particularly preferred are compounds of formula (II) in which the symbols have the following meanings:
R¹ is particularly preferred the same or different H, chloro, bromo, (C₁-C₈)-alkyl, (C₂-C₆)-alkenyl, (C₂-C₈)-alkynyl, phenyl, unsubstituted or substituted with one to three substituents from the group fluoro, chloro, methyl, ethyl, isopropyl, tert-butyl, trifluoromethyl, trifluoromethoxy, methoxy, ethoxy, NO₂, and CN, heterocyclyl, unsubstituted or substituted with fluoro, chloro, methyl, ethyl, isopropyl, tert-butyl, trifluoromethyl, trifluoromethoxy, methoxy, ethoxy, NO₂, or CN, (C₃-C₆)-cycloalkyl, (C₃-C8)-cycloalkenyl, both unsubstituted or substituted with one to three substituents from the group fluoro, chloro, methyl, trifluoromethyl, methoxy, NO₂, and CN, or the two substituents R¹ together form a 3 to 6 membered carbocyclic ring.
R² is particularly preferred methyl, ethyl, propyl, isopropyl, allyl, propargyl, cyclopropyl, cyclobutyl, cyclopentyl, cyclohexyl, phenyl, unsubstituted or substituted with one to three substituents from the group fluoro, chloro, methyl, ethyl, isopropyl, tert-butyl, trifluoromethyl, trifluoromethoxy, methoxy, ethoxy, NO₂, and CN, or heterocyclyl, unsubstituted or substituted with one to three substituents from the group fluoro, chloro, methyl, ethyl, trifluoromethyl, methoxy, NO₂, and CN.
R⁴ is particularly preferred H, unsubstituted or substituted (C₁-C₁₂)-alkyl, unsubstituted or substituted (C₃-C₁₀)-alkenyl, unsubstituted or substituted (C₃-C₆)-alkynyl, SO₂R′, COOR′, —COR′, CONR₂″ or G.
R⁷ is particulary preferred hydroxyl, mercapto, SCH₃, fluoro, chloro, bromo, methyl, ethyl, methoxy, trifluoromethoxy, ethoxy, —O—SO₂R′, —O—COOR′, —O—CONR₂″, CN, NR₂″ or O-G.
R⁸ is particularly preferred H, unsubstituted or substituted (C₁-C₄)-alkyl, unsubstituted or substituted (C₂-C₄)-alkenyl, unsubstituted or substituted (C₂-C₄)-alkynyl, unsubstituted or substituted (C₃-C₆)cycloalkyl, unsubstituted or substituted (C₃-C₈)-cycloalkenyl, phenyl, unsubstituted or substituted with one to three substituents from the group fluoro, chloro, methyl, ethyl, isopropyl, tert-butyl, trifluoromethyl, trifluoromethoxy, methoxy, ethoxy, NO₂ and CN, heterocyclyl, unsubstituted or substituted with one to three substituents from the group fluoro, chloro, methyl, ethyl, isopropyl, tert-butyl, trifluoromethyl, trifluoromethoxy, methoxy, ethoxy, NO₂ and CN;
or particularly preferred
R⁷ and R⁸ are together ═O or ═S.
R⁹ is particularly preferred H, halogen, unsubstituted or substituted (C₁-C₈)-alkyl, unsubstituted or substituted (C₂-C₆)-alkenyl, propargyl, unsubstituted or substituted (C₆-C₁₀)-aryl, unsubstituted or substituted heterocyclyl, unsubstituted or substituted (C₃-C₆)-cycloalkyl, unsubstituted or substituted (C₃-C₆)-cycloalkenyl;
or particularly preferred
R⁸ and R⁹ together form a 3 to 6 membered ring which is a carbocyclic ring or contains one or two heteroatom units.
G is particularly preferred SiMe₃, SiEt₃, SiMe₂t-Bu, SiMe(t-Bu)₂, Si-i-Pr₃, Si-t-BuPh₂, SiMePh₂, SiPh₃, SiMe₂(C(CH₃)₂—CH(CH₃)₂), SiEt₂i-Pr, SiMe₂i-Pr, SiMe₂i-Bu, SiBz₃, Si(CH₂—C₆H₄—CH₃)₃, SiPh₂(O-i-Pr), SiPh₂(O-t-Bu), Sit-BuPh(OMe), or SiMe₂(O—C₂H₄—OMe).
R′ is particulary preferred the same or different (C₁-C₆)-alkyl, (C₂-C₄)-alkenyl, (C₂-C₄)-alkynyl, (C₃-C₆)-cycloalkyl, phenyl, or heterocyclyl, all of which are unsubstituted or substituted.
R″ is particularly preferred the same or different H or R′

Particulary preferred are compounds of formula (IIa) in which all the symbols and the term “substituted” have the particulary preferred meanings.

In the formula (Ib) the symbols and indices preferably have the following meanings:
R¹ is preferably the same or different H, chloro, bromo, unsubstituted or substituted (C₁-C₁₂)-alkyl, unsubstituted or substituted (C₂-C₁₀)-alkenyl, unsubstituted or substituted (C₂-C₁₀)-alkynyl, unsubstituted or substituted (C₆-C₁₂)-aryl, unsubstituted or substituted heterocyclyl, unsubstituted or substituted (C₃-C₆)-cycloalkyl, unsubstituted or substituted (C₃-C₆)-cycloalkenyl, OR″, COOR″ or —CO—R″ or the two substituents R¹ together form a 3 to 6 membered ring which is a carbocyclic ring or contains one or two heteroatom units.
R² is preferably H, unsubstituted or substituted (C₁-C₅)-alkyl, unsubstituted or substituted (C₂-C₆)-alkenyl, unsubstituted or substituted (C₂-C₆)-alkynyl, unsubstituted or substituted (C₃-C₆)-cycloalkyl, unsubstituted or substituted phenyl, or unsubstituted or substituted heterocyclyl.
R³ is preferably H, unsubstituted or substituted (C₁-C₁₂)-alkyl, unsubstituted or substituted (C₂-C₁₂)-alkenyl, unsubstituted or substituted (C₂-C₁₂)-alkynyl, unsubstituted or substituted (C₃-C₆)-cycloalkyl, unsubstituted or substituted (C₆-C₁₂)-aryl, unsubstituted or substituted heterocyclyl, —CO—R″ or CONR₂″.
R⁴ is preferably H, unsubstituted or substituted (C₁-C₁₂)-alkyl, unsubstituted or substituted (C₃-C₁₀)-alkenyl, unsubstituted or substituted (C₃-C₁₀)-alkynyl, SO₂R′, COOR′, —COR′, CONR₂″ or G.
R⁵ is preferably H, unsubstituted or substituted (C₁-C₁₂)-alkyl, unsubstituted or substituted (C₂-C₁₂)-alkenyl, unsubstituted or substituted (C₂-C₁₂)-alkynyl, unsubstituted or substituted (C₃-C₈)-cycloalkyl, unsubstituted or substituted phenyl, unsubstituted or substituted heterocyclyl, SO₂R′, COR′, COOR′, COSR′, CSOR′, CONR₂″ or G.
G is Si[(C₁-C₆)-alkyl]ₓ[(CH₂)ₜ-phenyl-(CH₃)ᵤ]_{y}[(O)ᵥ—((C₁-C₄)-alkylene)-(O—(C₁-C₄)-alkylene)_{w}-H]_{z},
   where x, y, z are 0, 1, 2 or 3 and x+y+z=3, t, u are 0 or 1, v, w are 0 or 1 and v+w is 1 or 2.
R⁶ is preferably OR″, SR″ or NR₂″.
R′ is preferably the same or different (C₁-C₁₀)-alkyl, (C₂-C₁₀)-alkenyl, (C₂-C₁₀)-alkynyl, (C₃-C₆)-cycloalkyl, phenyl, or heterocyclyl, all of which are unsubstituted or is substituted.
R″ is preferably the same or different and is H or R′.

Preferred are compounds of formula (Ib) where all symbols have the preferred meanings and “substituted” has the preferred meanings.

More preferred compounds of formula (Ib) are compounds of formula (IIb), 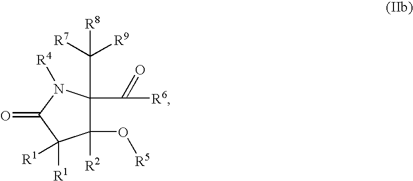
where the symbols have the following meanings:
R′ is the same or different H, chloro, bromo, unsubstituted or substituted (C₁-C₁₀)-alkyl, unsubstituted or substituted (C₂-C₈)-alkenyl, unsubstituted or substituted (C₂-C₈)-alkynyl, unsubstituted or substituted phenyl, unsubstituted or substituted heterocyclyl, unsubstituted or substituted (C₃-C₆)-cycloalkyl, COOR′, OR″ or —CO—R′ or the two substituents R together form a 3 to 6 membered carbocyclic ring;
R² H, unsubstituted or substituted (C₁-C₆)-alkyl, unsubstituted or substituted (C₂-C₄)-alkenyl, unsubstituted or substituted (C₂-C₄)-alkynyl, unsubstituted or substituted (C₃-C₆)-cycloalkyl, unsubstituted or substituted phenyl, or unsubstituted or substituted heterocyclyl;
R⁴ is H, unsubstituted or substituted (C₁-C₁₂)-alkyl, unsubstituted or substituted (C₃-C₆)-alkenyl, unsubstituted or substituted (C₃-C₆)-alkynyl, SO₂R′, COOR′, —COR′, CONR₂″, or G;
R⁵ is H, unsubstituted or substituted (C₁-C₈)-alkyl, unsubstituted or substituted (C₂-C₆)-alkenyl, unsubstituted or substituted (C₂-C₆)-alkynyl, unsubstituted or substituted (C₃-C₆)-cycloalkyl, SO₂R′, COOR′, —COR′, CONR₂″, unsubstituted or substituted phenyl, unsubstituted or substituted heterocyclyl, or G;
G is Si[(C₁-C₆)-alkyl]ₓ[(CH₂)ₜ-phenyl-(CH₃)ᵤ]_{y}[(O)ᵥ—((C₁-C₄)-alkylene)-(O—(C₁-C₄)-alkylene)_{w}-H]_{z},
where x, y, z are 0, 1, 2 or 3 and x+y+z=3, t, u are 0 or 1, v, w are 0 or 1 and v+w is 1 or 2;
R⁶ is OR″, SR″ or NR₂″;
R⁷ is H, fluoro, chloro, O—R″, SR″, —O—COR′, —S—COR′, —O—CSR′, —O—SO₂R′, NR₂″, —O—COOR′, —O—CSOR′, —O—CONR₂″, NO₂ or CN;
R⁸ is H, unsubstituted or substituted (C₁-C₄)-alkyl, unsubstituted or substituted (C₂-C₄)-alkenyl, unsubstituted or substituted (C₂-C₄)-alkynyl, unsubstituted or substituted (C₃-C₆)-cycloalkyl, unsubstituted or substituted phenyl, or unsubstituted or substituted heterocyclyl;
or p1 R⁷ and R⁸ are together ═O or ═S;
R⁹ is H, halogen, unsubstituted or substituted (C₁-C₁₂)-alkyl, unsubstituted or substituted (C2-C₁₀)-alkenyl, unsubstituted or substituted (C₂-C₁₀)-alkynyl, unsubstituted or substituted (C₆-C₁₂)-aryl, unsubstituted or substituted heterocyclyl, unsubstituted or substituted (C₃-C₈)-cycloalkyl, unsubstituted or substituted (C₃-C₈)-cycloalkenyl, O—R′, or SR′,
provided that if R⁷ and R⁸ together are ═O, R⁹ is not OR′;
or
R⁸ and R⁹ together form a 3 to 8 membered ring which is a carbocyclic ring or contains one or two heteroatom units,
R′ is the same or different (C₁-C₈)-alkyl, (C₂-C₆)-alkenyl, (C₂-C₆)-alkynyl, (C₃-C₆)-cycloalkyl, phenyl, or heterocyclyl, all of which are unsubstituted or substituted; and
R″ is the same or different H or R″;
G is SiMe₃, SiEt₃, SiMe₂t-Bu, SiMe(t-Bu)₂, Si-i-Pr₃, Si-t-BuPh₂, SiMePh₂, SiPh₃, SiMe₂(C(CH₃)₂—CH(CH₃)₂), SiEt₂i-Pr, SiMe₂i-Pr, SiMe₂i-Bu, SiBz₃, Si(CH₂—C₆H₄—CH₃)₃, SiPh₂(O-i-Pr), SiPh₂(O-t-Bu), Sit-BuPh(OMe), or SiMe₂(O—C₂H₄—OMe);
n is 0, 1 or 2.

Preferred are compounds of formula (IIb) in which “substituted” has the preferred meanings. More preferred are compounds of formula (IIb) in which “substituted” has the more preferred meanings.

Particularly preferred are compounds of formula (IIb) in which the symbols have the following meanings:
R¹ is particularly preferred the same or different H, chloro, bromo, (C₁-C₈)-alkyl, (C₂-C₆)-alkenyl, (C₂-C₈)-alkynyl, phenyl, unsubstituted or substituted with one to three substituents from the group fluoro, chloro, methyl, ethyl, isopropyl, tert-butyl, trifluoromethyl, trifluoromethoxy, methoxy, ethoxy, NO₂, and CN, heterocyclyl, unsubstituted or substituted with fluoro, chloro, methyl, ethyl, isopropyl, tert-butyl, trifluoromethyl, trifluoromethoxy, methoxy, ethoxy, NO₂, or CN, (C₃-C₆)-cycloalkyl, (C₃-C8)-cycloalkenyl, both unsubstituted or substituted with one to three substituents from the group fluoro, chloro, methyl, trifluoromethyl, methoxy, NO₂, and CN, or the two substituents R¹ together form a 3 to 6 membered carbocyclic ring.
R² is particularly preferred H, methyl, ethyl, propyl, isopropyl, allyl, propargyl, cyclopropyl, cyclobutyl, cyclopentyl, cyclohexyl, phenyl, unsubstituted or substituted with one to three substituents from the group fluoro, chloro, methyl, ethyl, isopropyl, tert-butyl, trifluoromethyl, trifluoromethoxy, methoxy, ethoxy, NO₂, and CN, or heterocyclyl, unsubstituted or substituted with one to three substituents from the group fluoro, chloro, methyl, ethyl, trifluoromethyl, methoxy, NO₂, and CN.
R⁴ is particularly preferred H, unsubstituted or substituted (C₁-C₁₂)-alkyl, unsubstituted or substituted (C₃-C₆)-alkenyl, unsubstituted or substituted (C₃-C₆)-alkynyl, SO₂R′, COOR′, —COR″, CONR₂″or G.
R⁵ is particularly preferred H, (C₁-C₆)-alkyl, (C₂-C₆)-alkenyl, (C₂-C₆)-alkynyl, unsubstituted or substituted (C₃-C₆)-cycloalkyl, SO₂R′, COOR′, —COR″, CONR₂″, or G.
R⁶ is particularly preferred OR″, SR″ or NR₂″,
R⁷ is particulary preferred hydroxyl, mercapto, SCH₃, fluoro, chloro, bromo, methyl, ethyl, methoxy, trifluoromethoxy, ethoxy, —O—SO₂R′, —O—COOR′, —O—CONR₂″, CN, NR₂″ or O-G.
R⁸ is particularly preferred H, unsubstituted or substituted (C₁-C₄)-alkyl, unsubstituted or substituted (C₂-C₄)-alkenyl, unsubstituted or substituted (C₂-C₄)-alkynyl, unsubstituted or substituted (C₃-C₆)cycloalkyl, unsubstituted or substituted (C₃-C₈)-cycloalkenyl, phenyl, unsubstituted or substituted with one to three substituents from the group fluoro, chloro, methyl, ethyl, isopropyl, tert-butyl, trifluoromethyl, trifluoromethoxy, methoxy, ethoxy, NO₂ and CN, heterocyclyl, unsubstituted or substituted with one to three substituents from
the group fluoro, chloro, methyl, ethyl, isopropyl, tert-butyl, trifluoromethyl, trifluoromethoxy, methoxy, ethoxy, NO₂ and CN;
or particularly preferred
R⁷ and R⁸ are together ═O or ═S.
R⁹ is particularly preferred H, halogen, unsubstituted or substituted (C₁-C₈)-alkyl, unsubstituted or substituted (C₂-C₆)-alkenyl, propargyl, unsubstituted or substituted (C₆-C₁₀)-aryl, unsubstituted or substituted heterocyclyl, unsubstituted or substituted (C₃-C₆)-cycloalkyl, unsubstituted or substituted (C₃-C₆)-cycloalkenyl;
or particularly preferred
R⁸ and R⁹ together form a 3 to 6 membered ring which is a carbocyclic ring or contains one or two heteroatom units.
G is particularly preferred SiMe₃, SiEt₃, SiMe₂t-Bu, SiMe(t-Bu)₂, Si-i-Pr₃, Si-t-BuPh₂, SiMePh₂, SiPh₃, SiMe₂(C(CH₃)₂—CH(CH₃)₂), SiEt₂i-Pr, SiMe₂i-Pr, SiMe₂i-Bu, SiBz₃, Si(CH₂—C₆H₄—CH₃)₃, SiPh₂(O-i-Pr), SiPh₂(O-t-Bu), Sit-BuPh(OMe), or SiMe₂(O—C₂H₄—OMe);
R′ is particulary preferred the same or different (C₁-C₆)-alkyl, (C₂-C₄)-alkenyl, (C₂-C₄)-alkynyl, (C₃-C₆)-cycloalkyl, phenyl, or heterocyclyl, all of which are unsubstituted or substituted.
R″ is particularly preferred the same or different H or R′

Particulary preferred are compounds of formula (IIb) in which all the symbols and the term “substituted” have the particulary preferred meanings.

The compounds according to the invention are prepared by methods which are known per se from the literature, as described in standard works on organic synthesis, for example Houben.-Weyl, Methoden der Organischen Chemie [Methods in Organic Chemistry], Georg-Thieme-Verlag, Stuttgart.

The preparation is carried out under reaction conditions which are known and suitable for the abovementioned reactions. Other variants which are known per se, but not illustrated here in greater detail, may also be used.

If desired, the starting materials may also be formed in situ, in such a way that they are not isolated from the reaction mixture but immediately reacted further to give the compounds of the formula (I).

In particular compounds of formula (I) can be prepared according to the following processes A to D:
Process A 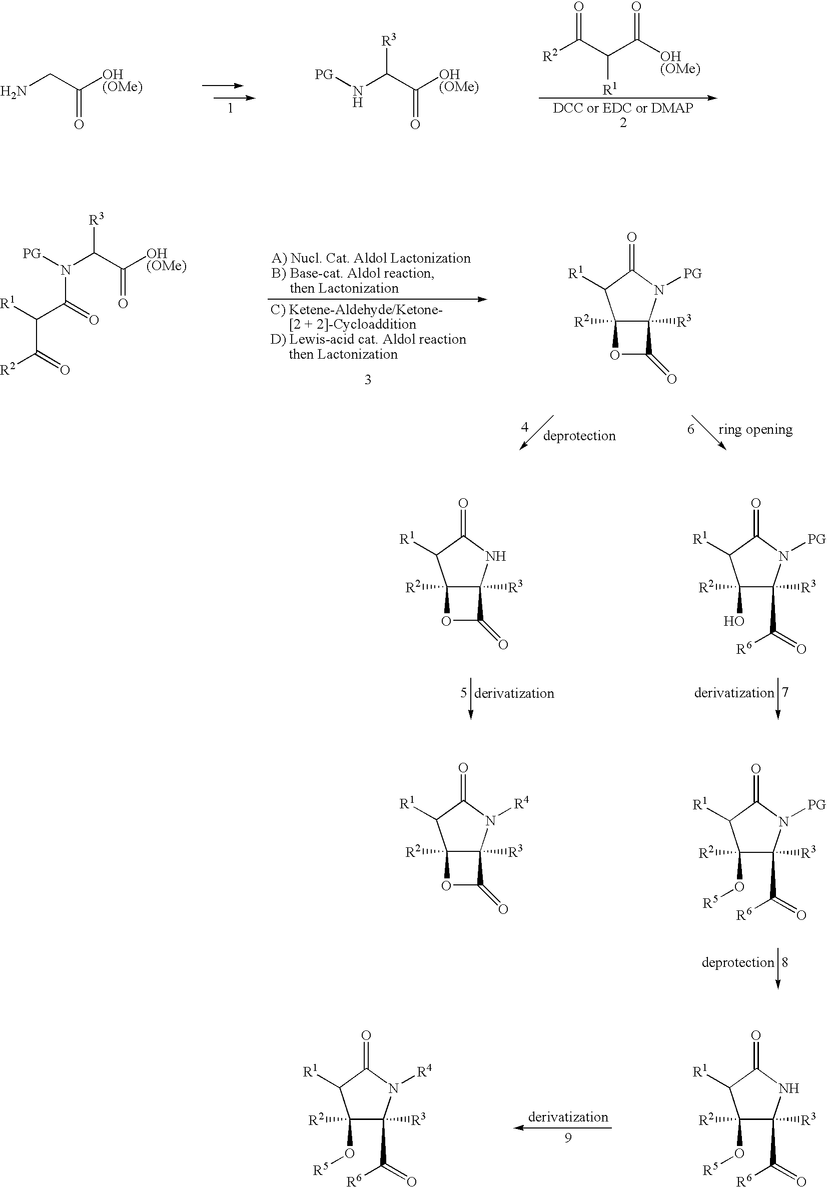
1

Following well established procedures known in the literature glycin or its methyl ester is converted into derivatized amino acid after protecting the free amine (e.g. D. Enders, Helv. Chim Acta 85 (2002) 3657-3677). R³ is introduced this way. 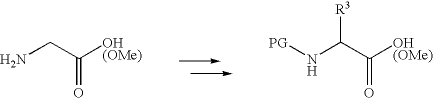
2

The protected amino acids is acylated by substituted beta-keto acids or their esters bearing R¹ and R². The acylation is performed by using dicyclohexylcarbodiimide (DCC), 1-Ethyl-3-(3-Dimethyl-aminopropyl)carbodiimide (EDC) or 4-dimethylaminopyridine (DMAP). 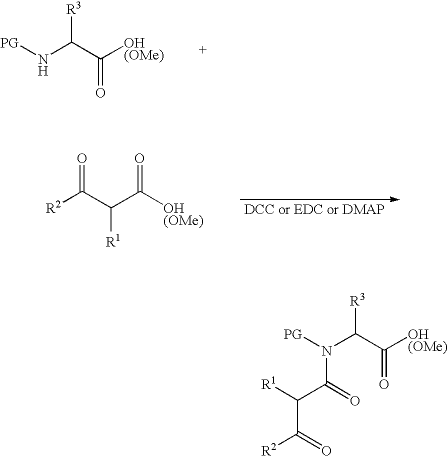
3

Key step of the route is the lactonization. It is performed either as a nucleophilic catalysed aldol lactonization (method A) described by Romo et al. (G. S, Cortez, R. L. Tennyson, D. Romo J. Am. Chem. Soc. 2001, 123, 7945) or as a standard base catalysed aldol reaction (method B) with a following lactonization (E. J. Corey et. al., Angew. Chem. 1998, 110, 1784). Furthermore the lactone can be obtained by using a ketene aldehyde [2+2]-cycloaddition (method C, C. Zhu, X. Shen, S. G. Nelson J. Am. Chem. Soc. 2004, 126, 5352). Lewis-acid catalyzed Aldol reaction with a following lactonization is a further possible method (method D). Methodologies for the catalytic aldol reaction with ketones have been reported for non-stereocontrolled reactions (e.g. Kobayashi, S. et al. Chem. Lett. 1988, 1491; Chen, J. et al. J. Org. Chem. 1988, 63, 9739) and for enantio-selective aldol reactions (Denmark, S. E.; Fan, Y. J. Am. Chem. Soc. 2002, 124, 4233; Oisaki, K. et al. J Am. Chem. Soc. 2003, 125, 5644). 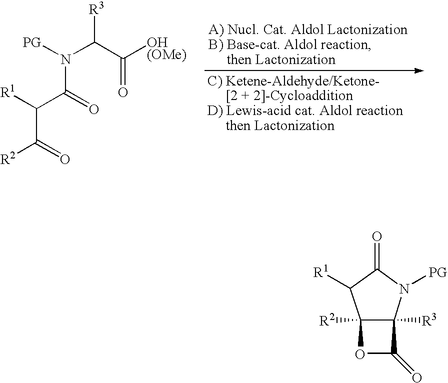
4+5

Compounds of formula (Ia) are synthesized following known literature procedures. The nitrogen is deprotected and derivatized with R⁴ if needed. 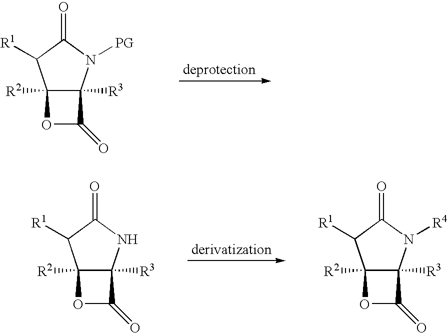
6

Compounds of formula (I) are synthesized following known literature procedures. In the first step the lactone ring is cleaved according to methods known in the literature. R⁶ is introduced in the molecule. 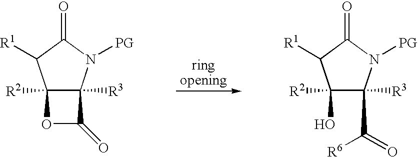
7+8+9

After derivatization of the free OH to introduce R⁵ the nitrogen is deprotected and derivatized with R⁴ if needed. 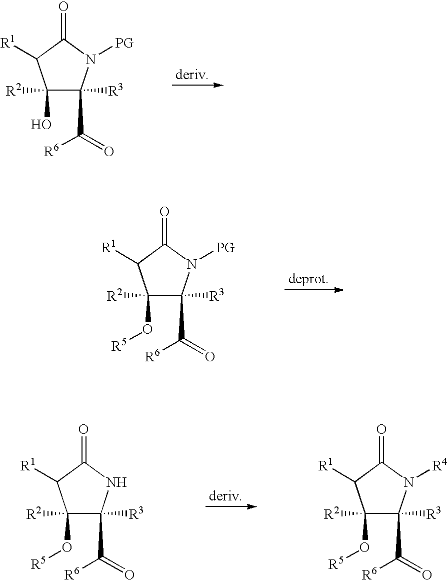
Process B

Compounds of of formula (II) are synthesized following a literature procedure (F. Souci, L. Grenier, M. L. Behnke, A. T. Destree, T. A. McCormack, J. Adams, L. Plamondon, J. Am. Chem. Soc. 1999, 121, 9967) via a multistep synthesis as shown in the following scheme.

This route can be performed to introduce all stereogenic centers with the required configuration. 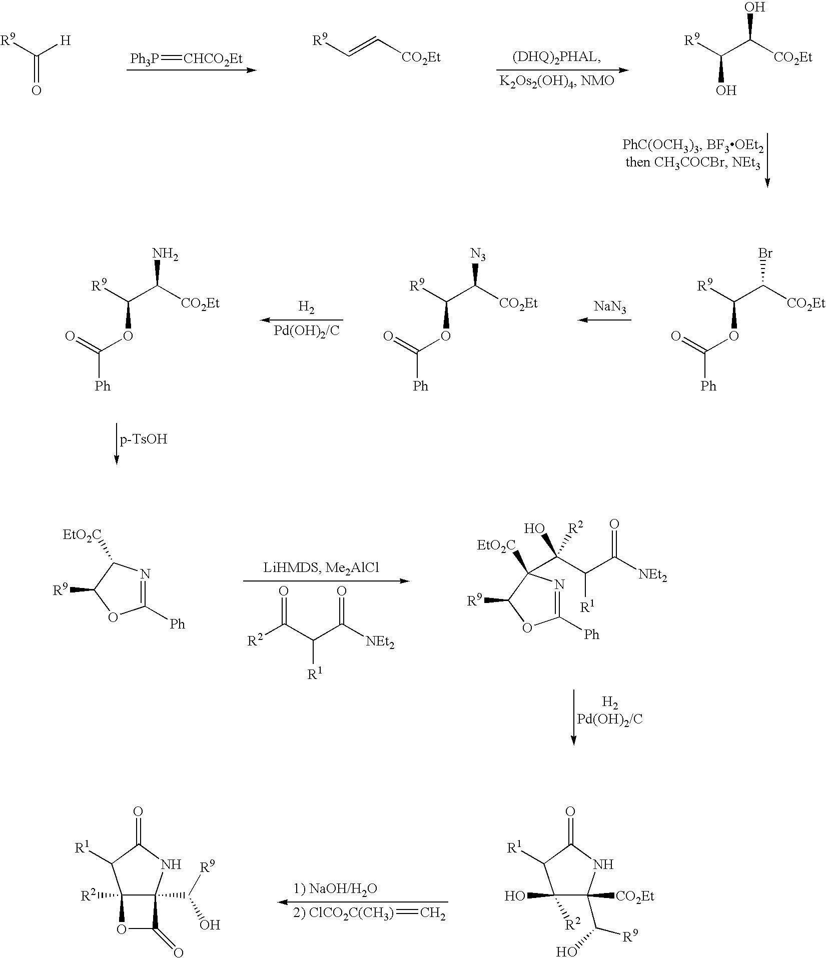
Process C 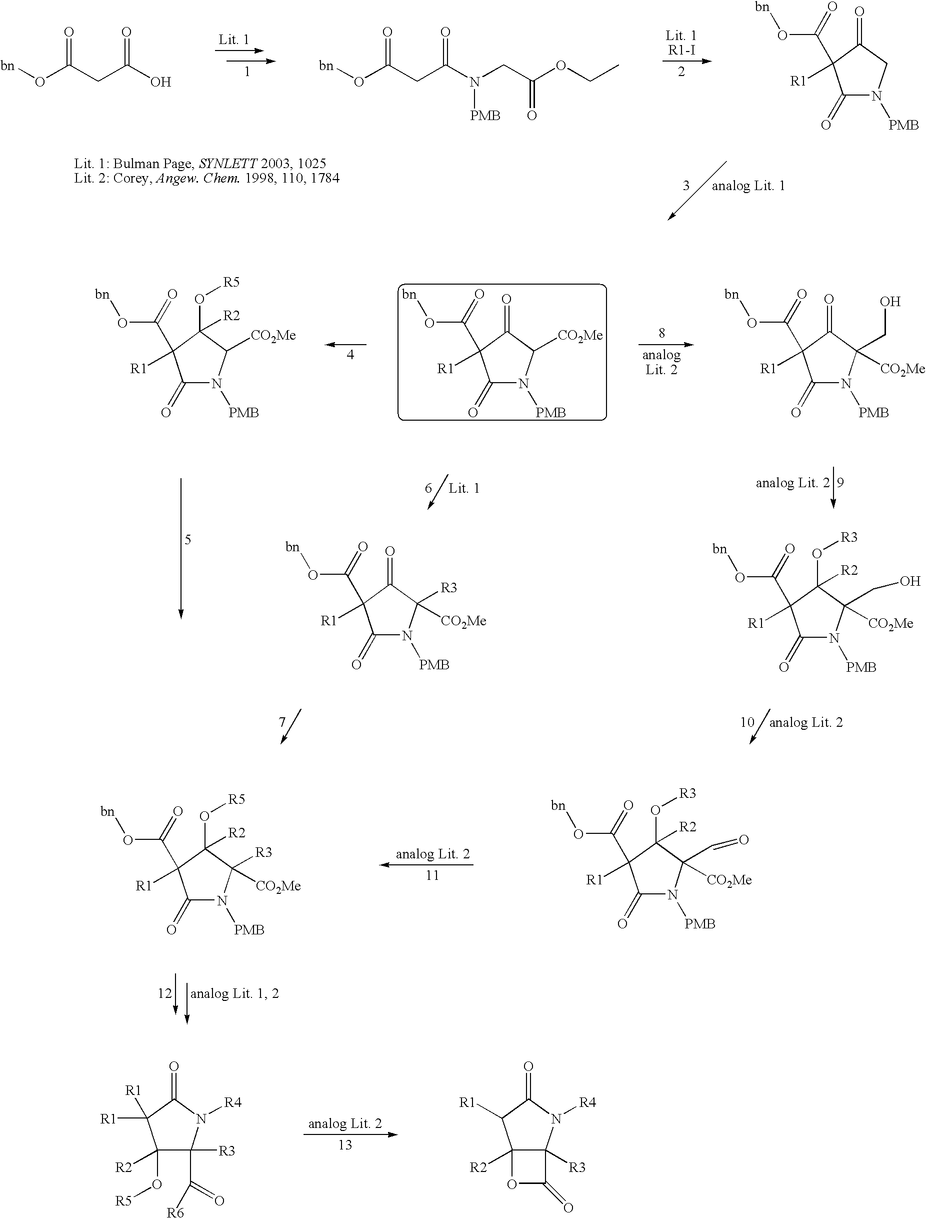
1

Following the literature procedure [1] monobenzyl malonate is converted into the acid chloride und subsequently treated with N-(p-methoxybenzyl)glycine ethyl ester.

R¹=H, R⁴=pmb

Possible variations (not published): R¹=Aryl, Alkyl; R⁴=Aryl, Alkyl 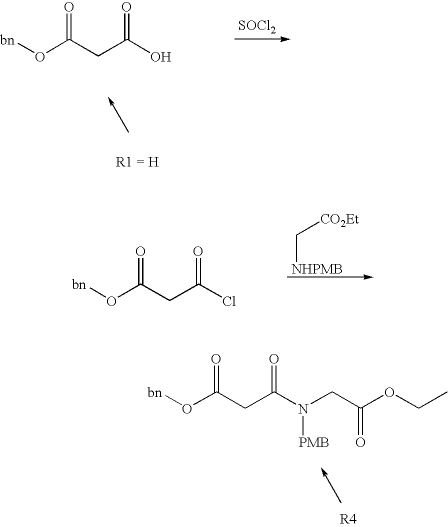
2

Cyclisation is induced by tetrabutylammoniumfluoride, alkylations (introduction of R¹) are performed using alkyl-, benzyl- or allyl iodides [1]. 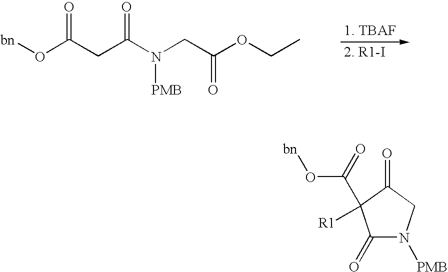
3

LHMDS (Lithiumhexamethyldisilazid) and DMPU in THF are employed for deprotonation, methyl cyanoformate for carboxylation [1]. 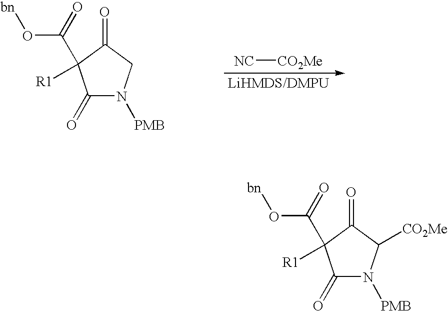
4

Ketone reduction can be performed analog [2] using NaBH(OAc)₃ (R⁵═R²═H). Introduction of substituents R₂ (e.g. alkyl, ally, aryl) may be performed by addition of a suitable organometallic reagent (e.g. Grignard). 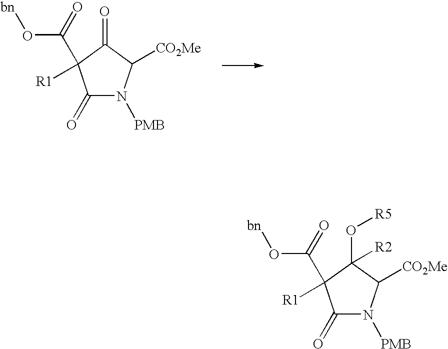
5

After O-protection (R⁵=silyl, benzyl) alkyls and aldehydes can be added.

(R³=alkyl, allyl, benzyl or R⁷=alkyl, allyl, benzyl, aryl, R⁸=OH) 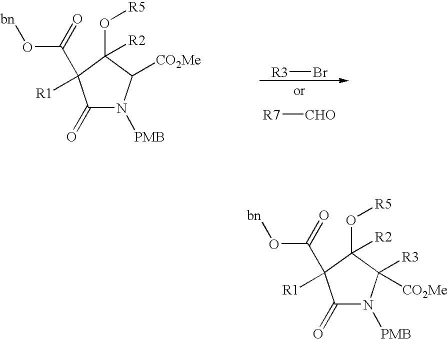
6

According to [1] allyl bromides react readily. 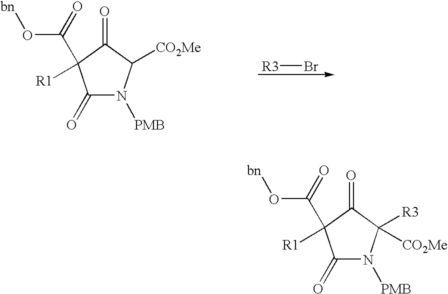
7
see 4
8-11

analog to [2]: addition of formaldehyd (8), ketone reduction (see (4)), protection of primary hydroxyl group as pivolyl ester, protection of secondary hydroxyl group as TBS ether, deprotection of pivolyl ester (9), Dess-Martin Oxidation (10), addition of Grignard reagents [2] or Zinc organometallics [3] (11) 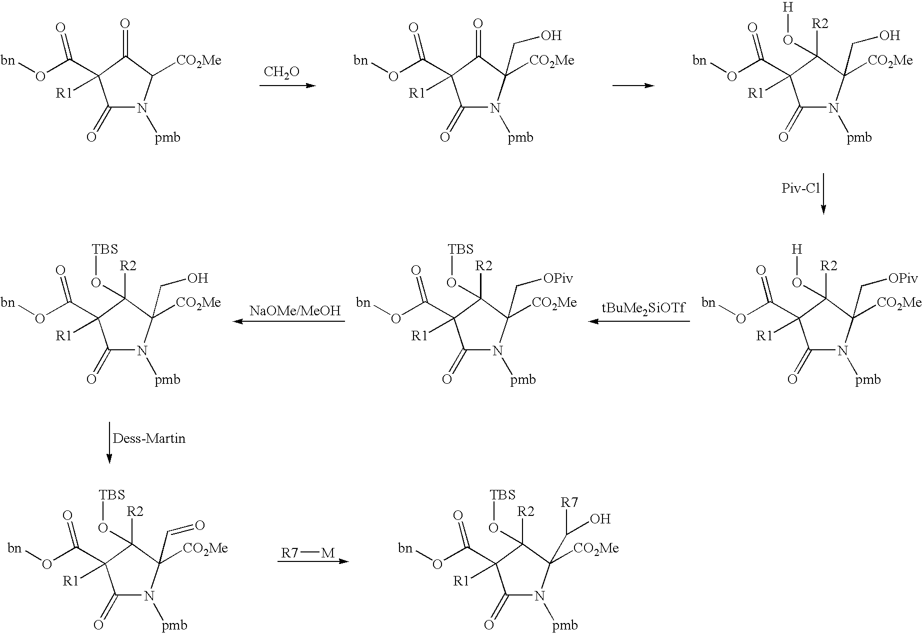
12

Possible transformations are

decarboxylation (R1 no more CO₂Bn): Pd-catalyzed hydrogenolysis gives debenzylation and induces decarboxylation [1]

N-deprotection (R4=H) utilizing Cer(ammonium)nitrat (CAN) if R4 is PMB [2]

transesterfication (R6=O-alkyl, O-allyl, O-benzyl)

saponification (R6=OH)

thioester formation (R6=S-alkyl)

amide formation (R6=NH-alkyl/Aryl, N-alkyl-aryl, N-dialkyl)

13

Lactonisation using BOP-Cl is described in [2].

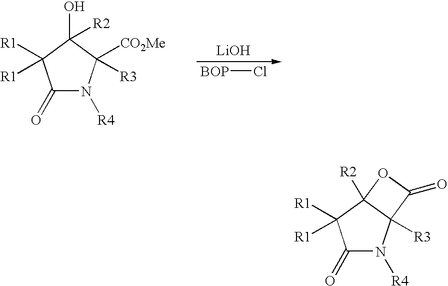

Literature:

[1] P. C. Bulman Page et. al., SYNLETT 2003, 1025.

[2] E. J. Corey et. al., Angew. Chem. 1998, 110, 1784.

[3] L. R. Reddy, P. Saravanan, E. J. Corey, J. Am. Chem. Soc., ASAP

Process D

Compounds of formula (I) with R²═H can be synthesized following a literature procedure (E. J. Corey, W. Li, T. Nagamitsu, Angew. Chem. IE 1998, 37, 1676-1679.) starting from enantiomerically pure substituted malonate via a multistep synthesis as shown in the process scheme. This synthetic route is highly flexible for R¹ and it can be carried out with complete stereocontrol. Key intermediate Z allows broad variation of R³. 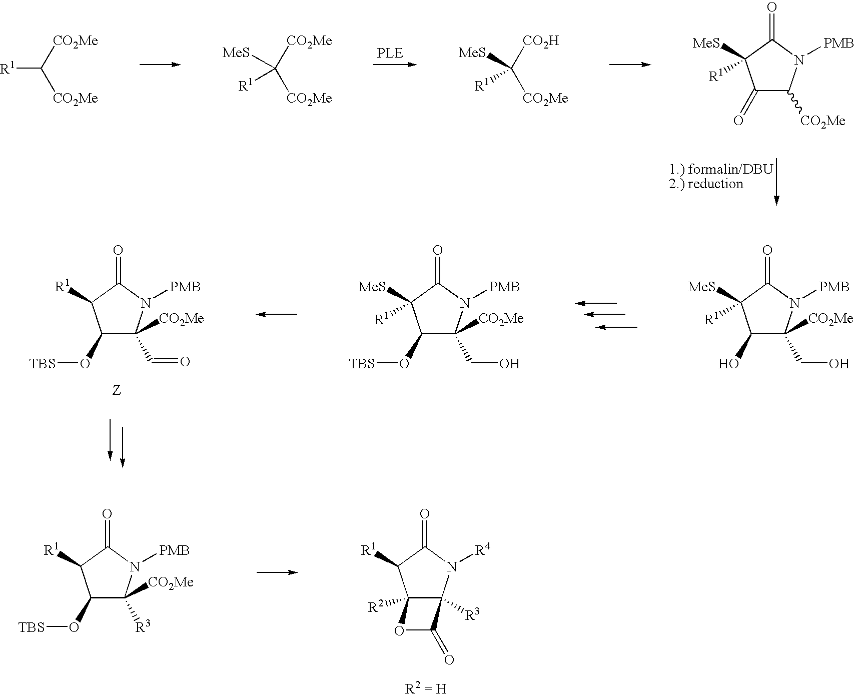
Process E

This process is based on a method described in the literature (Corey et al., J. Am. Chem. Soc. 2004, 126, 6230-6231) 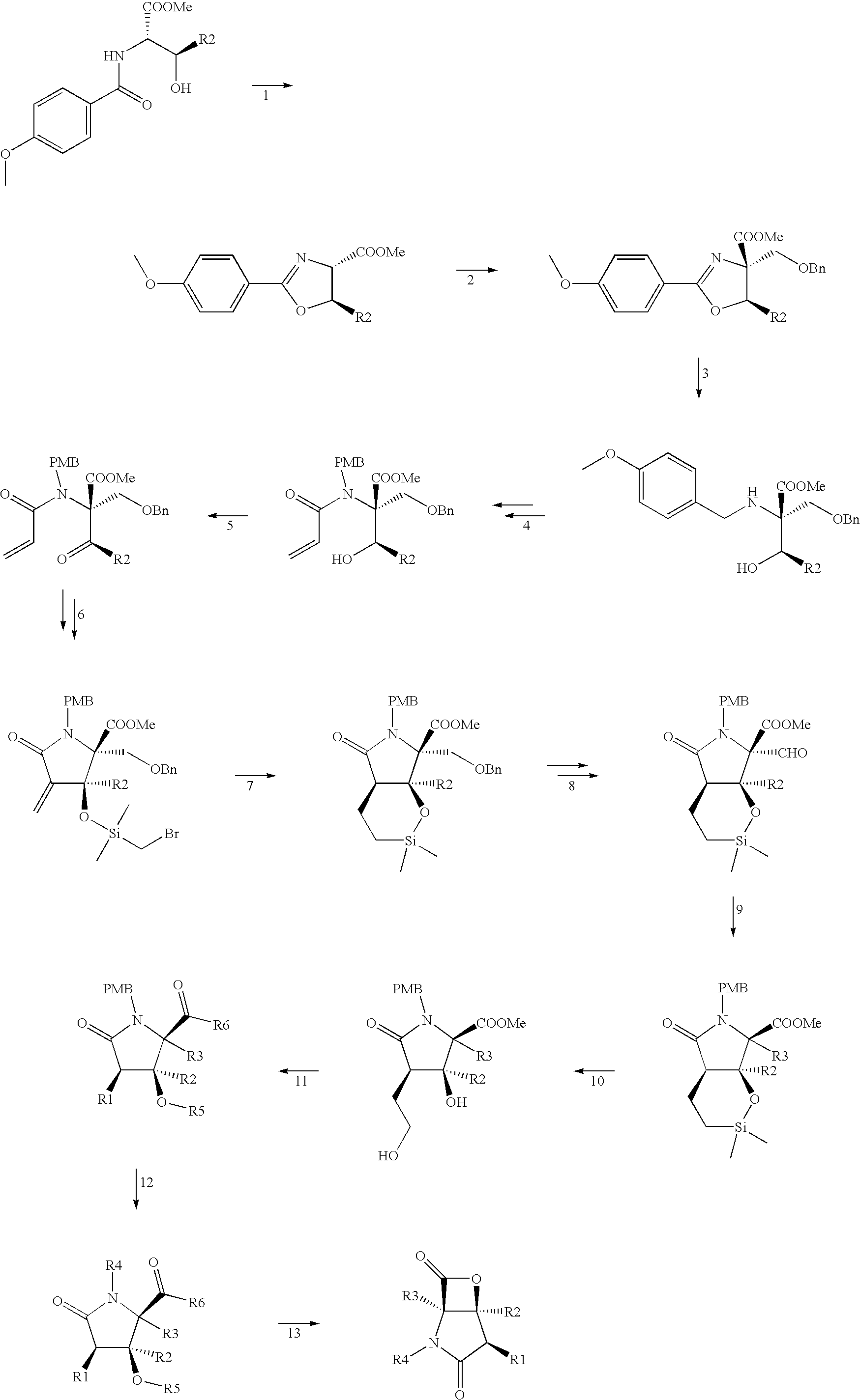
1

Following the literature procedure a derivative of serine methyl ester was N-acylated with 4-methoxybenzoyl chloride to form the amide which is cyclized to an oxazoline by heating with p-toluenesulfonic acid. 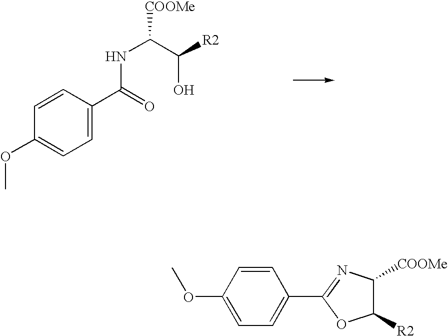
2

Deprotonation of the oxazoline with lithium diisopropylamide alkylation of the resulting enolate with chloromethyl benzyl ether affords the desired isomer. 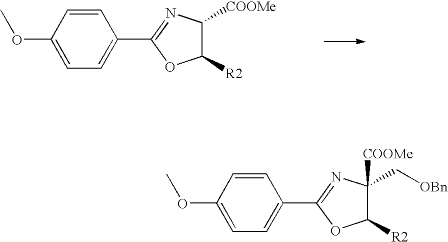
3-4

Reduction with sodium cyanoborohydride yields to a PMB-amine (PMB=4-methoxybenzyl) which can be transformed to the N-acrylyl-N-PMB derivative by reaction with Me3SiCl and Et3N to form the trimethyl silyl ether, followed by acylation with acrylyl chloride and acidic work up. 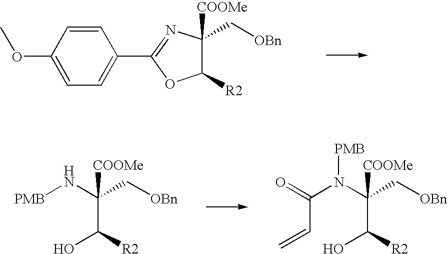
5

Dess-Martin periodinane oxidation produces the keto amide ester. 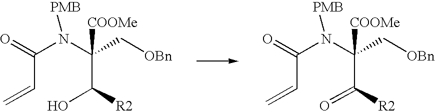
6

The keto amide ester can be cyclized to the γ-lactam by means of an internal Baylis-Hillman-aldol reaction using quinuclidine as the catalytic base. The cyclization product can be silylated with bromomethyldimethylsilyl chloride and separated by column chromatography. 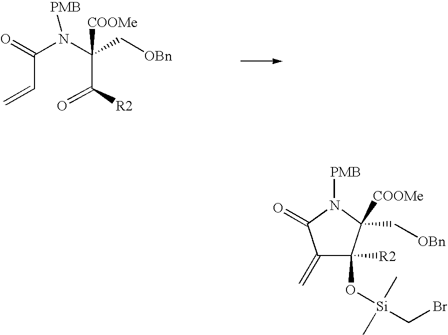
7

The γ-lactam core can be transformed by tri-n-butyltin hydride mediated radical-chain cyclization cleanly into the cis-fused bicyclic γ-lactam. 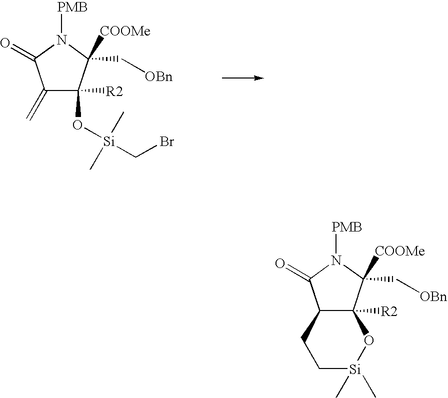
8

Cleavage of the benzyl ether with hydrogen on palladium/charcoal and Dess-Martin periodinane oxidation provide an aldehyde. 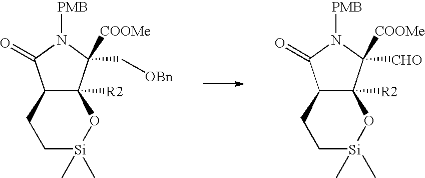
9

Conversion of this aldehyde into substituents R3 is possible either by addition of a suitable organometallic reagent, e.g. Grignard, or by performing a Wittig reaction followed by a hydration of the created alkene. 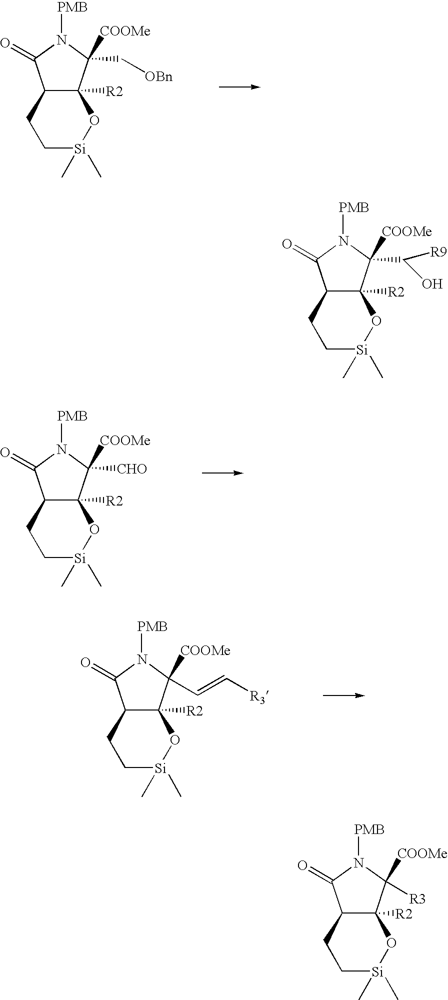
10

Tamao-Fleming oxidation of the bicycle affords the corresponding PMB protected diol ester. 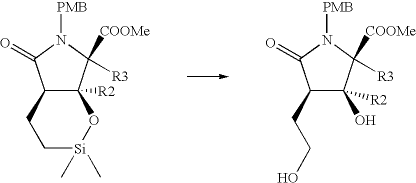
11

Possible transformations of this diol ester are
oxidation of the primary hydroxy group to the aldehyde following Wittig reaction and reduction of the created alkene to vary the substituent R1
derivatization (e.g. alkylation, acylation etc.) of the secondary hydroxy group (e.g. R5=alkyl, allyl, benzyl, acyl etc.)
transesterfication (e.g. R6=O-alkyl, O-allyl, O-benzyl)
saponification (e.g. R6=OH)
thioester formation (e.g. R6=S-alkyl)
amide formation (e.g. R6=NH-alkyl/aryl, N-alkyl-aryl, N-dialkyl) 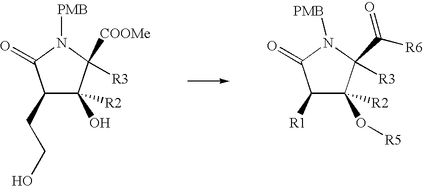
   12

After Ce(IV)-induced oxidative cleavage of the PMB group the amide can be N-alkylated (R4=alkyl, benzyl etc.) by using standard procedures if needed. 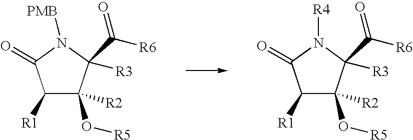
13

Lactonisation of the β-hydroxy acid (R4=R5=R6=H) to compounds of formula (Ia) proceeds by using bis-(2-oxo-3-oxazolidinyl)phosphinic chloride (BOPCl). If needed, the amide can be N-alkylated (R4=alkyl, allyl, benzyl etc.) at this stage, too. 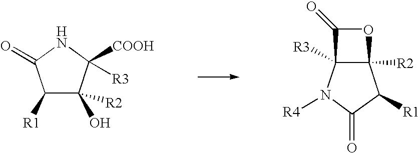
Process F

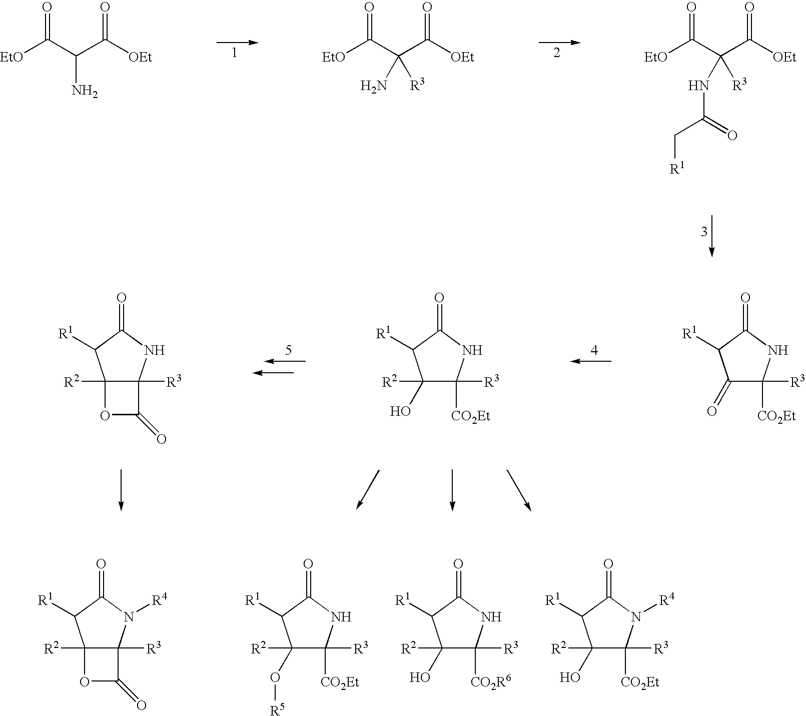

In a protection-alkylation-deprotection sequence substituted diethylaminomalonates are obtained. Possible variations: R3=alkyl, allyl, benzyl, subst. alkyl 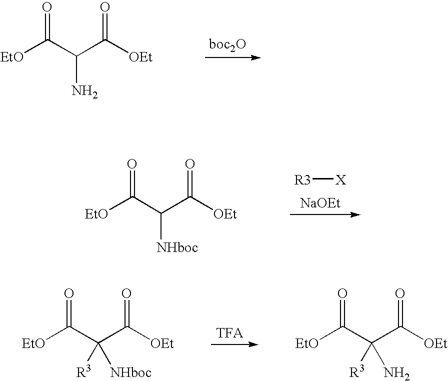
2

N-acylation proceeds under mild conditions. Possible variations: R1=no limitations 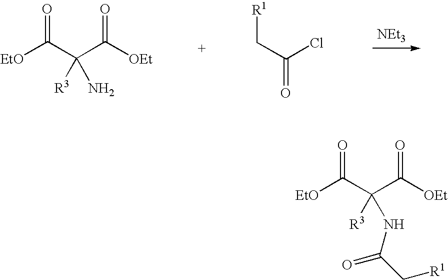
3

Base induzed cyclisation gives tetramic acids. 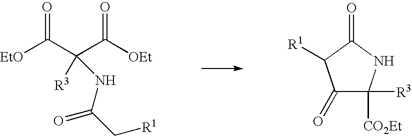
4

The next step is performed either by reduction or addition of a suitable organometallic reagent (e.g. Grignard).

Possible variations: R2=H, alkyl, benzyl, aryl 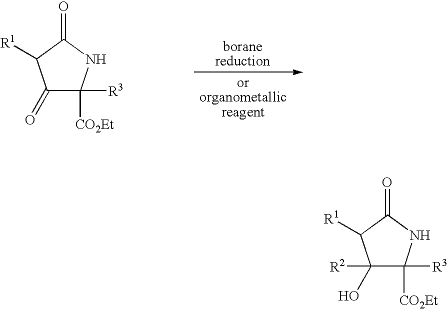
5

BOP-Cl is employed for lactonization.

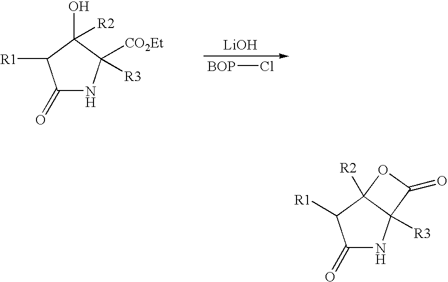

Possible transformations are:

introduction of R4=alkyl, benzyl, acyl, sulfonyl

introduction of R5=alkyl, benzyl, acyl, sulfonyl

transesterfication (R6=O-alkyl, O-allyl, O-benzyl)

saponification (R6=OH)

thioester formation (R6=S-alkyl)

or combinations thereof

Further, compounds of formula (II) can be prepared by

[A] a process for synthesizing the compounds of general formula

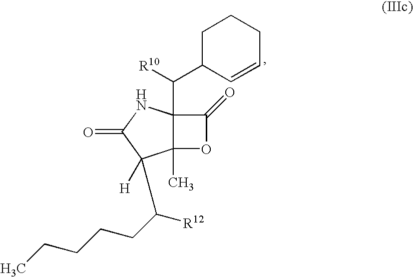

wherein

R¹⁰ and R¹² have the meaning described above,

characterized in that the compounds are prepared via fermentation and isolation from an Actinomycete of the genus Streptomyces,

or

[B] a process for synthesizing the compounds of general formula

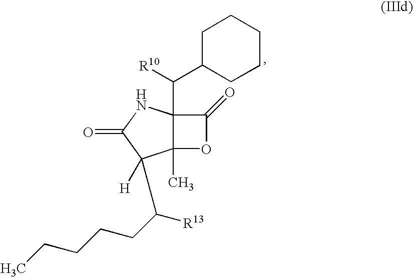

wherein

R¹⁰ and R¹³ have the meaning described above,

characterized in that the compounds are prepared via hydrogenation of the double bond in compounds of the formula (IIIc),

or

[C] a process for synthesizing the compounds of general formula

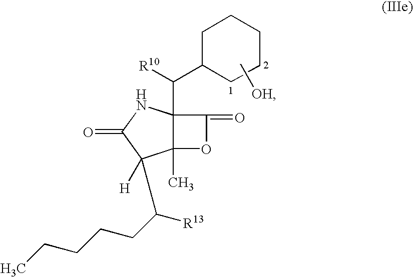

wherein

R¹⁰ and R¹³ have the meaning described above, and

the hydroxy-group is attached onto carbon atom 1 or 2,

characterized in that the compounds are prepared via hydration of the double bond in compounds of the formula (IIIc),

or

[D] a process for synthesizing the compounds of general formula

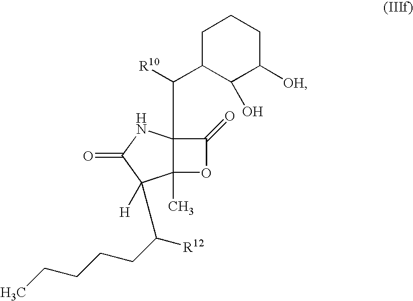

wherein

R¹⁰ and R¹² have the meaning described above,

characterized in that the compounds are prepared via oxidation of the double bond in compounds of the formula (IIIc),

or

[E] a process for synthesizing the compounds of general formula

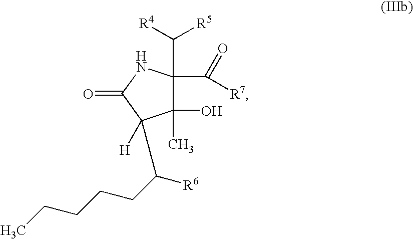

wherein

R¹³, R¹⁴, R¹⁵ and R¹⁶ have the meaning described above,

characterized in that the compounds are prepared via fermentation and isolation from an Actinomycete of the genus Streptomyces

Process [A] and [E] can be carried out as described in the experimental section.

The hydrogenation in process [B] can be carried out in the presence of an catalyst such as palladium/charcoal and hydrogen in a suitable solvent in a temperature range from 0° C. to +100° C., at normal pressure or at elevated pressure up to 3 bar.

Suitable solvents are i.e. ethers such as diethyl ether, methyl-t-butyl ether, dioxan or tetrahydro-furan, alcohols such as methanol, ethanol, n-propanol, iso-propanol, n-butanol or t-butanol, preferred is methanol, ethanol, iso-propanol or tetrahydrofuran.

The hydration in process [C] can be carried out by hydroboration with oxidative work-up using e.g. diborane (B₂H₆) in tetrahydrofuran followed by hydrogen peroxide. Alternatively an epoxide can be generated and opened by reduction methods. All processes can be carried out in a suitable solvent in a temperature range from −78° C. to +25° C., at normal pressure or at elevated pressure up to 3 bar.

Suitable solvents are i.e. tetrahydrofuran, diethyl ether, tert.-butyl-methyl ether, and related solvents.

The oxidation in process [D] can be carried out by chiral or achiral dihydroxylation methods using potassium permanganate (KMnO₄) or osmium tetroxide (OsO₄). In the case of osmium tetroxide, catalytical amounts may be sufficient, when tert. amine N-Oxides e.g. N-Methyl-morpholine-N-oxide or other oxidants like potassium ferricyanide (K₃FeCN₆) are used. All processes can be carried out in a suitable solvent in a temperature range from 0° C. to +100° C., at normal pressure or at elevated pressure up to 3 bar.

Suitable solvents are alcohols such as ethanol or t-butanol, with appropriate amounts of water added.

The compounds according to the present invention exhibit a strong microbicidal activity. Thus, they can be used for combating undesired microorganisms, such as phytopathogenic fungi and bacteriae, in agriculture and horticulture. The compounds are suitable for the direct control of undesired microorganisms as well as for generating resistance in plants against attack by undesired plant pathogens.

Resistance-inducing substances in the present context are to be understood as those substances which are capable of stimulating the defence system of plants such that the treated plants, when subsequently inoculated with undesirable microorganisms, display substantial resistance to these microorganisms.

Undesirable microorganisms in the present case are to be understood as phytopathogenic fungi and bacteriae. The substances according to the invention can thus be employed to generate resistance in plants against attack by the harmful organisms mentioned within a certain period of time after the treatment. The period of time within which resistance is brought about in general extends from 1 to 10 days, preferably 1 to 7 days, after treatment of the plants with the active compounds.

Generally, the compounds according to the invention can be used as fungicides for combating phytopathogenic fungi, such as Plasmodiophoromycetes, Oomycetes, Chytridiomycetes, Zygomycetes, Ascomycetes, Basidiomycetes and Deutero-mycetes, and can also be used as bactericides for combating bacteriae, such as Pseudomonadaceae, Rhizobiaceae, Enterobacteriaceae, Corynebacteriaceae, Streptomycetaceae, Proteobacteriae and Gram-positive groups.

Some pathogens causing fungal diseases which come under the generic names listed above are mentioned as examples, but not by way of limitation:

Erwinia species, such as, for example, Erwinia amylovora;

Pythium species, such as, for example, Pythium ultimum;

Phytophthora species, such as, for example, Phytophthora infestans;

Pseudoperonospora species, such as, for example, Pseudoperonospora humuli or Pseudoperonospora cubensis;

Plasmopara species, such as, for example, Plasmopara viticola;

Bremia species, such as, for example, Bremia Lactucae;

Peronospora species, such as, for example, Peronospora pisi or P. brassicae;

Erysiphe species, such as, for example, Erysiphe graminis;

Sphaerotheca species, such as, for example, Sphaerotheca fuliginea;

Podosphaera species, such as, for example, Podosphaera leucotricha;

Venturia species, such as, for example, Venturi inaequalis;

Pyrenophora species, such as, for example, Pyrenophora teres or P. graminea (conidia form: Drechslera, syn: Helminthosporium);

Cochliobolus species, such as, for example, Cochliobolus sativus (conidia form: Drechslera,syn: Helminthosporium);

Uromyces species, such as, for example, Uromyces appendiculatus;

Puccinia species, such as, for example, Puccinia recondita;

Sclerotinia species, such as, for example, Sclerotinia sclerotiorum;

Tilletia species, such as, for example, Tilletia caries;

Ustilago species, such as, for example, Ustilago nuda or Ustilago avenae;

Pellicularia species, such as, for example, Pellicularia sasakii;

Pyricularia species, such as, for example, Pyricularia oryzae;

Fusarium species, such as, for example, Fusarium culmorum;

Botrytis species, such as, for example, Botrytis cinerea;

Septoria species, such as, for example, Leptosphaeria nodorum;

Cercospora species, such as, for example, Cercospora canescens;

Alternaria species, such as, for example, Alternaria brassicae;

Pseudocercosporella species, such as, for example, Pseudocercosporella herpotrichoides; and

Phakopsora species, such as, for example Phakopsora pachyrhizi and Phakopsora meibomiae.

The compounds according to the present invention are particularly suitable for causing resistance against infection of plants by plant pathogens, such as Pyricularia oryzae, Phythophthora infestans etc.

The good toleration, by plants, of the active compounds, at the concentrations required for combating plants diseases, permits treatment of above-ground parts of plants, of vegetative propagation stock and seeds, and of the soil.

The compounds according to the present invention have a low toxicity against warm-blooded animals and therefore can be used safely.

The active compounds can be converted into the customary formulations, such as solutions, emulsions, wettable powders, suspensions, powders, foams, pastes, granules, tablets, aerosols, natural and synthetic materials impregnated with active compound, very fine capsules in polymeric substances, coating compositions for use on seed, and formulations used with burning equipment, such as fumigating cartridges, fumigating cans and fumigating coils, as well as ULV cold mist and warm mist formulations.

These formulations may be produced in known manner, for example by mixing the active compounds with extenders, that is to say liquid or liquefied gaseous or solid diluents or carriers, optionally with the use of surface-active agents, that is to say emulsifying agents and/or dispersing agents and/or foam-forming agents. In the case of the use of water as an extender, organic solvents can, for example, also be used as auxiliary solvents.

As liquid solvents diluents or carriers, there are suitable in the main, aromatic hydrocarbons such as xylene, toluene or alkyl naphthalenes, chlorinated aromatic or chlorinated aliphatic hydrocarbons, such as chlorobenzenes, chloroethylenes or methylene chloride, aliphatic hydrocarbons, such as cyclohexane or paraffins, for example mineral oil fractions, alcohols, such as butanol or glycol as well as their ethers and esters, ketones, such as acetone, methyl ethyl ketone, methyl-isobutyl ketone or cyclohexanone, or strongly polar solvents, such as dimethylformamide and dimethyl-sulphoxide, as well as water.

By liquefied gaseous diluents or carriers are meant liquids which would be gaseous at normal temperature and under normal pressure, for example aerosol propellants, such as halogenated hydrocarbons as well as butane, propane, nitrogen and carbon dioxide.

As solid carriers there may be used ground natural minerals, such as kaolings, clays, talc, chalk, quartz, attapulgite, montmorillonite or diatomaceous earth, and ground synthetic minerals, such as highly-dispersed silicic acid, alumina and silicates. As solid carriers for granules there may be used crushed and fractionated natural rocks such as calcite, marble, pumice, sepiolite and dolomite, as well as synthetic granules of inorganic and organic meals, and granules of organic material such as sawdust, coconut shells, maize cobs and tobacco stalks.

As emulsifying and/or foam-forming agents there may be used non-ionic and anionic emulsifiers, such as polyoxyethylene-fatty acid esters, polyoxyethylene-fatty alcohol ethers, for example alkylaryl polyglycol ethers, alkyl sulphonates, alkyl sulphates, aryl sulphonates as well as albumin hydrolysis products.

Dispersing agents include, for example, lignin sulphite waste liquors and methylcellulose.

Adhesives such as carboxymethylcellulose and natural and synthetic polymers in the form of powders, granules or latices, such as gum arabic, polyvinyl alcohol and polyvinyl acetate, can be used in the formulation.

It is possible to use colorants such as inorganic pigments, for example iron oxide, titanium oxide and Prussian Blue, and organic dyestuffs, such as alizarin dyestuffs, azo dyestuffs or metal phthalocyanine dyestuffs, and trace nutrients, such as salts of iron, manganese, boron, copper, cobalt, molybdenum and zinc.

The formulations in general contain from 0.1 to 95 per cent by weight of active compound, preferably from 0.5 to 90 per cent by weight.

The active compounds according to the invention can be present in the formulations or in the various use forms as a mixture with other known active compounds, such as fungicides, bactericides, insecticides, acaricides, nematicides, herbicides, bird repellents, growth factors, plant nutrients and agents for improving soil structure.

In many cases, synergistic effects are achieved, i.e. the activity of the mixture exceeds the activity of the individual components.

Examples of co-components in mixtures are the following compounds:

Fungicides:

1. Inhibition of Nucleic Acid Synthesis

1.1 benalaxyl, benalaxyl-M, bupirimate, chiralaxyl, clozylacon, dimethirimol, ethirimol, furalaxyl, hymexazol, metalaxyl-M, ofurace, oxadixyl, oxolinic acid

2. Inhibition of Mitosis and Cell Division:

2.1 benomyl, carbendazim, diethofencarb, fuberidazole, pencycuron, thiabendazole thiophanate-methyl, zoxamide

3. Inhibition of Respiration

3.1 CI: diflumetorim

3.2 CII: boscalid, carboxin, fenfuram, flutolanil, furametpyr, mepronil, oxycarboxine, penthiopyrad, thifluzamide

3.3 CII: azoxystrobin, cyazofamid, dimoxystrobin, enestrobin, famoxadone, fenamidone, fluoxastrobin, kresoxim-methyl, metominostrobin, orysastrobin, pyraclostrobin, picoxystrobin, trifloxystrobin,

3.4 Uncouplers: dinocap, fluazinam

3.5 Inhibition of ATP production: fentin acetate, fentin chloride, fentin hydroxide, silthiofam

4. Inhibition of AA and Protein Biosynthesis

4.1 andoprim, blasticidin-S, cyprodinil, kasugamycin, kasugamycin hydrochloride hydrate, mepanipyrim, pyrimethanil,

5. Inhibition of Signal Transduction

5.1 fenpiclonil, fludioxonil, quinoxyfen

6. Inhibition of Lipids and Membranes Synthesis

6.1 chlozolinate, iprodione, procymidone, vinclozolin

6.2 pyrazophos, edifenphos, iprobenfos (IBP), isoprothiolane

6.3 tolclofos-methyl, biphenyl

6.4 iodocarb, propamocarb, propamocarb hydrochloride

7. Inhibition of Ergosterol Biosynthesis

7.1 fenhexamid,

7.2 azaconazole, bitertanol, bromuconazole, cyproconazole, diclobutrazole, difenoconazole, diniconazole, diniconazole-M, epoxiconazole, etaconazole, fenbuconazole, fluquinconazole, flusilazole, flutriafol, furconazole, furconazole-cis, hexaconazole, imibenconazole, ipconazole, metconazole, myclobutanil, paclobutrazol, penconazole, propiconazole, prothioconazole, simeconazole, tebuconazole, tetraconazole, triadimefon, triadimenol, triticonazole, uniconazole, voriconazole, imazalil, imazalil sulfate, oxpoconazole, fenarimol, flurprimidol, nuarimol, pyrifenox, triforine, pefurazoate, prochloraz, triflumizole, viniconazole,

7.3 aldimorph, dodemorph, dodemorph acetate, fenpropimorph, tridemorph, fenpropidin, spiroxamine

7.4 naftifine, pyributicarb, terbinafine,

8. Inhibition of Cell Wall Synthesis

8.1 benthiavalicarb, bialaphos, dimethomorph, flumorph, iprovalicarb, polyoxins, polyoxorim, validamycin A

9. Inhibition of Melanine Biosynthesis

9.1 carpropamid, diclocymet, fenoxanil, phtalide, pyroquilon, tricyclazole,

10. Host Defence Inducer

10.1 acibenzolar-S-methyl, probenazole, tiadinil

11. Multisite

11.1 captafol, captan, chlorothalonil, copper preparations such as: copper hydroxide, copper naphthenate, copper oxychloride, copper sulphate, copper oxide, oxine-copper and Bordeaux mixture, dichlofluanid, dithianon, dodine, dodine free base, ferbam, fluorofolpet, folpet, guazatine, guazatien acetate, iminoctadine, iminoctadine albesilate, iminoctadine triacetate, mancopper, mancozeb, maneb, metiram, metiram zinc, propineb, sulphur and sulphur preparations including calcium polysulphide, thiram, tolylfluanid, zineb, ziram,

12. Unknown

12.1 amibromdole, benthiazole, bethoxazin, capsimycin, carvone, chinomethionat, chloropicrin, cufraneb, cyflufenamid, cymoxanil, dazomet, debacarb, diclomezine, dichlorophen, dicloran, difenzoquat, difenzoquat methylsulphate, diphenylamine, ethaboxam, ferimzone, flumetover, flusulfamide, fosetyl-aluminium, fosetyl-calcium, fosetyl-sodium, fluopicolide, fluoroimide, hexachlorobenzene, 8-hydroxyquinoline sulfate, irumamycin, methasulphocarb, metrafenone, methyl isothiocyanate, mildiomycin, natamycin, nickel dimethyldithiocarbamate, nitrothal-isopropyl, octhilinone, oxamocarb, oxyfenthiin, pentachlorophenol and salts, 2-phenylphenol and salts, phosphorous acid and its salts, piperalin, propanosine-sodium, proquinazid, pyrrolnitrine, quintozene, tecloftalam, tecnazene, triazoxide, trichlamide, zarilamid and 2,3,5,6-tetrachloro-4-(methylsulfonyl)-pyridine, N-(4-Chloro-2-nitrophenyl)-N-ethyl-4-methyl-benzenesulfonamide, 2-amino-4-methyl-N-phenyl-5-thiazolecarboxamide, 2-chloro-N-(2,3-dihydro-1,1,3-trimethyl-1H-inden-4-yl)-3-pyridincarboxamide, 3-[5-(4-chlorophenyl)-2,3-dimethylisoxazolidin-3-yl]pyridine, cis-1-(4-chlorophenyl)-2-(1H-1,2,4-triazole-1-yl)-cycloheptanol, methyl 1-(2,3-dihydro-2,2-dimethyl-1H-inden-1-yl)-1H-imidazole-5-carboxylate, 3,4,5-trichloro-2,6-pyridinedicarbonitrile, Methyl 2-[[[cyclopropyl[(4-methoxyphenyl)imino]methyl]thio]methyl]-.alpha.-(methoxy-methylene)-benzeneacetate, 4-Chloro-alpha-propynyloxy-N-[2-[3-methoxy-4-(2-propynyloxy)phenyl]ethyl]-benzeneacetamide, (2S)-N-[2-[4-[[3-(4-chlorophenyl)-2-propynyl]oxy]-3-methoxyphenyl]ethyl]-3-methyl-2-[(methylsulfonyl)amino]-butanamide, 5-chloro-7-(4-methylpiperidin-1-yl)-6-(2,4,6-trifluorophenyl)[1,2,4]triazolo[1,5-a]pyrimidine, 5-chloro-6-(2,4,6-trifluorophenyl)-N-[(1R)-1,2,2-trimethylpropyl][1,2,4]triazolo[1,5-a]pyrimidin-7-amine, 5-chloro-N-[(1R)-1,2-dimethylpropyl]-6-(2,4,6-trifluorophenyl)[1,2,4]triazolo[1,5-a]pyrimidin-7-amine, N-[1-(5-bromo-3-chloropyridin-2-yl)ethyl]-2,4-dichloronicotinamide, N-(5-bromo-3-chloropyridin-2-yl)methyl-2,4-dichloronicotinamide, 2-butoxy-6-iodo-3-propyl-benzopyranon-4-one, N-{(Z)-[(cyclopropylmethoxy)imino][6-(difluoromethoxy)-2,3-difluorophenyl]methyl}-2-phenyl-acetamide, N-(3-ethyl-3,5,5-trimethyl-cyclohexyl)-3-formylamino-2-hydroxy-benzamide, 2-[[[[1-[3(1Fluoro-2-phenylethyl)oxy]phenyl]ethylidene]amino]oxy]methyl]-alpha-(methoxyimino)-N-methyl-alphaE-benzeneacetamide, N-{2-[3-chloro-5-(trifluoromethyl)pyridin-2-yl]ethyl}-2-(trifluoromethyl)benzamide, N-(3′,4′-dichloro-5-fluorobiphenyl-2-yl)-3-(difluoromethyl)-1-methyl-1H-pyrazole-4-carboxamide, 1-[(4-methoxyphenoxy)methyl]-2,2-dimethylpropyl-1H-imidazole-1-carboxylic acid, O-[1-[(4-methoxyphenoxy)methyl]-2,2-dimethylpropyl]-1H-imidazole-1-carbothioic acid, 2-(2-{[6-(3-chloro-2-methylphenoxy)-5-fluoropyrimidin-4-yl]oxy}phenyl)-2-(methoxyimino)-N-methylacetamide

Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.

Insecticides/Acaricides/Nematicides:

1. Acetylcholinesterase (AChE) Inhibitors

1.1 carbamates (for example alanycarb, aldicarb, aldoxycarb, allyxycarb, aminocarb, azamethiphos, bendiocarb, benfuracarb, bufencarb, butacarb, butocarboxim, butoxycarboxim, carbaryl, carbofuran, carbosulfan, chloethocarb, coumaphos, cyanofenphos, cyanophos, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, triazamate, trimethacarb, XMC, xylylcarb)

1.2 organophosphates (for example acephate, azamethiphos, azinphos (-methyl, -ethyl), bromophos-ethyl, bromfenvinfos(-methyl), butathiofos, cadusafos, carbophenothion, chloroethoxyfos, chlorofenvinphos, chloromephos, chloropyrifos (-methyl/-ethyl), coumaphos, cyanofenphos, cyanophos, chlorofenvinphos, demeton-s-methyl, demeton-s-methylsulphon, dialifos, diazinon, dichlofenthion, dichlorovos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl o-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorovinphos, thiometon, triazophos, triclorfon, vamidothion)

2. Sodium Channel Modulators/Blockers of Voltage-Dependent Sodium Channels

2.1 pyrethroids (for example acrinathrin, allethrin (d-cis-trans, d-trans), beta-cyfluthrin, bifenthrin, bioallethrin, bioallethrin-S-cyclopentyl-isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, DDT, deltamethrin, empenthrin (1R-isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R-trans isomer), prallethrin, profluthrin, protrifenbute, pyresmethrin, resmethrin, RU 15525, silafluofen, tau-fluvalinate, tefluthrin, terallethrin, tetramethrin (1R-isomer), tralomethrin, transfluthrin, ZXI 8901, pyrethrins (pyrethrum))

2.2 Oxadiazines (for Example indoxacarb)

3. Acetylcholine Receptor Agonists/Antagonists

3.1 chloronicotinyls/neonicotinoids (for example acetamiprid, clothianidin, dinotefuran, imidacloprid, nitenpyram, nithiazine, thiacloprid, thiamethoxam)

3.2 nicotine, bensultap, cartap

4. Acetylcholine Receptor Modulators

4.1 spinosyns (for example spinosad)

5. Antagonists of GABA-Controlled Chloride Channels

5.1 cyclodiene organochlorines (for example camphechloro, chlorodane, endosulfan, gamma-HCH, HCH, heptachloro, lindane, methoxychloro

5.2 fiproles (for example acetoprole, ethiprole, fipronil, vaniliprole)

6. Chloride Channel Activators

6.1 mectins (for example abamectin, avermectin, emamectin, emamectin-benzoate, ivermectin, milbemectin, milbemycin)

7. Juvenile Hormone Mimetics

(for example diofenolan, epofenonane, fenoxycarb, hydroprene, kinoprene, methoprene, pyriproxifen, triprene)

8. Ecdyson Agonists/Disruptors

8.1 diacylhydrazines (for example chromafenozide, halofenozide, methoxyfenozide, tebufenozide)

9. Chitin Biosynthesis Inhibitors

9.1 benzoylureas (for example bistrifluron, chlofluazuron, diflubenzuron, fluazuron, flucycloxuron, flufenoxuron, hexaflumuron, lufenuron, novaluron, noviflumuron, penfluron, teflubenzuron, triflumuron)

9.2 buprofezin

9.3 cyromazine

10. Inhibitors of Oxidative Phosphorylation, ATP Disruptors

10.1 Diafenthiuron

10.2 organotins (for example azocyclotin, cyhexatin, fenbutatin-oxide)

11. Decouplers of Oxidative Phosphorylation Acting by Interrupting the H-proton Gradient

11.1 pyrroles (for example chlorofenapyr)

11.2 dinitrophenols (for example binapacryl, dinobuton, dinocap, DNOC)

12. Site-I Electron Transport Inhibitors

12.1 METIs (for example fenazaquin, fenpyroximate, pyrimidifen, pyridaben, tebufenpyrad, tolfenpyrad)

12.2 hydramethylnone

12.3 dicofol

13. Site-II Electron Transport Inhibitors

13.1 rotenone

14. Site-III Electron Transport Inhibitors

14.1 acequinocyl, fluacrypyrim

15. Microbial Disruptors of the Insect Gut Membrane

Bacillus thuringiensis strains

16. Inhibitors of Fat Synthesis

16.1 tetronic acids (for example spirodiclofen, spiromesifen) 16.2 tetramic acids [for example 3-(2,5-dimethylphenyl)-8-methoxy-2-oxo-1-azaspiro[4.5]dec-3-en-4-yl ethyl carbonate (alias: carbonic acid, 3-(2,5-dimethylphenyl)-8-methoxy-2-oxo-1-azaspiro[4.5]dec-3-en-4-yl ethyl ester, CAS Reg. No.: 382608-10-8) and carbonic acid, cis-3-(2,5-dimethylphenyl)-8-methoxy-2-oxo-1-azaspiro[4.5]dec-3-en-4-yl ethyl ester (CAS Reg. No.: 203313-25-1)]

17. Carboxamides

(for example flonicamid)

18. Octopaminergic Agonists

(for example amitraz)

19. Inhibitors of Magnesium-Stimulated ATPase

(for example propargite)

20. Phthalamides

(for example N²-[1,1-dimethyl-2-(methylsulphonyl)ethyl]-3-iodo-N′-[2-methyl-4-[1,2,2,2-tetrafluoro-1-(trifluoromethyl)ethyl]phenyl]-1,2-benzenedicarboxamide (CAS Reg. No.: 272451-65-7), flubendiamide)

21. Nereistoxin Analogues

(for example thiocyclam hydrogen oxalate, thiosultap-sodium)

22. Biologicals, Hormones or Pheromones

(for example azadirachtin, Bacillus spec., Beauveria spec., Codlemone, Metarrhizium spec., Paecilomyces spec., Thuringiensin, Verticillium spec.)

23. Active Compounds with Unknown or Unspecific Mechanisms of Action

23.1 fumigants (for example aluminium phosphide, methyl bromide, sulphuryl fluoroide)

23.2 selective antifeedants (for example cryolite, flonicamid, pymetrozine)

23.3 mite growth inhibitors (for example clofentezine, etoxazole, hexythiazox)

23.4 amidoflumet, benclothiaz, benzoximate, bifenazate, bromopropylate, buprofezin, chinomethionat, chlorodimeform, chlorobenzilate, chloropicrin, clothiazoben, cycloprene, cyflumetofen, dicyclanil, fenoxacrim, fentrifanil, flubenzimine, flufenerim, flutenzin, gossyplure, hydramethylnone, japonilure, metoxadiazone, petroleum, piperonyl butoxide, potassium oleate, pyrafluprole, pyridalyl, pyriprole, sulfluramid, tetradifon, tetrasul, triarathene, verbutin,

furthermore the compound 3-methylphenyl propylcarbamate (Tsumacide Z), the compound 3-(5-chloro-3-pyridinyl)-8-(2,2,2-trifluoroethyl)-8-azabicyclo[3.2.1]octane-3-carbonitrile (CAS Reg. No. 185982-80-3) and the corresponding 3-endo-isomer (CAS Reg. No. 185984-60-5) (cf. WO 96/37494, WO 98/25923), and preparations which comprise insecticidally active plant extracts, nematodes, fungi or viruses.

A mixture with other known active compounds, such as herbicides, or with fertilizers and growth regulators, safeners and/or semiochemicals is also possible.

The active compounds can be used as such or in the form of their formulations or the use forms prepared therefrom by further dilution, such as ready-to-use solutions, emulsions, suspensions, powders, tablets, pastes, microcapsules and granules. They are used in the customary manner, for example by watering, immersion, spraying, atomising, misting, vaporizing, injecting, forming a slurry, brushing on, dusting, scattering, dry dressing, moist dressing, wet dressing, slurry dressing or encrusting.

In the treatment of parts of plants, the active compounds concentration in the use forms can be varied within a substantial range. They are, in general, from 1 to 0.0001% by weight, preferably from 0.5 and 0.001%.

For the treatment of seed, amounts of active compound of 0.1 to 10 g, especially 1 to 5 g, are generally employed per 1 kilogram of seed.

For the treatment of soil, active compound concentrations, at the point of action, of 0.00001 to 0.1% by weight, especially of 0.0001 to 0.02%, are generally employed.

As already mentioned above, all plants and parts of plants can be treated according to the invention. In a preferred embodiment naturally occurring plant species and plant varieties or those obtained by conventional biological breeding methods, such as crossbreeding or protoplast fusion as well as parts of such plants are treated. In an additional preferred embodiment transgenic plants and plant varieties which have been obtained by genetic engineering methods, possibly in combination with conventional methods (genetically modified organisms) and parts of such plants are treated. The term “parts” or “parts of plants” or “plant parts” is explained above.

According to the invention plants of the plant varieties commercially available or used at any particular time are very preferably treated. Plant varieties are understood to be plants with specific properties (“traits”) which have been obtained both by conventional breeding, by mutagenesis or by recombinant DNA techniques. They can be varieties, biotypes or genotypes.

Depending on the species or varieties of plants, their location and growth conditions (the types of soil, climate, vegetation period and feed concerned), superadditive (“synergistic”) effects can occur as a result of the treatment according to the invention. Effects such as for example reduced application rates and/or broadening of the activity spectra and/or increased activity of the compounds and compositions usable according to the invention, improved plant growth, increased tolerance of high or low temperatures, increased tolerance of dry conditions or water or ground salt contents, increased flowering capacity, facilitated harvesting, acceleration of maturity, increased crop yields, higher quality and/or increased nutritional value of the harvested crops and increased storing quality and/or processibility of the harvested crops are possible, which are greater than those actually expected.

Preferred transgenic plants or plant varieties (obtained by genetic engineering) to be treated according to the invention include all plants which as a result of the genetic modification concerned have received genetic material which provides them with particularly advantageous valuable properties (“traits”). Examples of such properties are improved plant growth, increased tolerance of high or low temperatures, increased tolerance of dry conditions or water or ground salt contents, increased flowering capacity, facilitated harvesting, acceleration of maturity, increased crop yields, higher quality and/or increased nutritional value of the harvested crops and increased storing quality and/or processibility of the harvested crops. Additional and particularly noteworthy examples of such properties are increased resistance of the plants to animal and microbial pests, such as to insects, mites, phytopathogenic fungi, bacteria and/or viruses as well as increased tolerance by the plants of certain herbicidal active compounds. Examples which may be mentioned of transgenic plants are the important crop plants such as cereals (wheat and rice), corn, soybeans, potatoes, cotton, rape and fruit plants (producing apples, pears, citrus fruits and grapes), the crop plants corn, soybeans, potatoes, cotton and rape being particularly noteworthy. Particularly significant properties (“traits”) are increased resistance of the plants to insects due to the toxins forming in the plants, and in particular those which are produced in the plants (hereinafter referred to as “Bt plants”) by the genetic material obtained from Bacillus Thuringiensis (e.g. by the genes CrylA(a), CrylA(b), CrylA(c), CryllA, CryIIIB2, Cry9c Cry2Ab, Cry3Bb and CryIF and combinations thereof). Particularly significant properties (“traits”) are the increased resistance of plants to fungi, bacteria and viruses due to systemically acquired resistance (SAR), systemin, phytoalexins, elicitors and resistance genes and correspondingly expressed proteins and toxins. Particulary significant properties (“traits”) are also increased tolerance by the plants of certain herbicidal active compounds, such as for example imidazolinones, sulphonylureas, glyphosate or phosphinotricine (e.g. the “PAT” gene). The corresponding genes imparting the required properties (“traits”) can also occur in the transgenic plants in combination with each other. Examples which may be mentioned of “Bt plants” are varieties of corn, cotton, soybeans and potatoes which are sold under the trade names YIELD GARD® (e.g. corn, cotton, soybeans), KnockOut® (e.g. corn), StarLink® (e.g. corn), Bollgard® (cotton), Nucotn® (cotton) and NewLeaf® (potatoes). Examples which may be mentioned of herbicide-tolerant plants are varieties of corn, cotton and soybeans which are sold under the trade names Roundup Ready® (tolerance of glyphosate, e.g. corn, cotton, soybeans), Liberty Link® (tolerance of phosphinotricine, e.g. rape), IMI® (tolerance of imidazolinones) and STS® (tolerance of sulphonylureas, e.g. corn). Herbicide-resistant plants (bred for herbicide tolerance in the conventional manner) which may be mentioned are also the varieties (e.g. corn) sold under the name Clearfield®. The above statements do of course also apply to any plant varieties which may be developed in the future or launched onto the market in the future and which have the genetic properties (“traits”) described above or developed in the future.

According to the invention the above-mentioned plants can be particularly advantageously treated with the compounds of the general formula I or the active compound mixtures according to the invention. The preferred ranges mentioned above for the active compounds or mixtures also apply to the treatment of these plants. Particularly advantageous is the treatment of plants with the compounds or mixtures specifically listed in the present text.

The active compounds are further suitable for controlling animal pests. Harmful animals as used herein means harmful arthropods and helminths, in particular insects, arachnids and nematodes, which are encountered in agriculture, in forestry, in the protection of stored products and of materials, and in the hygiene sector, and have good plant tolerance and favourable toxicity to warm-blooded animals. They may be preferably employed as plant protection agents. They are active against normally sensitive and resistant species and against all or some stages of development. The abovementioned pests include:

From the order of the Isopoda, for example, Oniscus asellus, Armadillidium vulgare and Porcellio scaber.

From the order of the Diplopoda, for example, Blaniulus guttulatus.

From the order of the Chilopoda, for example, Geophilus carpophagus and Scutigera spp.

From the order of the Symphyla, for example, Scutigerella immaculata.

From the order of the Thysanura, for example, Lepisma saccharina.

From the order of the Collembola, for example, Onychiurus armatus.

From the order of the Orthoptera, for example, Acheta domesticus, Gryllotalpa spp., Locusta migratoria migratorioides, Melanoplus spp. and Schistocerca gregaria.

From the order of the Blattaria, for example, Blatta orientalis, Periplaneta americana, Leucophaea maderae, Blattella germanica.

From the order of the Dermaptera, for example, Forficula auricularia.

From the order of the Isoptera, for example, Reticulitermes spp.

From the order of the Phthiraptera, for example, Pediculus humanus corporis, Haematopinus spp., Linognathus spp., Trichodectes spp. and Damalinia spp.

From the order of the Thysanoptera, for example, Hercinothrips femoralis, Thrips tabaci, Thrips palmi and Frankliniella accidentalis.

From the order of the Heteroptera, for example, Eurygaster spp., Dysdercus intermedius, Piesma quadrata, Cimex lectularius, Rhodnius prolixus and Triatoma spp.

From the order of the Homoptera, for example, Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium corni, Saissetia oleae, Laodelphax striatellus, Nilaparvata lugens, Aonidiella aurantii, Aspidiotus hederae, Pseudococcus spp. and Psylla spp.

From the order of the Lepidoptera, for example, Pectinophora gossypiella, Bupalus piniarius, Cheimatobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofmannophila pseudospretella, Cacoecia podana, Capua reticulana, Choristoneura fumiferana, Clysia ambiguella, Homona magnanima, Tortrix viridana, Cnaphalocerus spp., Oulema oryzae.

From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica and Lissorhoptrus oryzophilus.

From the order of the Hymenoptera, for example, Diprion spp., Hoplocampa spp., Lasius spp., Monomorium pharaonis and Vespa spp.

From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, Hylemyia spp. and Liriomyza spp.

From the order of the Siphonaptera, for example, Xenopsylla cheopis and Ceratophyllus spp.

From the class of the Arachnida, for example, Scorpio maurus, Latrodectus mactans, Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Hemitarsonemus spp., Brevipalpus spp.

The phytoparasitic nematodes include, for example, Pratylenchus spp., Radopholus similis, Ditylenchus dipsaci, Tylenchulus semipenetrans, Heterodera spp., Globodera spp., Meloidogyne spp., Aphelenchoides spp., Longidorus spp., Xiphinema spp., Trichodorus spp., Bursaphelenchus spp.

If appropriate, the compounds according to the invention can, at certain concentrations or application rates, also be used as herbicides or microbicides, for example as fungicides, antimycotics and bactericides. If appropriate, they can also be employed as intermediates or precursors for the synthesis of other active compounds.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and recombinant methods or by combinations of these methods, including the transgenic plants and inclusive of the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offsets and seeds.

Treatment according to the invention of the plants and plant parts with the active compounds is carried out directly or by allowing the compounds to act on the surroundings, environment or storage space by the customary treatment methods, for example by immersion, spraying, evaporation, fogging, scattering, painting on and, in the case of propagation material, in particular in the case of seeds, also by applying one or more coats.

The active compounds can be converted to the customary formulations, such as solutions, emulsions, wettable powders, suspensions, powders, dusts, pastes, soluble powders, granules, suspension-emulsion concentrates, natural and synthetic materials impregnated with active compound and microencapsulations in polymeric substances.

These formulations are produced in a known manner, for example by mixing the active compounds with extenders, that is liquid solvents, and/or solid carriers, optionally with the use of surfactants, that is emulsifiers and/or dispersants, and/or foam-formers.

If the extender used is water, it is also possible to employ for example organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethylformamide and dimethyl sulphoxide, and also water.

As solid carriers there are suitable:

for example ammonium salts and ground natural minerals such as kaolins, clays, talc, chalk, quartz, attapulgite, montmorillonite or diatomaceous earth, and ground synthetic minerals, such as highly disperse silica, alumina and silicates; as solid carriers for granules there are suitable: for example crushed and fractionated natural rocks such as calcite, marble, pumice, sepiolite and dolomite, and also synthetic granules of inorganic and organic meals, and granules of organic material such as sawdust, coconut shells, maize cobs and tobacco stalks; as emulsifiers and/or foam-formers there are suitable: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and also protein hydrolysates; as dispersants there are suitable: for example lignin-sulphite waste liquors and methylcellulose.

Tackifiers such as carboxymethylcellulose and natural and synthetic polymers in the form of powders, granules or latices, such as gum arabic, polyvinyl alcohol and polyvinyl acetate, as well as natural phospholipids such as cephalins and lecithins, and synthetic phospholipids, can be used in the formulations. Other possible additives are mineral and vegetable oils.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95% by weight of active compound, preferably between 0.5 and 90%.

The active compounds according to the invention can be employed as such or in their formulations as a mixture with known fungicides, bactericides, acaricides, nematicides or insecticides in order to increase the activity spectrum or avoid the development of resistance. In many cases synergistic effects are achieved, ie. the efficacy of the mixture is greater than the efficacy of the individual components.

Favourable examples of co-components in mixtures are the compounds listed above as possible mixing partners in case of a fungicidal use of the compounds of formula (I).

It is also possible to admix other known active compounds, such as herbicides, fertilizers and growth regulators.

When used as insecticides, the active compounds according to the invention can furthermore be present in their commercially available formulations and in the use forms, prepared from these formulations, as a mixture with synergistic agents. Synergistic agents are compounds which increase the action of the active compounds, without it being necessary for the synergistic agent added to be active itself.

The active compound content of the use forms prepared from the commercially available formulations can vary within wide limits. The active compound concentration of the use forms can be from 0.0000001 to 95% by weight of active compound, preferably between 0.0001 and 1% by weight.

The compounds are employed in a customary manner appropriate for the use forms.

When used against hygiene pests and pests of stored products, the active compound is distinguished by an excellent residual action on wood and clay as well as by a good stability to alkali on limed substrates.

As already mentioned above, it is possible to treat all plants and their parts according to the invention. In a preferred embodiment, wild plant species and plant cultivars, or those obtained by conventional biological breeding, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof are treated. The term “parts” or “parts of plants” or “plant parts” has been explained above.

Particularly preferably, plants of the plant cultivars which are in each case commercially available or in use are treated according to the invention. Plant cultivars are understood as meaning plants with novel properties (“traits”) which are grown by conventional cultivation, by mutagenesis or by recombinant DNA techniques. These may be cultivars, biotypes or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or widenings of the activity spectrum and/or an increase in the activity of the substances and compositions to be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or plant cultivars (i.e. those obtained by genetic engineering) which are preferably to be treated according to the invention include all plants which, in the genetic modification, received genetic material which imparted particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, oilseed rape and also fruit plants (with the fruits apples, pears, citrus fruits and grapes), and particular emphasis is given to maize, soya beans, potatoes, cotton and oilseed rape. Traits that are emphasized are in particular increased defence of the plants against insects by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (hereinbelow referred to as “Bt plants”). Traits which are also particularly emphasized are the increased resistance of plants to fungi, bacteria and viruses by systemic acquired resistance (SAR), systemin, phytoalexins, elicitors and resistance genes and the correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucotn® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these genetic traits or genetic traits still to be developed, which cultivars will be developed and/or marketed in the future.

The plants listed can be treated according to the invention in a particularly advantageous manner with the compounds of the formula I or the active compound mixtures according to the invention. The preferred ranges stated above for the active compounds or mixtures also apply to the treatment of these plants. Particular emphasis is given to the treatment of plants with the compounds or mixtures specifically mentioned in the present text.

The active compounds according to the invention act not only against plant, hygiene and stored product pests, but also in the veterinary medicine sector against animal parasites (ectoparasites), such as hard ticks, soft ticks, mange mites, leaf mites, flies (biting and licking), parasitic fly larvae, lice, hair lice, feather lice and fleas. These parasites include:

From the order of the Anoplurida, for example, Haematopinus spp., Linognathus spp., Pediculus spp., Phtirus spp. and Solenopotes spp.

From the order of the Mallophagida and the suborders Amblycerina and Ischnocerina, for example, Trimenopon spp., Menopon spp., Trinoton spp., Bovicola spp., Werneckiella spp., Lepikentron spp., Damalina spp., Trichodectes spp. and Felicola spp.

From the order of the Diptera and the suborders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

From the order of the Siphonapterida, for example Pulex spp., Ctenocephalides spp., Xenopsylla spp. and Ceratophyllus spp.

From the order of the Heteropterida, for example, Cimex spp., Triatoma spp., Rhodnius spp. and Panstrongylus spp.

From the order of the Blattarida, for example Blatta orientalis, Periplaneta americana, Blattela germanica and Supella spp.

From the subclass of the Acaria (Acarida) and the orders of the Meta- and Mesostigmata, for example, Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.

From the order of the Actinedida (Prostigmata) und Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The active compounds of the formula (I) according to the invention are also suitable for controlling arthropods which infest agricultural productive livestock, such as, for example, cattle, sheep, goats, horses, pigs, donkeys, camels, buffalo, rabbits, chickens, turkeys, ducks, geese and bees, other pets, such as, for example, dogs, cats, caged birds and aquarium fish, and also so-called test animals, such as, for example, hamsters, guinea pigs, rats and mice. By controlling these arthropods, cases of death and reduction in productivity (for meat, milk, wool, hides, eggs, honey etc.) should be diminished, so that more economic and easier animal husbandry is possible by use of the active compounds according to the invention.

The active compounds according to the invention are used in the veterinary sector in a known manner by enteral administration in the form of, for example, tablets, capsules, potions, drenches, granules, pastes, boluses, the feed-through process and suppositories, by parenteral administration, such as, for example, by injection (intramuscular, subcutaneous, intravenous, intraperitoneal and the like), implants, by nasal administration, by dermal use in the form, for example, of dipping or bathing, spraying, pouring on and spotting on, washing and powdering, and also with the aid of moulded articles containing the active compound, such as collars, ear marks, tail marks, limb bands, halters, marking devices and the like.

When used for cattle, poultry, pets and the like, the active compounds of the formula (I) can be used as formulations (for example powders, emulsions, free-flowing compositions), which comprise the active compounds in an amount of 1 to 80% by weight, directly or after 100 to 10000-fold dilution, or they can be used as a chemical bath.

It has furthermore been found that the compounds according to the invention have a strong insecticidal action against insects which destroy industrial materials.

The following insects may be mentioned as examples and as preferred—but without limitation:

Beetles, such as

Hylotrupes bajulus, Chlorophorus pilosis, Anobium punctatum, Xestobium rufovillosum, Ptilinus pecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec. Tryptodendron spec. Apate monachus, Bostrychus capucins, Heterobostrychus brunneus, Sinoxylon spec. Dinoderus minutus.

Hymenopterons, such as

Sirex juvencus, Urocerus gigas, Urocerus gigas taignus, Urocerus augur.

Termites, such as

Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus.

Bristletails, such as Lepisma saccharina.

Industrial materials in the present connection are to be understood as meaning non-living materials, such as, preferably, plastics, adhesives, sizes, papers and cards, leather, wood and processed wood products and coating compositions.

Wood and processed wood products are materials to be protected, especially preferably, from insect infestation.

Wood and processed wood products which can be protected by the agents according to the invention or mixtures comprising these are to be understood as meaning, for example:

building timber, wooden beams, railway sleepers, bridge components, boat jetties, wooden vehicles, boxes, pallets, containers, telegraph poles, wood panelling, wooden windows and doors, plywood, chipboard, joinery or wooden products which are used quite generally in house-building or in building joinery.

The active compounds can be used as such, in the form of concentrates or generally customary formulations, such as powders, granules, solutions, suspensions, emulsions or pastes.

The formulations mentioned can be prepared in a manner known per se, for example by mixing the active compounds with at least one solvent or diluent, emulsifier, dispersing agent and/or binder or fixing agent, a water repellent, if appropriate siccatives and UV stabilizers and if appropriate dyestuffs and pigments, and also other processing auxiliaries.

The insecticidal compositions or concentrates used for the preservation of wood and wood-derived timber products comprise the active compound according to the invention in a concentration of 0.0001 to 95% by weight, in particular 0.001 to 60% by weight.

The amount of the compositions or concentrates employed depends on the nature and occurrence of the insects and on the medium. The optimum amount employed can be determined for the use in each case by a series of tests. In general, however, it is sufficient to employ 0.0001 to 20% by weight, preferably 0.001 to 10% by weight, of the active compound, based on the material to be preserved.

Solvents and/or diluents which are used are an organic chemical solvent or solvent mixture and/or an oily or oil-like organic chemical solvent or solvent mixture of low volatility and/or a polar organic chemical solvent or solvent mixture and/or water, and if appropriate an emulsifier and/or wetting agent.

Organic chemical solvents which are preferably used are oily or oil-like solvents having an evaporation number above 35 and a flashpoint above 30° C., preferably above 45° C. Substances which are used as such oily or oil-like water-insoluble solvents of low volatility are appropriate mineral oils or aromatic fractions thereof, or solvent mixtures containing mineral oils, preferably white spirit, petroleum and/or alkylbenzene.

Mineral oils having a boiling range from 170 to 220° C., white spirit having a boiling range from 170 to 220° C., spindle oil having a boiling range from 250 to 350° C., petroleum and aromatics having a boiling range from 160 to 280° C., terpentine oil and the like, are advantageously employed.

In a preferred embodiment, liquid aliphatic hydrocarbons having a boiling range from 180 to 210° C. or high-boiling mixtures of aromatic and aliphatic hydrocarbons having a boiling range from 180 to 220° C. and/or spindle oil and/or monochloronaphthalene, preferably α-monochloronaphthalene, are used.

The organic oily or oil-like solvents of low volatility which have an evaporation number above 35 and a flashpoint above 30° C., preferably above 45° C., can be replaced in part by organic chemical solvents of high or medium volatility, providing that the solvent mixture likewise has an evaporation number above 35 and a flashpoint above 30° C., preferably above 45° C., and that the insecticide/fungicide mixture is soluble or emulsifiable in this solvent mixture.

According to a preferred embodiment, some of the organic chemical solvent or solvent mixture or an aliphatic polar organic chemical solvent or solvent mixture is replaced. Aliphatic organic chemical solvents containing hydroxyl and/or ester and/or ether groups, such as, for example, glycol ethers, esters or the like, are preferably used.

Organic chemical binders which are used in the context of the present invention are the synthetic resins and/or binding drying oils which are known per se, are water-dilutable and/or are soluble or dispersible or emulsifiable in the organic chemical solvents employed, in particular binders consisting of or comprising an acrylate resin, a vinyl resin, for example polyvinyl acetate, polyester resin, polycondensation or polyaddition resin, polyurethane resin, alkyd resin or modified alkyd resin, phenolic resin, hydrocarbon resin, such as indene-cumarone resin, silicone resin, drying vegetable oils and/or drying oils and/or physically drying binders based on a natural and/or synthetic resin.

The synthetic resin used as the binder can be employed in the form of an emulsion, dispersion or solution. Bitumen or bituminous substances can also be used as binders in an amount of up to 10% by weight. Dyestuffs, pigments, water-repelling agents, odour correctants and inhibitors or anticorrosive agents and the like which are known per se can additionally be employed.

It is preferred according to the invention for the composition or concentrate to comprise, as the organic chemical binder, at least one alkyd resin or modified alkyd resin and/or one drying vegetable oil. Alkyd resins having an oil content of more than 45% by weight, preferably 50 to 68% by weight, are preferably used according to the invention.

All or some of the binder mentioned can be replaced by a fixing agent (mixture) or a plasticizer (mixture). These additives are intended to prevent evaporation of the active compounds and crystallization or precipitation. They preferably replace 0.01 to 30% of the binder (based on 100% of the binder employed).

The plasticizers originate from the chemical classes of phthalic acid esters, such as dibutyl, dioctyl or benzyl butyl phthalate, phosphoric acid esters, such as tributyl phosphate, adipic acid esters, such as di-(2-ethylhexyl) adipate, stearates, such as butyl stearate or amyl stearate, oleates, such as butyl oleate, glycerol ethers or higher molecular weight glycol ethers, glycerol esters and p-toluenesulphonic acid esters.

Fixing agents are based chemically on polyvinyl alkyl ethers, such as, for example, polyvinyl methyl ether or ketones, such as benzophenone or ethylenebenzophenone.

Possible solvents or diluents are, in particular, also water, if appropriate as a mixture with one or more of the abovementioned organic chemical solvents or diluents, emulsifiers and dispersing agents.

Particularly effective preservation of wood is achieved by impregnation processes on a large industrial scale, for example vacuum, double vacuum or pressure processes.

The ready-to-use compositions can also comprise other insecticides, if appropriate, and also one or more fungicides, if appropriate.

Possible additional mixing partners are, preferably, the insecticides and fungicides mentioned in WO 94/29 268. The compounds mentioned in this document are an explicit constituent of the present application.

Especially preferred mixing partners which may be mentioned are insecticides, such as chlorpyriphos, phoxim, silafluofin, alphamethrin, cyfluthrin, cypermethrin, deltamethrin, permethrin, imidacloprid, NI-25, flufenoxuron, hexaflumuron, transfluthrin, thiacloprid, methoxyphenoxide and triflumuron,

and also fungicides, such as epoxyconazole, hexaconazole, azaconazole, propiconazole, tebuconazole, cyproconazole, metconazole, imazalil, dichlorfluanid, tolylfluanid, 3-iodo-2-propinyl-butyl carbamate, N-octyl-isothiazolin-3-one and 4,5-dichloro-N-octylisothiazolin-3-one.

The compounds according to the invention can at the same time be employed for protecting objects which come into contact with saltwater or brackish water, such as hulls, screens, nets, buildings, moorings and signalling systems, against fouling.

Fouling by sessile Oligochaeta, such as Serpulidae, and by shells and species from the Ledamorpha group (goose barnacles), such as various Lepas and Scalpellum species, or by species from the Balanomorpha group (acorn barnacles), such as Balanus or Pollicipes species, increases the frictional drag of ships and, as a consequence, leads to a marked increase in operation costs owing to higher energy consumption and additionally frequent residence in the dry dock.

Apart from fouling by algae, for example Ectocarpus sp. and Ceramium sp., in particular fouling by sessile Entomostraka groups, which come under the generic term Cirripedia (cirriped crustaceans), is of particular importance.

Surprisingly, it has now been found that the compounds according to the invention, alone or in combination with other active compounds, have an outstanding antifouling action.

Using compounds according to the invention, alone or in combination with other active compounds, allows the use of heavy metals such as, for example, in bis(trialkyltin) sulphides, tri-n-butyltin laurate, tri-n-butyltin chloride, copper(I) oxide, triethyltin chloride, tri-n-butyl(2-phenyl-4-chlorophenoxy)tin, tributyltin oxide, molybdenum disulphide, antimony oxide, polymeric butyl titanate, phenyl-(bispyridine)bismuth chloride, tri-n-butyltin fluoride, manganese ethylenebisthiocarbamate, zinc dimethyldithiocarbamate, zinc ethylenebisthiocarbamate, zinc salts and copper salts of 2-pyridinethiol 1-oxide, bisdimethyldithiocarbamoylzinc ethylene-bisthiocarbamate, zinc oxide, copper(I) ethylene-bisdithiocarbamate, copper thiocyanate, copper naphthenate and tributyltin halides to be dispensed with, or the concentration of these compounds to be substantially reduced.

If appropriate, the ready-to-use antifouling paints can additionally comprise other active compounds, preferably algicides, fungicides, herbicides, molluscicides, or other antifouling active compounds.

Preferably suitable components in combinations with the antifouling compositions according to the invention are:

algicides such as

2-tert-butylamino-4-cyclopropylamino-6-methylthio-1,3,5-triazine, dichlorophen, diuron, endothal, fentin acetate, isoproturon, methabenzthiazuron, oxyfluorfen, quinoclamine and terbutryn;

fungicides such as

benzo[b]thiophenecarboxylic acid cyclohexylamide S,S-dioxide, dichlofluanid, fluorfolpet, 3-iodo-2-propinyl butylcarbamate, tolylfluanid and azoles such as

azaconazole, cyproconazole, epoxyconazole, hexaconazole, metconazole, propiconazole and tebuconazole;

molluscicides such as

fentin acetate, metaldehyde, methiocarb, niclosamid, thiodicarb and trimethacarb;

or conventional antifouling active compounds such as

4,5-dichloro-2-octyl-4-isothiazolin-3-one, diiodomethylparatryl sulphone, 2-(N,N-dimethylthio-carbamoylthio)-5-nitrothiazyl, potassium, copper, sodium and zinc salts of 2-pyridinethiol 1-oxide, pyridine-triphenylborane, tetrabutyldistannoxane, 2,3,5,6-tetrachloro-4-(methylsulphonyl)-pyridine, 2,4,5,6-tetrachloroisophthalonitrile, tetramethylthiuram disulphide and 2,4,6-trichlorophenylmaleinimide.

The antifouling compositions used comprise the active compound according to the invention of the compounds according to the invention in a concentration of 0.001 to 50% by weight, in particular 0.01 to 20% by weight.

Moreover, the antifouling compositions according to the invention comprise the customary components such as, for example, those described in Ungerer, Chem. Ind. 1985, 37, 730-732 and Williams, Antifouling Marine Coatings, Noyes, Park Ridge, 1973.

Besides the algicidal, fungicidal, molluscicidal active compounds and insecticidal active compounds according to the invention, antifouling paints comprise, in particular, binders.

Examples of recognized binders are polyvinyl chloride in a solvent system, chlorinated rubber in a solvent system, acrylic resins in a solvent system, in particular in an aqueous system, vinyl chloride/vinyl acetate copolymer systems in the form of aqueous dispersions or in the form of organic solvent systems, butadiene/styrene/acrylonitrile rubbers, drying oils such as linseed oil, resin esters or modified hardened resins in combination with tar or bitumens, asphalt and epoxy compounds, small amounts of chlorine rubber, chlorinated polypropylene and vinyl resins.

If appropriate, paints also comprise inorganic pigments, organic pigments or colorants which are preferably insoluble in salt water. Paints may furthermore comprise materials such as colophonium to allow controlled release of the active compounds. Furthermore, the paints may comprise plasticizers, modifiers which affect the rheological properties and other conventional constituents. The compounds according to the invention or the abovementioned mixtures may also be incorporated into self-polishing antifouling systems.

The active compounds are also suitable for controlling animal pests, in particular insects, arachnids and mites, which are found in enclosed spaces such as, for example, dwellings, factory halls, offices, vehicle cabins and the like. They can be employed alone or in combination with other active compounds and auxiliaries in domestic insecticide products for controlling these pests. They are active against sensitive and resistant species and against all developmental stages. These pests include:

From the order of the Scorpionidea, for example, Buthus occitanus.

From the order of the Acarina, for example, Argas persicus, Argas reflexus, Bryobia ssp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus, Dermatophagoides forinae.

From the order of the Araneae, for example, Aviculariidae, Araneidae.

From the order of the Opiliones, for example, Pseudoscorpiones chelifer, Pseudoscorpiones cheiridium, Opiliones phalangium.

From the order of the Isopoda, for example, Oniscus asellus, Porcellio scaber.

From the order of the Diplopoda, for example, Blaniulus guttulatus, Polydesmus spp.

From the order of the Chilopoda, for example, Geophilus spp.

From the order of the Zygentoma, for example, Ctenolepisma spp., Lepisma saccharina, Lepismodes inquilinus.

From the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa.

From the order of the Saltatoria, for example, Acheta domesticus.

From the order of the Dermaptera, for example, Forficula auricularia.

From the order of the Isoptera, for example, Kalotermes spp., Reticulitermes spp.

From the order of the Psocoptera, for example, Lepinatus spp., Liposcelis spp.

From the order of the Coleptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum.

From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga carnaria, Simulium spp., Stomoxys calcitrans, Tipula paludosa.

From the order of the Lepidoptera, for example, Achroia grisella, Galleria mellonella, Plodia interpunctella, Tinea cloacella, Tinea pellionella, Tineola bisselliella.

From the order of the Siphonaptera, for example, Ctenocephalides canis, Ctenocephalides felis, Pulex irritans, Tunga penetrans, Xenopsylla cheopis.

From the order of the Hymenoptera, for example, Camponotus herculeanus, Lasius fuliginosus, Lasius niger, Lasius umbratus, Monomorium pharaonis, Paravespula spp., Tetramorium caespitum.

From the order of the Anoplura, for example, Pediculus humanus capitis, Pediculus humanus corporis, Phthirus pubis.

From the order of the Heteroptera, for example, Cimex hemipterus, Cimex lectularius, Rhodinus prolixus, Triatoma infestans.

In the field of household insecticides, they are used alone or in combination with other suitable active compounds, such as phosphoric acid esters, carbamates, pyrethroids, growth regulators or active compounds from other known classes of insecticide.

They are used as aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free or passive evaporation systems, moth papers, moth bags and moth gels, as granules or dusts, in baits for spreading or in bait stations.

### EXAMPLES

The following abbreviations are used in the descriptions

| | |
|---|---|
| ACN | acetonitrile |
| aq. | aqueous |
| DCI | direct chemical ionisation |
| DCM | dichloromethane |
| DMF | N,N-dimethylformamide |
| DMSO | dimethylsulfoxide |
| EDTA | ethylenediamine tetra-acetic acid |
| ESI | electro-spray ionisation |
| FCS | Fetal calf serum |
| h | hour/hours |
| HPLC | high pressure liquid chromatography |
| LC/MS | liquid chromatography-coupled mass spectroscopy |
| min. | minute(s) |
| MS | mass spectroscopy |
| NMR | nuclear magnetic resonance spectroscopy |
| PBS | Phosphate buffered saline |
| RP | reverse phase (HPLC) |
| Rₜ | retention time (HPLC) |
| rt | room temperature |
| SDS | Sodium dodecyl sulphate |
| TFA | trifluoroacetic acid |
| THF | tetrahydrofuran |
| UV | ultraviolet |
| UV/Vis | ultraviolet-visual |
| % of th. | % of theoretical yield |

General Experimental Procedures

Chemicals are obtained in analytical grade from Merck (Darmstadt, Germany) or Sigma-Aldrich (Deisenhofen, Germany). NMR spectra are recorded in DMSO-d₆ using a Bruker DRX 500 spectrometer (operating at 500.13 MHz proton frequency).

HPLC-MS analyses are performed using a Agilent HP1100 liquid chromatograph coupled with a LCT mass spectrometer (Micromass, Manchester, UK) in the positive and negative electrospray ionisation (ESI) mode, based on slight modification of a previously described method (M. Stadler et al., Phytochemistry, 56, 787-793). A Waters symmetry column is used as stationary phase. Mobile phase A: 0.1% formic acid in water, mobile phase B: 0.1% formic acid in acetonitrile; gradient: 0-1 min. 100% A, from 1-6 min. to 90% B, from 6 to 8 min to 100% B, from 8-10 min 100% B. LC-MS spectra are recorded in the range of molecular weights between 150 and 1.600.

HPLC-UV/Vis analyses are carried out in analogy to M. Stadler et al., Mycol. Res., 2001, 105, 1190-1205 on a HP 1100 Series analytical HPLC system (Agilent, Waldbronn, Germany) comprising a G 1312A binary pump system, a G 1315A diode array detector, a G 1316A column compartment, a G 1322A degaser and a G 1313A autoinjector. As mobile phase, 0.01% phosphonic acid: acetonitrile is chosen, while a Merck (Darmstadt, Germany) Lichrospher RP 18 column (125×4 mm, particle size 7 μm) serves as stationary phase. Aliquots of the samples (representing 2-10 μg of methanol-soluble materials, according to the concentrations of main metabolites) are analysed at 40° C. with a flow of 1 ml/min in the following gradient: Linear from 0% acetonitrile to 100% acetonitrile in 10 min, thereafter isocratic conditions at 100% acetonitrile for 5 min; followed by regeneration of the column for 5 min. HPLC-UV chromatograms are recorded at 210 nm with a reference wavelength of 550 nm and a bandwidth of 80 nm. Diode array detection (DAD) is employed to record HPLC-UV/Vis spectra in the range of 210-600 nm. The HP ChemStation software allows for an automated search for calibrated standard compounds in crude extracts.

Preparative HPLC is performed at room temperature on a preparative HPLC system (Gilson Abimed, Ratingen, Germany), comprising Gilson Unipoint software, 306 binary pump system, 205 fraction collector, 119 UV-Vis detector, 806 manometric module, and 811C dynamic mixer, using different gradients and stationary phases as described below.

NMR spectra are recorded on a Bruker DMX500, operating at 500.13 MHz proton frequency. All spectra are measured in DMSO-d₆ solution at 302 K. The solvent peak is used as internal reference for both proton and carbon chemical shifts (δ_{H}: 2.50, δ_{C}: 39.5).

Characterisation and Maintenance of Strain JS360

Culture Media

Yeast-Malt-Glucose (YMG) medium: D-glucose 0.4%, malt extract 1%, yeast extract 0.4%, pH 7.2.

Q6 medium: D-glucose 0.5%, glycerol 2%, cotton seed meal 1%, calcium carbonate 0.1%, tap water, no pH adjustment.

C medium: D-glucose 1%, yeast extract 1%, NZ amine (Sheffield Chemicals, Sheffield, U.K., Lot ONA 20 2) 0.5%, soluble starch 2%, after pH adjustment to pH 7,2 with sodium carbonate addition of calcium carbonate 0.4%.

GS medium: D-glucose 2%, deoiled soymeal (Soyamin 50 T, Degussa, Düsseldorf, Germany) 2%, soluble starch 2%, calcium carbonate 0.5%, sodium chloride 0.25%, magnesium sulfate 0.05%, monopotassium phosphate 0.025%, pH adjustment to 6.5-6.8.

MC medium: D-glucose 1%, yeast extract 0.5%, deoiled soymeal (Soyamin 50 T, Degussa, Düsseldorf, Germany) 1%, soluble starch 1%, sodium chloride 0.5%, calcium carbonate 0.3%, pH adjustment to 7.2 (0.1N sodium hydroxide solution).

MCPM medium: Diamalt Maltzin hell (Meistermarken GmbH, Bremen, Germany) 3.5%, NZ amine (Sheffield Chemicals, Sheffield, U.K., Lot ONA 20 2) 1%, sodium chloride 0.3%, monopotassium phosphate 0.1%, magnesium sulfate 0.05%, ferrous sulfate 0.01%, pH 6.8.

MS medium: Mannitol 2%, Soymeal defatted (Soyamin 50 T, Degussa, Düsseldorf, Germany) 2%, calcium carbonate 0.3%, pH adjustment to 7.5.

SP medium: Mannitol 3%, yeast extract 0.75%, soluble starch 0.2%, soy peptone (Merck, Darmstadt, Germany #107212.0500)) 0.5%, pH adjustment to 6.0 (hydrochloric acid).

Strain JS360 is obtained from a soil sample collected in Japan. It is maintained at the Bayer AG culture collection (Wuppertal, Germany) in 10% glycerol under liquid nitrogen. It has also been deposited at DSMZ (Deutsche Sammlung für Mikroorganismen und Zellkulturen, Mascheroder Weg 1b, D-38124 Braunschweig, Germany), on Nov. 27, 2002 under the designation number DSM 15324.

On YMG medium, single colonies of strain JS360 attain a diameter of 24 mm after incubation for 12 days. The colonies develope a white, fluffy aerial mycelium, while the substrate mycelium is creamish. The reverse of the culture is reddish brown, and a reddish pigment is released into the medium.

Fermentation and Extraction of Strain JS360

1: Seed Culture

Two ml of a 10% glycerol culture are used to inoculate 1 l Erlenmeyer flasks containing 150 ml of sterile YMG medium and propagated on a rotary shaker at 28° C. and 240 rpm for 72-96 h.

2: Fermentation of Strain JS360 in Flask Scale

After inoculation from a well-grown YMG seed culture (10 ml inoculum per flask), strain JS360 is propagated in ten 1 l Erlenmeyer flasks containing 150 ml of Q6 medium (see above) and propagated on a rotary shaker at 28° C. and 240 rpm for 118 h. During fermentation, daily samples are taken. The pH is determined, and free glucose is estimated using Bayer Diastix Harnzuckerstreifen. The wet mycelium is separated from the fluid by centrifugation (10 min. at 3000×g) and extracted with 2 l of acetone. The acetone is evaporated in vacuo (40° C.). The remaining aqueous residue is diluted with water to 500 ml and extracted three times with equal amounts of ethyl acetate. The combined organic phases are dried over sodium sulfate and evaporated in vacuo (40° C.) to yield 830 mg of crude extract, which is thereafter subjected to preparative HPLC as described below (isolation).

The culture fluid is applied onto an adsorption column containing 500 ml of Bayer Lewapol CA 9225 resin and rinsed with 1 l water. The column is eluted with 1.5 l acetone:methanol 4:1. The solvent is evaporated in vacuo (40° C.). The remaining aqueous residue is diluted with water to 500 ml and extracted three times with equal amounts of ethyl acetate. The combined organic phases are dried over sodium sulfate and evaporated in vacuo (40° C.) to yield 650 mg of crude extract, which is thereafter subjected to preparative HPLC as described below (isolation).

3: Fermentation of Strain JS360 in 30 l Scale (Stirring Fermentor)

A 40 l Biostat P fermentor (Braun Bioengeneering, Melsungen, Germany) containing 30 l of Q6 medium is sterilized in situ (1 h at 121° C. and 1.1 bar) and inoculated with two well-grown 150 ml YMG seed cultures that have been propagated for 76 h. The production culture is grown under stirring (240 rpm) and aeration (0.3 vvm). The pH is determined, and free glucose is estimated using Bayer Diastix Harnzuckerstreifen. In addition, the fermentor is equipped with a Braun oxygen electrode to determine oxygen saturation of the culture broth. Analytical HPLC of crude extracts prepared from 50 ml samples taken under sterile conditions and extracted with equal amounts of ethyl acetate serve as a means of detection for example 1. Examples 2 and 3 are also detected during fermentation by HPLC-MS but cannot be estimated in the native crude extracts, due to limited amounts and co-eluting other metabolites with similar retention times in the employed HPLC system. The ethyl acetate extracts are dried over sodium sulfate, evaporated to dryness, redissolved in methanol and analyzed using the HPLC-UV systems described in General Experimental Procedures. While the culture is fully saturated as deduced from the oxygen saturation values, the pH drops to values of ca. 4.5. After the free glucose in the medium is consumed, production of example 1 as estimated by analytical HPLC methodology starts at about 60 h of fermentation and reaches an optimum after 114 h. Then, the culture is harvested because at later stages degradation of example 1 is observed. After harvest of the culture, the fluid is separated from the mycelium by centrifugation (10 min. at 3000×g) and applied onto a column filled with Bayer Lewapol CA 9225 adsorption resin and rinsed with 5 l water. The column is thereafter eluted with 6 l acetone:methanol 4:1. The eluates are evaporated in vacuo (40° C.) to yield an aqueous residue, which is diluted to 1 l with water and extracted three times with 1 l ethyl acetate. The organic phases are combined, dried over sodium sulfate and evaporated in vacuo (40° C.). The resulting extract (22.7 g) is thereafter subjected to preparative HPLC as described below (isolation).

The mycelium is extracted three times with each 5 l of acetone, and the acetone is evaporated in vacuo (40° C.) to yield an aqueous residue, which is diluted to 1 l with water and extracted three times with 1 l ethyl acetate. The organic phases are combined, dried over sodium sulfate and evaporated in vacuo (40° C.). The resulting extract (13.4 g) is thereafter subjected to preparative HPLC as described below (isolation).

4. Fermentation in Other Culture Media (Flask Scale)

Strain JS 360 is propagated in various other culture media in attempts to optimize production of example 1 and chemically related metabolites. For this purpose, shake flask fermentations are carried out in a similar manner as described for the one in Q6 medium (see 2. above). 1 l Erlenmeyer flasks containing 150 ml of the media are thus propagated on a rotary shaker at 28° C. and 240 rpm for up to 118 h. During fermentation, daily samples are taken. The pH is determined, and free glucose is estimated using Bayer Diastix Harnzuckerstreifen. Aliquots of the culture broth (50 ml) are extracted with ethyl acetate. These ethyl acetate extracts are dried over sodium sulfate, evaporated to dryness, redissolved in methanol and analyzed using the HPLC-UV and HPLC-MS systems described in General Experimental Procedures. By comparison of retention times and spectra, example 1 and related compounds are detected in the following culture media: YM medium, C medium, GS medium, MC medium, MCPM medium, MS medium, and SP medium after 72-96 hours of fermentation. The highest yields of example 1, however, are observed in Q6 and GS media.

### Example 1

### (1R,4R,5S)-1-[(S)-(1S)-2-cyclohexen-1-yl(hydroxy)methyl]-4-hexyl-5-methyl-6-oxa-2-azabicyclo[3.2.0]heptane-3,7-dione

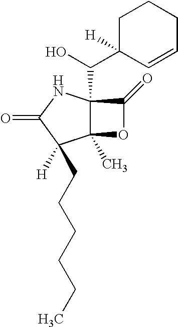

Preparation see below.

### Example 2

### (1R,4R,5S)-1-[(S)-(1S)-2-cyclohexen-1-yl(hydroxy)methyl]-4-[1-hydroxy-hexyl]-5-methyl-6-oxa-2-azabicyclo[3.2.0]heptane-3,7-dione

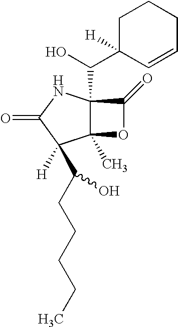

Preparation see below.

### Example 3

### (1R,4R,5S)-1-[(1R)-2-cyclohexen-1-ylmethyl]-4-hexyl-5-methyl-6-oxa-2-aza-bicyclo[3.2.0]heptane-3,7-dione

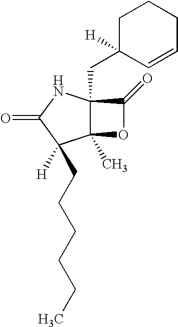

Preparation see below.

### Example 4

### (3S,4R)-2-[(S)-(1S)-2-cyclohexen-1-yl(hydroxy)methyl]-3-hydroxy-4-[1-hydroxy-hexyl]-3-methyl-5-oxo-D-proline

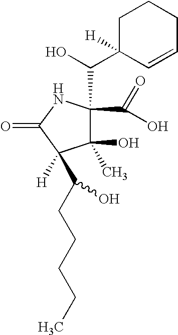

Preparation see below.

### Example 5

### N-acetyl-S-({(2R,3S,4R)-2-[(S)-(1S)-2-cyclohexen-1-yl(hydroxy)methyl]-4-hexyl-3-hydroxy-3-methyl-5-oxo-2-pyrrolidinyl}carbonyl)cysteine

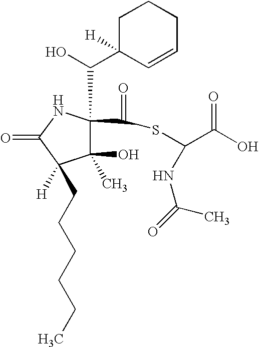

Preparation see below.

### Example 6

### methyl N-acetyl-S-({(2R,3S,4R)-2-[(S)-(1S)-2-cyclohexen-1-yl(hydroxy)methyl]-4-hexyl-3-hydroxy-3-methyl-5-oxo-2-pyrrolidinyl}carbonyl)cysteinate

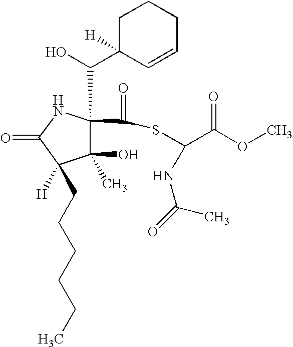

Preparation see below.

### Example 7

### (3S,4R)-2-[(S)-(1S)-2-cyclohexen-1-yl(hydroxy)methyl]-3-hydroxy-4-hexyl-3-methyl-5-oxo-D-proline

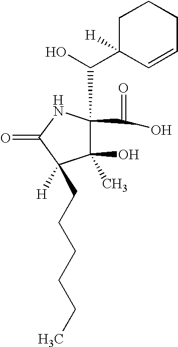

The stereochemistry of examples 2 to 7 is drawn in analogy to the structure of example 1 which is determined via X-ray analysis.

Isolation of Examples 1 to 7

1. Materials from Shake Flask Fermentations

The crude extracts (620 mg from the mycelium and 830 mg from the culture fluid, respectively) are dissolved in 5 ml of methanol, filtered through a Bond Elut C18 500 mg solid phase extraction cartridge (Baker, Deventer, The Netherlands) and applied onto a MZ Analysentechnik (Mainz, Germany) Kromasil RP 18 column (particle size, 7 μm; 250×40 mm). As mobile phase, a gradient of 0.01% TFA: acetonitrile is employed at a flow of 10 ml/min: 20% acetonitrile at t=0 min; linear gradient: 20% to 50% acetonitrile in 40 min; thereafter linear gradient from 50% to 100% acetonitrile in 20 min; thereafter isocratic conditions at 75% acetonitrile for 30 min, thereafter regeneration of the column. Fractions are combined according to UV adsorption at 210 nm. Example 1 is eluted at a retention time (Rₜ) of 80-83 min. and is obtained in amounts of 14 mg from the mycelial extract and 1.5 mg from the culture fluid extract, respectively. Examples 2 to 5 and 7 are located in minor intermediate fractions and not isolated to purity from this extract, while example 6 is not detected at all.

2: Materials from 30 l Scale Fermentation

Aliquots of 2.5-3 grams of the crude extracts which are prepared as described above (13.4 g from mycelium, 22.7 g from culture fluid) are dissolved in 5 ml of methanol, filtered through a Bond Elut C18 500 mg solid phase extraction cartridge (Baker, Deventer, The Netherlands) and applied onto a MZ Analysentechnik (Mainz, Germany) Kromasil RP 18 column (particle size, 7 μm; 250×40 mm; mobile phase, 0.01% TFA: acetonitrile). As mobile phase, a gradient of 0.01% TFA: acetonitrile is employed at a flow of 10 ml/min: 20% acetonitrile at t=0 min; linear gradient: 20% to 50% acetonitrile in 40 min; thereafter linear gradient from 50% to 100% acetonitrile in 20 min; thereafter isocratic conditions at 75% acetonitrile for 30 min, thereafter regeneration of the column. Fractions are combined according to UV adsorption at 210 nm. Five bioactive intermediate products are thus obtained. Their retention times (Rₜ) in this gradient system are observed as follows: 61-64 min. for intermediate product 1 (containing example 4 and 7), 65-71 min. for intermediate product 2 (containing examples 5 and 6), 72-79 min. for intermediate product 3 (containing example 2), 79-85 min. for intermediate product 4 (containing example 1) and 86-93 min. for intermediate product 5 (containing example 3).

Final purification of active components in intermediate products 1 to 5 is obtained by preparative reversed phase HPLC, using a flow of 7 ml/min and a MZ Analysentechnik Inertsil C18 column (7 μm; 250×30 mm) as stationary phase and the following gradient. Isocratic conditions from t=0 min=>t=30 min; thereafter linear gradient from 30% acetonitrile=>100% acetonitrile in 50 min, thereafter isocratic conditions (100% acetonitrile) for 20 min, thereafter regeneration of the column. Yields and Rₜ of examples 1 to 6 are summarized in table 1.

TABLE 1

| | Yield | Yield | |
|---|---|---|---|
| Example | (Culture fluid extract) | (Mycelial ectract) | Rₜ (min) |
| 1 | 14 mg | 160 mg | 59-65 |
| 2 | 29 mg | 43 mg | 33-35 |
| 3 | 5 mg | 18 mg | 77-80 |
| 4 | 12 mg | 17 mg | 51-54 |
| 5 | 2 mg | 14 mg | 69-71 |
| 6 | (not isolated) | 2 mg | 72-73 |
| 7 | 19 mg | (not isolated) | 55-58 |

Characterization of Example 1 to 7

Example 1 to 6 are detected by HPLC-UV and HPLC-MS using the methods described in General Experimental Procedures. Their characteristics in analytical HPLC systems are summarized in table 2. While examples 1, 2, 4 and 7 give conclusive results regarding their molecular peaks, the LC-MS of example 3 only reveals the molecular peak in the positive ESI mode, while due to loss of carbon dioxide in the negative ESI mode, a smaller major mass fragment is observed. In examples 5 and 6, dimers are readily formed under the employed HPLC-MS conditions, and the major LC-MS signal thus relates to these dimers, while the molecular peaks only constitute minor signals. These characteristics also serve to identify the examples by analytical HPLC in fermentation broths and intermediate fractions obtained during extraction, downstream processing and chromatography.

TABLE 2

| | Rₜ | Rₜ | | |
|---|---|---|---|---|
| | (HPLC-UV-Vis) | (HPLC-MS) | Molecular peak | Molecular peak |
| Example | [min] | [min] | m/z (pos. ESI) | m/z (neg. ESI) |
| 1 | 8.95-8.97 | 6.39-6.43 | 336 (M + H)⁺ | 334 (M − H)⁻ |
| 2 | 7.75-7.85 | 5.73-5.81 | 352 (M + H)⁺ | 350 (M − H)⁻ |
| 3 | 9.74-9.76 | 6.86-6.90 | 320 (M + H)⁺ | 276 (M − CO₂ − H)⁺ |
| 4 | 6.85-6.87 | 5.28-5.30 | 368 (M + H)⁺ | 370 (M − H)⁻ |
| 5 | 7.41-7.43 | 5.61-5.63 | 499 (M + H)⁺; | 497 (M − H)⁻; |
| | | | 997 (2M + H)⁺ | 995 (2M − H)⁻ |
| 6 | 8.00-8.05 | 5.81-5.84 | 513 (M + H)⁺; | 511 (M − H)⁻ |
| | | | 1025 (2M + H)⁺ | |
| 7 | 7.48-7.51 | 5.57-5.61 | 354 (M + H)⁺ | 352 (M − H)⁻ |

The structures of examples 1 to 7 are determined by low-resolution and high-resolution LC-MS spectrometry and by one- and two-dimensional NMR (nuclear magnetic resonance) spectroscopy. For instrumental parameters see General Experimental Procedures.

NMR data reveal the presence of a cis-double bond inside a cyclohexyl ring. The close analysis of HSQC, HMBC and COSY/TOCSY data allows to establish the bicyclic ring structure, which, together with the cyclohexenylcarbinol moiety, is identical to that found in Salinosporamide A. HSQC data point toward the presence of at least two methyl groups in each molecule. Together with TOCSY and HMBC, a non-branched hexyl moiety is identified. An unambiguous crosspeak in the COSY spectrum locates this chain at the 2-position in the heterocyclic ring system. Examples 7 (as compared to example 1) and example 4 (as compared to example 2), are revealed by NMR and MS data to constitute the respective seco-forms of the corresponding beta-lactone molecules. The presence of an additional hydroxyl group (compared to Salinosporamide A) at the 12-position (examples 2 and 4) becomes evident because of the multiplicity and the characteristic carbon and proton chemical shifts.

The NMR spectra of examples 5 and 6 show a complete new subset of signals that belong to an N-acylated cysteine moiety. The N-acetyl-cysteine is linked to the heterocylic ring structure via the carbonyl group of the former beta lactone ring, or the carboxyl group of example 7, respectively. The thioester link is identified by its carbonyl chemical shift (>200 ppm) and HMBC derived connectivity to the cysteine beta-hydrogens. All connectivities inside the cysteine residue are established by assigning the corresponding signals in HMBC and COSY spectra. Thus, the structures of examples 5 and 6 are analogous to that of lactacystin.

Spectroscopic Data

### Example 1

¹H-NMR (500 MHz, DMSO-d₆): δ=0.87 (t), 1.28 (m), 1.29 (m), 1.23 (m), 1.40 (m), 1.45 (m), 1.47 (m), 1.54 (m), 1.58 (m), 1.68 (m), 1.74 (s), 1.80 (m), 1.90 (m), 2.29 (m), 2.41 (t), 3.65 (m), 5.47 (m), 5.73 (m), 5.81 (m), 8.92 (s).

¹³C-NMR (DMSO-d₆): δ=13.8, 21.7, 21.8, 24.2, 25.2, 26.1, 26.8, 28.5, 30.8, 37.3, 47.5, 69.3, 78.4, 86.4, 127.8, 128.6, 169.1, 174.1.

### Example 2

¹H-NMR (500 MHz, DMSO-d₆): δ=0.88 (t), 1.27 (m), 1.29 (m), 1.35 (s), 1.37 (m), 1.49 (m), 1.58 (m), 1.59 (m), 1.71 (m), 1.75 (m), 1.93 (m), 2.35 (d), 2.76 (m), 3.49 (m), 4.00 (d), 4.68 (m), 5.70 (m), 5.91 (m), 6.11 (br.), 8.52 (s).

¹³C-NMR (DMSO-d₆): δ=13.4, 21.0, 21.4, 23.7, 24.2, 29.6, 30.1, 31.5, 36.1, 54.6, 69.4, 75.0, 76.0, 76.5, 127.9, 170.9, 172.3.

### Example 3

¹H-NMR (500 MHz, DMSO-d₆): δ=0.88 (t), 1.27 (m), 1.30 (m), 1.31 (m), 1.33 (m), 1.46 (m), 1.48 (m), 1.50 (m), 1.61 (s), 1.62 (m), 1.63 (m), 1.76 (m), 1.92 (m), 2.27 (m), 2.55 (t), 5.45 (m); 5.68 (m), 8.98 (s).

¹³C-NMR (DMSO-d₆): δ=13.3, 19.6, 21.4, 24.0, 24.2, 26.5, 28.2, 28.5, 29.9, 30.9, 32.5, 46.7, 74.0, 86.1, 127.7, 130.9, 170.4, 170.8.

### Example 4

¹H-NMR (500 MHz, DMSO-d₆): δ=0.82 (m), 1.17 (m), 1.20 (m), 1.24 (m), 1.32 (m), 1.47 (m), 1.60 (m), 1.62 (m), 1.63 (m), 1.84 (m), 2.08 (m) 2.44 (d), 3.68 (m), 3.80 (m), 5.60 (m), 5.76 (m).

¹³C-NMR (DMSO-d₆): δ=13.1, 20.1, 20.6, 21.0, 23.5, 23.6, 30.5, 33.5, 37.7, 52.7, 67.1, 73.6, 75.2, 80.1, 126.3, 128.6, 171.2, 176.5.

### Example 5

¹H-NMR (500 MHz, DMSO-d₆): δ=0.87 (m), 1.09 (m), 1.24 (m), 1.25 (m), 1.27 (m), 1.33 (m), 1.35 (m), 1.37 (m), 1.44 (m), 1.46 (m), 1.50 (m), 1.61 (m), 1.64 (m), 1.84 (s), 1.87 (m), 2.13 (m), 2.47 (t), 2.96 (m), 3.30 (m), 3.78 (m), 4.36 (m), 5.64 (m), 5.79 (m).

¹³C-NMR (DMSO-d₆): δ=13.9, 20.8, 21.7, 22.0, 22.1, 22.2, 23.4, 24.3, 26.8, 27.7, 28.7, 29.2, 31.1, 38.1, 50.3, 51.0, 75.3, 79.7, 80.6, 127.0, 129.3, 169.3, 178.9, 201.2.

### Example 6

¹H-NMR (500 MHz, DMSO-d₆): δ=0.87 (m), 1.09 (m), 1.24 (m), 1.25 (m), 1.27 (m), 1.33 (m), 1.35 (m), 1.37 (m), 1.44 (m), 1.46 (m), 1.50 (m), 1.61 (m), 1.64 (m), 1.84 (s), 1.87 (m), 2.13 (m), 2.47 (t), 3.00 (m), 3.24 (m), 3.64 (s), 3.78 (m), 4.39 (m), 5.64 (m), 5.79 (m).

¹³C-NMR (DMSO-d₆): δ=13.6, 20.8, 21.7, 22.0, 22.1, 23.4, 24.3, 26.8, 27.7, 28.7, 29.3, 31.1, 38.1, 50.3, 51.4, 51.8, 75.3, 79.7, 80.6, 127.0, 129.3, 169.3, 171.0, 178.9, 201.2.

### Example 7

¹H-NMR (500 MHz, DMSO-d₆): δ=0.86 (t), 1.25 (m), 1.26 (m), 1.33 (m), 1.34 (m), 1.36 (m), 1.44 (m), 1.46 (s), 1.51 (m), 1.66 (m), 1.67 (m), 1.88 (m), 2.12 (m), 2.45 (t), 3.77 (d), 5.64 (m), 5.82 (m), 7.66 (s).

¹³C-NMR (DMSO-d₆): δ=13.8, 20.3, 21.5, 21.7, 23.3, 24.4, 26.5, 27.9, 28.9, 31.0, 38.5, 50.5, 74.6, 75.5, 80.4, 127.4, 129.7, 177.5.

Interpretation of the NMR-Peak-Lists:

example 1: R¹=H, R²=OH, example 2: R¹═OH, R²═OH, example 3: R¹═H, R²═H.

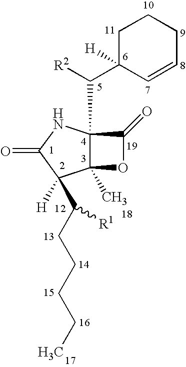

example 4, example 5: R⁸═H, example 6: R⁸═CH₃, example 7 (stereochemistry has not been determined by NMR).

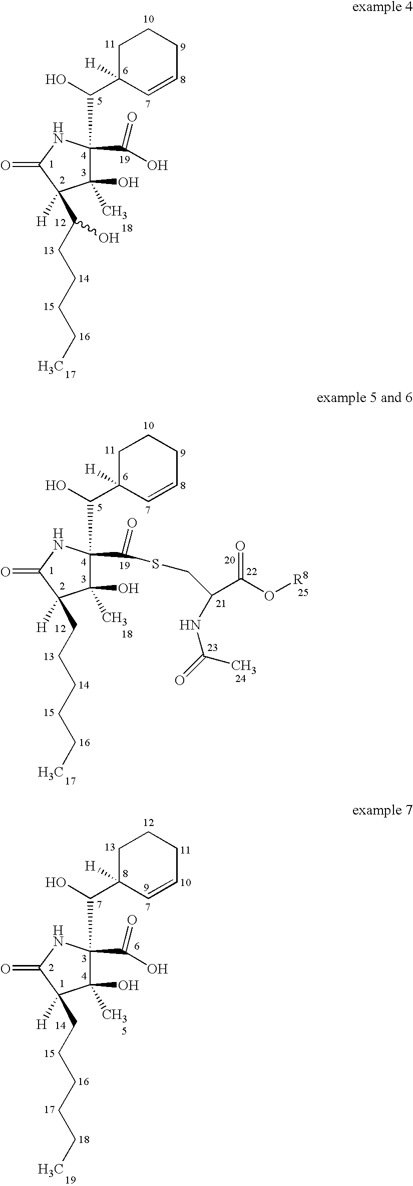

TABLE 3a
Chemical shifts for the examples 1 to 3, as measured at 500 MHz,
at 302 K in DMSO-d₆.

| carbon | example 1 | example 2 | example 3 |
|---|---|---|---|
| C-1 | 174.1 | 172.3 | 170.8 |
| C-2 | 47.5 | 54.6 | 46.7 |
| C-3 | 86.4 | 76.0⁺⁺ | 86.1 |
| C-4 | 78.4 | 69.4⁺⁺ | 74.0 |
| C-5 | 69.3 | 75.0 | 32.5 |
| C-6 | 37.3 | 36.1 | 29.9 |
| C-7 | 128.6 | 127.9 | 130.9 |
| C-8 | 127.8 | 127.9 | 127.7 |
| C-9 | 25.2 | 24.2 | 24.0 |
| C-10 | 21.7 | 21.0 | 19.6 |
| C-11 | 26.1 | 29.6 | 28.2 |
| C-12 | 24.2 | 76.5 | 24.2 |
| C-13 | 26.8 | 31.5 | 26.5 |
| C-14 | 28.5 | 23.7 | 28.5 |
| C-15 | 30.8 | 30.1 | 30.9 |
| C-16 | 21.8 | 21.4 | 21.4 |
| C-17 | 13.8 | 13.4 | 13.3 |
| C-18 | 21.8 | 21.4 | 21.4 |
| C-19 | 169.1 | 170.9 | 170.4 |

⁺⁺These resonance assignments can be interchanged

TABLE 3b
Chemical shifts for the examples 4 to 6, as measured at 500 MHz,
at 302 K in DMSO-d₆.

| carbon | example 4 | example 5 | example 6 |
|---|---|---|---|
| C-1 | 176.5 | 178.9 | 178.9 |
| C-2 | 52.7 | 50.3 | 50.3 |
| C-3 | 80.1 | 80.6 | 80.6 |
| C-4 | 75.2 | 79.7 | 79.7 |
| C-5 | 73.6 | 75.3 | 75.3 |
| C-6 | 37.7 | 38.1 | 38.1 |
| C-7 | 128.6 | 129.3 | 129.3 |
| C-8 | 126.3 | 127.0 | 127.0 |
| C-9 | 23.5 | 24.3 | 24.3 |
| C-10 | 20.6 | 21.7 | 21.7 |
| C-11 | 23.6 | 26.8 | 26.8 |
| C-12 | 67.1 | 23.4 | 23.4 |
| C-13 | 33.5 | 27.7 | 27.7 |
| C-14 | 23.6 | 28.7 | 28.7 |
| C-15 | 30.5 | 31.1 | 31.1 |
| C-16 | 21.0 | 22.0 | 22.0 |
| C-17 | 13.1 | 13.9 | 13.6 |
| C-18 | 20.1 | 20.8 | 20.8 |
| C-19 | 171.2 | 201.2 | 201.2 |
| C-20 | — | 29.2 | 29.3 |
| C-21 | — | 51.0 | 51.4 |
| C-22 | — | 169.3 | 171.0 |
| C-23 | — | 22.2 | 169.3 |
| C-24 | — | 22.1 | 22.1 |
| C-25 | — | — | 51.8 |

TABLE 3c
Chemical shifts for the example 7, as measured at 500 MHz,
at 302 K in DMSO-d₆.

| carbon | example 7 |
|---|---|
| C-1 | 50.5 |
| C-2 | 177.5 |
| C-3 | 75.5 |
| C-4 | 80.4 |
| C-5 | 20.3 |
| C-6 | — |
| C-7 | 74.6 |
| C-8 | 38.5 |
| C-9 | 129.7 |
| C-10 | 127.4 |
| C-11 | 24.4 |
| C-12 | 21.5 |
| C-13 | 26.5 |
| C-14 | 23.3 |
| C-15 | 27.9 |
| C-16 | 28.9 |
| C-17 | 31.0 |
| C-18 | 21.7 |
| C-19 | 13.8 |

TABLE 4a
Chemical shifts for the examples 1 to 3, as measured at 500 MHz,
at 302 K in DMSO-d₆.

| proton | example 1 | example 2 | example 3 |
|---|---|---|---|
| H-1 | — | — | — |
| H-2 | 2.41 t | 2.35 d | 2.55 t |
| H-3 | — | — | — |
| H-4 | — | — | — |
| H-5 | 3.65 m | 3.49 m | 1.76 m |
| H-6 | 2.29 m | 2.76 m | 2.27 m |
| H-7 | 5.81 m | 5.91 m | 5.45 m |
| H-8 | 5.73 m | 5.70 m | 5.68 m |
| H-9 | 1.90, 1.90 m | 1.93, 1.93 m | 1.92, 1.92 m |
| H-10 | 1.40, 1.68 m | 1.75, 1.49 m | 1.46, 1.63 m |
| H-11 | 1.23, 1.80 m | 1.59, 1.71 m | 1.33, 1.33 m |
| H-12 | 1.47, 1.58 m | 4.68 m | 1.62, 1.50 m |
| H-13 | 1.45, 1.54 m | 1.58, 1.58 m | 1.48, 1.48 m |
| H-14 | 1.29, 1.29 m | 1.37, 1.37 m | 1.31, 1.31 m |
| H-15 | 1.28, 1.28 m | 1.27, 1.27 m | 1.27, 1.27 m |
| H-16 | 1.28, 1.28 m | 1.29, 1.29 m | 1.30, 1.30 m |
| H-17 | 0.87 t | 0.88 t | 0.88 t |
| H-18 | 1.74 s | 1.35 s | 1.61 s |
| H-19 | — | — | — |
| H-N | 8.92 s | 8.52 s | 8.98 s |
| H-O-C-S | 5.47 | 4.00 d | |
| H-O-C-12 | — | 6.11 (tent.) | — |

TABLE 4b
Chemical shifts for the examples 4 to 6, as measured at 500 MHz,
at 302 K in DMSO-d₆.

| proton | example 4 | example 5 | example 6 |
|---|---|---|---|
| H-1 | — | — | — |
| H-2 | 2.44 d | 2.47 t | 2.47 t |
| H-3 | — | — | — |
| H-4 | — | — | — |
| H-5 | 3.68 m | 3.78 m | 3.78 m |
| H-6 | 2.08 m | 2.13 m | 2.13 m |
| H-7 | 5.76 m | 5.79 m | 5.79 m |
| H-8 | 5.60 m | 5.64 m | 5.64 m |
| H-9 | 1.84 m | 1.87 m | 1.87 m |
| H-10 | 1.62, 1.32 m | 1.61, 1.33 m | 1.61, 1.33 m |
| H-11 | 1.63, 1.17 m | 1.64, 1.09 m | 1.64, 1.09 m |
| H-12 | 3.80 m | 1.46, 1.37 m | 1.46, 1.37 m |
| H-13 | 1.60 m | 1.50, 1.35 m | 1.50, 1.35 m |
| H-14 | 1.32, 1.20 m | 1.24 m | 1.24 m |
| H-15 | 1.20 m | 1.25 m | 1.25 m |
| H-16 | 1.24 m | 1.27 m | 1.27 m |
| H-17 | 0.82 | 0.87 | 0.87 |
| H-18 | 1.47 | 1.44 | 1.44 |
| H-19 | — | — | — |
| H-20 | — | 3.30, 2.96 m | 3.24, 3.00 m |
| H-21 | — | 4.36 m | 4.39 m |
| H-22 | — | — | — |
| H-23 | — | — | — |
| H-24 | — | 1.84 s | 1.84 s |
| H-25 | — | — | 3.64 s |

TABLE 4c
Chemical shifts for the example 7, as measured at 500 MHz,
at 302 K in DMSO-d₆.

| proton | example 7 |
|---|---|
| H-1 | 2.45 t |
| H-5 | 1.46 s |
| H-7 | 3.77 d |
| H-8 | 2.12 m |
| H-9 | 5.82 m |
| H-10 | 5.64 m |
| H-11 | 1.88, 1.88 m |
| H-12 | 1.36, 1.66 m |
| H-13 | 1.67, 1.25 m |
| H-14 | 1.34, 1.44 m |
| H-15 | 1.33, 1.51 m |
| H-16 | 1.25 m |
| H-17 | 1.25 m |
| H-18 | 1.26 m |
| H-19 | 0.86 t |
| N-H | 7.66 s |

High Resolution Mass Spectrometry
example 1: ESI−; Mass found: 334.1977, calculated: 334.2014 (corresponding to a deviation of 4.1 mDa for the molecular formula C₁₉H₂₈NO₄)
example 2: ESI−; Mass found: 350.1968, calculated: 350.1967 (corresponding to a deviation of 0.1 mDa for the molecular formula C₁₉H₂₈NO₅)
example 3: ESI+; Mass found: 276.2388, calculated 276.2327 (corresponding to a deviation of 6.1 mDa for the molecular formula C₁₈H₃₀NO). Here, only the fragment (M-CO₂) could be observed under the ESI conditions.
example 4: ESI+; Mass found: 368.2107, calculated 368.2073 (corresponding to a deviation of 3.3 mDa for the molecular formula C₁₉H₃₁NO6
example 5: ESI−; Mass found: 497.2322, calculated: 497.2321 (corresponding to a deviation of 0.0 mDa for the molecular formula C₂₄H₃₈N₂O₇S)
example 6: ESI−; Mass found: 511.2594, calculated: 511.2478 (corresponding to a deviation of 11.6 mDa for the molecular formula C₂₅H₄₀N₂O₇S)
example 7: ESI+; Mass found: 354.2268, calculated 354.2280 (corresponding to a deviation of 1.3 mDa for the molecular formula C₁₉H₃₂NO₅).
   Examples for the Synthesis of Compounds According to the Invention According to Process B

Methyl 5S-isopropyl-2-phenyl-4,5-dihydro-1,3-oxazole-4R-carboxylate was synthesized by the method described in the literature (J. Am. Chem. Soc. 1999, 121, 9967-9976).

### Methyl 4-[2-hydroxy-3 -(methoxycarbonyl)-nonyl]-5S-isopropyl-2-phenyl-4,5-dihydro-1,3-oxazole-4R-carboxylate

To a solution of 2.00 g (8.10 mmol) methyl 5S-isopropyl-2-phenyl-4,5-dihydro-1,3-oxazole-4R-carboxylate in dry THF (45 mL) was added 16.2 ml LHMDS (1M in THF, 16.2 mmol) at −80° C. After 60 min 32.4 ml of dimethylaluminium chloride (1 M in hexane) was added dropwise over 45 min and stirring was continued for 90 min at −80° C. A solution of 4.86 g (24.3 mmol) methyl 2-acetyloctanoate in 10 ml THF was added over a period of 15 min. The reaction mixture was then warmed up to −20° C. and stirring was continued for 60 min. After recooling to −75° C. 20 ml saturated aqueous ammonium chloride was added. The mixture was poured to 100 ml saturated aqueous ammonium chloride, 20 ml of 6N HCl and 100 ml ethyl acetate. The aqueous layer was extracted with ethyl acetate and the combines organic layers were washed successively with 100 ml water, 100 ml saturated aqueous NaHCO3 and brine and finally dried over NaSO4. After evaporation of the solvent the residue was purified by chromatography on silica (cyclohexane/ethyl acetate) yielding the four diastereomers:

Methyl 4-[2S-hydroxy-3S-(methoxycarbonyl)-nonyl]-5S-isopropyl-2-phenyl-4,5-dihydro-1,3-oxazole-4R-carboxylate: 365 mg, log P 5.93;

An inseparable mixture of Methyl 4-[2S-hydroxy-3R-(methoxycarbonyl)-nonyl]-5S-isopropyl-2-phenyl-4,5-dihydro-1,3-oxazole-4R-carboxylate (MATA107-4-3) and Methyl 4-[2R-hydroxy-3S-(methoxycarbonyl)-nonyl]-5S-isopropyl-2-phenyl-4,5-dihydro-1,3-oxazole-4R-carboxylate: 560 mg, log P 5.48 and 5.56;

Methyl 4-[2R-hydroxy-3R-(methoxycarbonyl)-nonyl]-5S-isopropyl-2-phenyl-4,5-dihydro-1,3-oxazole-4R-carboxylate: 520 mg, log P 5.32.

### Methyl 4R-hexyl-3S-hydroxy-2R-[1S-hydroxy-2-methylpropyl]-3-methyl-5-oxo-pyrrolidine-2-carboxylate and Methyl 4S-hexyl-3R-hydroxy-2R-[1S-hydroxy-2-methylpropyl]-3-methyl-5-oxo-pyrrolidine-2-carboxylate

A solution of 500 mg of a inseparable mixture of methyl 4-[2R-hydroxy-3R-(methoxycarbonyl)-nonyl]-5S-isopropyl-2-phenyl-4,5-dihydro-1,3-oxazole-4R-carboxylate and 784 mg of 20% Pd(OH)2/C in 6 ml MeOH/AcOH (9:1) was stirred under 20 bar hydrogen for 72 h at ambient temperature. After filtration over silica the solvents were evaporated. The residue was dissolved in 20 ml ethyl acetate and washed successively with 10 ml saturated aqueous 4.5 M K2CO3 solution and 10 ml brine. The organic layer was dried over NaSO4 and evaporated in vacuo. The crude product was purified by chromatography on silica (cyclohexane/ethyl acetate) yielding Methyl 4R-hexyl-3S-hydroxy-2R-[1S-hydroxy-2-methylpropyl]-3-methyl-5-oxo-pyrrolidine-2-carboxylate and 4S-hexyl-3R-hydroxy-2R-[1S-hydroxy-2-methylpropyl]-3-methyl-5-oxo-pyrrolidine-2-carboxylate: (185 mg, log P 2.16 and 2.58).

### 4R-hexyl-3S-hydroxy-2R-[1S-hydroxy-2-methylpropyl]-3-methyl-5-oxo-pyrrolidine-2-carboxylic acid and 4S-hexyl-3R-hydroxy-2R-[1S-hydroxy-2-methylpropyl]-3-methyl-5-oxo-pyrrolidine-2-carboxylic acid

A solution of 180.0 mg Methyl 4R-hexyl-3S-hydroxy-2R-[1S-hydroxy-2-methylpropyl]-3-methyl-5-oxo-pyrrolidine-2-carboxylate, 4S-hexyl-3R-hydroxy-2R-[1S-hydroxy-2-methylpropyl]-3-methyl-5-oxo-pyrrolidine-2-carboxylate and 731 mg LiI in dry pyridine (18 mL) was heated to reflux for 3 h. After recooling the solvent was evaporated in vacuo. The residue was dissolved in water (20 mL) and extracted with ethyl acetate (10 mL). The aqueous layer was acidified with 0.5 M HCl to pH 2 and extracted with ethyl acetate (3×15 ml). The organic layer was washed with brine, dried over NaSO4 and concentrated, yielding 90 mg of a mixture of inseparable isomers (log P 1.86 and 1.98).

### Examples 8 and 9 (Table 1)

### (1R,4R,5S)-4-hexyl-1-[(1S)-1-hydroxy-2-methylpropyl]-5-methyl-6-oxa-2-azabicyclo[3.2.0]heptane-3,7-dione (MATA121-1) and (1R,4S,5S)-4-hexyl-1-[(1S)-1-hydroxy-2-methylpropyl]-5-methyl-6-oxa-2-azabicyclo[3.2.0]heptane-3,7-dione

To a cold (<5° C.) solution of 90 mg of a mixture of 4R-hexyl-3S-hydroxy-2-[1S-hydroxy-2-methylpropyl]-3-methyl-5-oxo-pyrrolidine-2-carboxylic acid and 4S-hexyl-3S-hydroxy-2-[1S-hydroxy-2-methylpropyl]-3-methyl-5-oxo-pyrrolidine-2-carboxylic acid in dry THF (4.5 mL), containing 60 μl triethylamine, was added dropwise isopropenyl chloroformate (38 mg). The reaction mixture was stirred for 60 min at <5° C. and additionally 60 min at ambient temperature. Addition of ethyl acetate (10 mL) afforded after 20 min stirring a suspension which was filtered and evaporated. The residue was purified by chromatography over silica (cyclhexane/ethyl acetate) yielding (1R,4S,5S)-4-hexyl-1-[(1S)-1-hydroxy-2-methylpropyl]-5-methyl-6-oxa-2-azabicyclo[3.2.0]heptane-3,7-dione (12 mg; log P 2,79) and (1R,4R,5S)-4-hexyl-1-[(1S)-1-hydroxy-2-methylpropyl]-5-methyl-6-oxa-2-azabicyclo[3.2.0]heptane-3,7-dione (16 mg; log P 2,87).

Examples for the Synthesis of Compounds According to the Invention According to Process F

Diethyl-2-(tert-butoxycarbonylamino)malonate is commercionally availible or may be synthesized by conventional boc-protection of diethylaminomalonate hydrochloride.

### 1. Diethyl 2-benzyl-2-(tert-butoxycarbonylamino)malonate

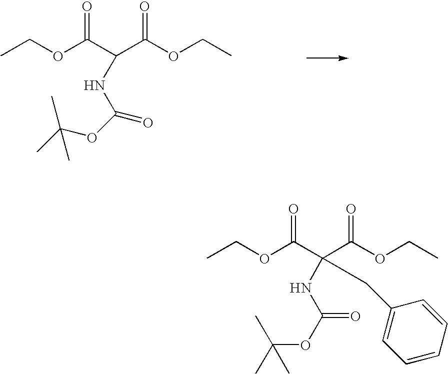

4.3 g (190 mmol) of sodium are dissolved in 600 ml of dry ethanol and slowly treated with a solution of 47 g (170 mmol) of diethyl-2-(tert-butoxycarbonylamino)malonate in 200 ml ethanol. After stirring for 30 min 29.2 g (170 mmol) of benzylbromide are added dropwise at room temperature. The reaction mixture is refluxed for 12 h followed by addition of aeq. HCl (pH 6-7), evaporation of the solvent and subsequent extraction with dichloromethane/water. The combined organic layers are dried over MgSO4 and the solvent is evaporated to yield the pure product in 88% yield (55 g).

¹H-NMR{400 MHz, DMSO-d₆}: 1.18 (t, ³J_{HH}=7 Hz, 6H, CH₃); 1.42 (s, 9H, CH₃); 3.44 (s, 2H, CH₂); 4.18 (m, 4H, CH₂); 6.2 (b, 1H, NH); 7.01 (b, 2H, Ph-H); 7.26 (b, 3H, Ph-H).

LC: 87% purity, MS: 266 (M-boc)

### 2. Diethyl benzyl[[3-benzyloxy-3-oxopropanoyl](tert-butoxycarbonyl)amino]malonate

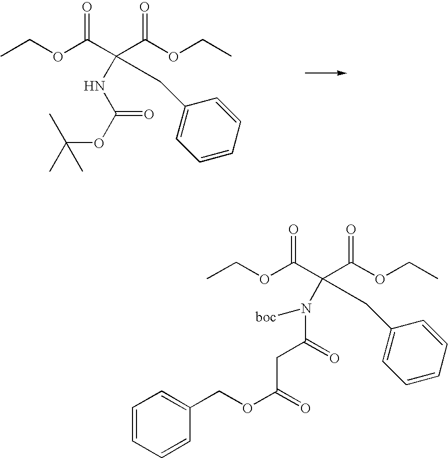

28.2 g (77 mmol) of diethyl 2-benzyl-2-(tert-butoxycarbonylamino)malonate, 15 g (77 mmol) of monobenzylmalonate and 12.5 g (97 mmol) of N,N-diisopropylethylamine are dissolved in 300 ml of dichloromethane and treated with 14.8 g (77 mmol) of 1-(3-dimethylaminopropyl)-3-ethylcarbodiimide hydrochloride at room temperature. The reaction mixture is stirred for 12 h, diluted with ethyl acetate, washed with aeq. 1N HCl (200 ml) and aeq. NaHCO₃ (2×200 ml) and dried over MgSO4. After evaporation of the solvent the product is obtained in 78% yield (32.5 g).

¹H-NMR{400 MHz, DMSO-d₆}: 1.18 (t, ³J_{HH}=7 Hz, 6H, CH₃); 1.43 (s, 9H, CH₃); 3.64 (s, 2H, CH₂); 4.17 (m, 4H, CH₂); 5.15 (s, 2H, CH₂); 7.00 (b, 2H, Ph-H); 7.26 (b, 3H, Ph-H); 7.35 (b, 5H, Ph-H).

### 3. 4-Benzyl-1-tert-butyl-2-ethyl-2-benzyl-3,5-dioxopyrrolidine-1,2,4-tricarboxylate

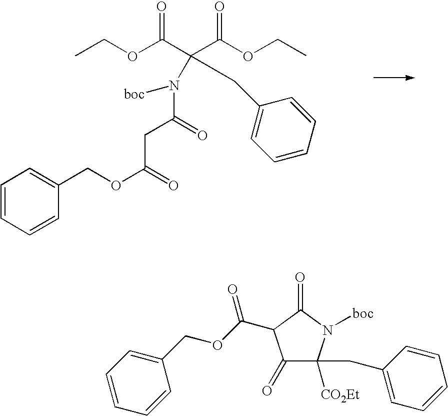

6.0 g (11 mmol) of diethyl benzyl[[3-benzyloxy-3-oxopropanoyl](tert-butoxycarbonyl)-amino]malonate are dissolved in 100 ml of dimethylformamide and treated with 3.1 g (27 mmol) of sodium tertbutylate at room temperature. The reaction mixture is stirred for 12 h at 50° C., diluted with dichloromethane, neutralized with aeq. 1N HCl, washed with water (1×50 ml) and aeq. NaHCO₃ (1×500 ml) and dried over MgSO4. After evaporation of the solvent the crude product is obtained as an oil which was purified by chromatography (cyclohexane:ethyl acetate 1:1) to yield 2.6 g (47%) of product as a mixture of diastereoisomers.

¹H-NMR{400 MHz, DMSO-d₆}: 1.13 and 1.18 (t, 3H, ³J_{HH}=7 Hz, CH₃); 1.39 and 1.43 (s, 9H, CH₃); 3.46 and 3.64 (s, 2H, CH₂); 4.15 (m, 2H, CH₂); 5.15 (s, 2H, CH₂); 7.03 (b, 2H, Ph-H); 7.26 (b, 3H, Ph-H); 7.35 (b, 5H, Ph-H).

### 4. 4-Benzyl-1-tert-butyl-2-ethyl-2-benzyl-4-[(2-E)-hex-2-en-1-yl]-3,5-dioxopyrrolidine-1,2,4-tri-carboxylate

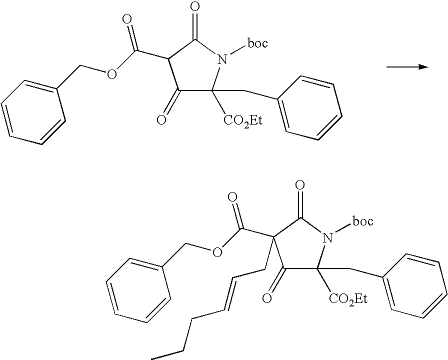

0.4 g (0.8 mmol) of 4-benzyl-1-tert-butyl-2-ethyl-2-benzyl-3,5-dioxopyrrolidine-1,2,4-tricarboxylate, 0.2 g (1.9 mmol) of trans-2-hexen-1-ol, 0.1 g (0.9 mmol) of triethylamine, 18 mg (0.08 mmol) of palladium acetate, 40 mg (0.16 mmol) of triphenylphosphine and 80 mg (0.8 mmol) of triethylborane are stirred in 20 ml of THF for 4 h. The reaction mixture is diluted with ethyl acetate, washed with aeq. 1N HCl (1×20 ml), water (2×20 ml) and aeq. NaHCO₃ (1×20 ml) and dried over MgSO4. After evaporation of the solvent the crude product is obtained as a mixture of diastereoisomers (0.43 g, 92%).

¹H-NMR{400 MHz, DMSO-d₆}: 0.88 (t, 3H, ³J_{HH}=7 Hz, CH₃); 1.13 and 1.18 (t, 3H, ³J_{HH}=7 Hz, CH₃); 1.20-1.35 (m, 4H, CH₂); 1.39 and 1.42 (s, 9H, CH₃); 1.90 (m, 2H, CH₂); 3.46 and 3.64 (s, 2H, CH₂); 4.15 (m, 2H, CH₂); 5.14 (s, 2H, CH₂); 5.54 (m, 2H, CH); 7.02 (b, 2H, Ph-H); 7.25 (b, 3H, Ph-H); 7.33 (b, 5H, Ph-H).

### 5. 4-Benzyl-1-tert-butyl-2-ethyl-2-benzyl-4-[(2-E)-hex-2-en-1-yl]-3-hydroxy-5-oxopyrrolidine-1,2,4-tricarboxylate (Example 462)

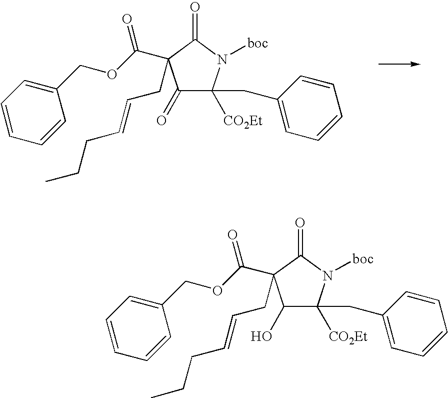

2.1 g (3.6 mmol) of 4-benzyl-1-tert-butyl-2-ethyl-2-benzyl-4-[(2-E)-hex-2-en-1-yl]-3,5-dioxopyrrolidine-1,2,4-tricarboxylate and 770 mg (3.6 mmol) of NaBH(OAc)₃ are stirred in 40 ml of glacial acetic acid at room temperature for 36 h. The reaction mixture is diluted with water and extracted with dichloromethane. The combined organic layers are washed with water (2×20 ml) and aeq. NaHCO₃ (1×20 ml) and dried over MgSO4. After evaporation of the solvent the crude product is obtained as a mixture of diastereoisomers (1.25 g, 60%).

¹H-NMR{400 MHz, DMSO-d₆}: 0.89 (t, 3H, ³J_{HH}=7 Hz, CH₃); 1.14 and 1.18 (t, 3H, ³J_{HH}=7 Hz, CH₃); 1.20-1.35 (m, 4H, CH₂); 1.39 and 1.42 (s, 9H, CH₃); 1.90 (m, 2H, CH₂); 3.42, 3.46 and 3.64 (s, 2H, CH₂); 4.15 (m, 2H, CH₂); 5.14 (s, 2H, CH₂); 5.54 (m, 2H, CH); 7.02 (b, 2H, Ph-H); 7.25 (b, 3H, Ph-H); 7.33 (b, 5H, Ph-H), OH not detected.

MS: 480 (M-boc)

According to the methods mentioned before the annelated lactames of the formula

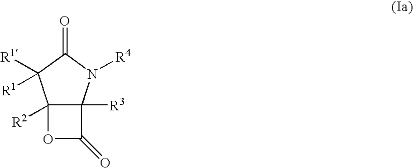

named in the following table can or have been obtained:

TABLE 5

| Ex- | | | | | |
|---|---|---|---|---|---|
| am- | | | | | |
| ple | R¹/R¹′ | R² | R³ | R⁴ | logP*) |
| 1 | n-hexyl/H (chiral) (R) | CH₃(chiral) (S) | 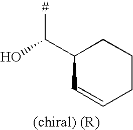 | H | |
| 2 | 1-hydroxy- hexyl/H (chiral) (R) | CH₃(chiral) (S) | 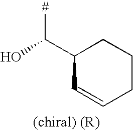 | H | |
| 3 | n-hexyl/H (chiral) (R) | CH₃(chiral) (S) | 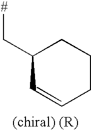 | H | |
| 8 | n-hexyl/H (chiral) (R) | CH₃(chiral) (S) | 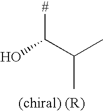 | H | 2.87 |
| 9 | n-hexyl/H (chiral) (S) | CH₃(chiral) (S) | 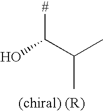 | H | 2.79 |
| 10 | CH₃/H (chiral) (R) | H (chiral) (S) | 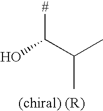 | H | |
| 11 | n-hexyl/H (chiral) (S) | CH₃(chiral) (S) | 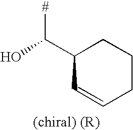 | H | |
| 12 | n-hexyl/H (chiral) (R) | CH₃(chiral) (R) | 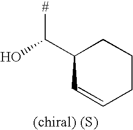 | H | |
| 13 | n-hexyl/H (chiral) (S) | CH₃(chiral) (R) | 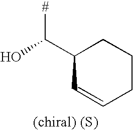 | H | |
| 14 | n-hexyl/H (chiral) (R) | CH₃(chiral) (S) | 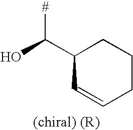 | H | |
| 15 | n-hexyl/H (chiral) (S) | CH₃(chiral) (S) | 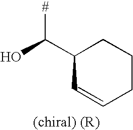 | H | |
| 16 | n-hexyl/H (chiral) (R) | CH₃(chiral) (R) | 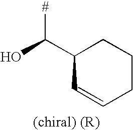 | H | |
| 17 | n-hexyl/H (chiral) (S) | CH₃(chiral) (R) | 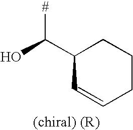 | H | |
| 18 | n-hexyl/H (chiral) (+/−) | CH₃(chiral) (+/−) | 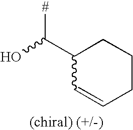 | H | |
| 19 | n-hexyl/H (chiral) (R) | CH₃(chiral) (R) | 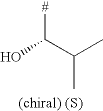 | H | |
| 20 | n-hexyl/H (chiral) (S) | CH₃(chiral) (R) | 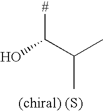 | H | |
| 21 | n-hexyl/H (chiral) (R) | CH₃(chiral) (S) | 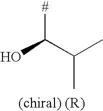 | H | |
| 22 | n-hexyl/H (chiral) (S) | CH₃(chiral) (S) | 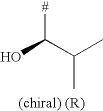 | H | |
| 23 | n-hexyl/H (chiral) (R) | CH₃(chiral) (R) | 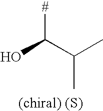 | H | |
| 24 | n-hexyl/H (chiral) (S) | CH₃(chiral) (R) | 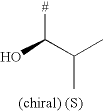 | H | |
| 25 | n-hexyl/H (chiral) (+/−) | CH₃(chiral) (+/−) | 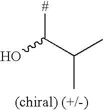 | H | |
| 26 | n-hexyl/H (chiral) (R) | CH₃(chiral) (S) | 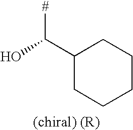 | H | |
| 27 | n-hexyl/H (chiral) (S) | CH₃(chiral) (S) | 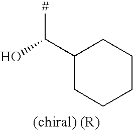 | H | |
| 28 | n-hexyl/H (chiral) (R) | CH₃(chiral) (R) | 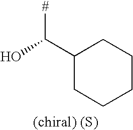 | H | |
| 29 | n-hexyl/H (chiral) (S) | CH₃(chiral) (R) | 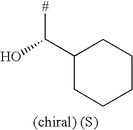 | H | |
| 30 | n-hexyl/H (chiral) (R) | CH₃(chiral) (S) | 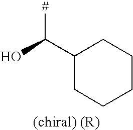 | H | |
| 31 | n-hexyl/H (chiral) (S) | CH₃(chiral) (S) | 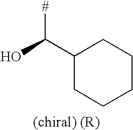 | H | |
| 32 | n-hexyl/H (chiral) (R) | CH₃(chiral) (R) | 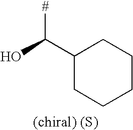 | H | |
| 33 | n-hexyl/H (chiral) (S) | CH₃(chiral) (R) | 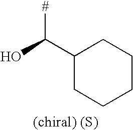 | H | |
| 34 | n-hexyl/H (chiral) (+/−) | CH₃(chiral) (+/−) | 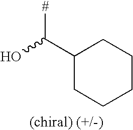 | H | |
| 35 | n-hexyl/H (chiral) (R) | CH₃(chiral) (S) | 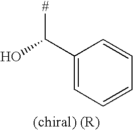 | H | |
| 36 | n-hexyl/H (chiral) (S) | CH₃(chiral) (S) | 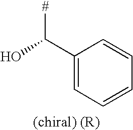 | H | |
| 37 | n-hexyl/H (chiral) (R) | CH₃(chiral) (R) | 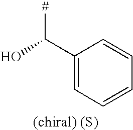 | H | |
| 38 | n-hexyl/H (chiral) (S) | CH₃(chiral) (R) | 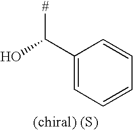 | H | |
| 39 | n-hexyl/H (chiral) (R) | CH₃(chiral) (S) | 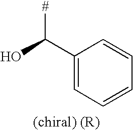 | H | |
| 40 | n-hexyl/H (chiral) (S) | CH₃(chiral) (S) | 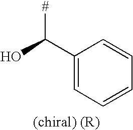 | H | |
| 41 | n-hexyl/H (chiral) (R) | CH₃(chiral) (R) | 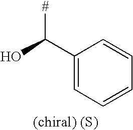 | H | |
| 42 | n-hexyl/H (chiral) (S) | CH₃(chiral) (R) | 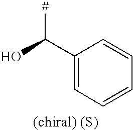 | H | |
| 43 | n-hexyl/H (chiral) (+/−) | CH₃(chiral) (+/−) | 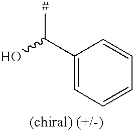 | H | |
| 44 | chloroethyl/ H (chiral) (R) | CH₃(chiral) (S) | 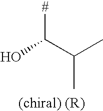 | H | |
| 45 | chloroethyl/ H (chiral) (S) | CH₃(chiral) (S) | 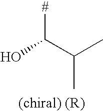 | H | |
| 46 | chloroethyl/ H (chiral) (R) | CH₃(chiral) (R) | 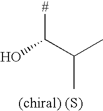 | H | |
| 47 | chloroethyl/ H (chiral) (S) | CH₃(chiral) (R) | 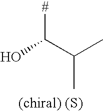 | H | |
| 48 | chloroethyl/ H (chiral) (+/−) | CH₃(chiral) (+/−) | 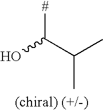 | H | |
| 49 | chloroethyl/ H (chiral) (R) | CH₃(chiral) (S) | 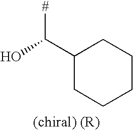 | H | |
| 50 | chloroethyl/ H (chiral) (S) | CH₃(chiral) (S) | 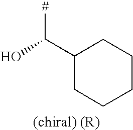 | H | |
| 51 | chloroethyl/ H (chiral) (R) | CH₃(chiral) (R) | 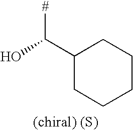 | H | |
| 52 | chloroethyl/ H (chiral) (S) | CH₃(chiral) (R) | 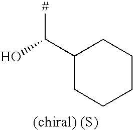 | H | |
| 53 | chloroethyl/ H (chiral) (+/−) | CH₃(chiral) (+/−) | 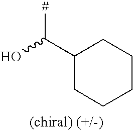 | H | |
| 54 | chloroethyl/ H (chiral) (R) | CH₃(chiral) (S) | 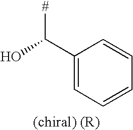 | H | |
| 55 | chloroethyl/ H (chiral) (S) | CH₃(chiral) (S) | 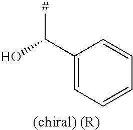 | H | |
| 56 | chloroethyl/ H (chiral) (R) | CH₃(chiral) (R) | 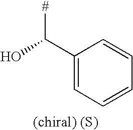 | H | |
| 57 | chloroethyl/ H (chiral) (S) | CH₃(chiral) (R) | 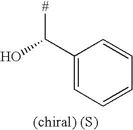 | H | |
| 58 | chloroethyl/ H (chiral) (+/−) | CH₃(chiral) (+/−) | 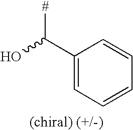 | H | |
| 59 | bromoethyl/ H (chiral) (R) | CH₃(chiral) (S) | 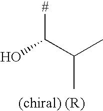 | H | |
| 60 | bromoethyl/ H (chiral) (S) | CH₃(chiral) (S) | 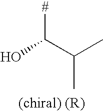 | H | |
| 61 | bromoethyl/ H (chiral) (R) | CH₃(chiral) (R) | 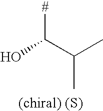 | H | |
| 62 | bromoethyl/ H (chiral) (S) | CH₃(chiral) (R) | 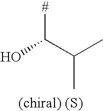 | H | |
| 63 | bromoethyl/ H (chiral) (+/−) | CH₃(chiral) (+/−) | 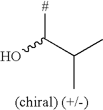 | H | |
| 64 | bromoethyl/ H (chiral) (R) | CH₃(chiral) (S) | 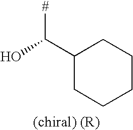 | H | |
| 65 | bromoethyl/ H (chiral) (S) | CH₃(chiral) (S) | 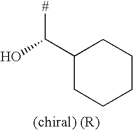 | H | |
| 66 | bromoethyl/ H (chiral) (R) | CH₃(chiral) (R) | 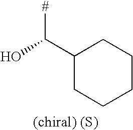 | H | |
| 67 | bromoethyl/ H (chiral) (S) | CH₃(chiral) (R) | 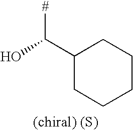 | H | |
| 68 | bromoethyl/ H (chiral) (+/−) | CH₃(chiral) (+/−) | 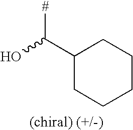 | H | |
| 69 | bromoethyl/ H (chiral) (R) | CH₃(chiral) (S) | 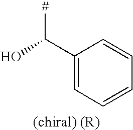 | H | |
| 70 | bromoethyl/ H (chiral) (S) | CH₃(chiral) (S) | 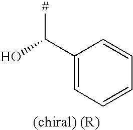 | H | |
| 71 | bromoethyl/ H (chiral) (R) | CH₃(chiral) (R) | 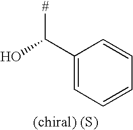 | H | |
| 72 | bromoethyl/ H (chiral) (S) | CH₃(chiral) (R) | 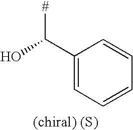 | H | |
| 73 | bromoethyl/ H (chiral) (+/−) | CH₃(chiral) (+/−) | 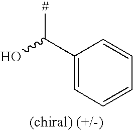 | H | |
| 74 | iodoethyl/H (chiral) (R) | CH₃(chiral) (S) | 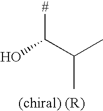 | H | |
| 75 | iodoethyl/H (chiral) (S) | CH₃(chiral) (S) | 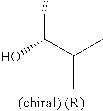 | H | |
| 76 | iodoethyl/H (chiral) (R) | CH₃(chiral) (R) | 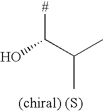 | H | |
| 77 | iodoethyl/H (chiral) (S) | CH₃(chiral) (R) | 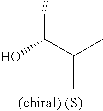 | H | |
| 78 | iodoethyl/H (chiral) (+/−) | CH₃(chiral) (+/−) | 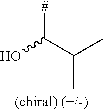 | H | |
| 79 | iodoethyl/H (chiral) (R) | CH₃(chiral) (S) | 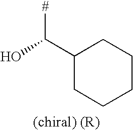 | H | |
| 80 | iodoethyl/H (chiral) (S) | CH₃(chiral) (S) | 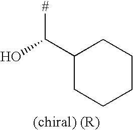 | H | |
| 81 | iodoethyl/H (chiral) (R) | CH₃(chiral) (R) | 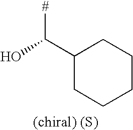 | H | |
| 82 | iodoethyl/H (chiral) (S) | CH₃(chiral) (R) | 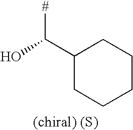 | H | |
| 83 | iodoethyl/H (chiral) (+/−) | CH₃(chiral) (+/−) | 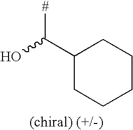 | H | |
| 84 | iodoethyl/H (chiral) (R) | CH₃(chiral) (S) | 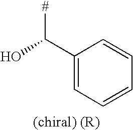 | H | |
| 85 | iodoethyl/H (chiral) (S) | CH₃(chiral) (S) | 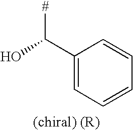 | H | |
| 86 | iodoethyl/H (chiral) (R) | CH₃(chiral) (R) | 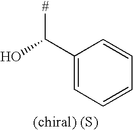 | H | |
| 87 | iodoethyl/H (chiral) (S) | CH₃(chiral) (R) | 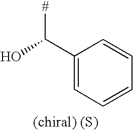 | H | |
| 88 | iodoethyl/H (chiral) (+/−) | CH₃(chiral) (+/−) | 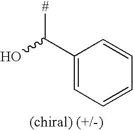 | H | |
| 89 | hydroxy- ethyl/H (chiral) (R) | CH₃(chiral) (S) | 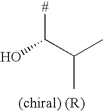 | H | |
| 90 | hydroxy- ethyl/H (chiral) (S) | CH₃(chiral) (S) | 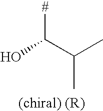 | H | |
| 91 | hydroxy- ethyl/H (chiral) (R) | CH₃(chiral) (R) | 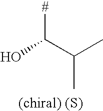 | H | |
| 92 | hydroxy- ethyl/H (chiral) (S) | CH₃(chiral) (R) | 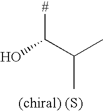 | H | |
| 93 | hydroxy- ethyl/H (chiral) (+/−) | CH₃(chiral) (+/−) | 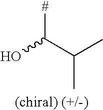 | H | |
| 94 | hydroxy- ethyl/H (chiral) (R) | CH₃(chiral) (S) | 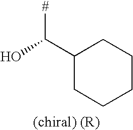 | H | |
| 95 | hydroxy- ethyl/H (chiral) (S) | CH₃(chiral) (S) | 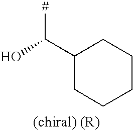 | H | |
| 96 | hydroxy- ethyl/H (chiral) (R) | CH₃(chiral) (R) | 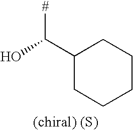 | H | |
| 97 | hydroxy- ethyl/H (chiral) (S) | CH₃(chiral) (R) | 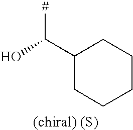 | H | |
| 98 | hydroxy- ethyl/H (chiral) (+/−) | CH₃(chiral) (+/−) | 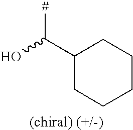 | H | |
| 99 | hydroxy- ethyl/H (chiral) (R) | CH₃(chiral) (S) | 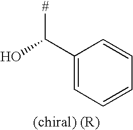 | H | |
| 100 | hydroxy- ethyl/H (chiral) (S) | CH₃(chiral) (S) | 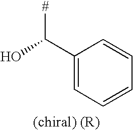 | H | |
| 101 | hydroxy- ethyl/H (chiral) (R) | CH₃(chiral) (R) | 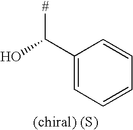 | H | |
| 102 | hydroxy- ethyl/H (chiral) (S) | CH₃(chiral) (R) | 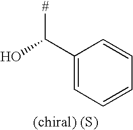 | H | |
| 103 | hydroxy- ethyl/H (chiral) (+/−) | CH₃(chiral) (+/−) | 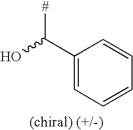 | H | |
| 104 | n-butyl/H (chiral) (R) | CH₃(chiral) (S) | 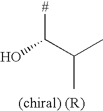 | H | |
| 105 | n-butyl/H (chiral) (S) | CH₃(chiral) (S) | 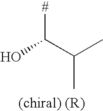 | H | |
| 106 | n-butyl/H (chiral) (R) | CH₃(chiral) (R) | 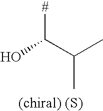 | H | |
| 107 | n-butyl/H (chiral) (S) | CH₃(chiral) (R) | 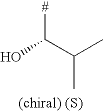 | H | |
| 108 | n-butyl/H (chiral) (+/−) | CH₃(chiral) (+/−) | 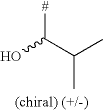 | H | |
| 109 | n-butyl/H (chiral) (R) | CH₃(chiral) (S) | 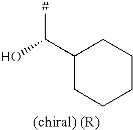 | H | |
| 110 | n-butyl/H (chiral) (S) | CH₃(chiral) (S) | 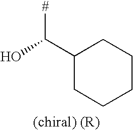 | H | |
| 111 | n-butyl/H (chiral) (R) | CH₃(chiral) (R) | 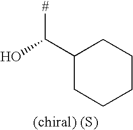 | H | |
| 112 | n-butyl/H (chiral) (S) | CH₃(chiral) (R) | 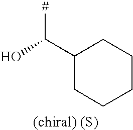 | H | |
| 113 | n-butyl/H (chiral) (+/−) | CH₃(chiral) (+/−) | 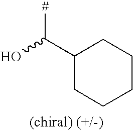 | H | |
| 114 | n-butyl/H (chiral) (R) | CH₃(chiral) (S) | 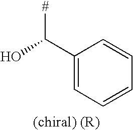 | H | |
| 115 | n-butyl/H (chiral) (S) | CH₃(chiral) (S) | 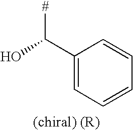 | H | |
| 116 | n-butyl/H (chiral) (R) | CH₃(chiral) (R) | 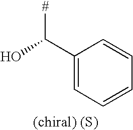 | H | |
| 117 | n-butyl/H (chiral) (S) | CH₃(chiral) (R) | 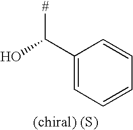 | H | |
| 118 | n-butyl/H (chiral) (+/−) | CH₃(chiral) (+/−) | 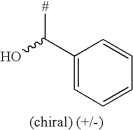 | H | |
| 119 | ethyl/H (chiral) (R) | CH₃(chiral) (S) | 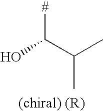 | H | |
| 120 | ethyl/H (chiral) (S) | CH₃(chiral) (S) | 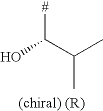 | H | |
| 121 | ethyl/H (chiral) (R) | CH₃(chiral) (R) | 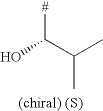 | H | |
| 122 | ethyl/H (chiral) (S) | CH₃(chiral) (R) | 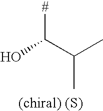 | H | |
| 123 | ethyl/H (chiral) (+/−) | CH₃(chiral) (+/−) | 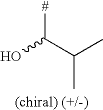 | H | |
| 124 | ethyl/H (chiral) (R) | CH₃(chiral) (S) | 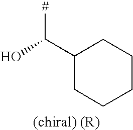 | H | |
| 125 | ethyl/H (chiral) (S) | CH₃(chiral) (S) | 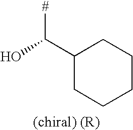 | H | |
| 126 | ethyl/H (chiral) (R) | CH₃(chiral) (R) | 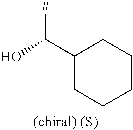 | H | |
| 127 | ethyl/H (chiral) (S) | CH₃(chiral) (R) | 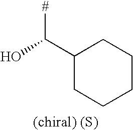 | H | |
| 128 | ethyl/H (chiral) (+/−) | CH₃(chiral) (+/−) | 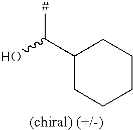 | H | |
| 129 | ethyl/H (chiral) (R) | CH₃(chiral) (S) | 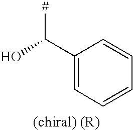 | H | |
| 130 | ethyl/H (chiral) (S) | CH₃(chiral) (S) | 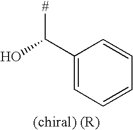 | H | |
| 131 | ethyl/H (chiral) (R) | CH₃(chiral) (R) | 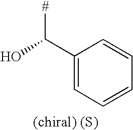 | H | |
| 132 | ethyl/H (chiral) (S) | CH₃(chiral) (R) | 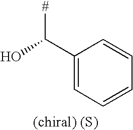 | H | |
| 133 | ethyl/H (chiral) (+/−) | CH₃(chiral) (+/−) | 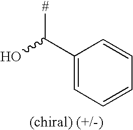 | H | |
| 134 | n-propyl/H (chiral) (R) | CH₃(chiral) (S) | 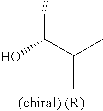 | H | |
| 135 | n-propyl/H (chiral) (S) | CH₃(chiral) (S) | 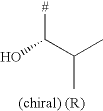 | H | |
| 136 | n-propyl/H (chiral) (R) | CH₃(chiral) (R) | 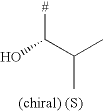 | H | |
| 137 | n-propyl/H (chiral) (S) | CH₃(chiral) (R) | 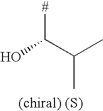 | H | |
| 138 | n-propyl/H (chiral) (+/−) | CH₃(chiral) (+/−) | 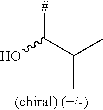 | H | |
| 139 | n-propyl/H (chiral) (R) | CH₃(chiral) (S) | 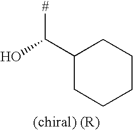 | H | |
| 140 | n-propyl/H (chiral) (S) | CH₃(chiral) (S) | 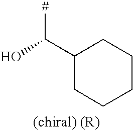 | H | |
| 141 | n-propyl/H (chiral) (R) | CH₃(chiral) (R) | 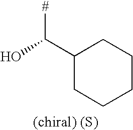 | H | |
| 142 | n-propyl/H (chiral) (S) | CH₃(chiral) (R) | 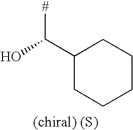 | H | |
| 143 | n-propyl/H (chiral) (+/−) | CH₃(chiral) (+/−) | 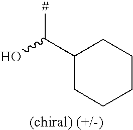 | H | |
| 144 | n-propyl/H (chiral) (R) | CH₃(chiral) (S) | 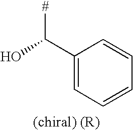 | H | |
| 145 | n-propyl/H (chiral) (S) | CH₃(chiral) (S) | 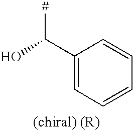 | H | |
| 146 | n-propyl/H (chiral) (R) | CH₃(chiral) (R) | 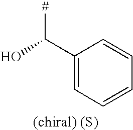 | H | |
| 147 | n-propyl/H (chiral) (S) | CH₃(chiral) (R) | 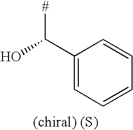 | H | |
| 148 | n-propyl/H (chiral) (+/−) | CH₃(chiral) (+/−) | 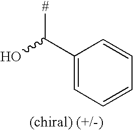 | H | |
| 149 | 3-chloro- n-propyl/H (chiral) (R) | CH₃(chiral) (S) | 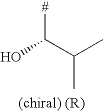 | H | |
| 150 | 3-chloro- n-propyl/H (chiral) (S) | CH₃(chiral) (S) | 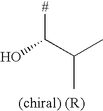 | H | |
| 151 | 3-chloro- n-propyl/H (chiral) (R) | CH₃(chiral) (R) | 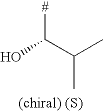 | H | |
| 152 | 3-chloro- n-propyl/H (chiral) (S) | CH₃(chiral) (R) | 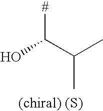 | H | |
| 153 | 3-chloro- n-propyl/H (chiral) (+/−) | CH₃(chiral) (+/−) | 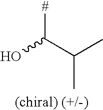 | H | |
| 154 | 3-chloro- n-propyl/H (chiral) (R) | CH₃(chiral) (S) | 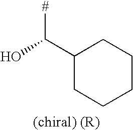 | H | |
| 155 | 3-chloro- n-propyl/H (chiral) (S) | CH₃(chiral) (S) | 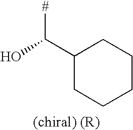 | H | |
| 156 | 3-chloro- n-propyl/H (chiral) (R) | CH₃(chiral) (R) | 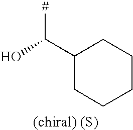 | H | |
| 157 | 3-chloro- n-propyl/H (chiral) (S) | CH₃(chiral) (R) | 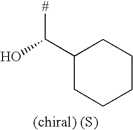 | H | |
| 158 | 3-chloro- n-propyl/H (chiral) (+/−) | CH₃(chiral) (+/−) | 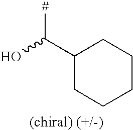 | H | |
| 159 | 3-chloro- n-propyl/H (chiral) (R) | CH₃(chiral) (S) | 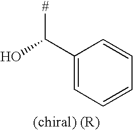 | H | |
| 160 | 3-chloro- n-propyl/H (chiral) (S) | CH₃(chiral) (S) | 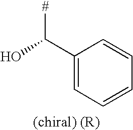 | H | |
| 161 | 3-chloro- n-propyl/H (chiral) (R) | CH₃(chiral) (R) | 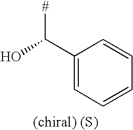 | H | |
| 162 | 3-chloro- n-propyl/H (chiral) (S) | CH₃(chiral) (R) | 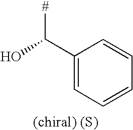 | H | |
| 163 | 3-chloro- n-propyl/H (chiral) (+/−) | CH₃(chiral) (+/−) | 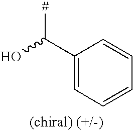 | H | |
| 164 | 4-chloro- n-butyl/H (chiral) (R) | CH₃(chiral) (S) | 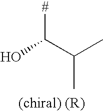 | H | |
| 165 | 4-chloro- n-butyl/H (chiral) (S) | CH₃(chiral) (S) | 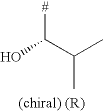 | H | |
| 166 | 4-chloro- n-butyl/H (chiral) (R) | CH₃(chiral) (R) | 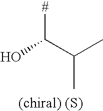 | H | |
| 167 | 4-chloro- n-butyl/H (chiral) (S) | CH₃(chiral) (R) | 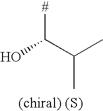 | H | |
| 168 | 4-chloro- n-butyl/H (chiral) (+/−) | CH₃(chiral) (+/−) | 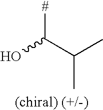 | H | |
| 169 | 4-chloro- n-butyl/H (chiral) (R) | CH₃(chiral) (S) | 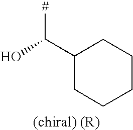 | H | |
| 170 | 4-chloro- n-butyl/H (chiral) (S) | CH₃(chiral) (S) | 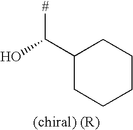 | H | |
| 171 | 4-chloro- n-butyl/H (chiral) (R) | CH₃(chiral) (R) | 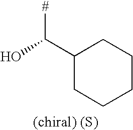 | H | |
| 172 | 4-chloro- n-butyl/H (chiral) (S) | CH₃(chiral) (R) | 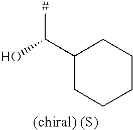 | H | |
| 173 | 4-chloro- n-butyl/H (chiral) (+/−) | CH₃(chiral) (+/−) | 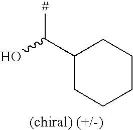 | H | |
| 174 | 4-chloro- n-butyl/H (chiral) (R) | CH₃(chiral) (S) | 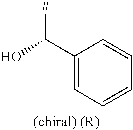 | H | |
| 175 | 4-chloro- n-butyl/H (chiral) (S) | CH₃(chiral) (S) | 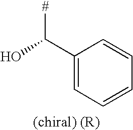 | H | |
| 176 | 4-chloro- n-butyl/H (chiral) (R) | CH₃(chiral) (R) | 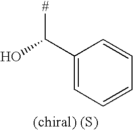 | H | |
| 177 | 4-chloro- n-butyl/H (chiral) (S) | CH₃(chiral) (R) | 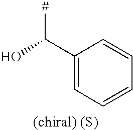 | H | |
| 178 | 4-chloro- n-butyl/H (chiral) (+/−) | CH₃(chiral) (+/−) | 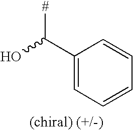 | H | |
| 179 | 2-prop- ionyloxy- ethyl/H (chiral) (R) | CH₃(chiral) (S) | 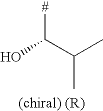 | H | |
| 180 | 2-prop- ionyloxy- ethyl/H (chiral) (S) | CH₃(chiral) (S) | 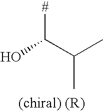 | H | |
| 181 | 2-prop- ionyloxy- ethyl/H (chiral) (R) | CH₃(chiral) (R) | 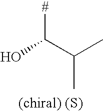 | H | |
| 182 | 2-prop- ionyloxy- ethyl/H (chiral) (S) | CH₃(chiral) (R) | 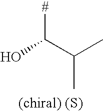 | H | |
| 183 | 2-prop- ionyloxy- ethyl/H (chiral) (+/−) | CH₃(chiral) (+/−) | 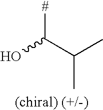 | H | |
| 184 | 2-prop- ionyloxy- ethyl/H (chiral) (R) | CH₃(chiral) (S) | 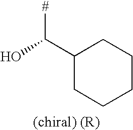 | H | |
| 185 | 2-prop- ionyloxy- ethyl/H (chiral) (S) | CH₃(chiral) (S) | 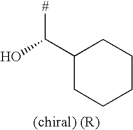 | H | |
| 186 | 2-prop- ionyloxy- ethyl/H (chiral) (R) | CH₃(chiral) (R) | 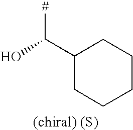 | H | |
| 187 | 2-prop- ionyloxy- ethyl/H (chiral) (S) | CH₃(chiral) (R) | 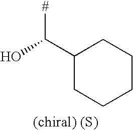 | H | |
| 188 | 2-prop- ionyloxy- ethyl/H (chiral) (+/−) | CH₃(chiral) (+/−) | 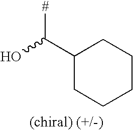 | H | |
| 189 | 2-prop- ionyloxy- ethyl/H (chiral) (R) | CH₃(chiral) (S) | 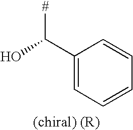 | H | |
| 190 | 2-prop- ionyloxy- ethyl/H (chiral) (S) | CH₃(chiral) (S) | 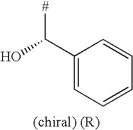 | H | |
| 191 | 2-prop- ionyloxy- ethyl/H (chiral) (R) | CH₃(chiral) (R) | 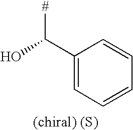 | H | |
| 192 | 2-prop- ionyloxy- ethyl/H (chiral) (S) | CH₃(chiral) (R) | 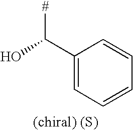 | H | |
| 193 | 2-prop- ionyloxy- ethyl/H (chiral) (+/−) | CH₃(chiral) (+/−) | 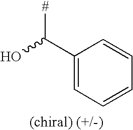 | H | |
| 194 | n-hexyl/H (chiral) (R) | CH₃(chiral) (S) | 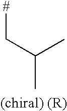 | H | |
| 195 | n-hexyl/H (chiral) (S) | CH₃(chiral) (S) | 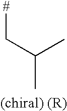 | H | |
| 196 | n-hexyl/H (chiral) (R) | CH₃(chiral) (R) | 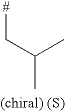 | H | |
| 197 | n-hexyl/H (chiral) (S) | CH₃(chiral) (R) | 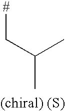 | H | |
| 198 | n-hexyl/H (chiral) (+/−) | CH₃(chiral) (+/−) | 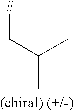 | H | |
| 199 | n-hexyl/H (chiral) (R) | CH₃(chiral) (S) | 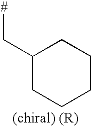 | H | |
| 200 | n-hexyl/H (chiral) (S) | CH₃(chiral) (S) | 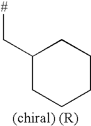 | H | |
| 201 | n-hexyl/H (chiral) (R) | CH₃(chiral) (R) | 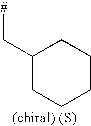 | H | |
| 202 | n-hexyl/H (chiral) (S) | CH₃(chiral) (R) | 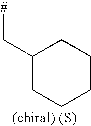 | H | |
| 203 | n-hexyl/H (chiral) (+/−) | CH₃(chiral) (+/−) | 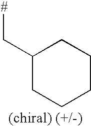 | H | |
| 204 | n-hexyl/H (chiral) (R) | CH₃(chiral) (S) | 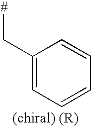 | H | |
| 205 | n-hexyl/H (chiral) (S) | CH₃(chiral) (S) | 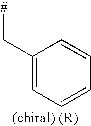 | H | |
| 206 | n-hexyl/H (chiral) (R) | CH₃(chiral) (R) | 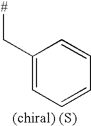 | H | |
| 207 | n-hexyl/H (chiral) (S) | CH₃(chiral) (R) | 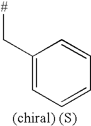 | H | |
| 208 | n-hexyl/H (chiral) (+/−) | CH₃(chiral) (+/−) | 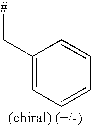 | H | |
| 209 | n-hexyl/H (chiral) (R) | H (chiral) (S) | 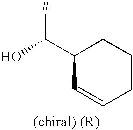 | H | |
| 210 | n-hexyl/H (chiral) (S) | H (chiral) (S) | 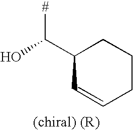 | H | |
| 211 | n-hexyl/H (chiral) (R) | H (chiral) (R) | 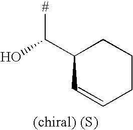 | H | |
| 212 | n-hexyl/H (chiral) (S) | H (chiral) (R) | 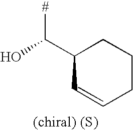 | H | |
| 213 | n-hexyl/H (chiral) (R) | H (chiral) (S) | 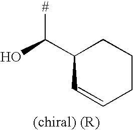 | H | |
| 214 | n-hexyl/H (chiral) (S) | H (chiral) (S) | 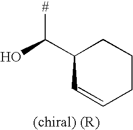 | H | |
| 215 | n-hexyl/H (chiral) (R) | H (chiral) (R) | 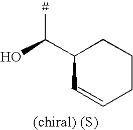 | H | |
| 216 | n-hexyl/H (chiral) (S) | H (chiral) (R) | 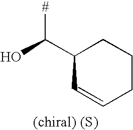 | H | |
| 217 | n-hexyl/H (chiral) (+/−) | H (chiral) (+/−) | 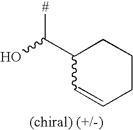 | H | |
| 218 | n-hexyl/H (chiral) (R) | H (chiral) (S) | 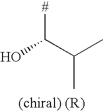 | H | |
| 219 | n-hexyl/H (chiral) (S) | H (chiral) (S) | 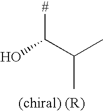 | H | |
| 220 | n-hexyl/H (chiral) (R) | H (chiral) (R) | 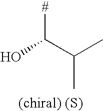 | H | |
| 221 | n-hexyl/H (chiral) (S) | H (chiral) (R) | 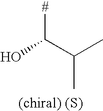 | H | |
| 222 | n-hexyl/H (chiral) (R) | H (chiral) (S) | 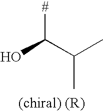 | H | |
| 223 | n-hexyl/H (chiral) (S) | H (chiral) (S) | 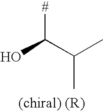 | H | |
| 224 | n-hexyl/H (chiral) (R) | H (chiral) (R) | 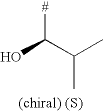 | H | |
| 225 | n-hexyl/H (chiral) (S) | H (chiral) (R) | 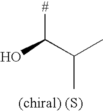 | H | |
| 226 | n-hexyl/H (chiral) (+/−) | H (chiral) (+/−) | 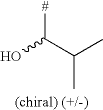 | H | |
| 227 | n-hexyl/H (chiral) (R) | H (chiral) (S) | 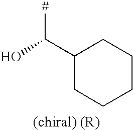 | H | |
| 228 | n-hexyl/H (chiral) (S) | H (chiral) (S) | 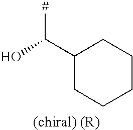 | H | |
| 229 | n-hexyl/H (chiral) (R) | H (chiral) (R) | 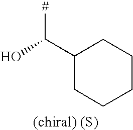 | H | |
| 230 | n-hexyl/H (chiral) (S) | H (chiral) (R) | 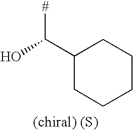 | H | |
| 231 | n-hexyl/H (chiral) (R) | H (chiral) (S) | 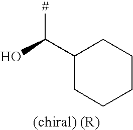 | H | |
| 232 | n-hexyl/H (chiral) (S) | H (chiral) (S) | 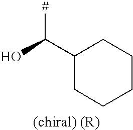 | H | |
| 233 | n-hexyl/H (chiral) (R) | H (chiral) (R) | 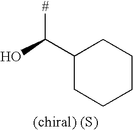 | H | |
| 234 | n-hexyl/H (chiral) (S) | H (chiral) (R) | 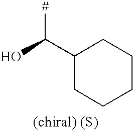 | H | |
| 235 | n-hexyl/H (chiral) (+/−) | H (chiral) (+/−) | 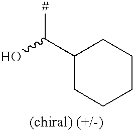 | H | |
| 236 | n-hexyl/H (chiral) (R) | H (chiral) (S) | 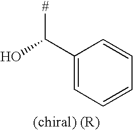 | H | |
| 237 | n-hexyl/H (chiral) (S) | H (chiral) (S) | 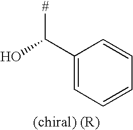 | H | |
| 238 | n-hexyl/H (chiral) (R) | H (chiral) (R) | 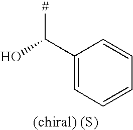 | H | |
| 239 | n-hexyl/H (chiral) (S) | H (chiral) (R) | 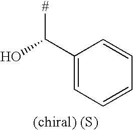 | H | |
| 240 | n-hexyl/H (chiral) (R) | H (chiral) (S) | 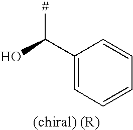 | H | |
| 241 | n-hexyl/H (chiral) (S) | H (chiral) (S) | 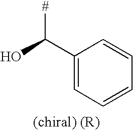 | H | |
| 242 | n-hexyl/H (chiral) (R) | H (chiral) (R) | 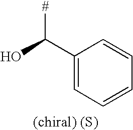 | H | |
| 243 | n-hexyl/H (chiral) (S) | H (chiral) (R) | 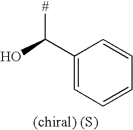 | H | |
| 244 | n-hexyl/H (chiral) (+/−) | H (chiral) (+/−) | 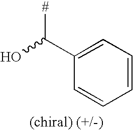 | H | |
| 245 | n-hexyl/H (chiral) (R) | H (chiral) (S) | 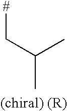 | H | |
| 246 | n-hexyl/H (chiral) (S) | H (chiral) (S) | 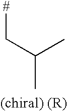 | H | |
| 247 | n-hexyl/H (chiral) (R) | H (chiral) (R) | 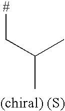 | H | |
| 248 | n-hexyl/H (chiral) (S) | H (chiral) (R) | 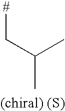 | H | |
| 249 | n-hexyl/H (chiral) (+/−) | H (chiral) (+/−) | 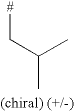 | H | |
| 250 | n-hexyl/H (chiral) (R) | H (chiral) (S) | 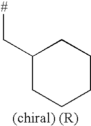 | H | |
| 251 | n-hexyl/H (chiral) (S) | H (chiral) (S) | 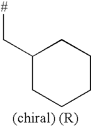 | H | |
| 252 | n-hexyl/H (chiral) (R) | H (chiral) (R) | 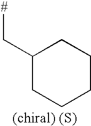 | H | |
| 253 | n-hexyl/H (chiral) (S) | H (chiral) (R) | 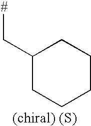 | H | |
| 254 | n-hexyl/H (chiral) (+/−) | H (chiral) (+/−) | 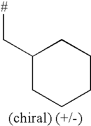 | H | |
| 255 | n-hexyl/H (chiral) (R) | H (chiral) (S) | 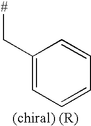 | H | |
| 256 | n-hexyl/H (chiral) (S) | H (chiral) (S) | 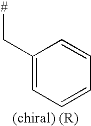 | H | |
| 257 | n-hexyl/H (chiral) (R) | H (chiral) (R) | 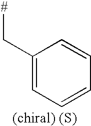 | H | |
| 258 | n-hexyl/H (chiral) (S) | H (chiral) (R) | 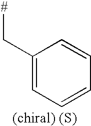 | H | |
| 259 | n-hexyl/H (chiral) (+/−) | H (chiral) (+/−) | 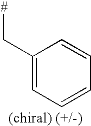 | H | |
| 260 | n-pentyl/H (chiral) (S) | CH₃(chiral) (S) | 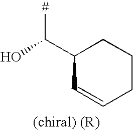 | H | |
| 261 | n-pentyl/H (chiral) (R) | CH₃(chiral) (R) | 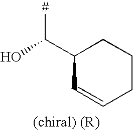 | H | |
| 262 | n-pentyl/H (chiral) (S) | CH₃(chiral) (R) | 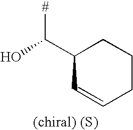 | H | |
| 263 | n-pentyl/H (chiral) (R) | CH₃(chiral) (S) | 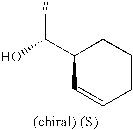 | H | |
| 264 | n-pentyl/H (chiral) (S) | CH₃(chiral) (S) | 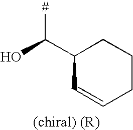 | H | |
| 265 | n-pentyl/H (chiral) (R) | CH₃(chiral) (R) | 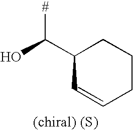 | H | |
| 266 | n-pentyl/H (chiral) (S) | CH₃(chiral) (R) | 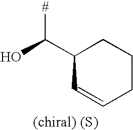 | H | |
| 267 | n-pentyl/H (chiral) (+/−) | CH₃(chiral) (+/−) | 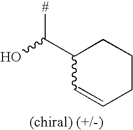 | H | |
| 268 | n-pentyl/H (chiral) (R) | CH₃(chiral) (S) | 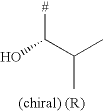 | H | |
| 269 | n-pentyl/H (chiral) (S) | CH₃(chiral) (S) | 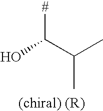 | H | |
| 270 | n-pentyl/H (chiral) (R) | CH₃(chiral) (R) | 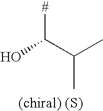 | H | |
| 271 | n-pentyl/H (chiral) (S) | CH₃(chiral) (R) | 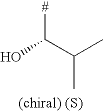 | H | |
| 272 | n-pentyl/H (chiral) (R) | CH₃(chiral) (S) | 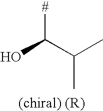 | H | |
| 273 | n-pentyl/H (chiral) (S) | CH₃(chiral) (S) | 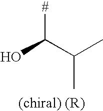 | H | |
| 274 | n-pentyl/H (chiral) (R) | CH₃(chiral) (R) | 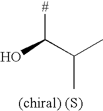 | H | |
| 275 | n-pentyl/H (chiral) (S) | CH₃(chiral) (R) | 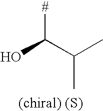 | H | |
| 276 | n-pentyl/H (chiral) (+/−) | CH₃(chiral) (+/−) | 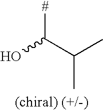 | H | |
| 277 | n-pentyl/H (chiral) (R) | CH₃(chiral) (S) | 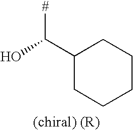 | H | |
| 278 | n-pentyl/H (chiral) (S) | CH₃(chiral) (S) | 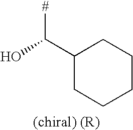 | H | |
| 279 | n-pentyl/H (chiral) (R) | CH₃(chiral) (R) | 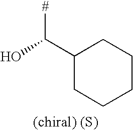 | H | |
| 280 | n-pentyl/H (chiral) (S) | CH₃(chiral) (R) | 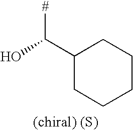 | H | |
| 281 | n-pentyl/H (chiral) (R) | CH₃(chiral) (S) | 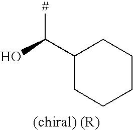 | H | |
| 282 | n-pentyl/H (chiral) (S) | CH₃(chiral) (S) | 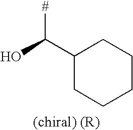 | H | |
| 283 | n-pentyl/H (chiral) (R) | CH₃(chiral) (R) | 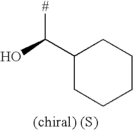 | H | |
| 284 | n-pentyl/H (chiral) (S) | CH₃(chiral) (R) | 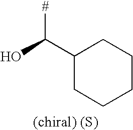 | H | |
| 285 | n-pentyl/H (chiral) (+/−) | CH₃(chiral) (+/−) | 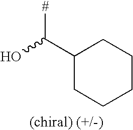 | H | |
| 286 | n-pentyl/H (chiral) (R) | CH₃(chiral) (S) | 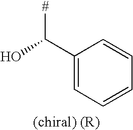 | H | |
| 287 | n-pentyl/H (chiral) (S) | CH₃(chiral) (S) | 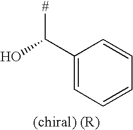 | H | |
| 288 | n-pentyl/H (chiral) (R) | CH₃(chiral) (R) | 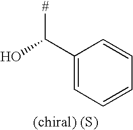 | H | |
| 289 | n-pentyl/H (chiral) (S) | CH₃(chiral) (R) | 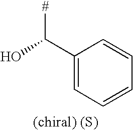 | H | |
| 290 | n-pentyl/H (chiral) (R) | CH₃(chiral) (S) | 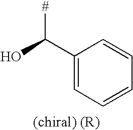 | H | |
| 291 | n-pentyl/H (chiral) (S) | CH₃(chiral) (S) | 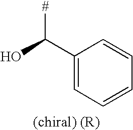 | H | |
| 292 | n-pentyl/H (chiral) (R) | CH₃(chiral) (R) | 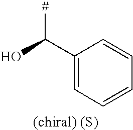 | H | |
| 293 | n-pentyl/H (chiral) (S) | CH₃(chiral) (R) | 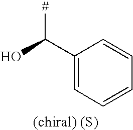 | H | |
| 294 | n-pentyl/H (chiral) (+/−) | CH₃(chiral) (+/−) | 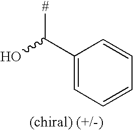 | H | |
| 295 | n-pentyl/H (chiral) (R) | CH₃(chiral) (S) | 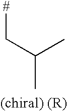 | H | |
| 296 | n-pentyl/H (chiral) (S) | CH₃(chiral) (S) | 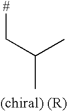 | H | |
| 297 | n-pentyl/H (chiral) (R) | CH₃(chiral) (R) | 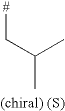 | H | |
| 298 | n-pentyl/H (chiral) (S) | CH₃(chiral) (R) | 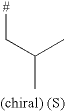 | H | |
| 299 | n-pentyl/H (chiral) (+/−) | CH₃(chiral) (+/−) | 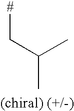 | H | |
| 300 | n-pentyl/H (chiral) (R) | CH₃(chiral) (S) | 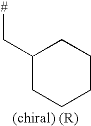 | H | |
| 301 | n-pentyl/H (chiral) (S) | CH₃(chiral) (S) | 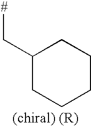 | H | |
| 302 | n-pentyl/H (chiral) (R) | CH₃(chiral) (R) | 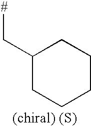 | H | |
| 303 | n-pentyl/H (chiral) (S) | CH₃(chiral) (R) | 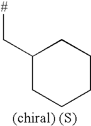 | H | |
| 304 | n-pentyl/H (chiral) (+/−) | CH₃(chiral) (+/−) | 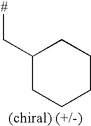 | H | |
| 305 | n-pentyl/H (chiral) (R) | CH₃(chiral) (S) | 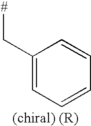 | H | |
| 306 | n-pentyl/H (chiral) (S) | CH₃(chiral) (S) | 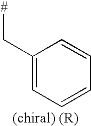 | H | |
| 307 | n-pentyl/H (chiral) (R) | CH₃(chiral) (R) | 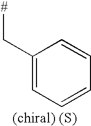 | H | |
| 308 | n-pentyl/H (chiral) (S) | CH₃(chiral) (R) | 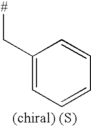 | H | |
| 309 | n-pentyl/H (chiral) (+/−) | CH₃(chiral) (+/−) | 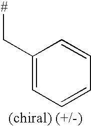 | H | |
| 310 | n-pentyl/H (chiral) (R) | H (chiral) (S) | 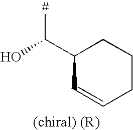 | H | |
| 311 | n-pentyl/H (chiral) (S) | H (chiral) (S) | 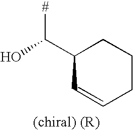 | H | |
| 312 | n-pentyl/H (chiral) (R) | H (chiral) (R) | 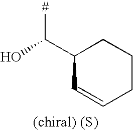 | H | |
| 313 | n-pentyl/H (chiral) (S) | H (chiral) (R) | 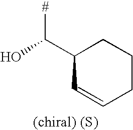 | H | |
| 314 | n-pentyl/H (chiral) (R) | H (chiral) (S) | 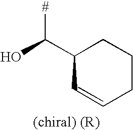 | H | |
| 315 | n-pentyl/H (chiral) (S) | H (chiral) (S) | 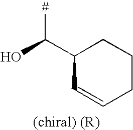 | H | |
| 316 | n-pentyl/H (chiral) (R) | H (chiral) (R) | 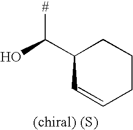 | H | |
| 317 | n-pentyl/H (chiral) (S) | H (chiral) (R) | 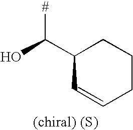 | H | |
| 318 | n-pentyl/H (chiral) (+/−) | H (chiral) (+/−) | 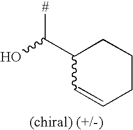 | H | |
| 319 | n-pentyl/H (chiral) (R) | H (chiral) (S) | 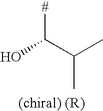 | H | |
| 320 | n-pentyl/H (chiral) (S) | H (chiral) (S) | 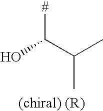 | H | |
| 321 | n-pentyl/H (chiral) (R) | H (chiral) (R) | 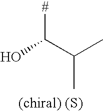 | H | |
| 322 | n-pentyl/H (chiral) (S) | H (chiral) (R) | 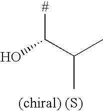 | H | |
| 323 | n-pentyl/H (chiral) (R) | H (chiral) (S) | 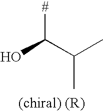 | H | |
| 324 | n-pentyl/H (chiral) (S) | H (chiral) (S) | 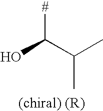 | H | |
| 325 | n-pentyl/H (chiral) (R) | H (chiral) (R) | 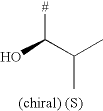 | H | |
| 326 | n-pentyl/H (chiral) (S) | H (chiral) (R) | 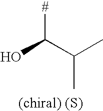 | H | |
| 327 | n-pentyl/H (chiral) (+/−) | H (chiral) (+/−) | 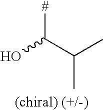 | H | |
| 328 | n-pentyl/H (chiral) (R) | H (chiral) (S) | 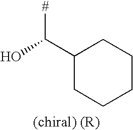 | H | |
| 329 | n-pentyl/H (chiral) (S) | H (chiral) (S) | 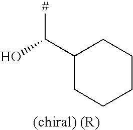 | H | |
| 330 | n-pentyl/H (chiral) (R) | H (chiral) (R) | 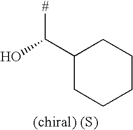 | H | |
| 331 | n-pentyl/H (chiral) (S) | H (chiral) (R) | 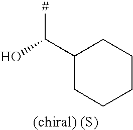 | H | |
| 332 | n-pentyl/H (chiral) (R) | H (chiral) (S) | 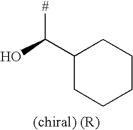 | H | |
| 333 | n-pentyl/H (chiral) (S) | H (chiral) (S) | 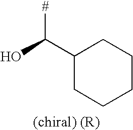 | H | |
| 334 | n-pentyl/H (chiral) (R) | H (chiral) (R) | 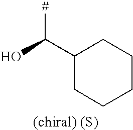 | H | |
| 335 | n-pentyl/H (chiral) (S) | H (chiral) (R) | 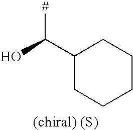 | H | |
| 336 | n-pentyl/H (chiral) (+/−) | H (chiral) (+/−) | 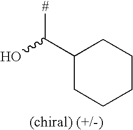 | H | |
| 337 | n-pentyl/H (chiral) (R) | H (chiral) (S) | 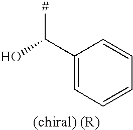 | H | |
| 338 | n-pentyl/H (chiral) (S) | H (chiral) (S) | 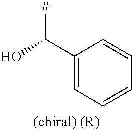 | H | |
| 339 | n-pentyl/H (chiral) (R) | H (chiral) (R) | 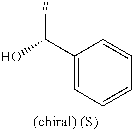 | H | |
| 340 | n-pentyl/H (chiral) (S) | H (chiral) (R) | 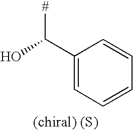 | H | |
| 341 | n-pentyl/H (chiral) (R) | H (chiral) (S) | 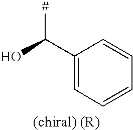 | H | |
| 342 | n-pentyl/H (chiral) (S) | H (chiral) (S) | 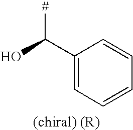 | H | |
| 343 | n-pentyl/H (chiral) (R) | H (chiral) (R) | 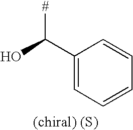 | H | |
| 344 | n-pentyl/H (chiral) (S) | H (chiral) (R) | 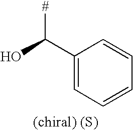 | H | |
| 345 | n-pentyl/H (chiral) (+/−) | H (chiral) (+/−) | 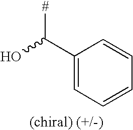 | H | |
| 346 | n-pentyl/H (chiral) (R) | H (chiral) (S) | 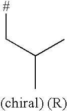 | H | |
| 347 | n-pentyl/H (chiral) (S) | H (chiral) (S) | 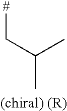 | H | |
| 348 | n-pentyl/H (chiral) (R) | H (chiral) (R) | 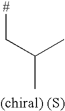 | H | |
| 349 | n-pentyl/H (chiral) (S) | H (chiral) (R) | 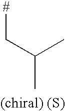 | H | |
| 350 | n-pentyl/H (chiral) (+/−) | H (chiral) (+/−) | 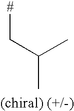 | H | |
| 351 | n-pentyl/H (chiral) (R) | H (chiral) (S) | 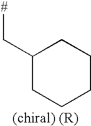 | H | |
| 352 | n-pentyl/H (chiral) (S) | H (chiral) (S) | 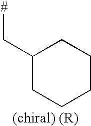 | H | |
| 353 | n-pentyl/H (chiral) (R) | H (chiral) (R) | 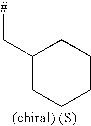 | H | |
| 354 | n-pentyl/H (chiral) (S) | H (chiral) (R) | 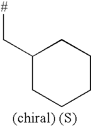 | H | |
| 355 | n-pentyl/H (chiral) (+/−) | H (chiral) (+/−) | 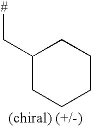 | H | |
| 356 | n-pentyl/H (chiral) (R) | H (chiral) (S) | 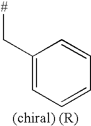 | H | |
| 357 | n-pentyl/H (chiral) (S) | H (chiral) (S) | 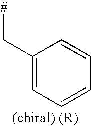 | H | |
| 358 | n-pentyl/H (chiral) (R) | H (chiral) (R) | 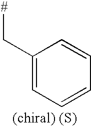 | H | |
| 359 | n-pentyl/H (chiral) (S) | H (chiral) (R) | 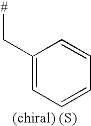 | H | |
| 360 | n-pentyl/H (chiral) (+/−) | H (chiral) (+/−) | 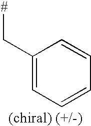 | H | |
| 361 | CH₃/H (chiral) (R) | CH₃(chiral) (S) | 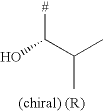 | H | |
| 362 | CH₃/H (chiral) (S) | CH₃(chiral) (S) | 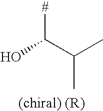 | H | |
| 363 | CH₃/H (chiral) (R) | CH₃(chiral) (R) | 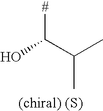 | H | |
| 364 | CH₃/H (chiral) (S) | CH₃(chiral) (R) | 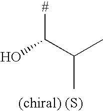 | H | |
| 365 | CH₃/H (chiral) (R) | CH₃(chiral) (S) | 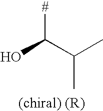 | H | |
| 366 | CH₃/H (chiral) (S) | CH₃(chiral) (S) | 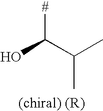 | H | |
| 367 | CH₃/H (chiral) (R) | CH₃(chiral) (R) | 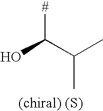 | H | |
| 368 | CH₃/H (chiral) (S) | CH₃(chiral) (R) | 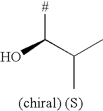 | H | |
| 369 | CH₃/H (chiral) (+/−) | CH₃(chiral) (+/−) | 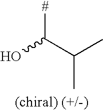 | H | |
| 370 | CH₃/H (chiral) (R) | CH₃(chiral) (S) | 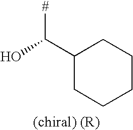 | H | |
| 371 | CH₃/H (chiral) (S) | CH₃(chiral) (S) | 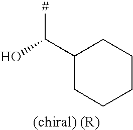 | H | |
| 372 | CH₃/H (chiral) (R) | CH₃(chiral) (R) | 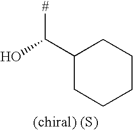 | H | |
| 373 | CH₃/H (chiral) (S) | CH₃(chiral) (R) | 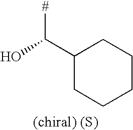 | H | |
| 374 | CH₃/H (chiral) (R) | CH₃(chiral) (S) | 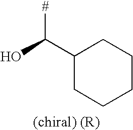 | H | |
| 375 | CH₃/H (chiral) (S) | CH₃(chiral) (S) | 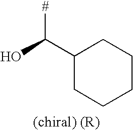 | H | |
| 376 | CH₃/H (chiral) (R) | CH₃(chiral) (R) | 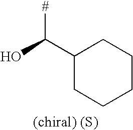 | H | |
| 377 | CH₃/H (chiral) (S) | CH₃(chiral) (R) | 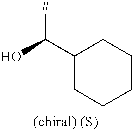 | H | |
| 378 | CH₃/H (chiral) (R) | CH₃(chiral) (+/−) | 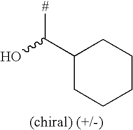 | H | |
| 379 | CH₃/H (chiral) (S) | CH₃(chiral) (S) | 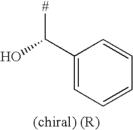 | H | |
| 380 | CH₃/H (chiral) (R) | CH₃(chiral) (S) | 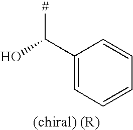 | H | |
| 381 | CH₃/H (chiral) (S) | CH₃(chiral) (R) | 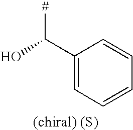 | H | |
| 382 | CH₃/H (chiral) (R) | CH₃(chiral) (R) | 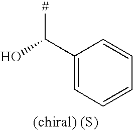 | H | |
| 383 | CH₃/H (chiral) (S) | CH₃(chiral) (S) | 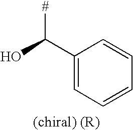 | H | |
| 384 | CH₃/H (chiral) (R) | CH₃(chiral) (S) | 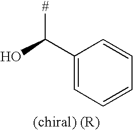 | H | |
| 385 | CH₃/H (chiral) (S) | CH₃(chiral) (R) | 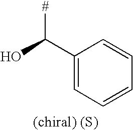 | H | |
| 386 | CH₃/H (chiral) (R) | CH₃(chiral) (R) | 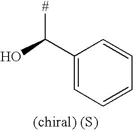 | H | |
| 387 | CH₃/H (chiral) (+/−) | CH₃(chiral) (+/−) | 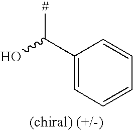 | H | |
| 388 | CH₃/H (chiral) (R) | CH₃(chiral) (S) | 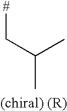 | H | |
| 389 | CH₃/H (chiral) (S) | CH₃(chiral) (S) | 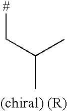 | H | |
| 390 | CH₃/H (chiral) (R) | CH₃(chiral) (R) | 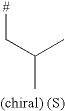 | H | |
| 391 | CH₃/H (chiral) (S) | CH₃(chiral) (R) | 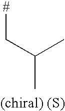 | H | |
| 392 | CH₃/H (chiral) (+/−) | CH₃(chiral) (+/−) | 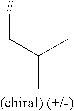 | H | |
| 393 | CH₃/H (chiral) (R) | CH₃(chiral) (S) | 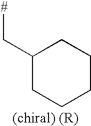 | H | |
| 394 | CH₃/H (chiral) (S) | CH₃(chiral) (S) | 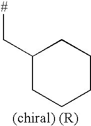 | H | |
| 395 | CH₃/H (chiral) (R) | CH₃(chiral) (R) | 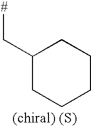 | H | |
| 396 | CH₃/H (chiral) (S) | CH₃(chiral) (R) | 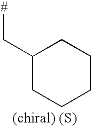 | H | |
| 397 | CH₃/H (chiral) (+/−) | CH₃(chiral) (+/−) | 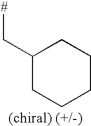 | H | |
| 398 | CH₃/H (chiral) (R) | CH₃(chiral) (S) | 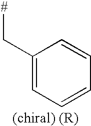 | H | |
| 399 | CH₃/H (chiral) (S) | CH₃(chiral) (S) | 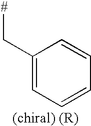 | H | |
| 400 | CH₃/H (chiral) (R) | CH₃(chiral) (R) | 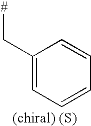 | H | |
| 401 | CH₃/H (chiral) (S) | CH₃(chiral) (R) | 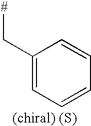 | H | |
| 402 | CH₃/H (chiral) (+/−) | CH₃(chiral) (+/−) | 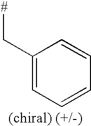 | H | |
| 403 | CH₃/H (chiral) (S) | H (chiral) (S) | 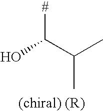 | H | |
| 404 | CH₃/H (chiral) (R) | H (chiral) (R) | 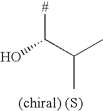 | H | |
| 405 | CH₃/H (chiral) (S) | H (chiral) (R) | 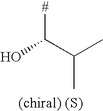 | H | |
| 406 | CH₃/H (chiral) (R) | H (chiral) (S) | 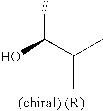 | H | |
| 407 | CH₃/H (chiral) (S) | H (chiral) (S) | 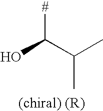 | H | |
| 408 | CH₃/H (chiral) (R) | H (chiral) (R) | 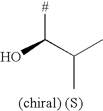 | H | |
| 409 | CH₃/H (chiral) (S) | H (chiral) (R) | 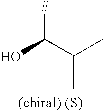 | H | |
| 410 | CH₃/H (chiral) (+/−) | H (chiral) (+/−) | 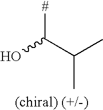 | H | |
| 411 | CH₃/H (chiral) (R) | H (chiral) (S) | 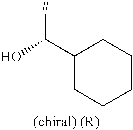 | H | |
| 412 | CH₃/H (chiral) (S) | H (chiral) (S) | 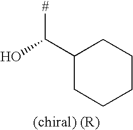 | H | |
| 413 | CH₃/H (chiral) (R) | H (chiral) (R) | 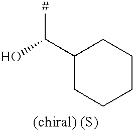 | H | |
| 414 | CH₃/H (chiral) (S) | H (chiral) (R) | 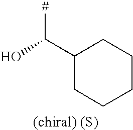 | H | |
| 415 | CH₃/H (chiral) (R) | H (chiral) (S) | 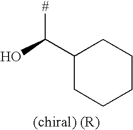 | H | |
| 416 | CH₃/H (chiral) (S) | H (chiral) (S) | 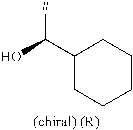 | H | |
| 417 | CH₃/H (chiral) (R) | H (chiral) (R) | 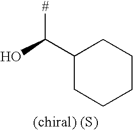 | H | |
| 418 | CH₃/H (chiral) (S) | H (chiral) (R) | 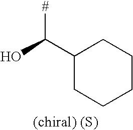 | H | |
| 419 | CH₃/H (chiral) (R) | H (chiral) (+/−) | 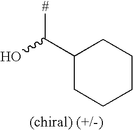 | H | |
| 420 | CH₃/H (chiral) (S) | H (chiral) (S) | 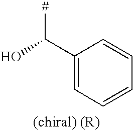 | H | |
| 421 | CH₃/H (chiral) (R) | H (chiral) (S) | 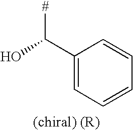 | H | |
| 422 | CH₃/H (chiral) (S) | H (chiral) (R) | 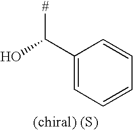 | H | |
| 423 | CH₃/H (chiral) (R) | H (chiral) (R) | 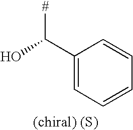 | H | |
| 424 | CH₃/H (chiral) (S) | H (chiral) (S) | 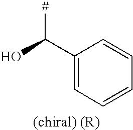 | H | |
| 425 | CH₃/H (chiral) (R) | H (chiral) (S) | 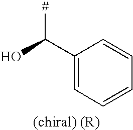 | H | |
| 426 | CH₃/H (chiral) (S) | H (chiral) (R) | 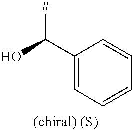 | H | |
| 427 | CH₃/H (chiral) (R) | H (chiral) (R) | 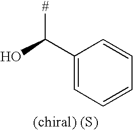 | H | |
| 428 | CH₃/H (chiral) (+/−) | H (chiral) (+/−) | 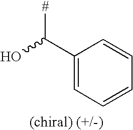 | H | |
| 429 | CH₃/H (chiral) (R) | H (chiral) (S) | 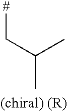 | H | |
| 430 | CH₃/H (chiral) (S) | H (chiral) (S) | 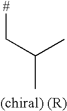 | H | |
| 431 | CH₃/H (chiral) (R) | H (chiral) (R) | 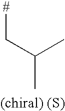 | H | |
| 432 | CH₃/H (chiral) (S) | H (chiral) (R) | 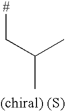 | H | |
| 433 | CH₃/H (chiral) (+/−) | H (chiral) (+/−) | 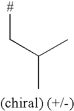 | H | |
| 434 | CH₃/H (chiral) (R) | H (chiral) (S) | 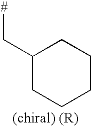 | H | |
| 435 | CH₃/H (chiral) (S) | H (chiral) (S) | 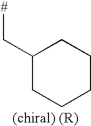 | H | |
| 436 | CH₃/H (chiral) (R) | H (chiral) (R) | 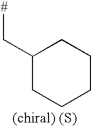 | H | |
| 437 | CH₃/H (chiral) (S) | H (chiral) (R) | 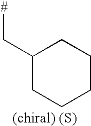 | H | |
| 438 | CH₃/H (chiral) (+/−) | H (chiral) (+/−) | 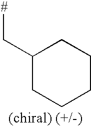 | H | |
| 439 | CH₃/H (chiral) (R) | H (chiral) (S) | 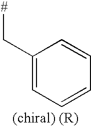 | H | |
| 440 | CH₃/H (chiral) (S) | H (chiral) (S) | 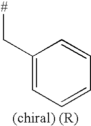 | H | |
| 441 | CH₃/H (chiral) (R) | H (chiral) (R) | 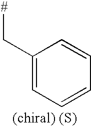 | H | |
| 442 | CH₃/H (chiral) (S) | H (chiral) (R) | 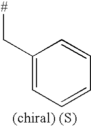 | H | |
| 443 | CH₃/H (chiral) (+/−) | H (chiral) (+/−) | 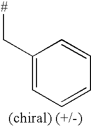 | H | |

Determination of logP-values was made according to EEC-Directive 79/831. Annex V. A8 by HPLC (gradient method, acetonitrile/0.1% aqueous phosphoric acid).

According to the methods mentioned before the lactames of the formula

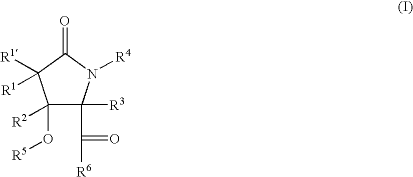

named in the following table can or have been obtained:

TABLE 6

| Example | R¹/R^{1′}* | R² | R³ | R⁴** | R⁵ | R⁶ | logP*) |
|---|---|---|---|---|---|---|---|
| 4 | 1-hydroxy- hexyl/H (chiral) (R) | CH₃(chiral) (S) | 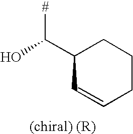 | H | H | OH | |
| 5 | n-hexyl/H (chiral) (R) | CH₃(chiral) (S) | 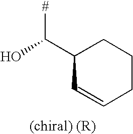 | H | H | 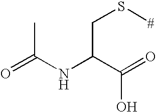 | |
| 6 | n-hexyl/H (chiral) (R) | CH₃(chiral) (S) | 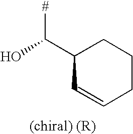 | H | H | 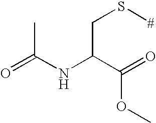 | |
| 7 | n-hexyl/H (chiral) (R) | CH₃(chiral) (S) | 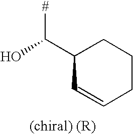 | H | H | OH | |
| 444 | n-hexyl/H (chiral) (R) | CH₃(chiral) (R) | 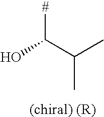 | H | H | OCH₃ | 2.30 |
| 445 | n-hexyl/H (chiral) (R) | CH₃(chiral) (R) | 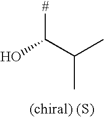 | H | H | OCH₃ | |
| 446 | n-hexyl/H (chiral) (R) | CH₃(chiral) (S) | 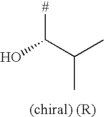 | H | H | OCH₃ | 2.58 |
| 447 | n-hexyl/H (chiral) (R) | CH₃(chiral) (S) | 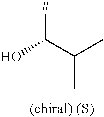 | H | H | OCH₃ | |
| 448 | n-hexyl/H (chiral) (S) | CH₃(chiral) (R) | 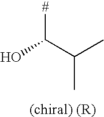 | H | H | OCH₃ | |
| 449 | n-hexyl/H (chiral) (S) | CH₃(chiral) (R) | 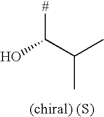 | H | H | OCH₃ | |
| 450 | n-hexyl/H (chiral) (S) | CH₃(chiral) (S) | 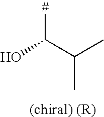 | H | H | OCH₃ | 2.16 |
| 451 | n-hexyl/H (chiral) (S) | CH₃(chiral) (S) | 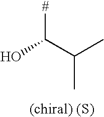 | H | H | OCH₃ | |
| 452 | n-hexyl/H (chiral) (R) | CH₃(chiral) (S) | 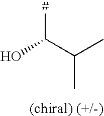 | H | H | OCH₃ | |
| 453 | n-hexyl/H (chiral) (R) | CH₃(chiral) (+/−) | 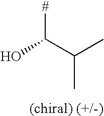 | H | H | OCH₃ | |
| 454 | n-hexyl/H (chiral) (+/−) | CH₃(chiral) (+/−) | 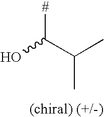 | H | H | OCH₃ | |
| 455 | n-hexenyl/ BnOCO (chiral) (+/−) | H (chiral) (+/−) | 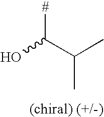 | BOC | H | OCH₃ | |
| 456 | n-hexenyl/ BnOCO (chiral) (+/−) | H (chiral) (+/−) | 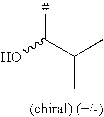 | BOC | H | OCH₂CH₃ | |
| 457 | n-hexenyl/ BnOCO (chiral) (+/−) | H (chiral) (+/−) | 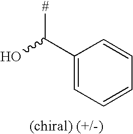 | BOC | H | OCH₃ | |
| 458 | n-hexenyl/ BnOCO (chiral) (+/−) | H (chiral) (+/−) | 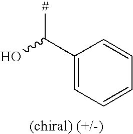 | BOC | H | OCH₂CH₃ | |
| 459 | n-hexenyl/ BnOCO (chiral) (+/−) | H (chiral) (+/−) | 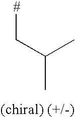 | BOC | H | OCH₃ | |
| 460 | n-hexenyl/ BnOCO (chiral) (+/−) | H (chiral) (+/−) | 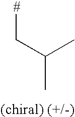 | BOC | H | OCH₂CH₃ | |
| 461 | n-hexenyl/ BnOCO (chiral) (+/−) | H (chiral) (+/−) | 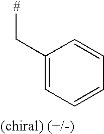 | BOC | H | OCH₃ | |
| 462 | n-hexenyl/ BnOCO (chiral) (+/−) | H (chiral) (+/−) | 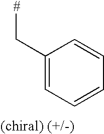 | BOC | H | OCH₂CH₃ | |
| 463 | n-hexyl/H (chiral) (R) | CH₃(chiral) (R) | 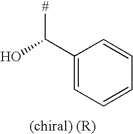 | H | H | OCH₃ | |
| 464 | n-hexyl/H (chiral) (R) | CH₃(chiral) (R) | 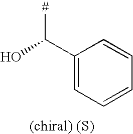 | H | H | OCH₃ | |
| 465 | n-hexyl/H (chiral) (R) | CH₃(chiral) (S) | 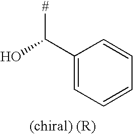 | H | H | OCH₃ | |
| 466 | n-hexyl/H (chiral) (R) | CH₃(chiral) (S) | 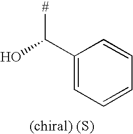 | H | H | OCH₃ | |
| 467 | n-hexyl/H (chiral) (S) | CH₃(chiral) (R) | 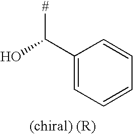 | H | H | OCH₃ | |
| 468 | n-hexyl/H (chiral) (S) | CH₃(chiral) (R) | 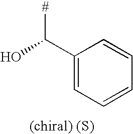 | H | H | OCH₃ | |
| 469 | n-hexyl/H (chiral) (S) | CH₃(chiral) (S) | 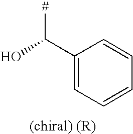 | H | H | OCH₃ | |
| 470 | n-hexyl/H (chiral) (S) | CH₃(chiral) (S) | 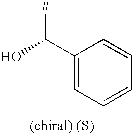 | H | H | OCH₃ | |
| 471 | n-hexyl/H (chiral) (R) | CH₃(chiral) (S) | 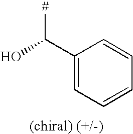 | H | H | OCH₃ | |
| 472 | n-hexyl/H (chiral) (R) | CH₃(chiral) (+/−) | 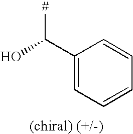 | H | H | OCH₃ | |
| 473 | n-hexyl/H (chiral) (+/−) | CH₃(chiral) (+/−) | 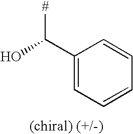 | H | H | OCH₃ | |
| 474 | n-hexyl/H (chiral) (R) | CH₃(chiral) (R) | 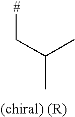 | H | H | OCH₃ | |
| 475 | n-hexyl/H (chiral) (R) | CH₃(chiral) (R) | 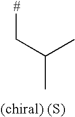 | H | H | OCH₃ | |
| 476 | n-hexyl/H (chiral) (R) | CH₃(chiral) (S) | 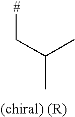 | H | H | OCH₃ | |
| 477 | n-hexyl/H (chiral) (R) | CH₃(chiral) (S) | 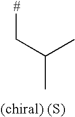 | H | H | OCH₃ | |
| 478 | n-hexyl/H (chiral) (S) | CH₃(chiral) (R) | 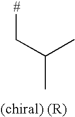 | H | H | OCH₃ | |
| 479 | n-hexyl/H (chiral) (S) | CH₃(chiral) (R) | 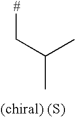 | H | H | OCH₃ | |
| 480 | n-hexyl/H (chiral) (S) | CH₃(chiral) (S) | 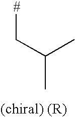 | H | H | OCH₃ | |
| 481 | n-hexyl/H (chiral) (S) | CH₃(chiral) (S) | 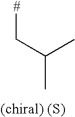 | H | H | OCH₃ | |
| 482 | n-hexyl/H (chiral) (R) | CH₃(chiral) (S) | 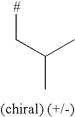 | H | H | OCH₃ | |
| 483 | n-hexyl/H (chiral) (R) | CH₃(chiral) (+/−) | 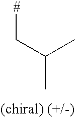 | H | H | OCH₃ | |
| 484 | n-hexyl/H (chiral) (+/−) | CH₃(chiral) (+/−) | 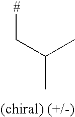 | H | H | OCH₃ | |
| 485 | n-hexyl/H (chiral) (R) | CH₃(chiral) (R) | 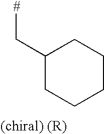 | H | H | OCH₃ | |
| 486 | n-hexyl/H (chiral) (R) | CH₃(chiral) (R) | 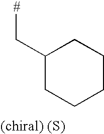 | H | H | OCH₃ | |
| 487 | n-exhyl/H (chiral) (R) | CH₃(chiral) (S) | 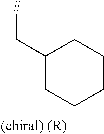 | H | H | OCH₃ | |
| 488 | n-hexyl/H (chiral) (R) | CH₃(chiral) (S) | 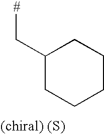 | H | H | OCH₃ | |
| 489 | n-hexyl/H (chiral) (S) | CH₃(chiral) (R) | 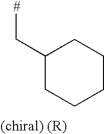 | H | H | OCH₃ | |
| 490 | n-hexyl/H (chiral) (S) | CH₃(chiral) (R) | 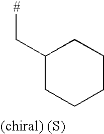 | H | H | OCH₃ | |
| 491 | n-hexyl/H (chiral) (S) | CH₃(chiral) (S) | 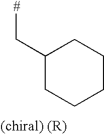 | H | H | OCH₃ | |
| 492 | n-hexyl/H (chiral) (S) | CH₃(chiral) (S) | 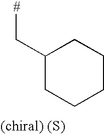 | H | H | OCH₃ | |
| 493 | n-hexyl/H (chiral) (R) | CH₃(chiral) (S) | 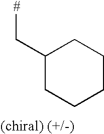 | H | H | OCH₃ | |
| 494 | n-hexyl/H (chiral) (R) | CH₃(chiral) (+/−) | 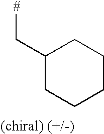 | H | H | OCH₃ | |
| 495 | n-hexyl/H (chiral) (+/−) | CH₃(chiral) (+/−) | 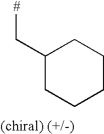 | H | H | OCH₃ | |
| 496 | n-hexyl/H (chiral) (R) | CH₃(chiral) (R) | 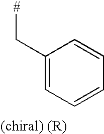 | H | H | OCH₃ | |
| 497 | n-hexyl/H (chiral) (R) | CH₃(chiral) (R) | 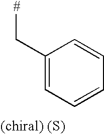 | H | H | OCH₃ | |
| 498 | n-hexyl/H (chiral) (R) | CH₃(chiral) (S) | 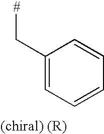 | H | H | OCH₃ | |
| 499 | n-hexyl/H (chiral) (R) | CH₃(chiral) (S) | 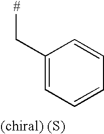 | H | H | OCH₃ | |
| 500 | n-hexyl/H (chiral) (S) | CH₃(chiral) (R) | 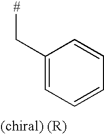 | H | H | OCH₃ | |
| 501 | n-hexyl/H (chiral) (S) | CH₃(chiral) (R) | 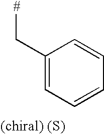 | H | H | OCH₃ | |
| 502 | n-hexyl/H (chiral) (S) | CH₃(chiral) (S) | 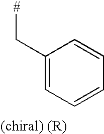 | H | H | OCH₃ | |
| 503 | n-hexyl/H (chiral) (S) | CH₃(chiral) (S) | 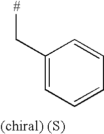 | H | H | OCH₃ | |
| 504 | n-hexyl/H (chiral) (R) | CH₃(chiral) (S) | 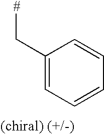 | H | H | OCH₃ | |
| 505 | n-hexyl/H (chiral) (R) | CH₃(chiral) (+/−) | 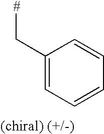 | H | H | OCH₃ | |
| 506 | n-hexyl/H (chiral) (+/−) | CH₃(chiral) (+/−) | 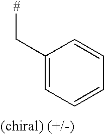 | H | H | OCH₃ | |
| 507 | n-hexyl/H (chiral) (R) | H (chiral) (R) | 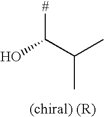 | H | H | OCH₃ | |
| 508 | n-hexyl/H (chiral) (R) | H (chiral) (R) | 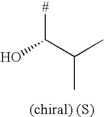 | H | H | OCH₃ | |
| 509 | n-hexyl/H (chiral) (R) | H (chiral) (S) | 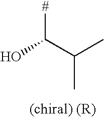 | H | H | OCH₃ | |
| 510 | n-hexyl/H (chiral) (R) | H (chiral) (S) | 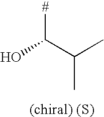 | H | H | OCH₃ | |
| 511 | n-hexyl/H (chiral) (S) | H (chiral) (R) | 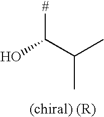 | H | H | OCH₃ | |
| 512 | n-hexyl/H (chiral) (S) | H (chiral) (R) | 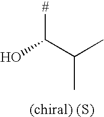 | H | H | OCH₃ | |
| 513 | n-hexyl/H (chiral) (S) | H (chiral) (S) | 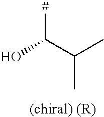 | H | H | OCH₃ | |
| 514 | n-hexyl/H (chiral) (S) | H (chiral) (S) | 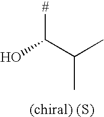 | H | H | OCH₃ | |
| 515 | n-hexyl/H (chiral) (R) | H (chiral) (S) | 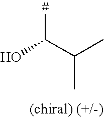 | H | H | OCH₃ | |
| 516 | n-hexyl/H (chiral) (R) | H (chiral) (+/−) | 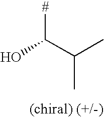 | H | H | OCH₃ | |
| 517 | n-hexyl/H (chiral) (+/−) | H (chiral) (+/−) | 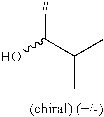 | H | H | OCH₃ | |
| 518 | n-hexyl/H (chiral) (R) | H (chiral) (R) | 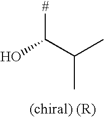 | H | H | OCH₃ | |
| 519 | n-hexyl/H (chiral) (R) | H (chiral) (R) | 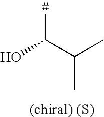 | H | H | OCH₃ | |
| 520 | n-hexyl/H (chiral) (R) | H (chiral) (S) | 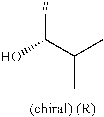 | H | H | OCH₃ | |
| 521 | n-hexyl/H (chiral) (R) | H (chiral) (S) | 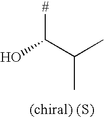 | H | H | OCH₃ | |
| 522 | n-hexyl/H (chiral) (S) | H (chiral) (R) | 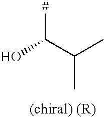 | H | H | OCH₃ | |
| 523 | n-hexyl/H (chiral) (S) | H (chiral) (R) | 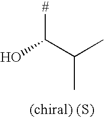 | H | H | OCH₃ | |
| 524 | n-hexyl/H (chiral) (S) | H (chiral) (S) | 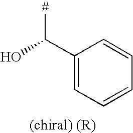 | H | H | OCH₃ | |
| 525 | n-hexyl/H (chiral) (S) | H (chiral) (S) | 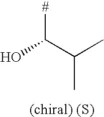 | H | H | OCH₃ | |
| 526 | n-hexyl/H (chiral) (R) | H (chiral) (S) | 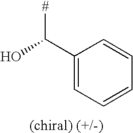 | H | H | OCH₃ | |
| 527 | n-hexyl/H (chiral) (R) | H (chiral) (+/−) | 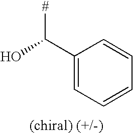 | H | H | OCH₃ | |
| 528 | n-hexyl/H (chiral) (+/−) | H (chiral) (+/−) | 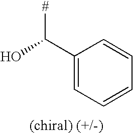 | H | H | OCH₃ | |
| 533 | n-hexyl/H (chiral) (S) | H (chiral) (R) | 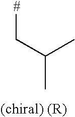 | H | H | OCH₃ | |
| 534 | n-hexyl/H (chiral) (S) | H (chiral) (R) | 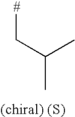 | H | H | OCH₃ | |
| 535 | n-hexyl/H (chiral) (S) | H (chiral) (S) | 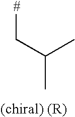 | H | H | OCH₃ | |
| 536 | n-hexyl/H (chiral) (S) | H (chiral) (S) | 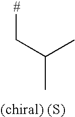 | H | H | OCH₃ | |
| 537 | n-hexyl/H (chiral) (R) | H (chiral) (S) | 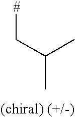 | H | H | OCH₃ | |
| 538 | n-hexyl/H (chiral) (R) | H (chiral) (+/−) | 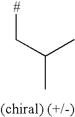 | H | H | OCH₃ | |
| 539 | n-hexyl/H (chiral) (+/−) | H (chiral) (+/−) | 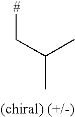 | H | H | OCH₃ | |
| 540 | n-hexyl/H (chiral) (R) | H (chiral) (R) | 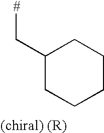 | H | H | OCH₃ | |
| 541 | n-hexyl/H (chiral) (R) | H (chiral) (R) | 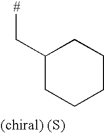 | H | H | OCH₃ | |
| 542 | n-hexyl/H (chiral) (R) | H (chiral) (S) | 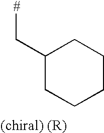 | H | H | OCH₃ | |
| 543 | n-hexyl/H (chiral) (R) | H (chiral) (S) | 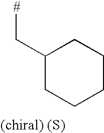 | H | H | OCH₃ | |
| 544 | n-hexyl/H (chiral) (S) | H (chiral) (R) | 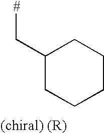 | H | H | OCH₃ | |
| 545 | n-hexyl/H (chiral) (S) | H (chiral) (R) | 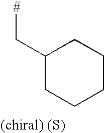 | H | H | OCH₃ | |
| 546 | n-hexyl/H (chiral) (S) | H (chiral) (S) | 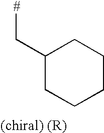 | H | H | OCH₃ | |
| 547 | n-hexyl/H (chiral) (S) | H (chiral) (S) | 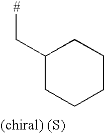 | H | H | OCH₃ | |
| 548 | n-hexyl/H (chiral) (R) | H (chiral) (S) | 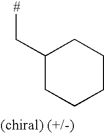 | H | H | OCH₃ | |
| 549 | n-hexyl/H (chiral) (R) | H (chiral) (+/−) | 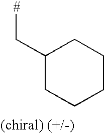 | H | H | OCH₃ | |
| 550 | n-hexyl/H (chiral) (+/−) | H (chiral) (+/−) | 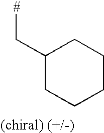 | H | H | OCH₃ | |
| 551 | n-hexyl/H (chiral) (R) | H (chiral) (R) | 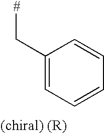 | H | H | OCH₃ | |
| 552 | n-hexyl/H (chiral) (R) | H (chiral) (R) | 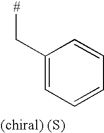 | H | H | OCH₃ | |
| 553 | n-hexyl/H (chiral) (R) | H (chiral) (S) | 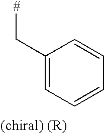 | H | H | OCH₃ | |
| 554 | n-hexyl/H (chiral) (R) | H (chiral) (S) | 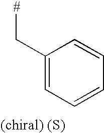 | H | H | OCH₃ | |
| 555 | n-hexyl/H (chiral) (S) | H (chiral) (R) | 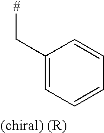 | H | H | OCH₃ | |
| 556 | n-hexyl/H (chiral) (S) | H (chiral) (R) | 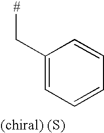 | H | H | OCH₃ | |
| 557 | n-hexyl/H (chiral) (S) | H (chiral) (S) | 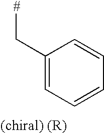 | H | H | OCH₃ | |
| 558 | n-hexyl/H (chiral) (S) | H (chiral) (S) | 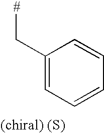 | H | H | OCH₃ | |
| 559 | n-hexyl/H (chiral) (R) | H (chiral) (S) | 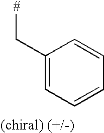 | H | H | OCH₃ | |
| 560 | n-hexyl/H (chiral) (R) | H (chiral) (+/−) | 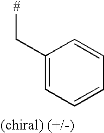 | H | H | OCH₃ | |
| 561 | n-hexyl/H (chiral) (+/−) | H (chiral) (+/−) | 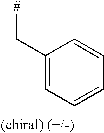 | H | H | OCH₃ | |
| 562 | n-pentyl/H (chiral) (R) | CH₃(chiral) (R) | 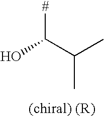 | H | H | OCH₃ | |
| 563 | n-pentyl/H (chiral) (R) | CH₃(chiral) (R) | 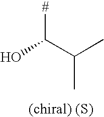 | H | H | OCH₃ | |
| 564 | n-pentyl/H (chiral) (R) | CH₃(chiral) (S) | 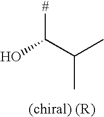 | H | H | OCH₃ | |
| 565 | n-pentyl/H (chiral) (R) | CH₃(chiral) (S) | 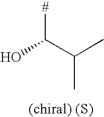 | H | H | OCH₃ | |
| 566 | n-pentyl/H (chiral) (S) | CH₃(chiral) (R) | 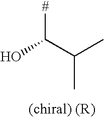 | H | H | OCH₃ | |
| 567 | n-pentyl/H (chiral) (S) | CH₃(chiral) (R) | 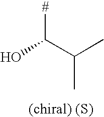 | H | H | OCH₃ | |
| 568 | n-pentyl/H (chiral) (S) | CH₃(chiral) (S) | 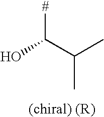 | H | H | OCH₃ | |
| 569 | n-pentyl/H (chiral) (S) | CH₃(chiral) (S) | 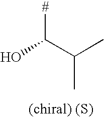 | H | H | OCH₃ | |
| 570 | n-pentyl/H (chiral) (S) | CH₃(chiral) (S) | 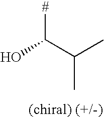 | H | H | OCH₃ | |
| 571 | n-pentyl/H (chiral) (R) | CH₂(chiral) (+/−) | 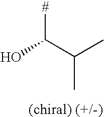 | H | H | OCH₃ | |
| 572 | n-pentyl/H (chiral) (+/−) | CH₃(chiral) (+/−) | 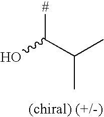 | H | H | OCH₃ | |
| 573 | n-pentyl/H (chiral) (R) | CH₃(chiral) (R) | 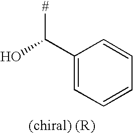 | H | H | OCH₃ | |
| 574 | n-pentyl/H (chiral) (R) | CH₃(chiral) (R) | 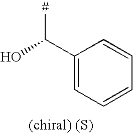 | H | H | OCH₃ | |
| 575 | n-pentyl/H (chiral) (R) | CH₃(chiral) (S) | 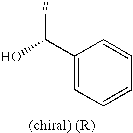 | H | H | OCH₃ | |
| 576 | n-pentyl/H (chiral) (R) | CH₃(chiral) (S) | 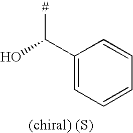 | H | H | OCH₃ | |
| 577 | n-pentyl/H (chiral) (S) | CH₃(chiral) (R) | 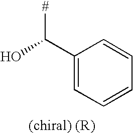 | H | H | OCH₃ | |
| 578 | n-pentyl/H (chiral) (S) | CH₃(chiral) (R) | 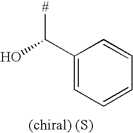 | H | H | OCH₃ | |
| 579 | n-pentyl/H (chiral) (S) | CH₃(chiral) (S) | 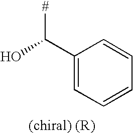 | H | H | OCH₃ | |
| 580 | n-pentyl/H (chiral) (S) | CH₃(chiral) (S) | 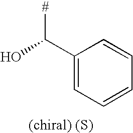 | H | H | OCH₃ | |
| 581 | n-pentyl/H (chiral) (R) | CH₃(chiral) (S) | 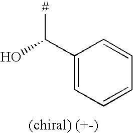 | H | H | OCH₃ | |
| 582 | n-pentyl/H (chiral) (R) | CH₃(chiral) (+/−) | 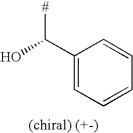 | H | H | OCH₃ | |
| 583 | n-pentyl/H (chiral) (+/−) | CH₃(chiral) (+/−) | 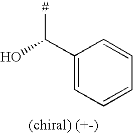 | H | H | OCH₃ | |
| 584 | n-pentyl/H (chiral) (R) | CH₃(chiral) (R) | 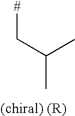 | H | H | OCH₃ | |
| 585 | n-pentyl/H (chiral) (R) | CH₃(chiral) (R) | 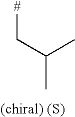 | H | H | OCH₃ | |
| 586 | n-pentyl/H (chiral) (R) | CH₃(chiral) (S) | 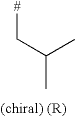 | H | H | OCH₃ | |
| 587 | n-pentyl/H (chiral) (R) | CH₃(chiral) (S) | 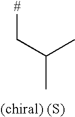 | H | H | OCH₃ | |
| 588 | n-pentyl/H (chiral) (S) | CH₃(chiral) (R) | 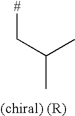 | H | H | OCH₃ | |
| 589 | n-pentyl/H (chiral) (S) | CH₃(chiral) (R) | 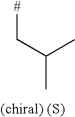 | H | H | OCH₃ | |
| 590 | n-pentyl/H (chiral) (S) | CH₃(chiral) (S) | 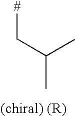 | H | H | OCH₃ | |
| 591 | n-pentyl/H (chiral) (S) | CH₃(chiral) (S) | 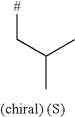 | H | H | OCH₃ | |
| 592 | n-pentyl/H (chiral) (R) | CH₃(chiral) (S) | 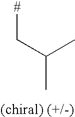 | H | H | OCH₃ | |
| 593 | n-pentyl/H (chiral) (R) | CH₃(chiral) (+/−) | 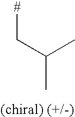 | H | H | OCH₃ | |
| 594 | n-pentyl/H (chiral) (+/−) | CH₃(chiral) (+/−) | 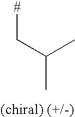 | H | H | OCH₃ | |
| 595 | n-pentyl/H (chiral) (R) | CH₃(chiral) (R) | 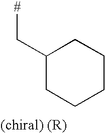 | H | H | OCH₃ | |
| 596 | n-pentyl/H (chiral) (R) | CH₃(chiral) (R) | 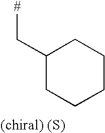 | H | H | OCH₃ | |
| 597 | n-pentyl/H (chiral) (R) | CH₃(chiral) (S) | 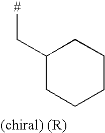 | H | H | OCH₃ | |
| 598 | n-pentyl/H (chiral) (R) | CH₃(chiral) (S) | 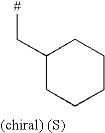 | H | H | OCH₃ | |
| 599 | n-pentyl/H (chiral) (S) | CH₃(chiral) (R) | 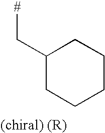 | H | H | OCH₃ | |
| 600 | n-pentyl/H (chiral) (S) | CH₃(chiral) (R) | 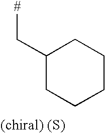 | H | H | OCH₃ | |
| 601 | n-pentyl/H (chiral) (S) | CH₃(chiral) (S) | 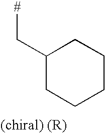 | H | H | OCH₃ | |
| 602 | n-pentyl/H (chiral) (S) | CH₃(chiral) (S) | 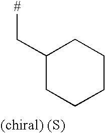 | H | H | OCH₃ | |
| 603 | n-pentyl/H (chiral) (R) | CH₃(chiral) (S) | 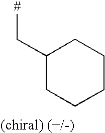 | H | H | OCH₃ | |
| 604 | n-pentyl/H (chiral) (R) | CH₃(chiral) (+/−) | 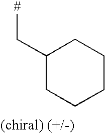 | H | H | OCH₃ | |
| 605 | n-pentyl/H (chiral) (+/−) | CH₃(chiral) (+/−) | 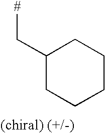 | H | H | OCH₃ | |
| 606 | n-pentyl/H (chiral) (R) | CH₃(chiral) (R) | 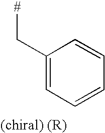 | H | H | OCH₃ | |
| 607 | n-pentyl/H (chiral) (R) | CH₃(chiral) (R) | 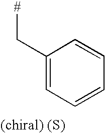 | H | H | OCH₃ | |
| 608 | n-pentyl/H (chiral) (R) | CH₃(chiral) (S) | 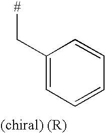 | H | H | OCH₃ | |
| 609 | n-pentyl/H (chiral) (R) | CH₃(chiral) (S) | 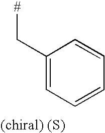 | H | H | OCH₃ | |
| 610 | n-pentyl/H (chiral) (S) | CH₃(chiral) (R) | 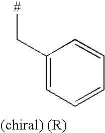 | H | H | OCH₃ | |
| 611 | n-pentyl/H (chiral) (S) | CH₃(chiral) (R) | 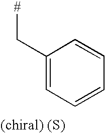 | H | H | OCH₃ | |
| 612 | n-pentyl/H (chiral) (S) | CH₃(chiral) (S) | 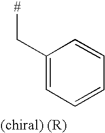 | H | H | OCH₃ | |
| 613 | n-pentyl/H (chiral) (S) | CH₃(chiral) (S) | 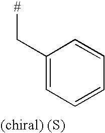 | H | H | OCH₃ | |
| 614 | n-pentyl/H (chiral) (R) | CH₃(chiral) (S) | 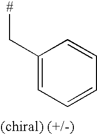 | H | H | OCH₃ | |
| 615 | n-pentyl/H (chiral) (R) | CH₂(chiral) (+/−) | 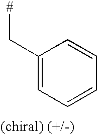 | H | H | OCH₃ | |
| 616 | n-pentyl/H (chiral) (+/−) | CH₃(chiral) (+/−) | 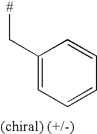 | H | H | OCH₃ | |
| 617 | n-pentyl/H (chiral) (R) | H (chiral) (R) | 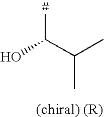 | H | H | OCH₃ | |
| 618 | n-pentyl/H (chiral) (R) | H (chiral) (R) | 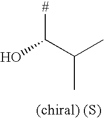 | H | H | OCH₃ | |
| 619 | n-pentyl/H (chiral) (R) | H (chiral) (S) | 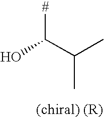 | H | H | OCH₃ | |
| 620 | n-pentyl/H (chiral) (R) | H (chiral) (S) | 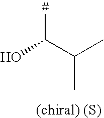 | H | H | OCH₃ | |
| 621 | n-pentyl/H (chiral) (S) | H (chiral) (R) | 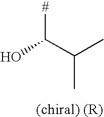 | H | H | OCH₃ | |
| 622 | n-pentyl/H (chiral) (S) | H (chiral) (R) | 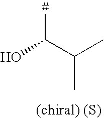 | H | H | OCH₃ | |
| 623 | n-pentyl/H (chiral) (S) | H (chiral) (S) | 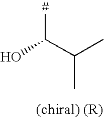 | H | H | OCH₃ | |
| 624 | n-pentyl/H (chiral) (S) | H (chiral) (S) | 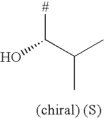 | H | H | OCH₃ | |
| 625 | n-pentyl/H (chiral) (R) | H (chiral) (S) | 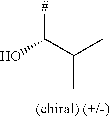 | H | H | OCH₃ | |
| 626 | n-pentyl/H (chiral) (R) | H (chiral) (+/−) | 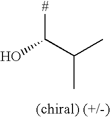 | H | H | OCH₃ | |
| 627 | n-pentyl/H (chiral) (+/−) | H (chiral) (+/−) | 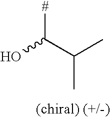 | H | H | OCH₃ | |
| 628 | n-pentyl/H (chiral) (R) | H (chiral) (R) | 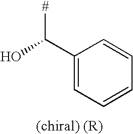 | H | H | OCH₃ | |
| 629 | n-pentyl/H (chiral) (R) | H (chiral) (R) | 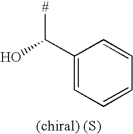 | H | H | OCH₃ | |
| 630 | n-pentyl/H (chiral) (R) | H (chiral) (S) | 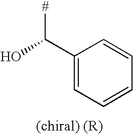 | H | H | OCH₃ | |
| 631 | n-pentyl/H (chiral) (R) | H (chiral) (S) | 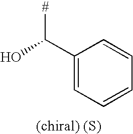 | H | H | OCH₃ | |
| 632 | n-pentyl/H (chiral) (S) | H (chiral) (R) | 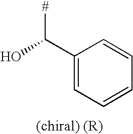 | H | H | OCH₃ | |
| 633 | n-pentyl/H (chiral) (S) | H (chiral) (R) | 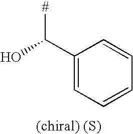 | H | H | OCH₃ | |
| 634 | n-pentyl/H (chiral) (S) | H (chiral) (S) | 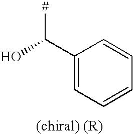 | H | H | OCH₃ | |
| 635 | n-pentyl/H (chiral) (S) | H (chiral) (S) | 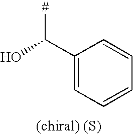 | H | H | OCH₃ | |
| 636 | n-pentyl/H (chiral) (R) | H (chiral) (S) | 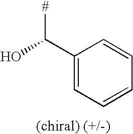 | H | H | OCH₃ | |
| 637 | n-pentyl/H (chiral) (R) | H (chiral) (+/−) | 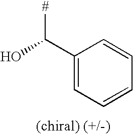 | H | H | OCH₃ | |
| 638 | n-pentyl/H (chiral) (+/−) | H (chiral) (+/−) | 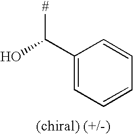 | H | H | OCH₃ | |
| 639 | n-pentyl/H (chiral) (R) | H (chiral) (R) | 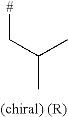 | H | H | OCH₃ | |
| 640 | n-pentyl/H (chiral) (R) | H (chiral) (R) | 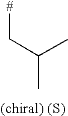 | H | H | OCH₃ | |
| 641 | n-pentyl/H (chiral) (R) | H (chiral) (S) | 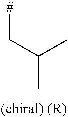 | H | H | OCH₃ | |
| 642 | n-pentyl/H (chiral) (R) | H (chiral) (S) | 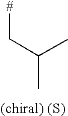 | H | H | OCH₃ | |
| 643 | n-pentyl/H (chiral) (S) | H (chiral) (R) | 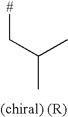 | H | H | OCH₃ | |
| 644 | n-pentyl/H (chiral) (S) | H (chiral) (R) | 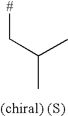 | H | H | OCH₃ | |
| 645 | n-pentyl/H (chiral) (S) | H (chiral) (S) | 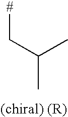 | H | H | OCH₃ | |
| 646 | n-pentyl/H (chiral) (S) | H (chiral) (S) | 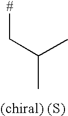 | H | H | OCH₃ | |
| 647 | n-pentyl/H (chiral) (R) | H (chiral) (S) | 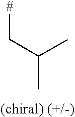 | H | H | OCH₃ | |
| 648 | n-pentyl/H (chiral) (R) | H (chiral) (+/−) | 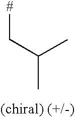 | H | H | OCH₃ | |
| 649 | n-pentyl/H (chiral) (+/−) | H (chiral) (+/−) | 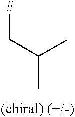 | | | | |
| 650 | n-pentyl/H (chiral) (R) | H (chiral) (R) | 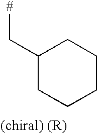 | H | H | OCH₃ | |
| 651 | n-pentyl/H (chiral) (R) | H (chiral) (R) | 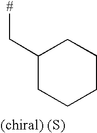 | H | H | OCH₃ | |
| 652 | n-pentyl/H (chiral) (R) | H (chiral) (S) | 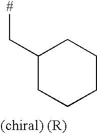 | H | H | OCH₃ | |
| 653 | n-pentyl/H (chiral) (R) | H (chiral) (S) | 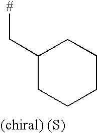 | H | H | OCH₃ | |
| 654 | n-pentyl/H (chiral) (S) | H (chiral) (R) | 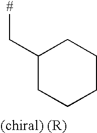 | H | H | OCH₃ | |
| 655 | n-pentyl/H (chiral) (S) | H (chiral) (R) | 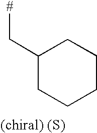 | H | H | OCH₃ | |
| 656 | n-pentyl/H (chiral) (S) | H (chiral) (S) | 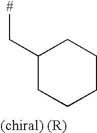 | H | H | OCH₃ | |
| 657 | n-pentyl/H (chiral) (S) | H (chiral) (S) | 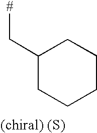 | H | H | OCH₃ | |
| 658 | n-pentyl/H (chiral) (R) | H (chiral) (S) | 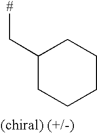 | H | H | OCH₃ | |
| 659 | n-pentyl/H (chiral) (R) | H (chiral) (+/−) | 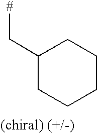 | H | H | OCH₃ | |
| 660 | n-pentyl/H (chiral) (+/−) | H (chiral) (+/−) | 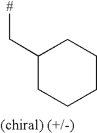 | H | H | OCH₃ | |
| 661 | n-pentyl/H (chiral) (R) | H (chiral) (R) | 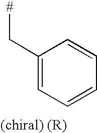 | H | H | OCH₃ | |
| 662 | n-pentyl/H (chiral) (R) | H (chiral) (R) | 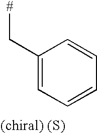 | H | H | OCH₃ | |
| 663 | n-pentyl/H (chiral) (R) | H (chiral) (S) | 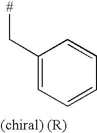 | H | H | OCH₃ | |
| 664 | n-pentyl/H (chiral) (R) | H (chiral) (S) | 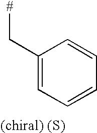 | H | H | OCH₃ | |
| 665 | n-pentyl/H (chiral) (S) | H (chiral) (R) | 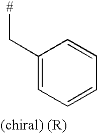 | H | H | OCH₃ | |
| 666 | n-pentyl/H (chiral) (S) | H (chiral) (R) | 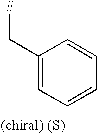 | H | H | OCH₃ | |
| 667 | n-pentyl/H (chiral) (S) | H (chiral) (S) | 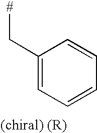 | H | H | OCH₃ | |
| 668 | n-pentyl/H (chiral) (S) | H (chiral) (S) | 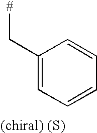 | H | H | OCH₃ | |
| 669 | n-pentyl/H (chiral) (R) | H (chiral) (S) | 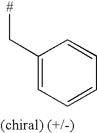 | H | H | OCH₃ | |
| 670 | n-pentyl/H (chiral) (R) | H (chiral) (+/−) | 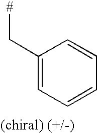 | H | H | OCH₃ | |
| 671 | n-pentyl/H (chiral) (+/−) | H (chiral) (+/−) | 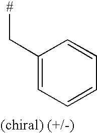 | H | H | OCH₃ | |
| 672 | n-hexyl/H (chiral) (R) | CH₃(chiral) (R) | 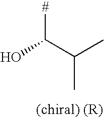 | H | H | OH | |
| 673 | n-hexyl/H (chiral) (R) | CH₃(chiral) (R) | 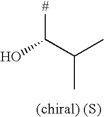 | H | H | OH | |
| 674 | n-hexyl/H (chiral) (R) | CH₃(chiral) (S) | 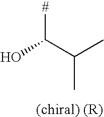 | H | H | OH | |
| 675 | n-hexyl | H (chiral) (R) | CH₃(chiral) (S) | 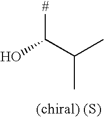 | H | H | OH |
| 676 | n-hexyl/H (chiral) (S) | CH₃(chiral) (R) | 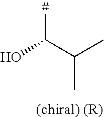 | H | H | OH | |
| 677 | n-hexyl/H (chiral) (S) | CH₃(chiral) (R) | 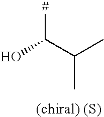 | H | H | OH | |
| 678 | n-hexyl/H (chiral) (S) | CH₃(chiral) (S) | 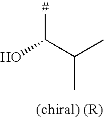 | H | H | OH | |
| 679 | n-hexyl/H (chiral) (S) | CH₃(chiral) (S) | 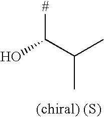 | H | H | OH | |
| 680 | n-hexyl/H (chiral) (R) | CH₃(chiral) (S) | 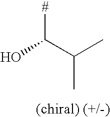 | H | H | OH | |
| 681 | n-hexyl/H (chiral) (R) | CH₃(chiral) (+/−) | 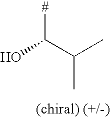 | H | H | OH | |
| 682 | n-hexyl/H (chiral) (+/−) | CH₃(chiral) (+/−) | 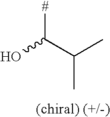 | H | H | OH | |
| 683 | n-hexyl/H (chiral) (R) | CH₃(chiral) (R) | 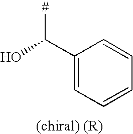 | H | H | OH | |
| 684 | n-hexyl/H (chiral) (R) | CH₃(chiral) (R) | 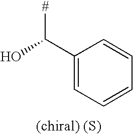 | H | H | OH | |
| 685 | n-hexyl/H (chiral) (R) | CH₃(chiral) (S) | 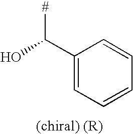 | H | H | OH | |
| 686 | n-hexyl/H (chiral) (R) | CH₃(chiral) (S) | 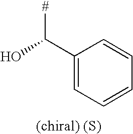 | H | H | OH | |
| 687 | n-hexyl/h (chiral) (S) | CH₃(chiral) (R) | 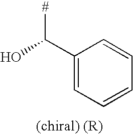 | H | H | OH | |
| 688 | n-hexyl/H (chiral) (S) | CH₃(chiral) (R) | 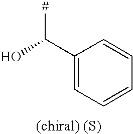 | H | H | OH | |
| 689 | n-hexyl/H (chiral) (S) | CH₃(chiral) (S) | 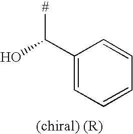 | H | H | OH | |
| 690 | n-hexyl/H (chiral) (S) | CH₃(chiral) (S) | 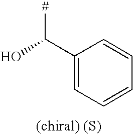 | H | H | OH | |
| 691 | n-hexyl/H (chiral) (R) | CH₃(chiral) (S) | 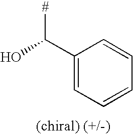 | H | H | OH | |
| 692 | n-hexyl/H (chiral) (R) | CH₃(chiral) (+/−) | 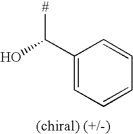 | H | H | OH | |
| 693 | n-hexyl/H (chiral) (+/−) | CH₃(chiral) (+/−) | 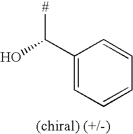 | H | H | OH | |
| 694 | n-hexyl/H (chiral) (R) | CH₃(chiral) (R) | 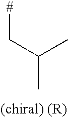 | H | H | OH | |
| 695 | n-hexyl/H (chiral) (R) | CH₃(chiral) (R) | 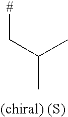 | H | H | OH | |
| 696 | n-hexyl/H (chiral) (R) | CH₃(chiral) (S) | 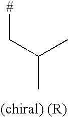 | H | H | OH | |
| 697 | n-hexyl/H (chiral) (R) | CH₃(chiral) (S) | 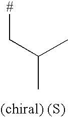 | H | H | OH | |
| 698 | n-hexyl/H (chiral) (S) | CH₃(chiral) (R) | 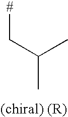 | H | H | OH | |
| 699 | n-hexyl/H (chiral) (S) | CH₃(chiral) (R) | 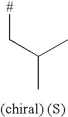 | H | H | OH | |
| 700 | n-hexyl/H (chiral) (S) | CH₃(chiral) (S) | 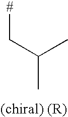 | H | H | OH | |
| 701 | n-hexyl/H (chiral) (S) | CH₃(chiral) (S) | 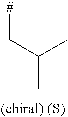 | H | H | OH | |
| 702 | n-hexyl/H (chiral) (R) | CH₃(chiral) (S) | 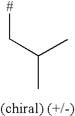 | H | H | OH | |
| 703 | n-hexyl/H (chiral) (R) | CH₃(chiral) (+/−) | 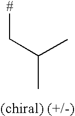 | H | H | OH | |
| 704 | n-hexyl/H (chiral) (+/−) | CH₃(chiral) (+/−) | 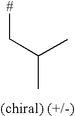 | H | H | OH | |
| 705 | n-hexyl/H (chiral) (R) | CH₃(chiral) (R) | 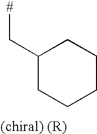 | H | H | OH | |
| 706 | n-hexyl/H (chiral) (R) | CH₃(chiral) (R) | 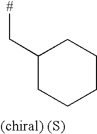 | H | H | OH | |
| 707 | n-hexyl/H (chiral) (R) | CH₃(chiral) (S) | 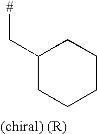 | H | H | OH | |
| 708 | n-hexyl/H (chiral) (R) | CH₃(chiral) (S) | 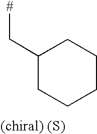 | H | H | OH | |
| 709 | n-hexyl/H (chiral) (S) | CH₃(chiral) (R) | 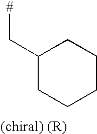 | H | H | OH | |
| 710 | n-hexyl/H (chiral) (S) | CH₃(chiral) (R) | 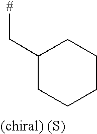 | H | H | OH | |
| 711 | n-hexyl/H (chiral) (S) | CH₃(chiral) (S) | 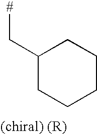 | H | H | OH | |
| 712 | n-hexyl/H (chiral) (S) | CH₃(chiral) (S) | 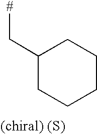 | H | H | OH | |
| 713 | n-hexyl/H (chiral) (R) | CH₃(chiral) (S) | 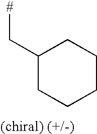 | H | H | OH | |
| 714 | n-hexyl/H (chiral) (R) | CH₃(chiral) (+/−) | 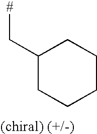 | H | H | OH | |
| 715 | n-hexyl/H (chiral) (+/−) | CH₃(chiral) (+/−) | 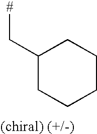 | H | H | OH | |
| 716 | n-hexyl/H (chiral) (R) | CH₃(chiral) (R) | 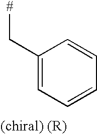 | H | H | OH | |
| 717 | n-hexyl/H (chiral) (R) | CH₃(chiral) (R) | 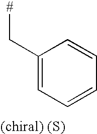 | H | H | OH | |
| 718 | n-hexyl/H (chiral) (R) | CH₃(chiral) (S) | 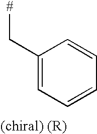 | H | H | OH | |
| 719 | n-hexyl/H (chiral) (R) | CH₃(chiral) (S) | 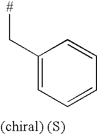 | H | H | OH | |
| 720 | n-hexyl/H (chiral) (S) | CH₃(chiral) (R) | 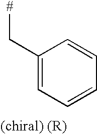 | H | H | OH | |
| 721 | n-hexyl/H (chiral) (S) | CH₃(chiral) (R) | 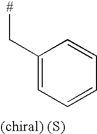 | H | H | OH | |
| 722 | n-hexyl/H (chiral) (S) | CH₂(chiral) (S) | 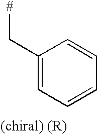 | H | H | OH | |
| 723 | n-hexyl/H (chiral) (S) | CH₃(chiral) (S) | 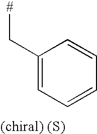 | H | H | OH | |
| 724 | n-hexyl/H (chiral) (R) | CH₃(chiral) (S) | 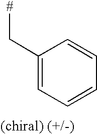 | H | H | OH | |
| 725 | n-hexyl/H (chiral) (R) | CH₃(chiral) (+/−) | 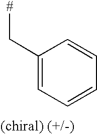 | H | H | OH | |
| 726 | n-hexyl/H (chiral) (+/−) | CH₃(chiral) (+/−) | 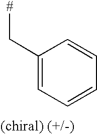 | H | H | OH | |
| 727 | n-hexyl/H (chiral) (R) | H (chiral) (R) | 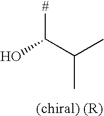 | H | H | OH | |
| 728 | n-hexyl/H (chiral) (R) | H (chiral) (R) | 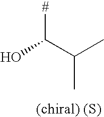 | H | H | OH | |
| 729 | n-hexyl/H (chiral) (R) | H (chiral) (S) | 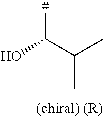 | H | H | OH | |
| 730 | n-hexyl/H (chiral) (R) | H (chiral) (S) | 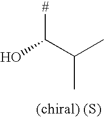 | H | H | OH | |
| 731 | n-hexyl/H (chiral) (S) | H (chiral) (R) | 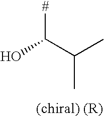 | H | H | OH | |
| 732 | n-hexyl/H (chiral) (S) | H (chiral) (R) | 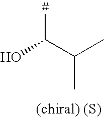 | H | H | OH | |
| 733 | n-hexyl/H (chiral) (S) | H (chiral) (S) | 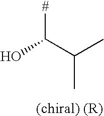 | H | H | OH | |
| 734 | n-hexyl/H (chiral) (S) | H (chiral) (S) | 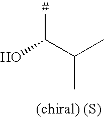 | H | H | OH | |
| 735 | n-hexyl/H (chiral) (R) | H (chiral) (S) | 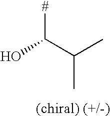 | H | H | OH | |
| 736 | n-hexyl/H (chiral) (R) | H (chiral) (+/−) | 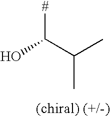 | H | H | OH | |
| 737 | n-hexyl/H (chiral) (+/−) | H (chiral) (+/−) | 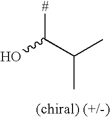 | H | H | OH | |
| 738 | n-hexyl/H (chiral) (R) | H (chiral) (R) | 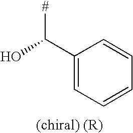 | H | H | OH | |
| 739 | n-hexyl/H (chiral) (R) | H (chiral) (R) | 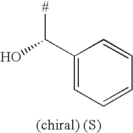 | H | H | OH | |
| 740 | n-hexyl/H (chiral) (R) | H (chiral) (S) | 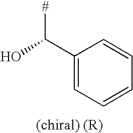 | H | H | OH | |
| 741 | n-hexyl/H (chiral) (R) | H (chiral) (S) | 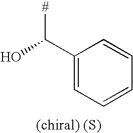 | H | H | OH | |
| 742 | n-hexyl/H (chiral) (S) | H (chiral) (R) | 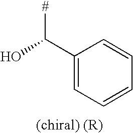 | H | H | OH | |
| 743 | n-hexyl/H (chiral) (S) | H (chiral) (R) | 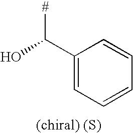 | H | H | OH | |
| 744 | n-hexyl/H (chiral) (S) | H (chiral) (S) | 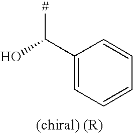 | H | H | OH | |
| 745 | n-hexyl/H (chiral) (S) | H (chiral) (S) | 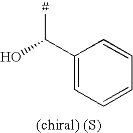 | H | H | OH | |
| 746 | n-hexyl/H (chiral) (R) | H (chiral) (S) | 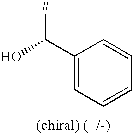 | H | H | OH | |
| 747 | n-hexyl/H (chiral) (R) | H (chiral) (+/−) | 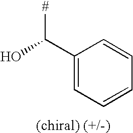 | H | H | OH | |
| 748 | n-hexyl/H (chiral) (+/−) | H (chiral) (+/−) | 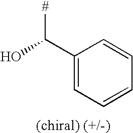 | H | H | OH | |
| 749 | n-hexyl/H (chiral) (R) | H (chiral) (R) | 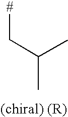 | H | H | OH | |
| 750 | n-hexyl/H (chiral) (R) | H (chiral) (R) | 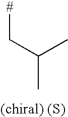 | H | H | OH | |
| 751 | n-hexyl/H (chiral) (R) | H (chiral) (S) | 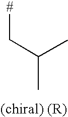 | H | H | OH | |
| 752 | n-hexyl/H (chiral) (R) | H (chiral) (S) | 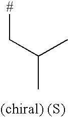 | H | H | OH | |
| 753 | n-hexyl/H (chiral) (S) | H (chiral) (R) | 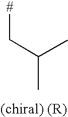 | H | H | OH | |
| 754 | n-hexyl/H (chiral) (S) | H (chiral) (R) | 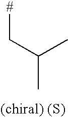 | H | H | OH | |
| 755 | n-hexyl/H (chiral) (S) | H (chiral) (S) | 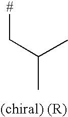 | H | H | OH | |
| 756 | n-hexyl/H (chiral) (S) | H (chiral) (S) | 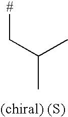 | H | H | OH | |
| 757 | n-hexyl/H (chiral) (R) | H (chiral) (S) | 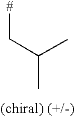 | H | H | OH | |
| 758 | n-hexyl/H (chiral) (R) | H (chiral) (+/−) | 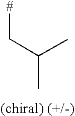 | H | H | OH | |
| 759 | n-hexyl/H (chiral) (+/−) | H (chiral) (+/−) | 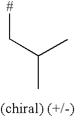 | H | H | OH | |
| 760 | n-hexyl/H (chiral) (R) | H (chiral) (R) | 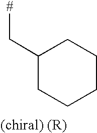 | H | H | OH | |
| 761 | n-hexyl/H (chiral) (R) | H (chiral) (R) | 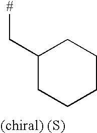 | H | H | OH | |
| 762 | n-hexyl/H (chiral) (R) | H (chiral) (S) | 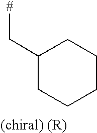 | H | H | OH | |
| 763 | n-hexyl/H (chiral) (R) | H (chiral) (S) | 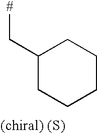 | H | H | OH | |
| 764 | n-hexyl/H (chiral) (S) | H (chiral) (R) | 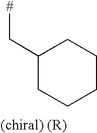 | H | H | OH | |
| 765 | n-hexyl/H (chiral) (S) | H (chiral) (R) | 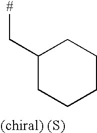 | H | H | OH | |
| 766 | n-hexyl/H (chiral) (S) | H (chiral) (S) | 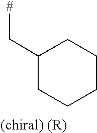 | H | H | OH | |
| 767 | n-hexyl/H (chiral) (S) | H (chiral) (S) | 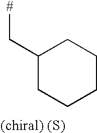 | H | H | OH | |
| 768 | n-hexyl/H (chiral) (R) | H (chiral) (S) | 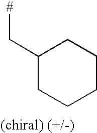 | H | H | OH | |
| 769 | n-hexyl/H (chiral) (R) | H (chiral) (+/−) | 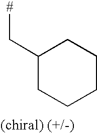 | H | H | OH | |
| 770 | n-hexyl/H (chiral) (+/−) | H (chiral) (+/−) | 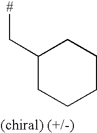 | H | H | OH | |
| 771 | n-hexyl/H (chiral) (R) | H (chiral) (R) | 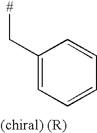 | H | H | OH | |
| 772 | n-hexyl/H (chiral) (R) | H (chiral) (R) | 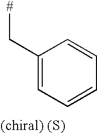 | H | H | OH | |
| 773 | n-hexyl/H (chiral) (R) | H (chiral) (S) | 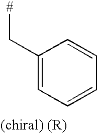 | H | H | OH | |
| 774 | n-hexyl/H (chiral) (R) | H (chiral) (S) | 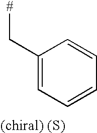 | H | H | OH | |
| 775 | n-hexyl/H (chiral) (S) | H (chiral) (R) | 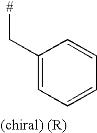 | H | H | OH | |
| 776 | n-hexyl/H (chiral) (S) | H (chiral) (R) | 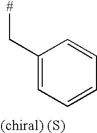 | H | H | OH | |
| 777 | n-hexyl/H (chiral) (S) | H (chiral) (S) | 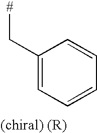 | H | H | OH | |
| 778 | n-hexyl/H (chiral) (S) | H (chiral) (S) | 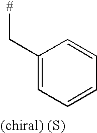 | H | H | OH | |
| 779 | n-hexyl/H (chiral) (R) | H (chiral) (S) | 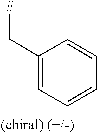 | H | H | OH | |
| 780 | n-hexyl/.H (chiral) (R) | H (chiral) (+/−) | 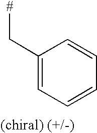 | H | H | OH | |
| 781 | n-hexyl/H (chiral) (+/−) | H (chiral) (+/−) | 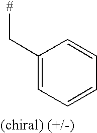 | H | H | OH | |
| 782 | n-pentyl/H (chiral) (R) | CH₃(chiral) (R) | 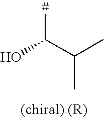 | H | H | OH | |
| 783 | n-pentyl/H (chiral) (R) | CH₃(chiral) (R) | 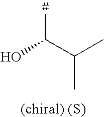 | H | H | OH | |
| 784 | n-pentyl/H (chiral) (R) | CH₃(chiral) (S) | 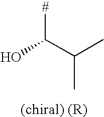 | H | H | OH | |
| 785 | n-pentyl/H (chiral) (R) | CH₃(chiral) (S) | 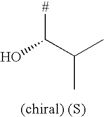 | H | H | OH | |
| 786 | n-pentyl/H (chiral) (S) | CH₃(chiral) (R) | 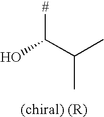 | H | H | OH | |
| 787 | n-pentyl/H (chiral) (S) | CH₃(chiral) (R) | 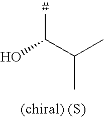 | H | H | OH | |
| 788 | n-pentyl/H (chiral) (S) | CH₃(chiral) (S) | 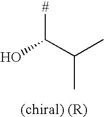 | H | H | OH | |
| 789 | n-pentyl/H (chiral) (S) | CH₃(chiral) (S) | 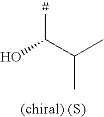 | H | H | OH | |
| 790 | n-pentyl/H (chiral) (R) | CH₃(chiral) (S) | 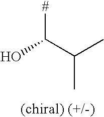 | H | H | OH | |
| 791 | n-pentyl/H (chiral) (R) | CH₃(chiral) (+/−) | 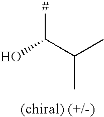 | H | H | OH | |
| 792 | n-pentyl/H (chiral) (+/−) | CH₃(chiral) (+/−) | 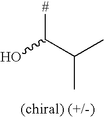 | H | H | OH | |
| 793 | n-pentyl/H (chiral) (R) | CH₃(chiral) (R) | 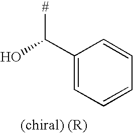 | H | H | OH | |
| 794 | n-pentyl/H (chiral) (R) | CH₃(chiral) (R) | 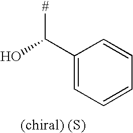 | H | H | OH | |
| 795 | n-pentyl/H (chiral) (R) | CH₃(chiral) (S) | 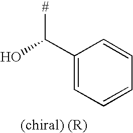 | H | H | OH | |
| 796 | n-pentyl/H (chiral) (R) | CH₃(chiral) (S) | 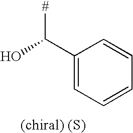 | H | H | OH | |
| 797 | n-pentyl/H (chiral) (S) | CH₃(chiral) (R) | 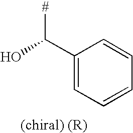 | H | H | OH | |
| 798 | n-pentyl/H (chiral) (S) | CH₃(chiral) (R) | 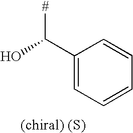 | H | H | OH | |
| 799 | n-pentyl/H (chiral) (S) | CH₃(chiral) (S) | 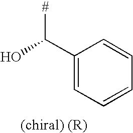 | H | H | OH | |
| 800 | n-pentyl/H (chiral) (S) | CH₃(chiral) (S) | 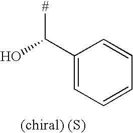 | H | H | OH | |
| 801 | n-pentyl/H (chiral) (R) | CH₃(chiral) (S) | 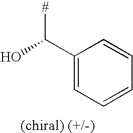 | H | H | OH | |
| 802 | n-pentyl/H (chiral) (R) | CH₃(chiral) (+/−) | 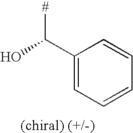 | H | H | OH | |
| 803 | n-pentyl/H (chiral) (+/−) | CH₃(chiral) (+/−) | 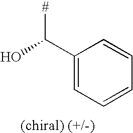 | H | H | OH | |
| 804 | n-pentyl/H (chiral) (R) | CH₃(chiral) (R) | 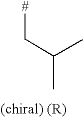 | H | H | OH | |
| 805 | n-pentyl/H (chiral) (R) | CH₃(chiral) (R) | 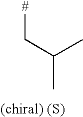 | H | H | OH | |
| 806 | n-pentyl/H (chiral) (R) | CH₃(chiral) (S) | 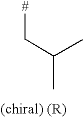 | H | H | OH | |
| 807 | n-pentyl/H (chiral) (R) | CH₃(chiral) (S) | 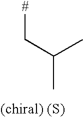 | H | H | OH | |
| 808 | n-pentyl/H (chiral) (S) | CH₃(chiral) (R) | 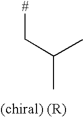 | H | H | OH | |
| 809 | n-pentyl/H (chiral) (S) | CH₃(chiral) (R) | 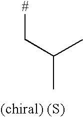 | H | H | OH | |
| 810 | n-pentyl/H (chiral) (S) | CH₃(chiral) (S) | 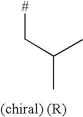 | H | H | OH | |
| 811 | n-pentyl/H (chiral) (S) | CH₃(chiral) (S) | 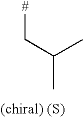 | H | H | OH | |
| 812 | n-pentyl/H (chiral) (R) | CH₃(chiral) (S) | 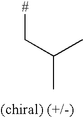 | H | H | OH | |
| 813 | n-pentyl/H (chiral) (R) | CH₃(chiral) (+/−) | 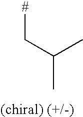 | H | H | OH | |
| 814 | n-pentyl/H (chiral) (+/−) | CH₃(chiral) (+/−) | 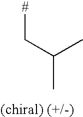 | H | H | OH | |
| 815 | n-pentyl/H (chiral) (R) | CH₃(chiral) (R) | 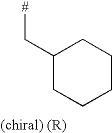 | H | H | OH | |
| 816 | n-pentyl/H (chiral) (R) | CH₃(chiral) (R) | 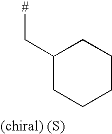 | H | H | OH | |
| 817 | n-pentyl/H (chiral) (R) | CH₃(chiral) (S) | 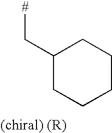 | H | H | OH | |
| 818 | n-pentyl/H (chiral) (R) | CH₃(chiral) (S) | 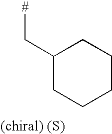 | H | H | OH | |
| 819 | n-pentyl/H (chiral) (S) | CH₃(chiral) (R) | 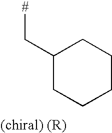 | H | H | OH | |
| 820 | n-pentyl/H (chiral) (S) | CH₃(chiral) (R) | 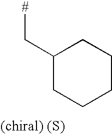 | H | H | OH | |
| 821 | n-pentyl/H (chiral) (S) | CH₃(chiral) (S) | 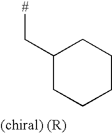 | H | H | OH | |
| 822 | n-pentyl/H (chiral) (S) | CH₃(chiral) (S) | 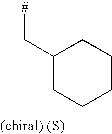 | H | H | OH | |
| 823 | n-pentyl/H (chiral) (R) | CH₃(chiral) (S) | 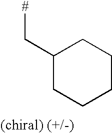 | H | H | OH | |
| 824 | n-pentyl/H (chiral) (R) | CH₃(chiral) (+/−) | 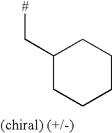 | H | H | OH | |
| 825 | n-pentyl/H (chiral) (+/−) | CH₃(chiral) (+/−) | 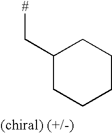 | H | H | OH | |
| 826 | n-pentyl/H (chiral) (R) | CH₃(chiral) (R) | 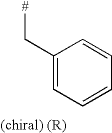 | H | H | OH | |
| 827 | n-pentyl/H (chiral) (R) | CH₃(chiral) (R) | 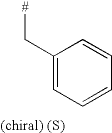 | H | H | OH | |
| 828 | n-pentyl/H (chiral) (R) | CH₃(chiral) (S) | 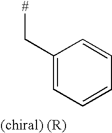 | H | H | OH | |
| 829 | n-pentyl/H (chiral) (R) | CH₃(chiral) (S) | 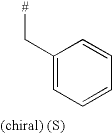 | H | H | OH | |
| 830 | n-pentyl/H (chiral) (S) | CH₃(chiral) (R) | 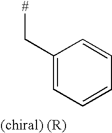 | H | H | OH | |
| 831 | n-pentyl/H (chiral) (S) | CH₃(chiral) (R) | 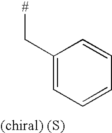 | H | H | OH | |
| 832 | n-pentyl/H (chiral) (S) | CH₃(chiral) (S) | 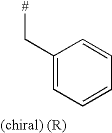 | H | H | OH | |
| 833 | n-pentyl/H (chiral) (S) | CH₃(chiral) (S) | 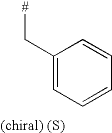 | H | H | OH | |
| 834 | n-pentyl/H (chiral) (R) | CH₃(chiral) (S) | 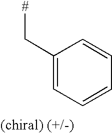 | H | H | OH | |
| 835 | n-pentyl/H (chiral) (R) | CH₃(chiral) (+/−) | 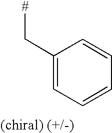 | H | H | OH | |
| 836 | n-pentyl/H (chiral) (+/−) | Ch₃(chiral) (+/−) | 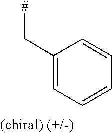 | H | H | OH | |
| 837 | n-pentyl/H (chiral) (R) | H (chiral) (R) | 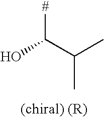 | H | H | OH | |
| 838 | n-pentyl/H (chiral) (R) | H (chiral) (R) | 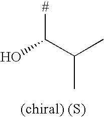 | H | H | OH | |
| 839 | n-pentyl/H (chiral) (R) | H (chiral) (S) | 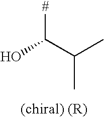 | H | H | OH | |
| 840 | n-pentyl/H (chiral) (R) | H (chiral) (S) | 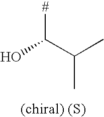 | H | H | OH | |
| 841 | n-pentyl/H (chiral) (S) | H (chiral) (R) | 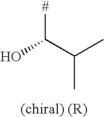 | H | H | OH | |
| 842 | n-pentyl/H (chiral) (S) | H (chiral) (R) | 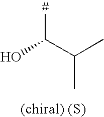 | H | H | OH | |
| 843 | n-pentyl/H (chiral) (S) | H (chiral) (S) | 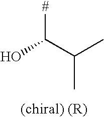 | H | H | OH | |
| 844 | n-pentyl/H (chiral) (S) | H (chiral) (S) | 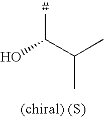 | H | H | OH | |
| 845 | n-pentyl/H (chiral) (R) | H (chiral) (S) | 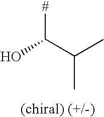 | H | H | OH | |
| 846 | n-pentyl/H (chiral) (R) | H (chiral) (+/−) | 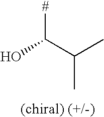 | H | H | OH | |
| 847 | n-pentyl/H (chiral) (+/−) | H (chiral) (+/−) | 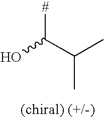 | H | H | OH | |
| 848 | n-pentyl/H (chiral) (R) | H (chiral) (R) | 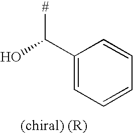 | H | H | OH | |
| 849 | n-pentyl/H (chiral) (R) | H (chiral) (R) | 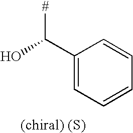 | H | H | OH | |
| 850 | n-pentyl/H (chiral) (R) | H (chiral) (S) | 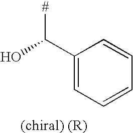 | H | H | OH | |
| 851 | n-pentyl/H (chiral) (R) | H (chiral) (S) | 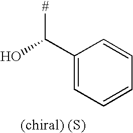 | H | H | OH | |
| 852 | n-pentyl/H (chiral) (S) | H (chiral) (R) | 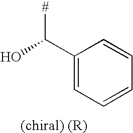 | H | H | OH | |
| 853 | n-pentyl/H (chiral) (S) | H (chiral) (R) | 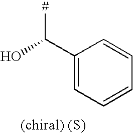 | H | H | OH | |
| 854 | n-pentyl/H (chiral) (S) | H (chiral) (S) | 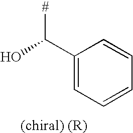 | H | H | OH | |
| 855 | n-pentyl/H (chiral) (S) | H (chiral) (S) | 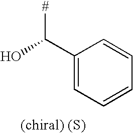 | H | H | OH | |
| 856 | n-pentyl/H (chiral) (R) | H (chiral) (S) | 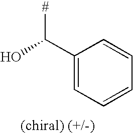 | H | H | OH | |
| 857 | n-pentyl/H (chiral) (R) | H (chiral) (+/−) | 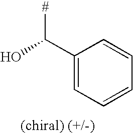 | H | H | OH | |
| 858 | n-pentyl/H (chiral) (+/−) | H (chiral) (+/−) | 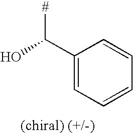 | H | H | OH | |
| 859 | n-pentyl/H (chiral) (R) | H (chiral) (R) | 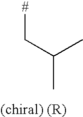 | H | H | OH | |
| 860 | n-pentyl/H (chiral) (R) | H (chiral) (R) | 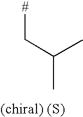 | H | H | OH | |
| 861 | n-pentyl/H (chiral) (R) | H (chiral) (S) | 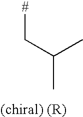 | H | H | OH | |
| 862 | n-pentyl/H (chiral) (R) | H (chiral) (S) | 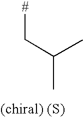 | H | H | OH | |
| 863 | n-pentyl/H (chiral) (S) | H (chiral) (R) | 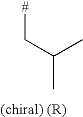 | H | H | OH | |
| 864 | n-pentyl/H (chiral) (S) | H (chiral) (R) | 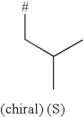 | H | H | OH | |
| 865 | n-pentyl/H (chiral) (S) | H (chiral) (S) | 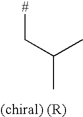 | H | H | OH | |
| 866 | n-pentyl/H (chiral) (S) | H (chiral) (S) | 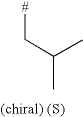 | H | H | OH | |
| 867 | n-pentyl/H (chiral) (S) | H (chiral) (S) | 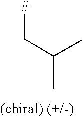 | H | H | OH | |
| 868 | n-pentyl/H (chiral) (R) | H (chiral) (+/−) | 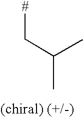 | H | H | OH | |
| 869 | n-pentyl/H (chiral) (+/−) | H (chiral) (+/−) | 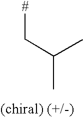 | H | H | OH | |
| 870 | n-pentyl/H (chiral) (R) | H (chiral) (R) | 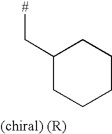 | H | H | OH | |
| 871 | n-pentyl/H (chiral) (R) | H (chiral) (R) | 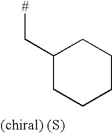 | H | H | OH | |
| 872 | n-pentyl/H (chiral) (R) | H (chiral) (S) | 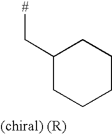 | H | H | OH | |
| 873 | n-pentyl/H (chiral) (R) | H (chiral) (S) | 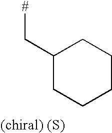 | H | H | OH | |
| 874 | n-pentyl/H (chiral) (S) | H (chiral) (R) | 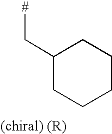 | H | H | OH | |
| 875 | n-pentyl/H (chiral) (S) | H (chiral) (R) | 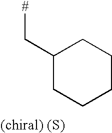 | H | H | OH | |
| 876 | n-pentyl/H (chiral) (S) | H (chiral) (S) | 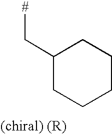 | H | H | OH | |
| 877 | n-pentyl/H (chiral) (S) | H (chiral) (S) | 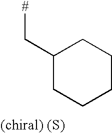 | H | H | OH | |
| 878 | n-pentyl/H (chiral) (R) | H (chiral) (S) | 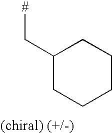 | H | H | OH | |
| 879 | n-pentyl/H (chiral) (R) | H (chiral) (+/−) | 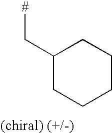 | H | H | OH | |
| 880 | n-pentyl/H (chiral) (+/−) | H (chiral) (+/−) | 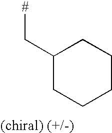 | H | H | OH | |
| 881 | n-pentyl/H (chiral) (R) | H (chiral) (R) | 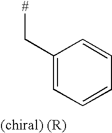 | H | H | OH | |
| 882 | n-pentyl/H (chiral) (R) | H (chiral) (R) | 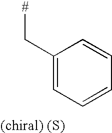 | H | H | OH | |
| 883 | n-pentyl/H (chiral) (R) | H (chiral) (S) | 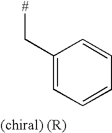 | H | H | OH | |
| 884 | n-pentyl/H (chiral) (R) | H (chiral) (S) | 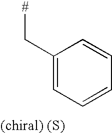 | H | H | OH | |
| 885 | n-pentyl/H (chiral) (S) | H (chiral) (R) | 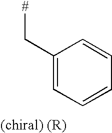 | H | H | OH | |
| 886 | n-pentyl/H (chiral) (S) | H (chiral) (R) | 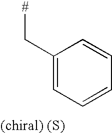 | H | H | OH | |
| 887 | n-pentyl/H (chiral) (S) | H (chiral) (S) | 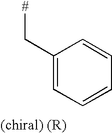 | H | H | OH | |
| 888 | n-pentyl/H (chiral) (S) | H (chiral) (S) | 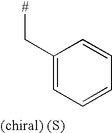 | H | H | OH | |
| 889 | n-pentyl/H (chiral) (R) | H (chiral) (S) | 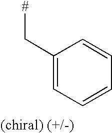 | H | H | OH | |
| 890 | n-pentyl/H (chiral) (R) | H (chiral) (+/−) | 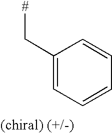 | H | H | OH | |
| 891 | n-pentyl/H (chiral) (+/−) | H (chiral) (+/−) | 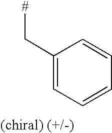 | H | H | OH | |
| 892 | n-hexyl/H (chiral) (R) | CH₃(chiral) (R) | 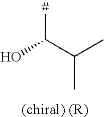 | H | H | OCH₂CH₃ | |
| 893 | n-hexyl/H (chiral) (R) | CH₃(chiral) (R) | 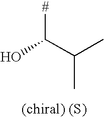 | H | H | OCH₂CH₃ | |
| 894 | n-hexyl/H (chiral) (R) | CH₃(chiral) (S) | 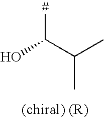 | H | H | OCH₂CH₃ | |
| 895 | n-hexyl/H (chiral) (R) | CH₃(chiral) (S) | 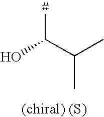 | H | H | OCH₂CH₃ | |
| 896 | n-hexyl/H (chiral) (S) | CH₃(chiral) (R) | 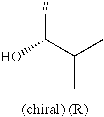 | H | H | OCH₂CH₃ | |
| 897 | n-hexyl/H (chiral) (S) | CH₃(chiral) (R) | 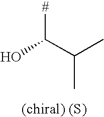 | H | H | OCH₂CH₃ | |
| 898 | n-hexyl/H (chiral) (S) | CH₃(chiral) (S) | 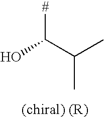 | H | H | OCH₂CH₃ | |
| 899 | n-hexyl/H (chiral) (S) | CH₃(chiral) (S) | 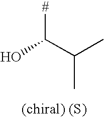 | H | H | OCH₂CH₃ | |
| 900 | n-hexyl/H (chiral) (R) | CH₃(chiral) (S) | 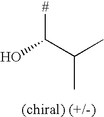 | H | H | OCH₂CH₃ | |
| 901 | n-hexyl/H (chiral) (R) | CH₃(chiral) (+/−) | 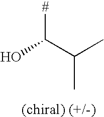 | H | H | OCH₂CH₃ | |
| 902 | n-hexyl/H (chiral) (+/−) | CH₃(chiral) (+/−) | 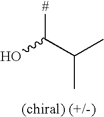 | H | H | OCH₂CH₃ | |
| 903 | n-hexyl/H (chiral) (R) | CH₃(chiral) (R) | 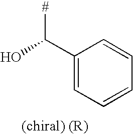 | H | H | OCH₂CH₃ | |
| 904 | n-hexyl/H (chiral) (R) | CH₃(chiral) (R) | 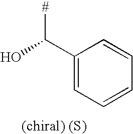 | H | H | OCH₂CH₃ | |
| 905 | n-hexyl/H (chiral) (R) | CH₃(chiral) (S) | 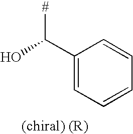 | H | H | OCH₂CH₃ | |
| 906 | n-hexyl/H (chiral) (R) | CH₃(chiral) (S) | 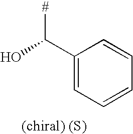 | H | H | OCH₂CH₃ | |
| 907 | n-hexyl/H (chiral) (S) | CH₃(chiral) (R) | 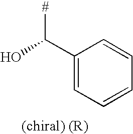 | H | H | OCH₂CH₃ | |
| 908 | n-hexyl/H (chiral) (S) | CH₃(chiral) (R) | 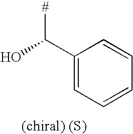 | H | H | OCH₂CH₃ | |
| 909 | n-hexyl/H (chiral) (S) | CH₃(chiral) (S) | 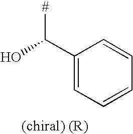 | H | H | OCH₂CH₃ | |
| 910 | n-hexyl/H (chiral) (S) | CH₃(chiral) (S) | 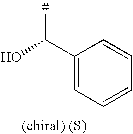 | H | H | OCH₂CH₃ | |
| 911 | n-hexyl/H (chiral) (R) | CH₃(chiral) (S) | 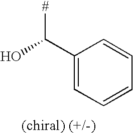 | H | H | OCH₂CH₃ | |
| 912 | n-hexyl/H (chiral) (R) | CH₃(chiral) (+/−) | 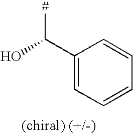 | H | H | OCH₂CH₃ | |
| 913 | n-hexyl/H (chiral) (+/−) | CH₃(chiral) (+/−) | 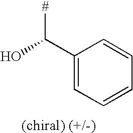 | H | H | OCH₂CH₃ | |
| 914 | n-hexyl/H (chiral) (R) | CH₃(chiral) (R) | 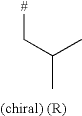 | H | H | OCH₂CH₃ | |
| 915 | n-hexyl/H (chiral) (R) | CH₃(chiral) (R) | 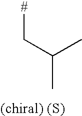 | H | H | OCH₂CH₃ | |
| 916 | n-hexyl/H (chiral) (R) | CH₃(chiral) (S) | 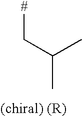 | H | H | OCH₂CH₃ | |
| 917 | n-hexyl/H (chiral) (R) | CH₃(chiral) (S) | 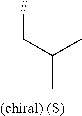 | H | H | OCH₂CH₃ | |
| 918 | n-hexyl/H (chiral) (S) | CH₃(chiral) (R) | 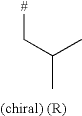 | H | H | OCH₂CH₃ | |
| 919 | n-hexyl/H (chiral) (S) | CH₃(chiral) (R) | 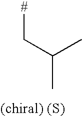 | H | H | OCH₂CH₃ | |
| 920 | n-hexyl/H (chiral) (S) | CH₃(chiral) (S) | 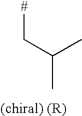 | H | H | OCH₂CH₃ | |
| 921 | n-hexyl/H (chiral) (S) | CH₃(chiral) (S) | 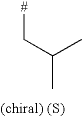 | H | H | OCH₂CH₃ | |
| 922 | n-hexyl/H (chiral) (R) | CH₃(chiral) (S) | 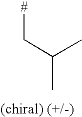 | H | H | OCH₂CH₃ | |
| 923 | n-hexyl/H (chiral) (R) | CH₃(chiral) (+/−) | 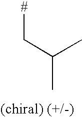 | H | H | OCH₂CH₃ | |
| 924 | n-hexyl/H (chiral) (+/−) | CH₃(chiral) (+/−) | 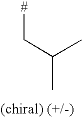 | H | H | OCH₂CH₃ | |
| 925 | n-hexyl/H (chiral) (R) | CH₃(chiral) (R) | 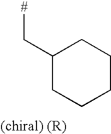 | H | H | OCH₂CH₃ | |
| 926 | n-hexyl/H (chiral) (R) | CH₃(chiral) (R) | 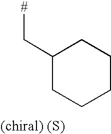 | H | H | OCH₂CH₃ | |
| 927 | n-hexyl/H (chiral) (R) | CH₃(chiral) (S) | 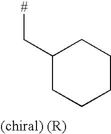 | H | H | OCH₂CH₃ | |
| 928 | n-hexyl/H (chiral) (R) | CH₃(chiral) (S) | 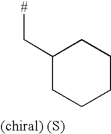 | H | H | OCH₂CH₃ | |
| 929 | n-hexyl/H (chiral) (S) | CH₃(chiral) (R) | 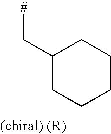 | H | H | OCH₂CH₃ | |
| 930 | n-hexyl/H (chiral) (S) | CH₃(chiral) (R) | 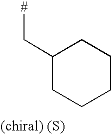 | H | H | OCH₂CH₃ | |
| 931 | n-hexyl/H (chiral) (S) | CH₃(chiral) (S) | 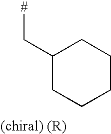 | H | H | OCH₂CH₃ | |
| 932 | n-hexyl/H (chiral) (S) | CH₃(chiral) (S) | 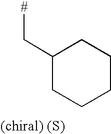 | H | H | OCH₂CH₃ | |
| 933 | n-hexyl/H (chiral) (R) | CH₃(chiral) (S) | 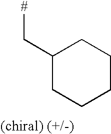 | H | H | OCH₂CH₃ | |
| 934 | n-hexyl/H (chiral) (R) | CH₃(chiral) (+/−) | 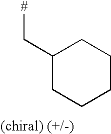 | H | H | OCH₂CH₃ | |
| 935 | n-hexyl/H (chiral) (+/−) | CH₃(chiral) (+/−) | 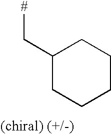 | H | H | OCH₂CH₃ | |
| 936 | n-hexyl/H (chiral) (R) | CH₃(chiral) (R) | 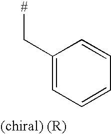 | H | H | OCH₂CH₃ | |
| 937 | n-hexyl/H (chiral) (R) | CH₃(chiral) (R) | 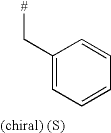 | H | H | OCH₂CH₃ | |
| 938 | n-hexyl/H (chiral) (R) | CH₃(chiral) (S) | 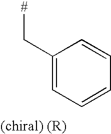 | H | H | OCH₂CH₃ | |
| 939 | n-hexyl/H (chiral) (R) | CH₃(chiral) (S) | 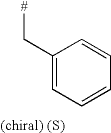 | H | H | OCH₂CH₃ | |
| 940 | n-hexyl/H (chiral) (S) | CH₃(chiral) (R) | 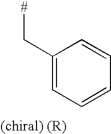 | H | H | OCH₂CH₃ | |
| 941 | n-hexyl/H (chiral) (S) | CH₃(chiral) (R) | 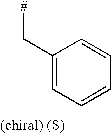 | H | H | OCH₂CH₃ | |
| 942 | n-hexyl/H (chiral) (S) | CH₃(chiral) (S) | 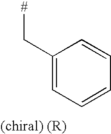 | H | H | OCH₂CH₃ | |
| 943 | n-hexyl/H (chiral) (S) | CH₃(chiral) (S) | 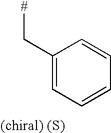 | H | H | OCH₂CH₃ | |
| 944 | n-hexyl/H (chiral) (R) | CH₃(chiral) (S) | 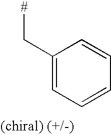 | H | H | OCH₂CH₃ | |
| 945 | n-hexyl/H (chiral) (R) | CH₃(chiral) (+/−) | 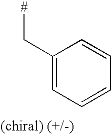 | H | H | OCH₂CH₃ | |
| 946 | n-hexyl/H (chiral) (+/−) | CH₃(chiral) (+/−) | 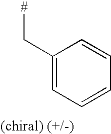 | H | H | OCH₂CH₃ | |
| 947 | n-hexyl/H (chiral) (R) | H (chiral) (R) | 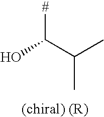 | H | H | OCH₂CH₃ | |
| 948 | n-hexyl/H (chiral) (R) | H (chiral) (R) | 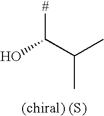 | H | H | OCH₂CH₃ | |
| 949 | n-hexyl/H (chiral) (R) | H (chiral) (S) | 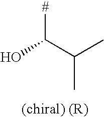 | H | H | OCH₂CH₃ | |
| 950 | n-hexyl/H (chiral) (R) | H (chiral) (S) | 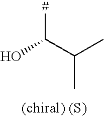 | H | H | OCH₂CH₃ | |
| 951 | n-hexyl/H (chiral) (S) | H (chiral) (R) | 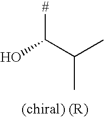 | H | H | OCH₂CH₃ | |
| 952 | n-hexyl/H (chiral) (S) | H (chiral) (R) | 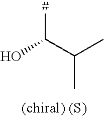 | H | H | OCH₂CH₃ | |
| 953 | n-hexyl/H (chiral) (S) | H (chiral) (S) | 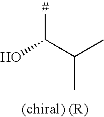 | H | H | OCH₂CH₃ | |
| 954 | n-hexyl/H (chiral) (S) | H (chiral) (S) | 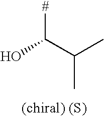 | H | H | OCH₂CH₃ | |
| 955 | n-hexyl/H (chiral) (R) | H (chiral) (S) | 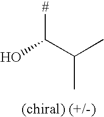 | H | H | OCH₂CH₃ | |
| 956 | n-hexyl/H (chiral) (R) | H (chiral) (+/−) | 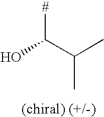 | H | H | OCH₂CH₃ | |
| 957 | n-hexyl/H (chiral) (+/−) | H (chiral) (+/−) | 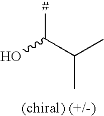 | H | H | OCH₂CH₃ | |
| 958 | n-hexyl/H (chiral) (R) | H (chiral) (R) | 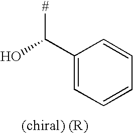 | H | H | OCH₂CH₃ | |
| 959 | n-hexyl/H (chiral) (R) | H (chiral) (R) | 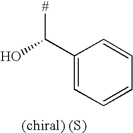 | H | H | OCH₂CH₃ | |
| 960 | n-hexyl/H (chiral) (R) | H (chiral) (S) | 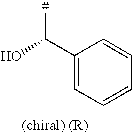 | H | H | OCH₂CH₃ | |
| 961 | n-hexyl/H (chiral) (R) | H (chiral) (S) | 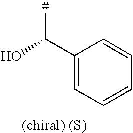 | H | H | OCH₂CH₃ | |
| 962 | n-hexyl/H (chiral) (S) | H (chiral) (R) | 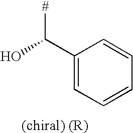 | H | H | OCH₂CH₃ | |
| 963 | n-hexyl/H (chiral) (S) | H (chiral) (R) | 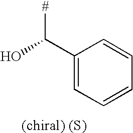 | H | H | OCH₂CH₃ | |
| 964 | n-hexyl/H (chiral) (S) | H (chiral) (S) | 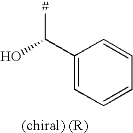 | H | H | OCH₂CH₃ | |
| 965 | n-hexyl/H (chiral) (S) | H (chiral) (S) | 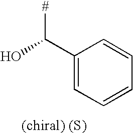 | H | H | OCH₂CH₃ | |
| 966 | n-hexyl/H (chiral) (R) | H (chiral) (S) | 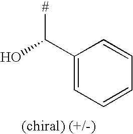 | H | H | OCH₂CH₃ | |
| 967 | n-hexyl/H (chiral) (R) | H (chiral) (+/−) | 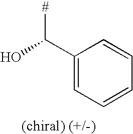 | H | H | OCH₂CH₃ | |
| 968 | n-hexyl/H (chiral) (+/−) | H (chiral) (+/−) | 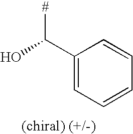 | H | H | OCH₂CH₃ | |
| 969 | n-hexyl/H (chiral) (R) | H (chiral) (R) | 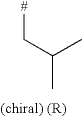 | H | H | OCH₂CH₃ | |
| 970 | n-hexyl/H (chiral) (R) | H (chiral) (R) | 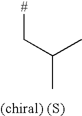 | H | H | OCH₂CH₃ | |
| 971 | n-hexyl/H (chiral) (R) | H (chiral) (S) | 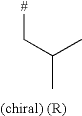 | H | H | OCH₂CH₃ | |
| 972 | n-hexyl/H (chiral) (R) | H (chiral) (S) | 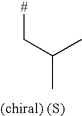 | H | H | OCH₂CH₃ | |
| 973 | n-hexyl/H (chiral) (S) | H (chiral) (R) | 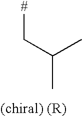 | H | H | OCH₂CH₃ | |
| 974 | n-hexyl/H (chiral) (S) | H (chiral) (R) | 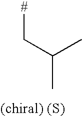 | H | H | OCH₂CH₃ | |
| 975 | n-hexyl/H (chiral) (S) | H (chiral) (S) | 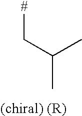 | H | H | OCH₂CH₃ | |
| 976 | n-hexyl/H (chiral) (S) | H (chiral) (S) | 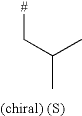 | H | H | OCH₂CH₃ | |
| 977 | n-hexyl/H (chiral) (R) | H (chiral) (S) | 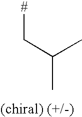 | H | H | OCH₂CH₃ | |
| 978 | n-hexyl/H (chiral) (R) | H (chiral) (+/−) | 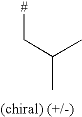 | H | H | OCH₂CH₃ | |
| 979 | n-hexyl/H (chiral) (+/−) | H (chiral) (+/−) | 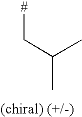 | H | H | OCH₂CH₃ | |
| 980 | n-hexyl/H (chiral) (R) | H (chiral) (R) | 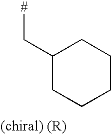 | H | H | OCH₂CH₃ | |
| 981 | n-hexyl/H (chiral) (R) | H (chiral) (R) | 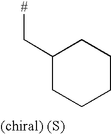 | H | H | OCH₂CH₃ | |
| 982 | n-hexyl/H (chiral) (R) | H (chiral) (S) | 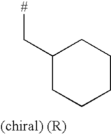 | H | H | OCH₂CH₃ | |
| 983 | n-hexyl/H (chiral) (R) | H (chiral) (S) | 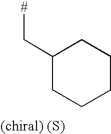 | H | H | OCH₂CH₃ | |
| 984 | n-hexyl/H (chiral) (S) | H (chiral) (R) | 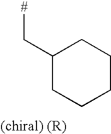 | H | H | OCH₂CH₃ | |
| 985 | n-hexyl/H (chiral) (S) | H (chiral) (R) | 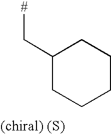 | H | H | OCH₂CH₃ | |
| 986 | n-hexyl/H (chiral) (S) | H (chiral) (S) | 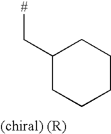 | H | H | OCH₂CH₃ | |
| 987 | n-hexyl/H (chiral) (S) | H (chiral) (S) | 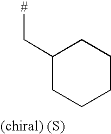 | H | H | OCH₂CH₃ | |
| 988 | n-hexyl/H (chiral) (R) | H (chiral) (S) | 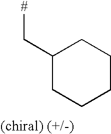 | H | H | OCH₂CH₃ | |
| 989 | n-hexyl/H (chiral) (R) | H (chiral) (+/−) | 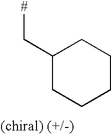 | H | H | OCH₂CH₃ | |
| 990 | n-hexyl/H (chiral) (+/−) | H (chiral) (+/−) | 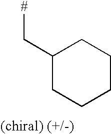 | H | H | OCH₂CH₃ | |
| 991 | n-hexyl/H (chiral) (R) | H (chiral) (R) | 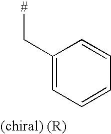 | H | H | OCH₂CH₃ | |
| 992 | n-hexyl/H (chiral) (R) | H (chiral) (R) | 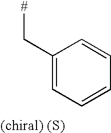 | H | H | OCH₂CH₃ | |
| 993 | n-hexyl/H (chiral) (R) | H (chiral) (S) | 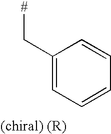 | H | H | OCH₂CH₃ | |
| 994 | n-hexyl/H (chiral) (R) | H (chiral) (S) | 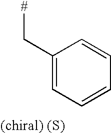 | H | H | OCH₂CH₃ | |
| 995 | n-hexyl/H (chiral) (S) | H (chiral) (R) | 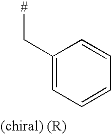 | H | H | OCH₂CH₃ | |
| 996 | n-hexyl/H (chiral) (S) | H (chiral) (R) | 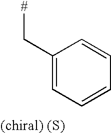 | H | H | OCH₂CH₃ | |
| 997 | n-hexyl/H (chiral) (S) | H (chiral) (S) | 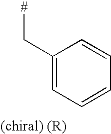 | H | H | OCH₂CH₃ | |
| 998 | n-hexyl/H (chiral) (S) | H (chiral) (S) | 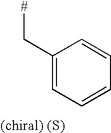 | H | H | OCH₂CH₃ | |
| 999 | n-hexyl/H (chiral) (R) | H (chiral) (S) | 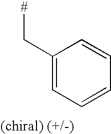 | H | H | OCH₂CH₃ | |
| 1000 | n-hexyl/H (chiral) (R) | H (chiral) (+/−) | 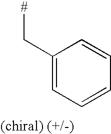 | H | H | OCH₂CH₃ | |
| 1001 | n-hexyl/H (chiral) (+/−) | H (chiral) (+/−) | 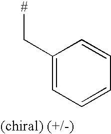 | H | H | OCH₂CH₃ | |
| 1002 | n-pentyl/H (chiral) (R) | CH₃(chiral) (R) | 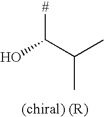 | H | H | OCH₂CH₃ | |
| 1003 | n-pentyl/H (chiral) (R) | CH₃(chiral) (R) | 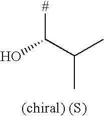 | H | H | OCH₂CH₃ | |
| 1004 | n-pentyl/H (chiral) (R) | CH₃(chiral) (S) | 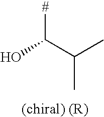 | H | H | OCH₂CH₃ | |
| 1005 | n-pentyl/H (chiral) (R) | CH₃(chiral) (S) | 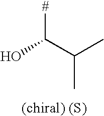 | H | H | OCH₂CH₃ | |
| 1006 | n-pentyl/H (chiral) (S) | CH₃(chiral) (R) | 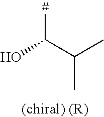 | H | H | OCH₂CH₃ | |
| 1007 | n-pentyl/H (chiral) (S) | CH₃(chiral) (S) | 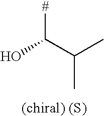 | H | H | OCH₂CH₃ | |
| 1008 | n-pentyl/H (chiral) (S) | CH₃(chiral) (S) | 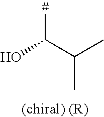 | H | H | OCH₂CH₃ | |
| 1009 | n-pentyl/H (chiral) (S) | CH₃(chiral) (S) | 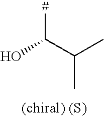 | H | H | OCH₂CH₃ | |
| 1010 | n-pentyl/H (chiral) (R) | CH₃(chiral) (S) | 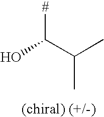 | H | H | OCH₂CH₃ | |
| 1011 | n-pentyl/H (chiral) (R) | CH₃(chiral) (+/−) | 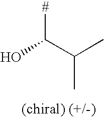 | H | H | OCH₂CH₃ | |
| 1012 | n-pentyl/H (chiral) (+/−) | CH₃(chiral) (+/−) | 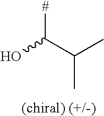 | H | H | OCH₂CH₃ | |
| 1013 | n-pentyl/H (chiral) (R) | CH₃(chiral) (R) | 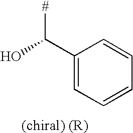 | H | H | OCH₂CH₃ | |
| 1014 | n-pentyl/H (chiral) (R) | CH₃(chiral) (R) | 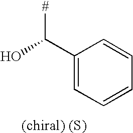 | H | H | OCH₂CH₃ | |
| 1015 | n-pentyl/H (chiral) (R) | CH₃(chiral) (S) | 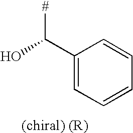 | H | H | OCH₂CH₃ | |
| 1016 | n-pentyl/H (chiral) (R) | CH₃(chiral) (S) | 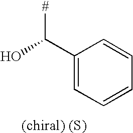 | H | H | OCH₂CH₃ | |
| 1017 | n-pentyl/H (chiral) (S) | CH₃(chiral) (R) | 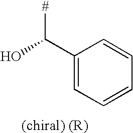 | H | H | OCH₂CH₃ | |
| 1018 | n-pentyl/H (chiral) (S) | CH₃(chiral) (R) | 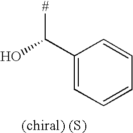 | H | H | OCH₂CH₃ | |
| 1019 | n-pentyl/H (chiral) (S) | CH₃(chiral) (S) | 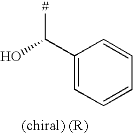 | H | H | OCH₂CH₃ | |
| 1020 | n-pentyl/H (chiral) (S) | CH₃(chiral) (S) | 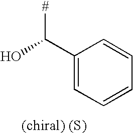 | H | H | OCH₂CH₃ | |
| 1021 | n-pentyl/H (chiral) (R) | CH₃(chiral) (S) | 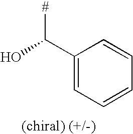 | H | H | OCH₂CH₃ | |
| 1022 | n-pentyl/H (chiral) (R) | CH₃(chiral) (+/−) | 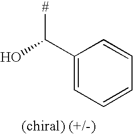 | H | H | OCH₂CH₃ | |
| 1023 | n-pentyl/H (chiral) (+/−) | CH₃(chiral) (+/−) | 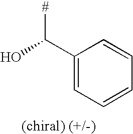 | H | H | OCH₂CH₃ | |
| 1024 | n-pentyl/H (chiral) (R) | CH₃(chiral) (R) | 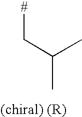 | H | H | OCH₂CH₃ | |
| 1025 | n-pentyl/H (chiral) (R) | CH₃(chiral) (R) | 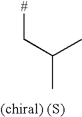 | H | H | OCH₂CH₃ | |
| 1026 | n-pentyl/H (chiral) (R) | CH₃(chiral) (S) | 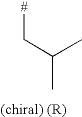 | H | H | OCH₂CH₃ | |
| 1027 | n-pentyl/H (chiral) (R) | CH₃(chiral) (S) | 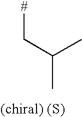 | H | H | OCH₂CH₃ | |
| 1028 | n-pentyl/H (chiral) (S) | CH₃(chiral) (R) | 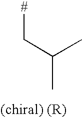 | H | H | OCH₂CH₃ | |
| 1029 | n-pentyl/H (chiral) (S) | CH₃(chiral) (R) | 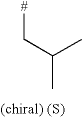 | H | H | OCH₂CH₃ | |
| 1030 | n-pentyl/H (chiral) (S) | CH₃(chiral) (S) | 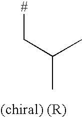 | H | H | OCH₂CH₃ | |
| 1031 | n-pentyl/H (chiral) (S) | CH₃(chiral) (S) | 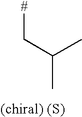 | H | H | OCH₂CH₃ | |
| 1032 | n-pentyl/H (chiral) (R) | CH₃(chiral) (S) | 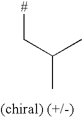 | H | H | OCH₂CH₃ | |
| 1033 | n-pentyl/H (chiral) (R) | CH₃(chiral) (+/−) | 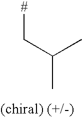 | H | H | OCH₂CH₃ | |
| 1034 | n-pentyl/H (chiral) (+/−) | CH₃(chiral) (+/−) | 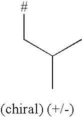 | H | H | OCH₂CH₃ | |
| 1035 | n-pentyl/H (chiral) (R) | CH₃(chiral) (R) | 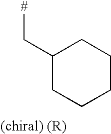 | H | H | OCH₂CH₃ | |
| 1036 | n-pentyl/H (chiral) (R) | CH₃(chiral) (R) | 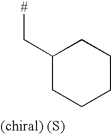 | H | H | OCH₂CH₃ | |
| 1037 | n-pentyl/H (chiral) (R) | CH₃(chiral) (S) | 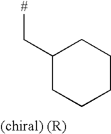 | H | H | OCH₂CH₃ | |
| 1038 | n-pentyl/H (chiral) (R) | CH₃(chiral) (S) | 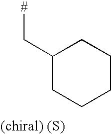 | H | H | OCH₂CH₃ | |
| 1039 | n-pentyl/H (chiral) (S) | CH₃(chiral) (R) | 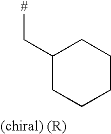 | H | H | OCH₂CH₃ | |
| 1040 | n-pentyl/H (chiral) (S) | CH₃(chiral) (R) | 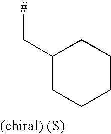 | H | H | OCH₂CH₃ | |
| 1041 | n-pentyl/H (chiral) (S) | CH₃(chiral) (S) | 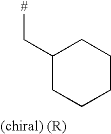 | H | H | OCH₂CH₃ | |
| 1042 | n-pentyl/H (chiral) (S) | CH₃(chiral) (S) | 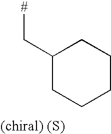 | H | H | OCH₂CH₃ | |
| 1043 | n-pentyl/H (chiral) (R) | CH₃(chiral) (S) | 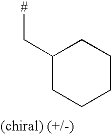 | H | H | OCH₂CH₃ | |
| 1044 | n-pentyl/H (chiral) (R) | CH₃(chiral) (+/−) | 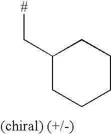 | H | H | OCH₂CH₃ | |
| 1045 | n-pentyl/H (chiral) (+/−) | CH₃(chiral) (+/−) | 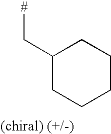 | H | H | OCH₂CH₃ | |
| 1046 | n-pentyl/H (chiral) (R) | CH₃(chiral) (R) | 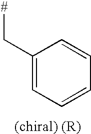 | H | H | OCH₂CH₃ | |
| 1047 | n-pentyl/H (chiral) (R) | CH₃(chiral) (R) | 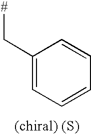 | H | H | OCH₂CH₃ | |
| 1048 | n-pentyl/H (chiral) (R) | CH₃(chiral) (S) | 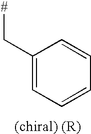 | H | H | OCH₂CH₃ | |
| 1049 | n-pentyl/H (chiral) (R) | CH₃(chiral) (S) | 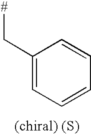 | H | H | OCH₂CH₃ | |
| 1050 | n-pentyl/H (chiral) (S) | CH₃(chiral) (R) | 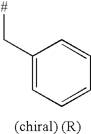 | H | H | OCH₂CH₃ | |
| 1051 | n-pentyl/H (chiral) (S) | CH₃(chiral) (R) | 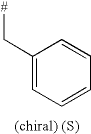 | H | H | OCH₂CH₃ | |
| 1052 | n-pentyl/H (chiral) (S) | CH₃(chiral) (S) | 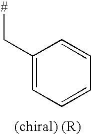 | H | H | OCH₂CH₃ | |
| 1053 | n-pentyl/H (chiral) (S) | CH₃(chiral) (S) | 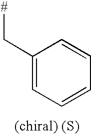 | H | H | OCH₂CH₃ | |
| 1054 | n-pentyl/H (chiral) (R) | CH₃(chiral) (S) | 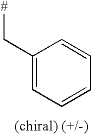 | H | H | OCH₂CH₃ | |
| 1055 | n-pentyl/H (chiral) (R) | CH₃(chiral) (+/−) | 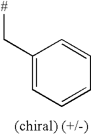 | H | H | OCH₂CH₃ | |
| 1056 | n-pentyl/H (chiral) (+/−) | CH₃(chiral) (+/−) | 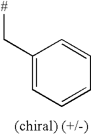 | H | H | OCH₂CH₃ | |
| 1057 | n-pentyl/H (chiral) (R) | H (chiral) (R) | 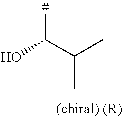 | H | H | OCH₂CH₃ | |
| 1058 | n-pentyl/H (chiral) (R) | H (chiral) (R) | 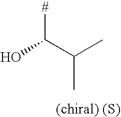 | H | H | OCH₂CH₃ | |
| 1059 | n-pentyl/H (chiral) (R) | H (chiral) (S) | 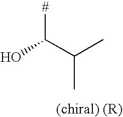 | H | H | OCH₂CH₃ | |
| 1060 | n-pentyl/H (chiral) (R) | H (chiral) (S) | 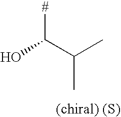 | H | H | OCH₂CH₃ | |
| 1061 | n-pentyl/H (chiral) (R) | H (chiral) (S) | 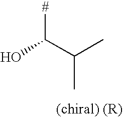 | H | H | OCH₂CH₃ | |
| 1062 | n-pentyl/H (chiral) (S) | H (chiral) (R) | 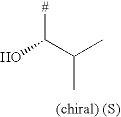 | H | H | OCH₂CH₃ | |
| 1063 | n-pentyl/H (chiral) (S) | H (chiral) (S) | 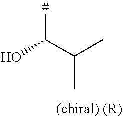 | H | H | OCH₂CH₃ | |
| 1064 | n-pentyl/H (chiral) (S) | H (chiral) (S) | 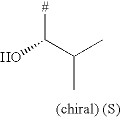 | H | H | OCH₂CH₃ | |
| 1065 | n-pentyl/H (chiral) (R) | H (chiral) (S) | 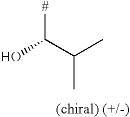 | H | H | OCH₂CH₃ | |
| 1066 | n-pentyl/H (chiral) (R) | H (chiral) (+/−) | 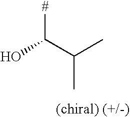 | H | H | OCH₂CH₃ | |
| 1067 | n-pentyl/H (chiral) (+/−) | H (chiral) (+/−) | 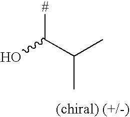 | H | H | OCH₂CH₃ | |
| 1068 | n-pentyl/H (chiral) (R) | H (chiral) (R) | 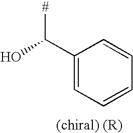 | H | H | OCH₂CH₃ | |
| 1069 | n-pentyl/H (chiral) (R) | H (chiral) (R) | 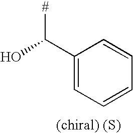 | H | H | OCH₂CH₃ | |
| 1070 | n-pentyl/H (chiral) (R) | H (chiral) (S) | 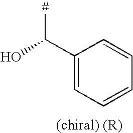 | H | H | OCH₂CH₃ | |
| 1071 | n-pentyl/H (chiral) (R) | H (chiral) (S) | 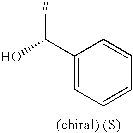 | H | H | OCH₂CH₃ | |
| 1072 | n-pentyl/H (chiral) (S) | H (chiral) (R) | 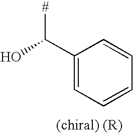 | H | H | OCH₂CH₃ | |
| 1073 | n-pentyl/H (chiral) (S) | H (chiral) (R) | 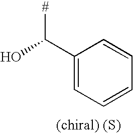 | H | H | OCH₂CH₃ | |
| 1074 | n-pentyl/H (chiral) (S) | H (chiral) (S) | 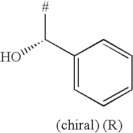 | H | H | OCH₂CH₃ | |
| 1075 | n-pentyl/H (chiral) (S) | H (chiral) (S) | 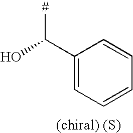 | H | H | OCH₂CH₃ | |
| 1076 | n-pentyl/H (chiral) (R) | H (chiral) (S) | 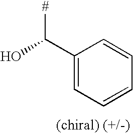 | H | H | OCH₂CH₃ | |
| 1077 | n-pentyl/H (chiral) (R) | H (chiral) (+/−) | 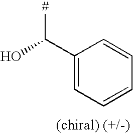 | H | H | OCH₂CH₃ | |
| 1078 | n-pentyl/H (chiral) (+/−) | H (chiral) (+/−) | 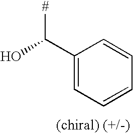 | H | H | OCH₂CH₃ | |
| 1079 | n-pentyl/H (chiral) (R) | H (chiral) (R) | 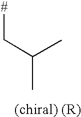 | H | H | OCH₂CH₃ | |
| 1080 | n-pentyl/H (chiral) (R) | H (chiral) (R) | 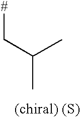 | H | H | OCH₂CH₃ | |
| 1081 | n-pentyl/H (chiral) (R) | H (chiral) (S) | 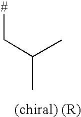 | H | H | OCH₂CH₃ | |
| 1082 | n-pentyl/H (chiral) (R) | H (chiral) (S) | 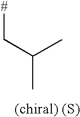 | H | H | OCH₂CH₃ | |
| 1083 | n-pentyl/H (chiral) (S) | H (chiral) (R) | 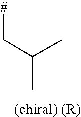 | H | H | OCH₂CH₃ | |
| 1084 | n-pentyl/H (chiral) (S) | H (chiral) (R) | 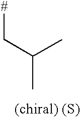 | H | H | OCH₂CH₃ | |
| 1085 | n-pentyl/H (chiral) (S) | H (chiral) (S) | 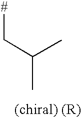 | H | H | OCH₂CH₃ | |
| 1086 | n-pentyl/H (chiral) (S) | H (chiral) (S) | 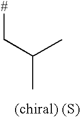 | H | H | OCH₂CH₃ | |
| 1087 | n-pentyl/H (chiral) (R) | H (chiral) (S) | 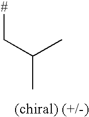 | H | H | OCH₂CH₃ | |
| 1088 | n-pentyl/H (chiral) (R) | H (chiral) (+/−) | 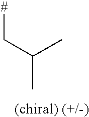 | H | H | OCH₂CH₃ | |
| 1089 | n-pentyl/H (chiral) (+/−) | H (chiral) (+/−) | 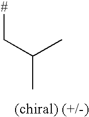 | H | H | OCH₂CH₃ | |
| 1090 | n-pentyl/H (chiral) (R) | H (chiral) (R) | 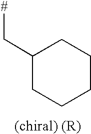 | H | H | OCH₂CH₃ | |
| 1091 | n-pentyl/H (chiral) (R) | H (chiral) (R) | 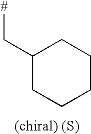 | H | H | OCH₂CH₃ | |
| 1092 | n-pentyl/H (chiral) (R) | H (chiral) (S) | 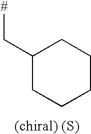 | H | H | OCH₂CH₃ | |
| 1093 | n-pentyl/H (chiral) (R) | H (chiral) (S) | 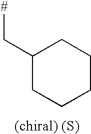 | H | H | OCH₂CH₃ | |
| 1094 | n-pentyl/H (chiral) (S) | H (chiral) (R) | 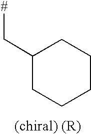 | H | H | OCH₂CH₃ | |
| 1095 | n-pentyl/H (chiral) (S) | H (chiral) (R) | 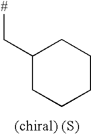 | H | H | OCH₂CH₃ | |
| 1096 | n-pentyl/H (chiral) (S) | H (chiral) (S) | 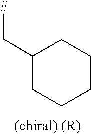 | H | H | OCH₂CH₃ | |
| 1097 | n-pentyl/H (chiral) (S) | H (chiral) (S) | 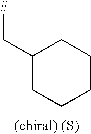 | H | H | OCH₂CH₃ | |
| 1098 | n-pentyl/H (chiral) (R) | H (chiral) (S) | 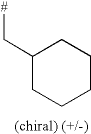 | H | H | OCH₂CH₃ | |
| 1099 | n-pentyl/H (chiral) (R) | H (chiral) (+/−) | 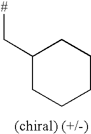 | H | H | OCH₂CH₃ | |
| 1100 | n-pentyl/H (chiral) (+/−) | H (chiral) (+/−) | 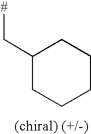 | H | H | OCH₂CH₃ | |
| 1101 | n-pentyl/H (chiral) (R) | H (chiral) (R) | 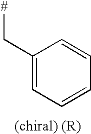 | H | H | OCH₂CH₃ | |
| 1102 | n-pentyl/H (chiral) (R) | H (chiral) (R) | 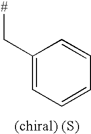 | H | H | OCH₂CH₃ | |
| 1103 | n-pentyl/H (chiral) (R) | H (chiral) (S) | 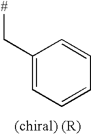 | H | H | OCH₂CH₃ | |
| 1104 | n-pentyl/H (chiral) (R) | H (chiral) (S) | 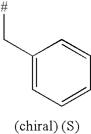 | H | H | OCH₂CH₃ | |
| 1105 | n-pentyl/H (chiral) (S) | H (chiral) (R) | 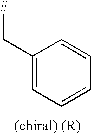 | H | H | OCH₂CH₃ | |
| 1106 | n-pentyl/H (chiral) (S) | H (chiral) (R) | 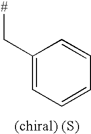 | H | H | OCH₂CH₃ | |
| 1107 | n-pentyl/H (chiral) (S) | H (chiral) (S) | 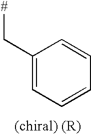 | H | H | OCH₂CH₃ | |
| 1108 | n-pentyl/H (chiral) (S) | H (chiral) (S) | 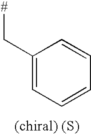 | H | H | OCH₂CH₃ | |
| 1109 | n-pentyl/H (chiral) (R) | H (chiral) (S) | 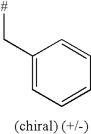 | H | H | OCH₂CH₃ | |
| 1110 | n-pentyl/H (chiral) (R) | H (chiral) (+/−) | 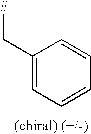 | H | H | OCH₂CH₃ | |
| 1111 | n-pentyl/H (chiral) (+/−) | H (chiral) (+/−) | 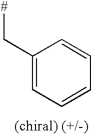 | H | H | OCH₂CH₃ | |
| 1112 | | | | | | | |
| 1113 | n-hexyl/H (chiral) (R) | CH₃(chiral) (R) | 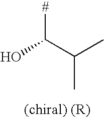 | H | H | SCH₃ | |
| 1114 | n-hexyl/H (chiral) (R) | CH₃(chiral) (R) | 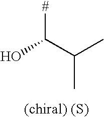 | H | H | SCH₃ | |
| 1115 | n-hexyl/H (chiral) (R) | CH₃(chiral) (S) | 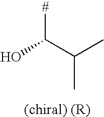 | H | H | SCH₃ | |
| 1116 | n-hexyl/H (chiral) (R) | CH₃(chiral) (S) | 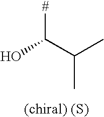 | H | H | SCH₃ | |
| 1117 | n-hexyl/H (chiral) (S) | CH₃(chiral) (R) | 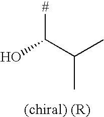 | H | H | SCH₃ | |
| 1118 | n-hexyl/H (chiral) (S) | CH₃(chiral) (R) | 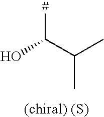 | H | H | SCH₃ | |
| 1119 | n-hexyl/H (chiral) (S) | CH₃(chiral) (S) | 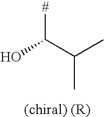 | H | H | SCH₃ | |
| 1120 | n-hexyl/H (chiral) (S) | CH₃(chiral) (S) | 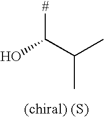 | H | H | SCH₃ | |
| 1121 | n-hexyl/H (chiral) (R) | CH₃(chiral) (S) | 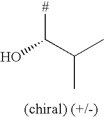 | H | H | SCH₃ | |
| 1122 | n-hexyl/H (chiral) (R) | CH₃(chiral) (+/−) | 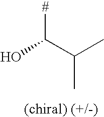 | H | H | SCH₃ | |
| 1123 | n-hexyl/H (chiral) (+/−) | CH₃(chiral) (+/−) | 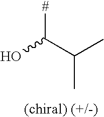 | H | H | SCH₃ | |
| 1124 | n-hexyl/H (chiral) (R) | CH₃(chiral) (R) | 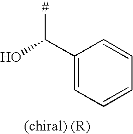 | H | H | SCH₃ | |
| 1125 | n-hexyl/H (chiral) (R) | CH₃(chiral) (R) | 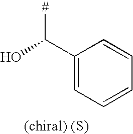 | H | H | SCH₃ | |
| 1126 | n-hexyl/H (chiral) (R) | CH₃(chiral) (S) | 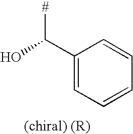 | H | H | SCH₃ | |
| 1127 | n-hexyl/H (chiral) (R) | CH₃(chiral) (S) | 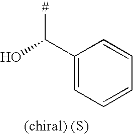 | H | H | SCH₃ | |
| 1128 | n-hexyl/H (chiral) (S) | CH₃(chiral) (R) | 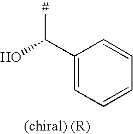 | H | H | SCH₃ | |
| 1129 | n-hexyl/H (chiral) (S) | CH₃(chiral) (R) | 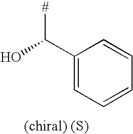 | H | H | SCH₃ | |
| 1130 | n-hexyl/H (chiral) (S) | CH₃9chiral) (S) | 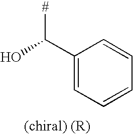 | H | H | SCH₃ | |
| 1131 | n-hexyl/H (chiral) (S) | CH₃(chiral) (S) | 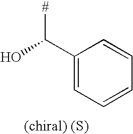 | H | H | SCH₃ | |
| 1132 | n-hexyl/H (chiral) (R) | CH₃(chiral) (S) | 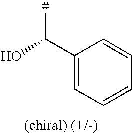 | H | H | SCH₃ | |
| 1133 | n-hexyl/H (chiral) (R) | CH₃(chiral) (+/−) | 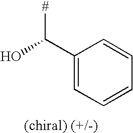 | H | H | SCH₃ | |
| 1134 | n-hexyl/H (chiral) (+/−) | CH₃(chiral) (+/−) | 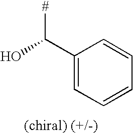 | H | H | SCH₃ | |
| 1135 | n-hexyl/H (chiral) (R) | CH₃(chiral) (R) | 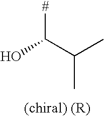 | H | H | SCH₃ | |
| 1136 | n-hexyl/H (chiral) (R) | CH₃(chiral) (R) | 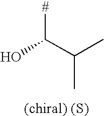 | H | H | SCH₃ | |
| 1137 | n-hexyl/H (chiral) (R) | CH₃(chiral) (S) | 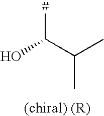 | H | H | SCH₃ | |
| 1138 | n-hexyl/H (chiral) (R) | CH₃(chiral) (S) | 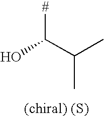 | H | H | SCH₃ | |
| 1139 | n-hexyl/H (chiral) (S) | CH₃(chiral) (R) | 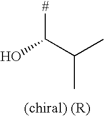 | H | H | SCH₃ | |
| 1140 | n-hexyl/H (chiral) (S) | CH₃(chiral) (R) | 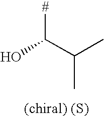 | H | H | SCH₃ | |
| 1141 | n-hexyl/H (chiral) (S) | CH₃(chiral) (S) | 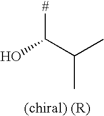 | H | H | SCH₃ | |
| 1142 | n-hexyl/H (chiral) (R) | CH₃(chiral) (S) | 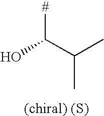 | H | H | SCH₃ | |
| 1143 | n-hexyl/H (chiral) (R) | CH₃(chiral) (S) | 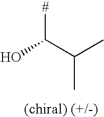 | H | H | SCH₃ | |
| 1144 | n-hexyl/H (chiral) (R) | CH₃(chiral) (+/−) | 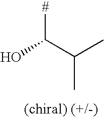 | H | H | SCH₃ | |
| 1145 | n-hexyl/H (chiral) (+/−) | CH₃(chiral) (+/−) | 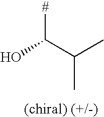 | H | H | SCH₃ | |
| 1146 | n-hexyl/H (chiral) (R) | CH₃(chiral) (R) | 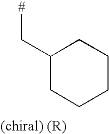 | H | H | SCH₃ | |
| 1147 | n-hexyl/H (chiral) (R) | CH₃(chiral) (R) | 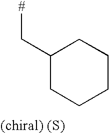 | H | H | SCH₃ | |
| 1148 | n-hexyl/H (chiral) (R) | CH₃(chiral) (S) | 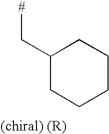 | H | H | SCH₃ | |
| 1149 | n-hexyl/H (chiral) (R) | CH₃(chiral) (S) | 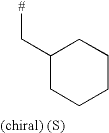 | H | H | SCH₃ | |
| 1150 | n-hexyl/H (chiral) (S) | CH₃(chiral) (R) | 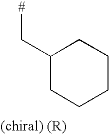 | H | H | SCH₃ | |
| 1151 | n-hexyl/H (chiral) (S) | CH₃(chiral) (R) | 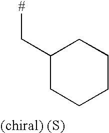 | H | H | SCH₃ | |
| 1152 | n-hexyl/H (chiral) (S) | CH₃(chiral) (S) | 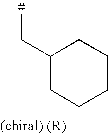 | H | H | SCH₃ | |
| 1153 | n-hexyl/H (chiral) (S) | CH₃(chiral) (S) | 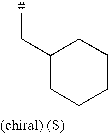 | H | H | SCH₃ | |
| 1154 | n-hexyl/H (chiral) (R) | CH₃(chiral) (S) | 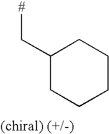 | H | H | SCH₃ | |
| 1155 | n-hexyl/H (chiral) (R) | CH₃(chiral) (+/−) | 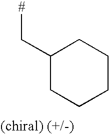 | H | H | SCH₃ | |
| 1156 | n-hexyl/H (chiral) (+/−) | CH₃(chiral) (+/−) | 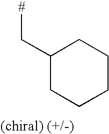 | H | H | SCH₃ | |
| 1157 | n-hexyl/H (chiral) (R) | CH₃(chiral) (R) | 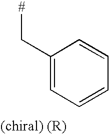 | H | H | SCH₃ | |
| 1158 | n-hexyl/H (chiral) (R) | CH₃(chiral) (R) | 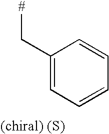 | H | H | SCH₃ | |
| 1159 | n-hexyl/H (chiral) (R) | CH₃(chiral) (S) | 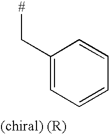 | H | H | SCH₃ | |
| 1160 | n-hexyl/H (chiral) (R) | CH₃(chiral) (S) | 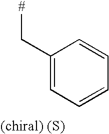 | H | H | SCH₃ | |
| 1161 | n-hexyl/H (chiral) (S) | CH₃(chiral) (R) | 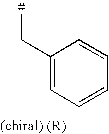 | H | H | SCH₃ | |
| 1162 | n-hexyl/H (chiral) (S) | CH₃(chiral) (R) | 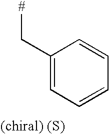 | H | H | SCH₃ | |
| 1163 | n-hexyl/H (chiral) (S) | CH₃(chiral) (S) | 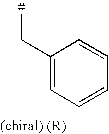 | H | H | SCH₃ | |
| 1164 | n-hexyl/H (chiral) (S) | CH₃(chiral) (S) | 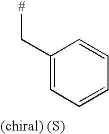 | H | H | SCH₃ | |
| 1165 | n-hexyl/H (chiral) (R) | CH₃(chiral) (S) | 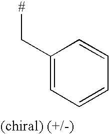 | H | H | SCH₃ | |
| 1166 | n-hexyl/H (chiral) (R) | CH₃(chiral) (+/−) | 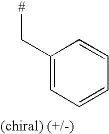 | H | H | SCH₃ | |
| 1167 | n-hexyl/H (chiral) (+/−) | CH₃(chiral) (+/−) | 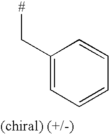 | H | H | SCH₃ | |
| 1168 | n-hexyl/H (chiral) (R) | H (chiral) (R) | 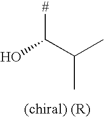 | H | H | SCH₃ | |
| 1169 | n-hexyl/H (chiral) (R) | H (chiral) (R) | 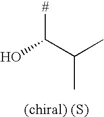 | H | H | SCH₃ | |
| 1170 | n-hexyl/H (chiral) (R) | H (chiral) (S) | 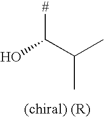 | H | H | SCH₃ | |
| 1171 | n-hexyl/H (chiral) (R) | H (chiral) (S) | 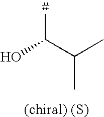 | H | H | SCH₃ | |
| 1172 | n-hexyl/H (chiral) (S) | H (chiral) (R) | 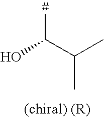 | H | H | SCH₃ | |
| 1173 | n-hexyl/H (chiral) (S) | H (chiral) (R) | 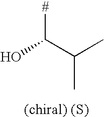 | H | H | SCH₃ | |
| 1174 | n-hexyl/H (chiral) (S) | H (chiral) (S) | 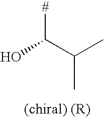 | H | H | SCH₃ | |
| 1175 | n-hexyl/H (chiral) (S) | H (chiral) (S) | 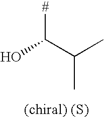 | H | H | SCH₃ | |
| 1176 | n-hexyl/H (chiral) (R) | H (chiral) (S) | 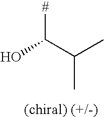 | H | H | SCH₃ | |
| 1177 | n-hexyl/H (chiral) (R) | H (chiral) (+/−) | 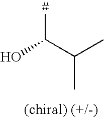 | H | H | SCH₃ | |
| 1178 | n-hexyl/H (chiral) (+/−) | H (chiral) (+/−) | 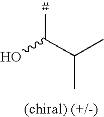 | H | H | SCH₃ | |
| 1179 | n-hexyl/H (chiral) (R) | H (chiral) (R) | 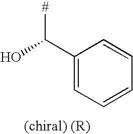 | H | H | SCH₃ | |
| 1180 | n-hexyl/H (chiral) (R) | H (chiral) (R) | 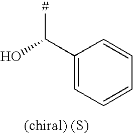 | H | H | SCH₃ | |
| 1181 | n-hexyl/H (chiral) (R) | H (chiral) (S) | 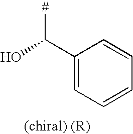 | H | H | SCH₃ | |
| 1182 | n-hexyl/H (chiral) (R) | H (chiral) (S) | 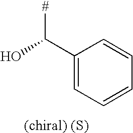 | H | H | SCH₃ | |
| 1183 | n-hexyl/H (chiral) (S) | H (chiral) (R) | 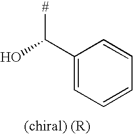 | H | H | SCH₃ | |
| 1184 | n-hexyl/H (chiral) (S) | H (chiral) (R) | 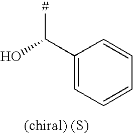 | H | H | SCH₃ | |
| 1185 | n-hexyl/H (chiral) (S) | H (chiral) (S) | 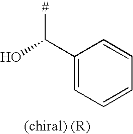 | H | H | SCH₃ | |
| 1186 | n-hexyl/H (chiral) (S) | H (chiral) (S) | 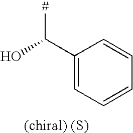 | H | H | SCH₃ | |
| 1187 | n-hexyl/H (chiral) (R) | H (chiral) (S) | 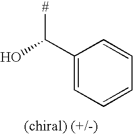 | H | H | SCH₃ | |
| 1188 | n-hexyl/H (chiral) (R) | H (chiral) (+/−) | 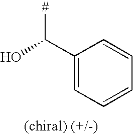 | H | H | SCH₃ | |
| 1189 | n-hexyl/H (chiral) (+/−) | H (chiral) (+/−) | 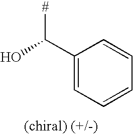 | H | H | SCH₃ | |
| 1190 | n-hexyl/H (chiral) (R) | H (chiral) (R) | 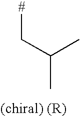 | H | H | SCH₃ | |
| 1191 | n-hexyl/H (chiral) (R) | H (chiral) (R) | 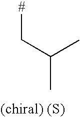 | H | H | SCH₃ | |
| 1192 | n-hexyl/H (chiral) (R) | H (chiral) (S) | 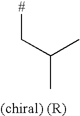 | H | H | SCH₃ | |
| 1193 | n-hexyl/H (chiral) (R) | H (chiral) (S) | 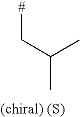 | H | H | SCH₃ | |
| 1194 | n-hexyl/H (chiral) (S) | H (chiral) (R) | 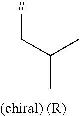 | H | H | SCH₃ | |
| 1195 | n-hexyl/H (chiral) (S) | H (chiral) (R) | 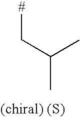 | H | H | SCH₃ | |
| 1196 | n-hexyl/H (chiral) (S) | H (chiral) (S) | 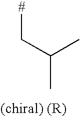 | H | H | SCH₃ | |
| 1197 | n-hexyl/H (chiral) (S) | H (chiral) (S) | 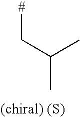 | H | H | SCH₃ | |
| 1198 | n-hexyl/H (chiral) (R) | H (chiral) (S) | 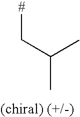 | H | H | SCH₃ | |
| 1199 | n-hexyl/H (chiral) (R) | H (chiral) (+/−) | 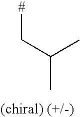 | H | H | SCH₃ | |
| 1200 | n-hexyl/H (chiral) (+/−) | H (chiral) (+/−) | 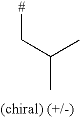 | H | H | SCH₃ | |
| 1201 | n-hexyl/H (chiral) (R) | H (chiral) (R) | 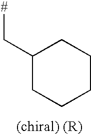 | H | H | SCH₃ | |
| 1202 | n-hexyl/H (chiral) (R) | H (chiral) (R) | 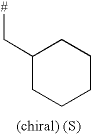 | H | H | SCH₃ | |
| 1203 | n-hexyl/H (chiral) (R) | H (chiral) (S) | 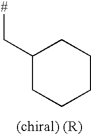 | H | H | SCH₃ | |
| 1204 | n-hexyl/H (chiral) (R) | H (chiral) (S) | 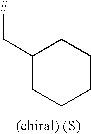 | H | H | SCH₃ | |
| 1205 | n-hexyl/H (chiral) (S) | H (chiral) (R) | 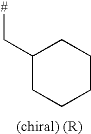 | H | H | SCH₃ | |
| 1206 | n-hexyl/H (chiral) (S) | H (chiral) (R) | 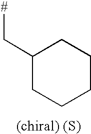 | H | H | SCH₃ | |
| 1207 | n-hexyl/H (chiral) (S) | H (chiral) (S) | 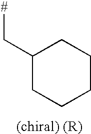 | H | H | SCH₃ | |
| 1208 | n-hexyl/H (chiral) (S) | H (chiral) (S) | 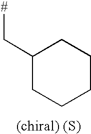 | H | H | SCH₃ | |
| 1209 | n-hexyl/H (chiral) (R) | H (chiral) (S) | 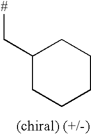 | H | H | SCH₃ | |
| 1210 | n-hexyl/H (chiral) (R) | H (chiral) (+/−) | 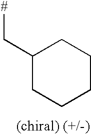 | H | H | SCH₃ | |
| 1211 | n-hexyl/H (chiral) (+/−) | H (chiral) (+/−) | 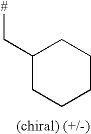 | H | H | SCH₃ | |
| 1212 | n-hexyl/H (chiral) (R) | H (chiral) (R) | 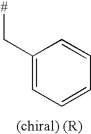 | H | H | SCH₃ | |
| 1213 | n-hexyl/H (chiral) (R) | H (chiral) (R) | 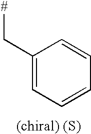 | H | H | SCH₃ | |
| 1214 | n-hexyl/H (chiral) (R) | H (chiral) (S) | 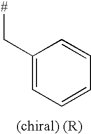 | H | H | SCH₃ | |
| 1215 | n-hexyl/H (chiral) (R) | H (chiral) (S) | 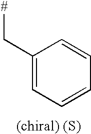 | H | H | SCH₃ | |
| 1216 | n-hexyl/H (chiral) (S) | H (chiral) (R) | 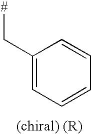 | H | H | SCH₃ | |
| 1217 | n-hexyl/H (chiral) (S) | H (chiral) (R) | 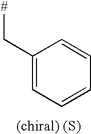 | H | H | SCH₃ | |
| 1218 | n-hexyl/H (chiral) (S) | H (chiral) (S) | 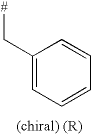 | H | H | SCH₃ | |
| 1219 | n-hexyl/H (chiral) (S) | H (chiral) (S) | 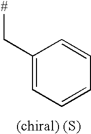 | H | H | SCH₃ | |
| 1220 | n-hexyl/H (chiral) (R) | H (chiral) (S) | 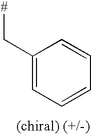 | H | H | SCH₃ | |
| 1221 | n-hexyl/H (chiral) (R) | H (chiral) (+/−) | 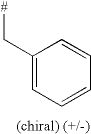 | H | H | SCH₃ | |
| 1222 | n-hexyl/H (chiral) (+/−) | H (chiral) (+/−) | 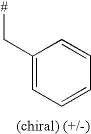 | H | H | SCH₃ | |
| 1223 | n-pentyl/H (chiral) (R) | CH₃(chiral) (R) | 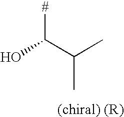 | H | H | SCH₃ | |
| 1224 | n-pentyl/H (chiral) (R) | CH₃(chiral) (R) | 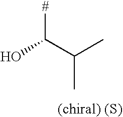 | H | H | SCH₃ | |
| 1225 | n-pentyl/H (chiral) (R) | CH₃(chiral) (S) | 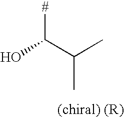 | H | H | SCH₃ | |
| 1226 | n-pentyl/H (chiral) (R) | CH₃(chiral) (S) | 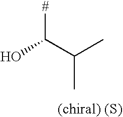 | H | H | SCH₃ | |
| 1227 | n-pentyl/H (chiral) (S) | CH₃(chiral) (R) | 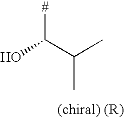 | H | H | SCH₃ | |
| 1228 | n-pentyl/H (chiral) (S) | CH₃(chiral) (R) | 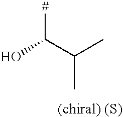 | H | H | SCH₃ | |
| 1229 | n-pentyl/H (chiral) (S) | CH₃(chiral) (S) | 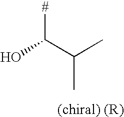 | H | H | SCH₃ | |
| 1230 | n-pentyl/H (chiral) (S) | CH₃(chiral) (S) | 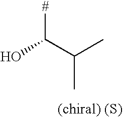 | H | H | SCH₃ | |
| 1231 | n-pentyl/H (chiral) (R) | CH₃(chiral) (S) | 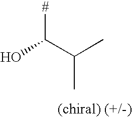 | H | H | SCH₃ | |
| 1232 | n-pentyl/H (chiral) (R) | CH₃(chiral) (+/−) | 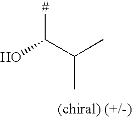 | H | H | SCH₃ | |
| 1233 | n-pentyl/H (chiral) (+/−) | CH₃(chiral) (+/−) | 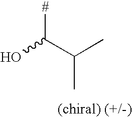 | H | H | SCH₃ | |
| 1234 | n-pentyl/H (chiral) (R) | CH₃(chiral) (R) | 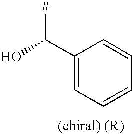 | H | H | SCH₃ | |
| 1235 | n-pentyl/H (chiral) (R) | CH₃(chiral) (R) | 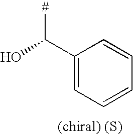 | H | H | SCH₃ | |
| 1236 | n-pentyl/H (chiral) (R) | CH₃(chiral) (S) | 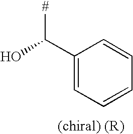 | H | H | SCH₃ | |
| 1237 | n-pentyl/H (chiral) (R) | CH₃(chiral) (S) | 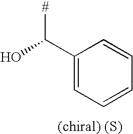 | H | H | SCH₃ | |
| 1238 | n-pentyl/H (chiral) (S) | CH₃(chiral) (R) | 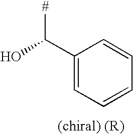 | H | H | SCH₃ | |
| 1239 | n-pentyl/H (chiral) (S) | CH₃(chiral) (R) | 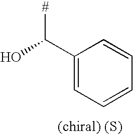 | H | H | SCH₃ | |
| 1240 | n-pentyl/H (chiral) (S) | CH₃(chiral) (S) | 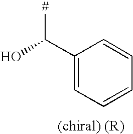 | H | H | SCH₃ | |
| 1241 | n-pentyl/H (chiral) (S) | CH₃(chiral) (S) | 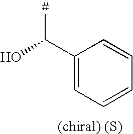 | H | H | SCH₃ | |
| 1242 | n-pentyl/H (chiral) (R) | CH₃(chiral) (S) | 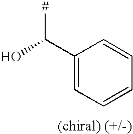 | H | H | SCH₃ | |
| 1243 | n-pentyl/H (chiral) (R) | CH₃(chiral) (+/−) | 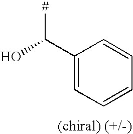 | H | H | SCH₃ | |
| 1244 | n-pentyl/H (chiral) (+/−) | CH₃(chiral) (+/−) | 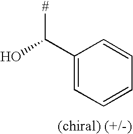 | H | H | SCH₃ | |
| 1245 | n-pentyl/H (chiral) (R) | CH₃(chiral) (R) | 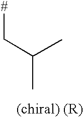 | H | H | SCH₃ | |
| 1246 | n-pentyl/H (chiral) (R) | CH₃(chiral) (R) | 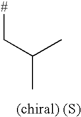 | H | H | SCH₃ | |
| 1247 | n-pentyl/H (chiral) (R) | CH₃(chiral) (S) | 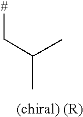 | H | H | SCH₃ | |
| 1248 | n-pentyl/H (chiral) (R) | CH₃(chiral) (S) | 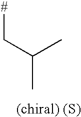 | H | H | SCH₃ | |
| 1249 | n-pentyl/H (chiral) (S) | CH₃(chiral) (R) | 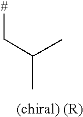 | H | H | SCH₃ | |
| 1250 | n-pentyl/H (chiral) (S) | CH₃(chiral) (R) | 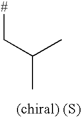 | H | H | SCH₃ | |
| 1251 | n-pentyl/H (chiral) (S) | CH₃(chiral) (S) | 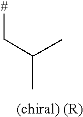 | H | H | SCH₃ | |
| 1252 | n-pentyl/H (chiral) (S) | CH₃(chiral) (S) | 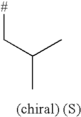 | H | H | SCH₃ | |
| 1253 | n-pentyl/H (chiral) (R) | CH₃(chiral) (S) | 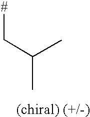 | H | H | SCH₃ | |
| 1254 | n-pentyl/H (chiral) (R) | CH₃(chiral) (+/−) | 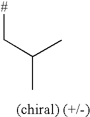 | H | H | SCH₃ | |
| 1255 | n-pentyl/H (chiral) (+/−) | CH₃(chiral) (+/−) | 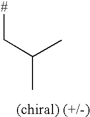 | H | H | SCH₃ | |
| 1256 | n-pentyl/H (chiral) (R) | CH₃(chiral) (R) | 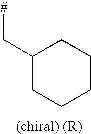 | H | H | SCH₃ | |
| 1257 | n-pentyl/H (chiral) (R) | CH₃(chiral) (R) | 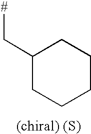 | H | H | SCH₃ | |
| 1258 | n-pentyl/H (chiral) (R) | CH₃(chiral) (S) | 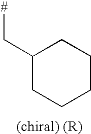 | H | H | SCH₃ | |
| 1259 | n-pentyl/H (chiral) (R) | CH₃(chiral) (S) | 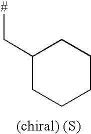 | H | H | SCH₃ | |
| 1260 | n-pentyl/H (chiral) (S) | CH₃(chiral) (R) | 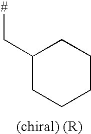 | H | H | SCH₃ | |
| 1261 | n-pentyl/H (chiral) (S) | CH₃(chiral) (R) | 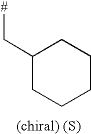 | H | H | SCH₃ | |
| 1262 | n-pentyl/H (chiral) (S) | CH₃(chiral) (S) | 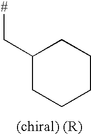 | H | H | SCH₃ | |
| 1263 | n-pentyl/H (chiral) (S) | CH₃(chiral) (S) | 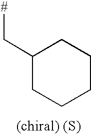 | H | H | SCH₃ | |
| 1264 | n-pentyl/H (chiral) (R) | CH₃(chiral) (S) | 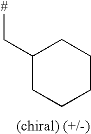 | H | H | SCH₃ | |
| 1265 | n-pentyl/H (chiral) (R) | CH₃(chiral) (+/−) | 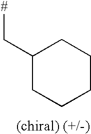 | H | H | SCH₃ | |
| 1266 | n-pentyl/H (chiral) (+/−) | CH₃(chiral) (+/−) | 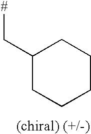 | H | H | SCH₃ | |
| 1267 | n-pentyl/H (chiral) (R) | CH₃(chiral) (R) | 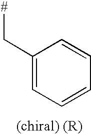 | H | H | SCH₃ | |
| 1268 | n-pentyl/H (chiral) (R) | CH₃(chiral) (R) | 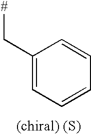 | H | H | SCH₃ | |
| 1269 | n-pentyl/H (chiral) (R) | CH₃(chiral) (S) | 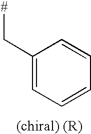 | H | H | SCH₃ | |
| 1270 | n-pentyl/H (chiral) (R) | CH₃(chiral) (S) | 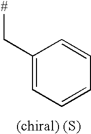 | H | H | SCH₃ | |
| 1271 | n-pentyl/H (chiral) (S) | CH₃(chiral) (R) | 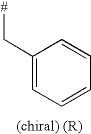 | H | H | SCH₃ | |
| 1272 | n-pentyl/H (chiral) (S) | CH₃(chiral) (R) | 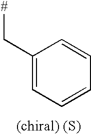 | H | H | SCH₃ | |
| 1273 | n-pentyl/H (chiral) (S) | CH₃(chiral) (S) | 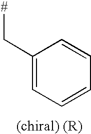 | H | H | SCH₃ | |
| 1274 | n-pentyl/H (chiral) (S) | CH₃(chiral) (S) | 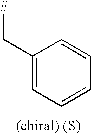 | H | H | SCH₃ | |
| 1275 | n-pentyl/H (chiral) (R) | CH₃(chiral) (S) | 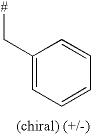 | H | H | SCH₃ | |
| 1276 | n-pentyl/H (chiral) (R) | CH₃(chiral) (+/−) | 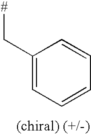 | H | H | SCH₃ | |
| 1277 | n-pentyl/H (chiral) (+/−) | CH₃(chiral) (+/−) | 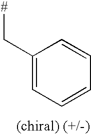 | H | H | SCH₃ | |
| 1278 | n-pentyl/H (chiral) (R) | H (chiral) (R) | 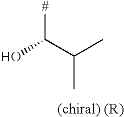 | H | H | SCH₃ | |
| 1279 | n-pentyl/H (chiral) (R) | H (chiral) (R) | 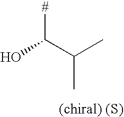 | H | H | SCH₃ | |
| 1280 | n-pentyl/H (chiral) (R) | H (chiral) (S) | 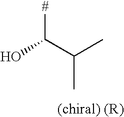 | H | H | SCH₃ | |
| 1281 | n-pentyl/H (chiral) (R) | H (chiral) (S) | 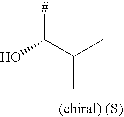 | H | H | SCH₃ | |
| 1282 | n-pentyl/H (chiral) (S) | H (chiral) (R) | 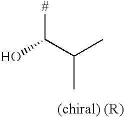 | H | H | SCH₃ | |
| 1283 | n-pentyl/H (chiral) (S) | H (chiral) (R) | 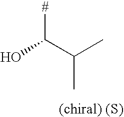 | H | H | SCH₃ | |
| 1284 | n-pentyl/H (chiral) (S) | H (chiral) (S) | 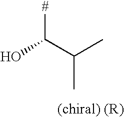 | H | H | SCH₃ | |
| 1285 | n-pentyl/H (chiral) (S) | H (chiral) (S) | 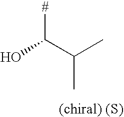 | H | H | SCH₃ | |
| 1286 | n-pentyl/H (chiral) (R) | H (chiral) (S) | 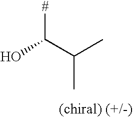 | H | H | SCH₃ | |
| 1287 | n-pentyl/H (chiral) (R) | H (chiral) (+/−) | 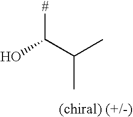 | H | H | SCH₃ | |
| 1288 | n-pentyl/H (chiral) (+/−) | H (chiral) (+/−) | 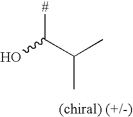 | H | H | SCH₃ | |
| 1289 | n-pentyl/H (chiral) (R) | H (chiral) (R) | 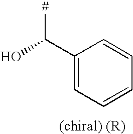 | H | H | SCH₃ | |
| 1290 | n-pentyl/H (chiral) (R) | H (chiral) (R) | 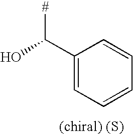 | H | H | SCH₃ | |
| 1291 | n-pentyl/H (chiral) (R) | H (chiral) (S) | 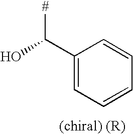 | H | H | SCH₃ | |
| 1292 | n-pentyl/H (chiral) (R) | H (chiral) (S) | 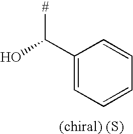 | H | H | SCH₃ | |
| 1293 | n-pentyl/H (chiral) (S) | H (chiral) (R) | 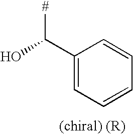 | H | H | SCH₃ | |
| 1294 | n-pentyl/H (chiral) (S) | H (chiral) (R) | 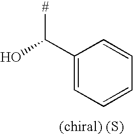 | H | H | SCH₃ | |
| 1295 | n-pentyl/H (chiral) (S) | H (chiral) (S) | 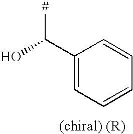 | H | H | SCH₃ | |
| 1296 | n-pentyl/H (chiral) (S) | H (chiral) (S) | 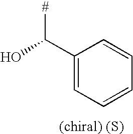 | H | H | SCH₃ | |
| 1297 | n-pentyl/H (chiral) (R) | H (chiral) (S) | 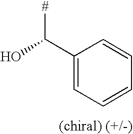 | H | H | SCH₃ | |
| 1298 | n-pentyl/H (chiral) (R) | H (chiral) (+/−) | 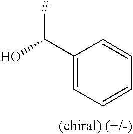 | H | H | SCH₃ | |
| 1299 | n-pentyl/H (chiral) (+/−) | H (chiral) (+/−) | 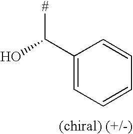 | H | H | SCH₃ | |
| 1300 | n-pentyl/H (chiral) (R) | H (chiral) (R) | 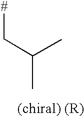 | H | H | SCH₃ | |
| 1301 | n-pentyl/H (chiral) (R) | H (chiral) (R) | 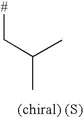 | H | H | SCH₃ | |
| 1302 | n-pentyl/H (chiral) (R) | H (chiral) (S) | 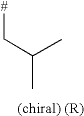 | H | H | SCH₃ | |
| 1303 | n-pentyl/H (chiral) (R) | H (chiral) (S) | 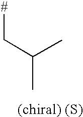 | H | H | SCH₃ | |
| 1304 | n-pentyl/H (chiral) (S) | H (chiral) (R) | 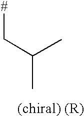 | H | H | SCH₃ | |
| 1305 | n-pentyl/H (chiral) (S) | H (chiral) (R) | 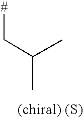 | H | H | SCH₃ | |
| 1306 | n-pentyl/H (chiral) (S) | H (chiral) (S) | 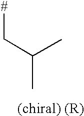 | H | H | SCH₃ | |
| 1307 | n-pentyl/H (chiral) (S) | H (chiral) (S) | 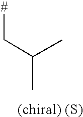 | H | H | SCH₃ | |
| 1308 | n-pentyl/H (chiral) (R) | H (chiral) (S) | 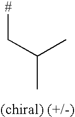 | H | H | SCH₃ | |
| 1309 | n-pentyl/H (chiral) (R) | H (chiral) (+/−) | 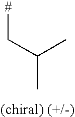 | H | H | SCH₃ | |
| 1310 | n-pentyl/H (chiral) (+/−) | H (chiral) (+/−) | 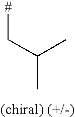 | H | H | SCH₃ | |
| 1311 | n-pentyl/H (chiral) (R) | H (chiral) (R) | 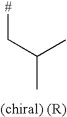 | H | H | SCH₃ | |
| 1312 | n-pentyl/H (chiral) (R) | H (chiral) (R) | 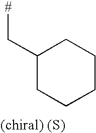 | H | H | SCH₃ | |
| 1313 | n-pentyl/H (chiral) (R) | H (chiral) (S) | 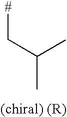 | H | H | SCH₃ | |
| 1314 | n-pentyl/H (chiral) (R) | H (chiral) (S) | 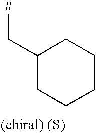 | H | H | SCH₃ | |
| 1315 | n-pentyl/H (chiral) (S) | H (chiral) (R) | 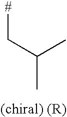 | H | H | SCH₃ | |
| 1316 | n-pentyl/H (chiral) (S) | H (chiral) (R) | 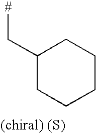 | H | H | SCH₃ | |
| 1317 | n-pentyl/H (chiral) (S) | H (chiral) (S) | 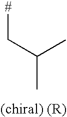 | H | H | SCH₃ | |
| 1318 | n-pentyl/H (chiral) (S) | H (chiral) (S) | 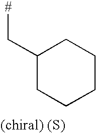 | H | H | SCH₃ | |
| 1319 | n-pentyl/H (chiral) (R) | H (chiral) (S) | 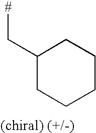 | H | H | SCH₃ | |
| 1320 | n-pentyl/H (chiral) (R) | H (chiral) (+/−) | 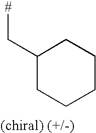 | H | H | SCH₃ | |
| 1321 | n-pentyl/H (chiral) (+/−) | H (chiral) (+/−) | 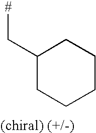 | H | H | SCH₃ | |
| 1322 | n-pentyl/H (chiral) (R) | H (chiral) (R) | 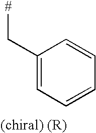 | H | H | SCH₃ | |
| 1323 | n-pentyl/H (chiral) (R) | H (chiral) (R) | 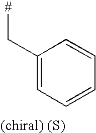 | H | H | SCH₃ | |
| 1324 | n-pentyl/H (chiral) (R) | H (chiral) (S) | 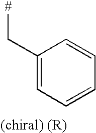 | H | H | SCH₃ | |
| 1325 | n-pentyl/H (chiral) (R) | H (chiral) (S) | 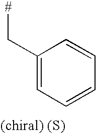 | H | H | SCH₃ | |
| 1326 | n-pentyl/H (chiral) (S) | H (chiral) (R) | 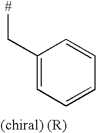 | H | H | SCH₃ | |
| 1327 | n-pentyl/H (chiral) (S) | H (chiral) (R) | 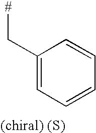 | H | H | SCH₃ | |
| 1328 | n-pentyl/H (chiral) (S) | H (chiral) (S) | 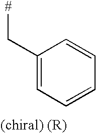 | H | H | SCH₃ | |
| 1329 | n-pentyl/H (chiral) (S) | H (chiral) (S) | 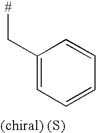 | H | H | SCH₃ | |
| 1330 | n-pentyl/H (chiral) (R) | H (chiral) (S) | 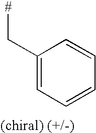 | H | H | SCH₃ | |
| 1331 | n-pentyl/H (chiral) (R) | H (chiral) (+/−) | 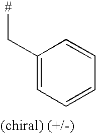 | H | H | SCH₃ | |
| 1332 | n-pentyl/H (chiral) (+/−) | H (chiral) (+/−) | 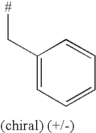 | H | H | SCH₃ | |

Determination of logP-values was made according to EEC-Directive 79/831. Annex V. A8 by HPLC (gradient method, acetonitrile/0.1% aqueous phosphoric acid).
*BnOCO = benzyloxycarbonyl
**BOC = tert-butoxycarbonyl

B. Biological Test Examples

### EXAMPLE 1

Podosphaera Test (Apple)/Protective

| | |
|---|---|
| Solvent: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated rate of application. After the spray coating has dried on, the plants are inoculated with an aqueous spore suspension of the causal agent of apple mildew (Podosphaera leucotricha). The plants are then placed in a greenhouse at approximately 23° C. and a relative atmospheric humidity of approximately 70%.

The test is evaluated 7 days after the inoculation. 0% means an efficacy which corresponds to that of the control, while an efficacy of 100% means that no disease is observed.

The compound of example 1 showed an efficacy of 100% at an application rate of 100 g/ha.

### EXAMPLE 2

Venturia Test (Apples)/Protective

| | |
|---|---|
| Solvent: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated rate of application. After the spray coating has dried on, the plants are inoculated with an aqueous conidia suspension of the causal agent of apple scab (Venturia inaequalis) and then remain for 1 day in an incubation cabinet at approximately 20° C. and a relative atmospheric humidity of 100%.

The plants are then placed in a greenhouse at approximately 21° C. and a relative atmospheric humidity of approximately 90%.

The test is evaluated 10 days after the inoculation. 0% means an efficacy which corresponds to that of the control, while an efficacy of 100% means that no disease is observed.

The compound of example 1 showed an efficacy of 100% at an application rate of 100 g/ha.

### EXAMPLE 3

Botrytis Test (Beans)/Protective

| | |
|---|---|
| Solvent: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound. After the spray coating has dried on, 2 small pieces of agar covered with growth of Botrytis cinerea are placed on each leaf. The inoculated plants are placed in a darkened chamber at 20° C. and a relative atmospheric humidity of 100%.

2 days after the inoculation, the size of the lesions on the leaves is evaluated. 0% means an efficacy which corresponds to that of the control, while an efficacy of 100% means that no disease is observed.

The compound of example 1 showed an efficacy of more than 90% at an application rate of 500 g/ha.

### EXAMPLE 4

Sphaerotheca-Test (Cucumber)

| | |
|---|---|
| Solvent: | 49 parts by weight of N,N-dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated rate of application. One day after treatment, the plants are inoculated with an aqueous spore suspension of the causal agent of sphaerotheca fuliginea. The plants are then placed in a greenhouse at approximately 23° C. and a relative atmospheric humidity of approximately 70%.

The test is evaluated 7 days after the inoculation. 0% means an efficacy which corresponds to that of the control, while an efficacy of 100% means that no disease is observed.

The compounds of examples 1, 8 and 10 showed an efficacy of 70% at an application rate of 500 ppm.

### EXAMPLE 5

In Vitro-Test for the Calculation of the ED₅₀-Value with Phytophthora infestans as Test Organism.

Wells of 96-hole microtitre plates are filled with 10[l of a solution of the test compound in methanol together with the emulsifier alkylaryl polyglycol ether. Thereafter, the solvent is evaporated in a hood. At the next step, into each well 200 μl of liquid potato dextrose medium is given that has been amended with an appropriate concentration of spores or mycelium suspension of Phytophthora infestans.

The resulting concentrations of the test compounds in the microtitre well are 50, 5, 0,5 and 0,05 ppm. The resulting concentration of the emulsifier in all wells is constantly 300 ppm.

With the aid of a photometer the extinction in all wells is measured at the wavelength of 620 nm.

The microtiter plates are then transferred for 3-5 days onto a shaker at 20° C. and 85% relative humidity.

At the end of the incubation time the growth of the test organism is measured again photometrically at the wavelength of 620 nm. The difference between the two extinction values (taken before and after incubation) is proportional to the growth of the test organism.

Based on the Δextinction data from the different test concentrations and that of the untreated test organism (control) a dose-response curve is calculated. The concentration that is necessary to give 50% growth inhibition is defined and reported as ED₅₀-value (=Effective Dose that causes 50% growth inhibition) in ppm (=mg /1).

The compounds of examples 1, 3 and 4 showed an ED₅₀ value of less than 0,05.

### EXAMPLE 6

Spodoptera frugiperda-Test (Sensible Strain)

| | |
|---|---|
| Method: | 1 mg compound disolved in 100 μl acetone; diluted with |
| | 900 μl aqueous Triton X-100 0.02% (w/v) |

Pieces of cabbage leaves (Brassica oleracea) are treated by being dipped into the preparation of the active compound of the desired concentration and are infested with larvae of the fall army worm (Spodoptera frugiperda) as long as the leaves are still moist.

After six days the mortality in % is determined. 100% means that all the caterpillars have been killed; 0% means that none of the caterpillars have been killed.

In this test e.g. the compound of example 1 showed an efficacy of 100%.

1. A method for controlling phytopathogenic microorganisms or harmful animals, comprising contacting a phytopathogenic microorganism or a harmful animal with a compound of formula (I) or a salt thereof, 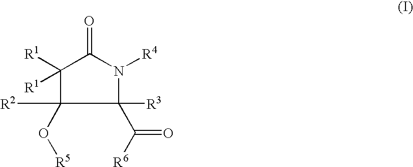
wherein:
R¹ is the same or different H, halogen, unsubstituted or substituted (C₁-C₂₀)-alkyl, unsubstituted or substituted (C₂-C₁₂)-alkenyl, unsubstituted or substituted (C₂-C₁₂)-alkynyl, unsubstituted or substituted (C₆-C₁₂)-aryl, unsubstituted or substituted heterocyclyl, unsubstituted or substituted (C₃-C₈)-cycloalkyl, unsubstituted or substituted (C₃-C₈)-cycloalkenyl, O—R″, S(O)ₙR″, SO₂NR₂″, COOR″, COSR′, CSOR′, —O—COR″, —O—CSR′, —CO—R″ or CONR₂″, or the two substituents R¹ together form a 3 to 8 membered ring which is a carbocyclic ring or contains one or two heteroatom units;
R′ is the same or different (C₁-C₁₂)-alkyl, (C₂-C₁₂)-alkenyl, (C₂-C₁₂)-alkynyl, (C₃-C₈)-cycloalkyl, (C₃-C₈)-cycloalkenyl, (C₆-C₁₂)-aryl, or heterocyclyl, all of which are unsubstituted or substituted;
R″ is the same or different H or R′;
n is 0, 1 or 2;
R² is H, unsubstituted or substituted (C₁-C₈)-alkyl, unsubstituted or substituted (C₂-C₈)-alkenyl, unsubstituted or substituted (C₂-C₈)-alkynyl, unsubstituted or substituted (C₃-C₆)-cycloalkyl, unsubstituted or substituted (C₃-C₈)-cycloalkenyl, unsubstituted or substituted (C₆-C₁₂)-aryl, or unsubstituted or substituted heterocyclyl;
R³ is H, unsubstituted or substituted (C₁-C₁₂)-alkyl, unsubstituted or substituted (C₂-C₁₂)-alkenyl, unsubstituted or substituted (C₂-C₁₂)-alkynyl, unsubstituted or substituted (C₃-C₆)-cycloalkyl, unsubstituted or substituted (C₃-C8)-cycloalkenyl, unsubstituted or substituted (C₆-C₁₂)-aryl, unsubstituted or substituted heterocyclyl, COOR″, CSOR″, COSR″, —CO—R″ or —CONR₂″;
R⁴ H, unsubstituted or substituted (C₁-C₁₂)-alkyl, unsubstituted or substituted (C₃-C₁₂)-alkenyl, unsubstituted or substituted (C₃-C₁₂)-alkynyl, unsubstituted (C₆-C₁₂)-aryl, unsubstituted or substituted heterocyclyl, unsubstituted or substituted (C₃-C8)-cycloalkyl, unsubstituted or substituted (C₃-C₈)-cycloalkenyl, S(O)ₙR′, COOR′, CSOR′, COSR′, —CO—R′, CONR₂″ or G;
R⁵ is H, unsubstituted or substituted (C₁-C₁₂)-alkyl, unsubstituted or substituted (C₂-C₁₂)-alkenyl, unsubstituted or substituted (C₂-C₁₂)-alkynyl, unsubstituted or substituted (C₃-C₈)-cycloalkyl, unsubstituted or substituted (C₃-C₈)-cycloalkenyl, unsubstituted or substituted (C₆-C₁₂)-aryl, or unsubstituted or substituted heterocyclyl, SO₂R′, COR′, COOR′, COSR′, CSOR′, CONR₂″ or G;
G is Si[(C₁-C₆)-alkyl]ₓ[(CH₂)ₜ-phenyl-(CH₃)ᵤ]_{y}[(O)ᵥ—((C₁-C₄)-alkylene)-(O—(C₁-C₄)-alkylene)_{w}-H]_{z},
where x, y, z are 0, 1, 2 or 3 and x+y+z=3, t, u are 0 or 1, v, w are 0 or 1 and v+w is 1 or 2;
R⁶ is OR″, SR″ or NR₂″
or
R⁵ and R⁶ together form a bond.

2. The method according to claim 1,
wherein;
R¹ is the same or different H, chloro, bromo, unsubstituted or substituted (C₁-C₁₂)-alkyl, unsubstituted or substituted (C₂-C₁₀)-alkenyl, unsubstituted or substituted (C₂-C₁₀)-alkynyl, unsubstituted or substituted (C₆-C₁₂)-aryl, unsubstituted or substituted heterocyclyl, unsubstituted or substituted (C₃-C₆)-cycloalkyl, OR″, COOR″ or —CO—R″ or the two substituents R¹ together form a 3 to 6 membered ring which is a carbocyclic ring or contains one or two heteroatom units;
R² is H, unsubstituted or substituted (C₁-C₆)-alkyl, unsubstituted or substituted (C₂-C₆)-alkenyl, unsubstituted or substituted (C₂-C₆ )-alkynyl, unsubstituted or substituted (C₃-C₆ )-cycloalkyl, unsubstituted or substituted phenyl, or unsubstituted or substituted heterocyclyl;
R³ is H, unsubstituted or substituted (C₁-C₁₂)-alkyl, unsubstituted or substituted (C₂-C₁₂)-alkenyl, unsubstituted or substituted (C₂-C₁₂)-alkynyl, unsubstituted or substituted (C₃-C₆)-cycloalkyl, unsubstituted or substituted (C₃-C₆)-cycloalkenyl, unsubstituted or substituted (C₆-C₁₂)-aryl, unsubstituted or substituted heterocyclyl, COOR″, —CO—R″ or CONR₂″;
R⁴ is H, unsubstituted or substituted (C₁-C₁₂)-alkyl, unsubstituted or substituted (C₃-C₁₀)-alkenyl, unsubstituted or substituted (C₃-C₁₀)-alkynyl, SO₂R′, COOR′, —COR′, CONR₂″ or G;
R⁵ is H, unsubstituted or substituted (C₁-C₁₂)-alkyl, unsubstituted or substituted (C₂-C₁₂)-alkenyl, unsubstituted or substituted (C₂-C₁₂)-alkynyl, unsubstituted or substituted (C₃-C₈)-cycloalkyl, unsubstituted or substituted phenyl, unsubstituted or substituted heterocyclyl, SO₂R′, COR′, COOR′, COSR′, CSOR′, CONR₂″ or G;
G is Si[(C₁-C₆)-alkyl]ₓ[(CH₂)ₜ-phenyl-(CH₃)ᵤ]_{y}[(O)ᵥ—((C₁-C₄)-alkylene)-(O—(C₁-C₄)-alkylene)_{w}-H]_{z},
where x, y, z are 0, 1, 2 or 3 and x+y+z=3, t, u are 0 or 1, v, w are 0 or 1 and v+w is 1 or 2;
R⁶ is OR″, SR″ or NR₂″
or
R⁵ and R⁶ together form a bond;
R′ is the same or different (C₁-C₁₀)-alkyl, (C₂-C₁₀)-alkenyl, (C₂-C₁₀)-alkynyl, (C₃-C₆)-cycloalkyl, (C₃-C₆)-cycloalkenyl, phenyl, or heterocyclyl, all of which are unsubstituted or substituted;
R″ is the same or different and is H or R′; and
n is 0, 1 or 2.

3. The method according to claim 1 where the compounds of formula (I) are compounds of formula (II), 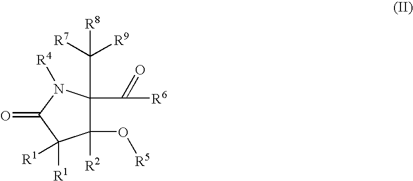
wherein:
R¹ is the same or different H, chloro, bromo, unsubstituted or substituted (C₁-C₁₀)-alkyl, unsubstituted or substituted (C₂-C₈)-alkenyl, unsubstituted or substituted (C₂-C₈)-alkynyl, unsubstituted or substituted phenyl, unsubstituted or substituted heterocyclyl, unsubstituted or substituted (C₃-C₆)-cycloalkyl, COOR″, OR″ or —CO—R″ or the two substituents R¹ together form a 3 to 6 membered carbocyclic ring;
R2 H, unsubstituted or substituted (C₁-C₆)-alkyl, unsubstituted or substituted (C₂-C₄)-alkenyl, unsubstituted or substituted (C₂-C₄)-alkynyl, unsubstituted or substituted (C₃-C₆)-cycloalkyl, unsubstituted or substituted phenyl, or unsubstituted or substituted heterocyclyl;
R⁴ is H, unsubstituted or substituted (C₁-C₁₂)-alkyl, unsubstituted or substituted (C₃-C₆)-alkenyl, unsubstituted or substituted (C₃-C₆)-alkynyl, SO₂R′, COOR′, —COR′ or G;
R⁵ is H, unsubstituted or substituted (C¹-C⁸)-alkyl, unsubstituted or substituted (C2-C₆)-alkenyl, unsubstituted or substituted (C₂-C₆)-alkynyl, unsubstituted or substituted (C₃-C₆)-cycloalkyl, SO₂R′, COOR′, —COR′, CONR₂″, unsubstituted or substituted phenyl, unsubstituted or substituted heterocyclyl, or G;
G is Si[(C₁-C₆)-alkyl]ₓ[(CH₂)ₜ-phenyl-(CH₃)ᵤ]_{y}[(O)ᵥ—((C₁-C₄)-alkylene)-(O—(C₁-C₄)-alkylene)_{w}-H]_{z},
where x, y, z are 0, 1, 2 or 3 and x+y+z=3, t, u are 0 or 1, v, w are 0 or 1 and v+w is 1 or 2;
R⁶ is OR″, SR″ or NR₂″;
or
R⁵ and R⁶ together form a bond;
R⁷ is H, fluoro, chloro, O—R″, SR″, NR″₂, —O—COR′, —S—COR′, —O—CSR′, —O—SO₂R′, —O—COOR′, —O—CSOR′, —O—CONR₂″, NO₂, or CN;
R⁸ is H, unsubstituted or substituted (C₁-C₄)-alkyl, unsubstituted or substituted (C₂-C₄)-alkenyl, unsubstituted or substituted (C₂-C₄)-alkynyl, unsubstituted or substituted (C₃-C₆)-cycloalkyl, unsubstituted or substituted phenyl, or unsubstituted or substituted heterocyclyl;
or
R⁷ and R⁸ are together ═O or ═S;
R⁹ is H, halogen, unsubstituted or substituted (C₁-C₁₂)-alkyl, unsubstituted or substituted (C₂-C₁₀)-alkenyl, unsubstituted or substituted (C₂-C₁₀)-alkynyl, unsubstituted or substituted (C₆-C₁₂)-aryl, unsubstituted or substituted heterocyclyl, unsubstituted or substituted (C₃-C₈)-cycloalkyl, unsubstituted or substituted (C₃-C₈)-cycloalkenyl, O—R′, or SR′;
or
R⁸ and R⁹ together form a 3 to 8 membered ring which is a carbocyclic ring or contains one or two heteroatom units,
R′ is the same or different (C₁-C₈)-alkyl, (C₂-C₆)-alkenyl, (C₂-C₆)-alkynyl, (C₃-C₆)-cycloalkyl, phenyl, or heterocyclyl, all of which are unsubstituted or substituted;
and
R″ is the same or different H or R″;
G SiMe₃, SiEt₃, SiMe₂t-Bu, SiMe(t-Bu)₂, Si-i-Pr₃, Si-t-BuPh₂, SiMePh₂, SiPh₃, SiMe₂(C(CH₃)₂—CH(CH₃)₂), SiEt₂i-Pr, SiMe₂i-Pr, SiMe₂i-Bu, SiBz₃, Si(CH₂—C₆H₄—CH₃)₃, SiPh₂(O-i-Pr), SiPh₂(O-t-Bu), Sit-BuPh(OMe), or SiMe₂(O—C₂H₄—OMe); and
n is 0, 1 or 2.

4. The method according to claim 3,
wherein:
R¹ is the same or different H, chloro, bromo, (C₁-C₈)-alkyl, (C₂-C₆)-alkenyl, (C₂-C₈)-alkynyl, phenyl, wherein the last four groups are unsubstituted or substituted with one to three substituents from the group fluoro, chloro, methyl, ethyl, isopropyl, tert-butyl, trifluoromethyl, trifluoromethoxy, methoxy, ethoxy, NO₂, and CN, heterocyclyl, unsubstituted or substituted with fluoro, chloro, methyl, ethyl, isopropyl, tert-butyl, trifluoromethyl, trifluoromethoxy, methoxy, ethoxy, NO₂, or CN, (C₃-C₆)-cycloalkyl, (C₃-C₈)-cycloalkenyl, both unsubstituted or substituted with one to three substituents from the group fluoro, chloro, methyl, trifluoromethyl, methoxy, NO₂, and CN, or the two substituents R¹ together form a 3 to 6 membered carbocyclic ring;
R² is H, methyl, ethyl, propyl, isopropyl, allyl, propargyl, cyclopropyl, cyclobutyl, cyclopentyl, cyclohexyl, phenyl, unsubstituted or substituted with one to three substituents from the group fluoro, chloro, methyl, ethyl, isopropyl, tert-butyl, trifluoromethyl, trifluoromethoxy, methoxy, ethoxy, NO₂, and CN, or heterocyclyl, unsubstituted or substituted with one to three substituents from the group fluoro, chloro, methyl, ethyl, trifluoromethyl, methoxy, NO₂, and CN;
R⁴ is H, unsubstituted or substituted (C₁-C₁₂)-alkyl, unsubstituted or substituted (C₃-C₆)-alkenyl, unsubstituted or substituted (C₃-C₆)-alkynyl, SO₂R′, COOR′, —COR′, CONR₂″ or G;
R⁵ is H, (C₁-C₆)-alkyl, (C₂-C₆)-alkenyl, (C₂-C₈)-alkynyl, unsubstituted or substituted (C₃-C₆)-cycloalkyl, SO₂R′, COOR′, —COR′, CONR₂″, or G;
R⁶ is OR″, SR″ or NR₂″,
or
R⁵ and R⁶ together form a bond;
R⁷ is hydroxyl, mercapto, SCH₃, fluoro, chloro, bromo, methyl, ethyl, methoxy, trifluoromethoxy, ethoxy, —O—SO₂R′, —O—COOR′, —O—CONR₂″, CN, NR″ or O-G;
R⁸ is H, unsubstituted or substituted (C₁-C₄)-alkyl, unsubstituted or substituted (C₂-C₄)-alkenyl, unsubstituted or substituted (C₂-C₄)-alkynyl, unsubstituted or substituted (C₃-C₆)cycloalkyl, phenyl, unsubstituted or substituted with one to three substituents from the group fluoro, chloro, methyl, ethyl, isopropyl, tert-butyl, trifluoromethyl, trifluoromethoxy, methoxy, ethoxy, NO₂ and CN, heterocyclyl, unsubstituted or substituted with one to three substituents from the group fluoro, chloro, methyl, ethyl, isopropyl, tert-butyl, trifluoromethyl, trifluoromethoxy, methoxy, ethoxy, NO₂ and CN;
or
R⁷ and R³ are together ═O or ═S;
R⁹ is H, halogen, unsubstituted or substituted (C₁-C₈)-alkyl, unsubstituted or substituted (C₂-C₆)-alkenyl, propargyl, unsubstituted or substituted (C₆-C₁₀)-aryl, unsubstituted or substituted heterocyclyl, unsubstituted or substituted (C₃-C₆)-cycloalkyl, unsubstituted or substituted (C₃-C₆)-cycloalkenyl;
or
R⁸ and R⁹ together form a 3 to 6 membered ring which is a carbocyclic ring or contains one or two heteroatom units;
G is SiMe₃, SiEt₃, SiMe₂t-Bu, SiMe(t-Bu)₂, Si-i-Pr₃, Si-t-BuPh₂, SiMePh₂, SiPh₃, SiMe₂(C(CH₃)₂—CH(CH₃)₂), SiEt₂i-Pr, SiMe₂i-Pr, SiMe₂i-Bu, SiBz₃, Si(CH₂—C₆H₄—CH₃)₃, SiPh₂(O-i-Pr), SiPh₂(O-t-Bu), Sit-BuPh(OMe), or SiMe₂(O—C₂H₄—OMe);
R′is the same or different (C₁-C₆)-alkyl, (C₂-C₄)-alkenyl, (C₂-C₄)-alkynyl, (C₃-C₆)-cyclo-alkyl, phenyl, or heterocyclyl, all of which are unsubstituted or substituted; and
R″ is the same or different H or R′.

5. The method according to claim 1, where the compound of formula (I) is used together with one or more agrochemically active compounds.

6. The method according to claim 3, where the compound of formula (II) is applied as a seed dressing.

7. The method according to claim 3, where the compound of formula (II) is applied to crops of transgenic plants.

8. A process for controlling phytopathogenic microorganisms and harmful animals, comprising: applying a compound of formula (I) according to claim 1 to the phytopathogenic microorganism or harmful animal or their habitat.

9. A compound of formula (Ia) or a salt thereof, 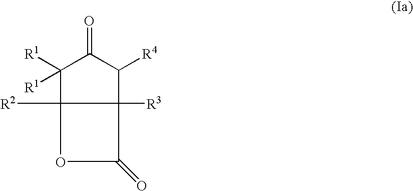
wherein:
R¹ is the same or different H, halogen, unsubstituted or substituted (C₁-C₂₀)-alkyl, unsubstituted or substituted (C₂-C₁₂)-alkenyl, unsubstituted or substituted (C₂-C₁₂)-alkynyl, unsubstituted or substituted (C₆-C₁₂)-aryl, unsubstituted or substituted heterocyclyl, substituted or unsubstituted (C₃-C₈)-cycloalkyl, unsubstituted or substituted (C₃-C₈)-cycloalkenyl, O—R″, S(O)ₙR″, COOR″, CSOR′, COSR′, —O—COR′, —O—CSR′, —CO—R″, CONR₂″, SO₂NR₂″, or the two substituents R¹ together form a 3 to 8 membered ring which is a carbocyclic ring or contains one or two heteroatom units;
n is 0, 1 or 2;
R′ is the same or different (C₁-C₁₂)-alkyl, (C₂-C₁₂)-alkenyl, (C₂-C₁₂)-alkynyl, (C₃-C₈)-cycloalkyl, (C₃-C₈)-cycloalkenyl, (C₆-C₁₂)-aryl, or heterocyclyl, all of which are unsubstituted or substituted;
R″ is the same or different H or R′;
R² is unsubstituted or substituted (C₁-C₆)-alkyl, unsubstituted or substituted (C₂-C₆)-alkenyl, unsubstituted or substituted (C₂-C₆)-alkynyl, unsubstituted or substituted (C₃-C₆)-cycloalkyl, unsubstituted or substituted (C₃-C₈)-cycloalkenyl, unsubstituted or substituted (C₆-C₁₂)-aryl, or unsubstituted or substituted heterocyclyl;
R³ is H, unsubstituted or substituted (C₁-C₆)-alkyl, unsubstituted or substituted (C₂-C₆)-alkenyl, unsubstituted or substituted (C₂-C₆)-alkynyl, unsubstituted or substituted (C₃-C₆)-cycloalkyl, unsubstituted or substituted (C₃-C₈)-cycloalkenyl, unsubstituted or substituted (C₆-C₁₂)-aryl, or unsubstituted or substituted heterocyclyl, COOR″, CSOR′, COSR′, —CO—R′ or CONR₂″;
and
R⁴ is H, unsubstituted or substituted (C₁-C₂₀)-alkyl, unsubstituted or substituted (C₂-C₁₂)-alkenyl, unsubstituted or substituted (C₂-C₁₂)-alkynyl, unsubstituted or substituted (C₆-C₁₂)-aryl, unsubstituted or substituted heterocyclyl, unsubstituted or substituted (C₃-C₈)-cycloalkyl, unsubstituted or substituted (C₃-C₆)-cycloalkenyl, S(O)ₙR′, COOR′, CSOR′, COSR′, —CO—R′, CONR₂″ or G; and
G is Si[(C₁-C₆)-alkyl]ₓ[(CH₂)ₜ-phenyl-(CH₃)ᵤ]_{y}[(O)ᵥ—((C₁-C₄)-alkylene)-(O-(C₁-C₄)-alkylene)_{w}-H]_{z},
where x, y, z are 0, 1, 2 or 3 and x+y+z=3, t, u are 0 or 1, v, w are 0 or 1 and v+w is 1 or 2;
with the proviso, that the following compounds are excluded:

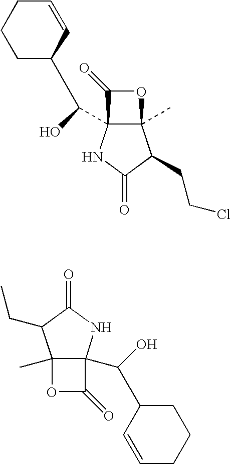

10. A compound of formula (Ib) or a salt thereof, 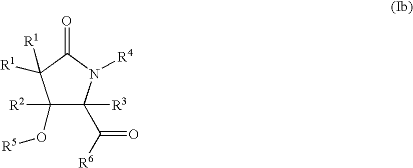
‘wherein:
R1 is the same or different H, halogen, unsubstituted or substituted (C₁-C₂₀)-alkyl, unsubstituted or substituted (C₂-C₁₂)-alkenyl, unsubstituted or substituted (C₂-C₁₂)-alkynyl, unsubstituted or substituted (C₆-C₁₂)-aryl, unsubstituted or substituted heterocyclyl, substituted or unsubstituted (C₃-C₈)-cycloalkyl, unsubstituted or substituted (C₃-C₈)-cycloalkenyl, O—R″, S(O)ₙR″, COOR″, CSOR′, COSR′, —O—COR′, —O—CSR′, —CO—R″ or CONR₂″, or the two substituents R¹ together form a 3 to 8 membered ring which is a carbocyclic ring or contains one or two heteroatom units;
n is 0, 1 or 2;
R′ is the same or different (C₁-C₁₂)-alkyl, (C₂-C₁₂)-alkenyl, (C₂-C₁₂)-alkynyl, (C₃-C₈)-cycloalkyl, (C₃-C₈)-cycloalkenyl, (C₆-C₁₂)-aryl, or heterocyclyl, all of which are unsubstituted or substituted;
R″ is the same or different H or R′;
R² is unsubstituted or substituted (C₁-C₅)-alkyl, unsubstituted or substituted (C₂-C₆)-alkenyl, unsubstituted or substituted (C₂-C₆)-alkynyl, unsubstituted or substituted (C₃-C₆)-cycloalkyl, unsubstituted or substituted (C₃-C₆)-cycloalkenyl, unsubstituted or substituted (C₆-C₁₂)-aryl, or unsubstituted or substituted heterocyclyl;
R³ is H, unsubstituted or substituted (C₁-C₆)-alkyl, unsubstituted or substituted (C₂-C₆)-alkenyl, unsubstituted or substituted (C₂-C₆)-alkynyl, unsubstituted or substituted (C₃-C₆)-cycloalkyl, unsubstituted or substituted (C₃-C₆)-cycloalkenyl, unsubstituted or substituted (C₆-C₁₂)-aryl, or unsubstituted or substituted heterocyclyl, CSOR′, COSR′, —CO—R″ or CONR₂″;
R⁴ is H, unsubstituted or substituted (C₁-C₁₂)-alkyl, unsubstituted or substituted (C₂-C₁₂)-alkenyl, unsubstituted or substituted (C₂-C₁₂)-alkynyl, unsubstituted or substituted (C₆-C₁₂)-aryl, unsubstituted or substituted heterocyclyl, unsubstituted or substituted (C₃-C₈)-cycloalkyl, unsubstituted or substituted (C₃-C₆)-cycloalkenyl, S(O)ₙR′, COOR′, CSOR′, —CO—R′, CONR₂″ or G;
R⁵ is H, unsubstituted or substituted (C₁-C₁₂)-alkyl, unsubstituted or substituted (C₂-C₁₂)-alkenyl, unsubstituted or substituted (C₂-C₁₂)-alkynyl, unsubstituted or substituted (C₃-C₈)-cycloalkyl, unsubstituted or substituted (C₃-C₈)-cycloalkenyl, unsubstituted or substituted (C₆-C₁₂)-aryl, unsubstituted or substituted heterocyclyl, SO₂R′, COR′, COOR′, COSR′, CSOR′, CONR₂″ or G.
G is Si[(C₁-C₆)-alkyl]ₓ[(CH₂)ₜ-phenyl-(CH₃)ᵤ]_{y}[(O)ᵥ—((C₁-C₄)-alkylene)-(O—(C₁-C₄)-alkylene)_{w}-H]_{z},
where x, y, z are 0, 1, 2 or 3 and x+y+z=3, t, u are 0 or 1, v, w are 0 or 1 and v+w is 1 or 2;
R⁶ is OR″, SR″ or NR₂″;
excluding compounds where
R¹═H, R²═CH₃, R³═H, R⁴═H, C₆H₅, CH₂—C₆H₅, or C₂H₅, R⁵═H and R⁶═NH₂, NHC₆H₅, or NHCH₂C₆H₅.

11. The method according to claim 3, where the compound of formula (II) is used together with one or more agrochemically active compounds.

12. The method according to claim 1, where the compound of formula (I) is applied as a seed dressing.

13. The method according to claim 1, where the compound of formula (I) is applied to crops of transgenic plants.
